(19) United States
(12) Patent Application Publication
(10) Pub. No.: US 2026/0117236 A1
(43) Pub. Date: Apr. 30, 2026

(54) METHODS AND COMPOSITIONS FOR MODULATING TRICHOME DENSITY AND FLAVOR MOLECULE SECRETION

(71) Applicant: Altria Client Services LLC, Richmond, VA (US)

(72) Inventors: Sreepriya Pramod, Fredericksburg, VA (US); Jesse Frederick, Richmond, VA (US); Andrew C. Adams, Midlothian, VA (US); Marcos F. G. Lusso, Chesterfield, VA (US)

(73) Assignee: Altria Client Services LLC, Richmond, VA (US)

(21) Appl. No.: 19/009,709

(22) Filed: Jan. 3, 2025

Related U.S. Application Data
(60) Provisional application No. 63/617,925, filed on Jan. 5, 2024.

(51) Int. Cl. C12N 15/82; A24B 3/12; A24B 13/00

(52) U.S. Cl. CPC C12N 15/82; A24B 3/12; A24B 13/00

(57) ABSTRACT

Glandular trichomes are epidermal outgrowths in plants that are the site of metabolic compound synthesis and storage. Modulating the secretion of metabolite compounds in tobacco and tea leaves can alter the taste profile in products made from these plants. The present disclosure relates to methods for modulating secretion from glandular trichomes and compositions comprising altered trichome secretion of metabolites.

### CROSS-REFERENCE TO RELATED APPLICATION AND INCORPORATION OF SEQUENCE LISTING

This application claims the benefit of U.S. Provisional Application No. 63/617,925, filed Jan. 5, 2024, which is incorporated by reference herein in its entirety. A sequence listing contained in the file named “P35070WO00_SL.xml” which is 263,609 bytes (measured in operating system MS-Windows®), created on Jan. 3, 2025, containing a total number of 90 SEQ ID NOs, starting from SEQ ID NO:1 to SEQ ID NO:90, is filed electronically herewith and incorporated by reference in its entirety.

### FIELD

The present disclosure relates to methods for modulating secretion from glandular trichomes and compositions comprising altered trichome secretion of metabolites and their uses in plants including tobacco and tea. Also provided are methods for modulating the profile of metabolites secreted from glandular trichomes and compositions comprising altered trichome secretion of metabolites active as flavor molecules and their uses in plants including tobacco and tea.

### BACKGROUND

Glandular trichomes are epidermal outgrowths in plants that are the site of metabolic compound synthesis and storage. Their presence on stem, leaf, and floral tissues provides protection for plants against various biotic and abiotic stresses. Glandular trichomes also play a role in the biosynthesis, storage, and secretion of specialized or secondary metabolites. Other trichomes, such as non-glandular trichomes, can protect plants from predators (e.g., by providing a physical barrier) or ultraviolet light. Trichomes can be found on adaxial (upper) and abaxial (lower) leaf surfaces, albeit at different densities in some plant species.

Metabolites produced and secreted by glandular trichomes are often hydrophobic (e.g., fatty acid derivatives, flavonoids, terpenoids). Terpenoids constitute the largest and most diverse class of plant metabolites. The olefinic backbone of terpenoids is made of multiples of the five-carbon (C) isoprene unit, with the major groups being monoterpenes (10C), sesquiterpenes (15C), and diterpenes (20C). These terpenoids are produced through the condensation of five-carbon isoprene units (dimethylallyl diphosphate [DMAPP] and isopentenyl diphosphate [IPP]) most often by the sequential head-to-tail addition of DMAPP to IPP.

The amount of secondary metabolites produced is often tightly correlated to the glandular trichome density present on the plant epidermis (Chalvin et al., Cell, 25:477-487 (2020)). One way to increase the amount of secondary metabolite production in plants is to increase the density of trichomes present on the plant epidermis. Transcriptional regulation of trichome initiation has been shown to involve members of MYB and C2H2 zinc-finger family of transcription factors. Transgenic overexpression of Artemisia annua MYB1 (AaMYB1) was shown to increase trichome density and subsequently the production of artemisin (Matias-Hernandez et al., Plant Journal, 90:520-534 (2017)).

Lipid transfer proteins (LTPs) are important in the transport of specialized metabolites in glandular trichomes. Studies have shown that overexpression of LTPs leads to an increase of exudates in plants glandular trichomes (Choi et al., Plant Journal, 70:480-491 (2012)).

Due to the important role of glandular trichomes in the biosynthesis and secretion of terpenoids, there is a need for a greater understanding of the genes, regulatory factors, and signaling mechanisms involved in the control of trichome initiation and development in plants. It is also important to understand the mode of secretion of these specialized metabolites into the cuticle of trichomes. In this disclosure, candidate genes are provided that can be used to modify trichome density as well as metabolite secretion profiles in plants. Modification of trichome density will also improve transport of specialized metabolites in glandular trichomes. Altering metabolite secretion profiles will provide the ability to modulate the flavor profile of cured tobacco and cured tobacco products with the goal of increasing consumer satisfaction.

### SUMMARY

In one aspect, this disclosure provides a modified tobacco plant, or part thereof, comprising at least one non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 21 to 40.

In one aspect, this disclosure provides a modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40.

In one aspect, this disclosure provides a modified tobacco plant, or part thereof, comprising a recombinant DNA construct comprising a heterologous promoter operably linked to a nucleic acid sequence encoding a polypeptide comprising an amino acid sequence at least 80% identical or similar to an amino acid sequence selected from the group consisting of SEQ ID NOs: 21 to 40.

In one aspect, this disclosure provides a method of producing a modified tobacco plant comprising inducing a non-natural mutation in at least one tobacco cell in an endogenous nucleic acid sequence encoding a polypeptide comprising an amino acid sequence at least 80% identical or similar to an amino acid sequence selected from the group consisting of SEQ ID NOs: 21 to 40; selecting at least one tobacco cell comprising said non-natural mutation; and regenerating at least one modified tobacco plant from said at least one tobacco cell that was selected.

In one aspect, this disclosure provides a method of producing a modified tobacco plant comprising: introducing a recombinant DNA construct to at least one tobacco cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40; selecting at least one tobacco cell comprising said recombinant DNA construct; and regenerating at least one modified tobacco plant from said at least one selected tobacco cell.

In one aspect, this disclosure provides a method of producing a modified tobacco plant comprising: introducing a recombinant DNA construct to at least one tobacco cell, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40; selecting at least one tobacco cell comprising said recombinant DNA construct; and regenerating at least one modified tobacco plant from said at least selected one tobacco cell.

In one aspect, this disclosure provides a method comprising preparing a tobacco product using cured tobacco material from a modified tobacco plant, wherein said modified tobacco plant comprises a non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 21 to 40.

In one aspect, this disclosure provides a method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40.

In one aspect, this disclosure provides a method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40.

In one aspect, this disclosure provides a method comprising transforming a tobacco cell with a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40.

In one aspect, this disclosure provides a method comprising transforming a tobacco cell with a recombinant DNA construct, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40.

In one aspect, this disclosure provides a method for producing a modified tobacco plant comprising: crossing at least one tobacco plant of a first tobacco variety with at least one tobacco plant of a second tobacco variety to produce at least one progeny tobacco seed, wherein said at least one tobacco plant of said first tobacco variety comprises a non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 21 to 40, wherein said non-natural mutation is not present in said endogenous nucleic acid sequence in a control tobacco plant of said first tobacco variety; and selecting for at least one progeny tobacco seed, or a plant germinated therefrom, wherein said at least one tobacco seed or plant germinated therefrom comprises said non-natural mutation.

In one aspect, this disclosure provides a method for producing a modified tobacco plant comprising: crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40, wherein said recombinant DNA construct is not present in said endogenous nucleic acid sequence in a control tobacco plant of the same variety; and selecting for at least one progeny tobacco seed, or a plant germinated therefrom, wherein said at least one tobacco seed or plant germinated therefrom comprises said recombinant DNA construct.

In one aspect, this disclosure provides a method for producing a modified tobacco plant comprising: crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40, wherein said recombinant DNA construct is not present in said nucleic acid sequence in a control tobacco plant of the first tobacco variety; and selecting for at least one progeny tobacco seed, or a plant germinated therefrom, wherein said at least one tobacco seed or plant germinated therefrom comprises said recombinant DNA construct.

In one aspect, this disclosure provides a modified tobacco plant, or part thereof, comprising at least one non-natural mutation in an endogenous nucleic acid sequence that modulates the expression or functional activity of a gene, wherein said gene encodes a polypeptide comprising an amino acid sequence at least 80% identical or similar to an amino acid sequence selected from the group consisting of SEQ ID NOs: 21 to 40.

In one aspect, this disclosure provides a modified tobacco plant, or part thereof, comprising: (a) a genetic modification in a gene; or (b) a genetic modification targeting said gene; wherein said genetic modification downregulates the expression or activity of said gene, wherein said gene encodes a nucleic acid sequence having at least 80% identity to a polynucleotide sequence selected from the group consisting of SEQ ID NOs: 1 to 20.

In one aspect, this disclosure provides a modified Camellia plant, or part thereof, comprising at least one non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 21 to 40.

In one aspect, this disclosure provides a modified Camellia plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40.

In one aspect, this disclosure provides a modified Camellia plant, or part thereof, comprising a recombinant DNA construct comprising a heterologous promoter operably linked to a nucleic acid sequence encoding a polypeptide comprising an amino acid sequence at least 80% identical or similar to an amino acid sequence selected from the group consisting of SEQ ID NOs: 21 to 40.

In one aspect, this disclosure provides a method of producing a modified Camellia plant comprising: inducing a non-natural mutation in at least one Camellia cell in an endogenous nucleic acid sequence encoding a polypeptide comprising an amino acid sequence at least 80% identical or similar to an amino acid sequence selected from the group consisting of SEQ ID NOs: 21 to 40; selecting at least one Camellia cell comprising said non-natural mutation; and regenerating at least one modified tobacco plant from said at least one selected Camellia cell.

In one aspect, this disclosure provides a method of producing a modified Camellia plant comprising: introducing a recombinant DNA construct to at least one Camellia cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40; selecting at least one Camellia cell comprising said recombinant DNA construct; and regenerating at least one modified Camellia plant from said at least one selected tobacco cell.

In one aspect, this disclosure provides a method of producing a modified Camellia plant comprising: introducing a recombinant DNA construct to at least one Camellia cell, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40; selecting at least one Camellia cell comprising said recombinant DNA construct; and regenerating at least one modified Camellia plant from said at least one selected tobacco cell.

In one aspect, this disclosure provides a method for producing a modified Camellia plant comprising: crossing at least one Camellia plant of a first Camellia variety with at least one Camellia plant of a second Camellia variety to produce at least one progeny Camellia seed, wherein said at least one Camellia plant of said first Camellia variety comprises a non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 21 to 40, wherein said non-natural mutation is not present in said endogenous nucleic acid sequence in a control Camellia plant of said first Camellia variety; and selecting for at least one progeny Camellia seed, or a plant germinated therefrom, wherein said at least one Camellia seed or plant germinated therefrom comprises said non-natural mutation.

In one aspect, this disclosure provides a method for producing a modified Camellia plant comprising: crossing at least one Camellia plant of a first Camellia variety with at least one tobacco plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40, wherein said recombinant DNA construct is not present in said endogenous nucleic acid sequence in a control Camellia plant of the same variety; and selecting for at least one progeny Camellia seed, or a plant germinated therefrom, wherein said at least one Camellia seed or plant germinated therefrom comprises said recombinant DNA construct.

In one aspect, this disclosure provides a method for producing a modified Camellia plant comprising: crossing at least one Camellia plant of a first Camellia variety with at least one Camellia plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40, wherein said recombinant DNA construct is not present in said nucleic acid sequence in a control tobacco plant of the first Camellia variety; and selecting for at least one progeny tobacco seed, or a plant germinated therefrom, wherein said at least one tobacco seed or plant germinated therefrom comprises said recombinant DNA construct.

### BRIEF DESCRIPTION OF THE SEQUENCES

SEQ ID NOS: 1 to 20 set forth sequences of examples of nucleotide coding sequences for trichome density and flavor associated genes.

SEQ ID NOs: 21 to 40 set forth sequences of examples of amino acid sequences for trichome density and flavor associated genes.

SEQ ID NOs: 41 to 60 set forth sequences of examples of genomic DNA sequences for trichome density and flavor associated genes.

SEQ ID NO: 61 sets forth the sequence of the ALCS1 binary genetic construct used plant transformation. The construct comprises a pCSVMV promoter, multiple cloning site, NOS terminator, ACTII promoter, NPTII resistance gene, and a NOS terminator.

SEQ ID NOs: 62 to 69 set forth nucleotide sequences of examples of promoters for trichome specific or preferred expression.

SEQ ID NOs: 70 to 80 set forth sequences of examples of promoters for topping responsive root specific or preferred expression.

SEQ ID NOs: 81 to 90 set forth sequences of examples of promoters for topping responsive leaf specific or preferred expression.

### BRIEF DESCRIPTION OF THE DRAWINGS

FIG. 1 depicts a light micrograph at 100× magnification of a Nicotiana tabacum leaf highlighting the structures of a glandular trichome. Glandular trichome measurements are taken by measuring the trichome gland width and the length of the trichome stalk.

FIG. 2 depicts selected trichome measurements from GR139 Secretor(S) Nicotiana tabacum leaves compared to GR139-Non-Secretor (NS) Nicotiana tabacum leaves. FIG. 2A provides the total number of trichomes for both abaxial (lower) and adaxial (upper) sides of the 5th leaf. FIG. 2B provides the average trichome length in microns for both abaxial and adaxial sides of the 5th leaf. FIG. 2C provides the average trichome width in microns for both abaxial and adaxial sides of the 5th leaf. Standard deviations are shown as error bars.

FIG. 3 depicts a light micrograph at 100× magnification of a Nicotiana tabacum leaf. Both the adaxial and abaxial surfaces can be seen with the adaxial surface on the top. FIG. 3A depicts a GR139 Secretor(S) Nicotiana tabacum 5th leaf compared to FIG. 3B which depicts GR139-Non-Secretor (NS) Nicotiana tabacum 5th leaf.

FIG. 4 depicts a quantification of the number of trichomes in GR139 S, KDH-960, Hoja Parado (Galpão), KDH-959, KDH-926, and AA-37-1 tobacco varieties. FIG. 4A shows the average number of adaxial trichomes. FIG. 4B shows the average number of abaxial trichomes. Standard deviations are shown as error bars.

FIG. 5 depicts a volcano plot of the results of targeted RT-qPCR of the top 10 Candidate genes in a GR139 S Nicotiana tabacum background compared to a KDH959 Nicotiana tabacum background. G82265, g58563, g58333, g58504, g58508, g82264, and g58509 are significantly upregulated. G82257 has a flat expression pattern and g82268 shows an insignificant change.

FIG. 6 depicts a volcano plot of the results of targeted RT-qPCR of the top 10 Candidate genes in a KDH926 Nicotiana tabacum background compared to a KDH959 Nicotiana tabacum background. G82265, g58563, g58333, g58504, g58508, g82264, and g58509 are significantly upregulated. G82257 is significantly downregulated and g82268 shows an insignificant change.

FIG. 7 depicts a volcano plot of the results of targeted RT-qPCR of the top 10 Candidate genes in a KDH960 Nicotiana tabacum background compared to a KDH959 Nicotiana tabacum background. G82265, g58563, g58333, g58504, g58508, g82264, and g58509 are significantly upregulated. G82257 is significantly downregulated and g82268 shows an insignificant change.

FIG. 8 depicts the results of targeted RT-qPCR of 16 Candidate genes in a GR139NS GIS overexpressing tobacco compared to the GR139NS Control. Actin is the control and is set as zero. Fold change compared to actin is shown according to the Y axis. FIG. 8A and FIG. 8B represent independent experiments.

FIG. 9 depicts the results of targeted RT-qPCR of 16 Candidate genes in a GR139NS MYB86 overexpressing tobacco compared to the GR139NS Control. Actin is the control and is set as zero. Fold change compared to actin is shown according to the Y axis. FIG. 9A and FIG. 9B represent independent experiments.

FIG. 10 depicts the results of targeted RT-qPCR of 16 Candidate genes in a Galpão MYB86 overexpressing tobacco compared to the Galpão Control. Actin is the control and is set as zero. Fold change compared to actin is shown according to the Y axis. FIG. 10A, FIG. 10B, and FIG. 10C represent independent experiments.

FIG. 11 depicts the results of targeted RT-qPCR of 16 Candidate genes in a KDH960 MYB86 overexpressing tobacco compared to the KDH960 Control. Actin is the control and is set as zero. Fold change compared to actin is shown according to the Y axis. FIG. 11A, FIG. 11B, and FIG. 11C represent independent experiments.

FIG. 12 depicts the results of targeted RT-qPCR of 16 Candidate genes in a TN90 GIS overexpressing tobacco compared to the TN90 Control. Actin is the control and is set as zero. Fold change compared to actin is shown according to the Y axis. FIG. 12A, FIG. 12B, and FIG. 12C represent independent experiments.

FIG. 13 depicts the results of targeted RT-qPCR of 16 Candidate genes in a TN90 MYB86 overexpressing tobacco compared to the TN90 Control. Actin is the control and is set as zero. Fold change compared to actin is shown according to the Y axis. FIG. 13A, FIG. 13B, and FIG. 13C represent independent experiments.

FIG. 14 depicts a principal component analysis (PCA) plot of 11 secretor and non-secretor tobacco varieties calculated using only compounds associated with the following secondary metabolites: Alkaloids and alkaloid derivatives, Benzenoids, Flavonoids, Phenylpropanoids, and Terpenoids.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to inclusive of the edges of the range as well as any number between the defined edges of the range. For example, “between 1 and 10” includes any number between 1 and 10, as well as the number 1 and the number 10.

When the term “about” is used in reference to a number, it is understood to mean plus or minus 10%. For example, “about 100” would include from 90 to 110.

As used herein, the singular form “a,” “an,” and “the” include plural references unless the context clearly dictates otherwise. For example, the term “a compound” or “at least one compound” may include a plurality of compounds, including mixtures thereof.

Any tobacco plant, or part thereof, provided herein is specifically envisioned for use with any method provided herein. Similarly, any modified tobacco plant, or part thereof, is specifically envisioned for use with any method provided herein. Any nucleic acid sequence, amino acid sequence, or other composition provided herein is specifically envisioned for use with any method or tobacco plant, modified or not modified, provided herein.

This disclosure provides a seed obtained from any modified tobacco plant provided herein. This disclosure provides a cell of any modified tobacco plant provided herein.

Trichomes, in general, are hair-like epidermal outgrowths covering most aerial plant tissues. Trichomes tend to be multicellular, but unicellular trichomes are known as well. Multiple types of trichomes can be found on an individual plant, and trichomes vary in shape, size, and cellular organization. An individual trichome can be classified as a glandular trichome or a non-glandular trichome.

Glandular trichomes are characterized by the presence of a head made of cells that can secrete or store large quantities of specialized metabolites (e.g., without being limiting, terpenoids, phenylpropanoids, flavonoids, methyl ketones, acylsugars). Within the group of glandular trichomes, a trichome can be further characterized as being peltate or capitate. A capitate glandular trichome typically possesses a stalk with a length that is more than twice the height of the head, and the number of cells in the trichome is highly variable. A peltate trichome is a short-stalked trichome with a large head made of between four and eighteen cells arranged in one or two concentric circles.

Non-glandular trichomes typically provide physical protection for plants against biotic and abiotic stresses. For example, they can form a physical barrier against low humidity, high light intensity (e.g., ultraviolet light), high or low temperatures, and feeding and/or egg-laying by insects or arachnids (e.g., mites).

In an aspect, a trichome is a glandular trichome. In an aspect, a glandular trichome is a capitate glandular trichome. In an aspect, a glandular trichome is a peltate glandular trichome. In an aspect, a glandular trichome is selected from the group consisting of a capitate glandular trichome and a peltate glandular trichome. In an aspect, a trichome is a non-glandular trichome.

In some instances, it may be desirable to increase the total number of trichomes on a plant or plant part. It may also be desirable to increase the density of trichomes on a plant or plant part. Without being limited by any scientific theory, a plant comprising an increased number of trichomes can produce an increased amount of one or more desired compounds as compared to a control. See, for example, U.S. Patent Application Publication Nos. 2022/0243214 A1; 2022/0243215 A1; and 2022/0243216 A1.

### Introgression

As used herein, “introgression” refers to the transmission of a desired allele of a genetic locus from one genetic background (e.g., genotype) to another.

As used herein, a “trichome-associated single nucleotide polymorphism” refers to a polymorphism that segregates with a trichome-associated QTL.

As used herein, a “genotype” refers to the genetic constitution of a plant or cell. Two tobacco plants from different tobacco lines or varieties would be understood to have different genotypes. Alternatively, plants of an inbred line typically comprise identical genotypes. However, without being limiting, if the trichome-associated QTL has been introgressed into a single TN 90 plant, the single TN 90 plant comprising the trichome-associated QTL would have a different genotype than all TN 90 plants lacking the trichome-associated QTL.

As used herein, a “genetic modification” refers to a change in the genetic makeup of a plant or plant genome. A genetic modification can be introduced by methods including, but not limited to, mutagenesis, genome editing, genetic transformation, or a combination thereof. A genetic modification includes, for example, a mutation (e.g., a non-natural mutation) in a gene or a transgene targeting a gene (e.g., an arginine decarboxylase (ADC) transgene targets an ADC gene). As used here, “targeting” refers to either directly upregulating or directly downregulating the expression or activity of a gene. As used here, “directly”, in the context of a transgene impacting the expression or activity of a gene, refers to the impact being exerted over the gene via a physical contact or chemical interaction between the gene (e.g., a promoter region or a UTR region) or a product encoded therein (e.g., a mRNA molecule or a polypeptide) and a product encoded by the transgene (e.g., a small RNA molecule or a protein such as a transcription factor or a dominant negative polypeptide variant). In an aspect, a transgene impacts the expression or activity of a target gene without involving a transcription factor (e.g., the transgene does not encode a transcription factor and/or does not suppress the expression or activity of a transcription factor that in turn regulates the target gene).

### Mutations

As used herein, “modified,” in the context of a plant, refers to a plant comprising a genetic alteration introduced for certain purposes and beyond natural polymorphisms. Without being limiting, a modified plant can comprise a non-natural mutation or a recombinant DNA construct. In an aspect, a modified tobacco plant comprises a non-natural mutation. In another aspect, a modified tobacco plant comprises a recombinant DNA construct. In another aspect, a modified tobacco plant comprises a genetic modification. Without being limiting, a modified plant, seed, or plant part comprises a recombinant nucleic acid molecule. As used herein, a “recombinant nucleic acid construct” refers to a nucleic acid molecule formed by laboratory methods of genetic recombination, such as, without being limiting, molecular cloning. In another aspect, a modified plant, seed, or plant part comprises a genetic modification. In an aspect, a modified plant, seed, or plant part is a transgenic plant, seed, or plant part.

As used herein, a “mutation” refers to an inheritable genetic modification introduced into a gene to alter the expression or activity of a product encoded by a reference sequence of the gene. A mutation in a certain gene, such as, for example, an arginine decarboxylase (ADC) is referred to as an ADC mutant. Such a modification can be in any sequence region of a gene, for example, in a promoter, 5′-untranslated region (UTR), exon, intron, 3′-UTR, or terminator region. In an aspect, a mutation reduces, inhibits, or eliminates the expression or activity of a gene product. In another aspect, a mutation increases, elevates, strengthens, or augments the expression or activity of a gene product.

In an aspect, mutations are not natural polymorphisms that exist in a particular tobacco variety or cultivar. In an aspect, a mutation is a “non-natural” or “non-naturally occurring” mutation. As used herein, a “non-natural” or “non-naturally occurring” mutation refers to a non-spontaneous mutation generated via human intervention, and does not correspond to a spontaneous mutation generated without human intervention. Non-limiting examples of human intervention include mutagenesis (e.g., chemical mutagenesis, ionizing radiation mutagenesis) and targeted genetic modifications (e.g., CRISPR-based methods, TALEN-based methods, zinc finger-based methods). Non-natural mutations and non-naturally occurring mutations do not include spontaneous mutations that arise naturally (e.g., via aberrant DNA replication in a germ line of a plant).

It will be appreciated that, when identifying a mutation, the reference DNA sequence should be from the same variety of tobacco. For example, if a modified tobacco plant comprising a mutation is from the variety TN90, then the endogenous reference sequence must be the endogenous TN90 sequence, not a homologous sequence from a different tobacco variety (e.g., K326). Similarly, if a modified tobacco cell comprising a mutation is a TN90 cell, then the endogenous reference sequence must be the endogenous TN90 sequence, not a homologous sequence from a tobacco cell from a different tobacco variety (e.g., K326).

In an aspect, a tobacco plant, or part thereof, is homozygous for at least one non-natural mutation. In another aspect, a tobacco plant, or part thereof, is heterozygous for at least one non-natural mutation. In another aspect, a tobacco plant, or part thereof, is homozygous for an introduced recombinant DNA construct. In another aspect, a tobacco plant, or part thereof, is hemizygous for an introduced recombinant DNA construct. In a further aspect, a tobacco plant, or part thereof, is heterozygous for an introduced recombinant DNA construct.

In an aspect, a mutation provided herein creates a dominant allele of the mutated locus. Dominant alleles are alleles that mask the contribution of a second allele at the same locus. A dominant allele can be a “dominant negative allele” or a “dominant positive allele.” Dominant negative alleles, or antimorphs, are alleles that act in opposition to normal allelic function. A dominant negative allele typically does not function normally and either directly inhibits the activity of a wild-type protein (e.g., through dimerization) or inhibits the activity of a second protein that is required for the normal function of the wild-type protein (e.g., an activator or a downstream component of a pathway). For example, a dominant negative allele abrogates or reduces the normal function of an allele in a heterozygous or homozygous state. Dominant positive alleles can increase normal gene function (e.g., a hypermorph) or provide new functions for a gene (e.g., a neomorph). A semi-dominant allele occurs when penetrance of a linked phenotype in individuals heterozygous for the allele is less than that which is observed in individuals homozygous for the allele. In another aspect, a mutation provided herein creates a dominant positive allele of a mutated locus.

As used herein, “inducing” a mutation refers to generating a mutation in a polynucleotide sequence via human intervention. Many suitable methods for inducing mutations in tobacco are known in the art. Non-limiting examples of such methods include use of chemical mutagens, use of irradiation, use of nucleases, use of transposons, and use of Agrobacterium. In an aspect, inducing a mutation comprises the use of an agent selected from the group consisting of a chemical mutagen, irradiation, a transposon, Agrobacterium, and a nuclease.

In an aspect, inducing a mutation comprises the use of a chemical mutagen. In an aspect, a chemical mutagen comprises ethyl methanesulfonate (EMS).

In another aspect, inducing a mutation comprises the use of irradiation. In an aspect, irradiation comprises gamma rays, X-rays, ionizing radiation, or fast neutrons.

In an aspect, inducing a mutation comprises the use of a transposon. In another aspect, inducing a mutation comprises the use of Agrobacterium.

In a further aspect, inducing a mutation comprises the use of a nuclease. In an aspect, a nuclease is selected from the group consisting of a meganuclease, a zinc-finger nuclease, a transcription activator-like effector nuclease, a CRISPR/Cas9 nuclease, a CRISPR/Cpf1 nuclease, a CRISPR/CasX nuclease, a CRISPR/CasY nuclease, and a Csm1 nuclease. In an aspect, inducing a mutation comprises the use of a CRISPR/Cas9 nuclease. In an aspect, inducing a mutation comprises the use of a CRISPR/Cpf1 nuclease. In an aspect, inducing a mutation comprises the use of a CRISPR/CasX nuclease. In an aspect, inducing a mutation comprises the use of a CRISPR/CasY nuclease. In an aspect, inducing a mutation comprises the use of a Csm1 nuclease.

In an aspect, inducing a mutation comprises the use of a base editor. As used herein, a “base editor” refers to a catalytically impaired Cas nuclease fused to a nucleotide deaminase. In some aspects, base editors further comprise DNA repair proteins. In an aspect, a base editor is a cytosine base editor. A cytosine base editor enables C-G to T-A transitions. In an aspect, a base editor is an adenine base editor. An adenine base editor enables A-T to G-C conversion. In an aspect, a base editor is a C-to-G base editor.

In an aspect, inducing a mutation comprises the use of a prime editor. As used herein, a “prime editor” refers to a Cas nickase fused to an engineered reverse transcriptase. Prime editors can introduce all 12 transition and transversion mutations and small insertions or deletions, as well as combinations thereof. Prime editors use a prime editing guide RNA (pegRNA) that specifics the target site for editing and encodes the desired edit. In an aspect, a pegRNA is provided to a tobacco cell. In an aspect, a pegRNA comprises a nucleic acid sequence that binds to a nucleic acid sequence selected from the group consisting of SEQ ID NOs: 4 to 9.

Additional information about base editors and prime editors, and their use in plants, can be found in Molla et al., Nature Plants, 7:1166-1187 (2021). See also Anzalone et al., Nature, 576:149-157 (2019); Komor et al., Nature, 533:420-424 (2016); and Gaudelli et al., Nature, 551:464-471 (2017).

Several types of mutations are known in the art. In an aspect, a mutation comprises an insertion. An “insertion” refers to the addition of one or more nucleotides or amino acids to a given polynucleotide or amino acid sequence, respectively, as compared to an endogenous reference polynucleotide or amino acid sequence. In another aspect, a mutation comprises a deletion. A “deletion” refers to the removal of one or more nucleotides or amino acids to a given polynucleotide or amino acid sequence, respectively, as compared to an endogenous reference polynucleotide or amino acid sequence. In another aspect, a mutation comprises a substitution. A “substitution” refers to the replacement of one or more nucleotides or amino acids to a given polynucleotide or amino acid sequence, respectively, as compared to an endogenous reference polynucleotide or amino acid sequence. In another aspect, a mutation comprises an inversion. An “inversion” refers to when a segment of a polynucleotide or amino acid sequence is reversed end-to-end. A “duplication” refers to when a segment of a polynucleotide or amino acid sequence is repeated. The repeated segment can immediately follow the original segment, or it can be separated from the original segment by one or more nucleotides or amino acids. In an aspect, a mutation provided herein comprises a mutation selected from the group consisting of an insertion, a deletion, a substitution, a duplication, and an inversion.

In an aspect, a non-natural mutation comprises a mutation selected from the group consisting of a substitution, a deletion, an insertion, a duplication, and an inversion of one or more nucleotides relative to an endogenous nucleic acid sequence selected from the group consisting of SEQ ID NOs: 1 to 20 and 41 to 60.

In an aspect, a non-natural mutation comprises a mutation selected from the group consisting of a substitution, a deletion, an insertion, a duplication, and an inversion of one or more nucleotides relative to an endogenous nucleic acid sequence encoding an amino acid sequence selected from the group consisting of SEQ ID NOs: 21 to 40.

In an aspect, a non-natural mutation comprises one or more mutation types selected from the group consisting of a nonsense mutation, a missense mutation, a frameshift mutation, a splice-site mutation, and any combinations thereof. As used herein, a “nonsense mutation” refers to a mutation to a nucleic acid sequence that introduces a premature stop codon to an amino acid sequence by the nucleic acid sequence. As used herein, a “missense mutation” refers to a mutation to a nucleic acid sequence that causes a substitution within the amino acid sequence encoded by the nucleic acid sequence. As used herein, a “frameshift mutation” refers to an insertion or deletion to a nucleic acid sequence that shifts the frame for translating the nucleic acid sequence to an amino acid sequence. A “splice-site mutation” refers to a mutation in a nucleic acid sequence that causes an intron to be retained for protein translation, or, alternatively, for an exon to be excluded from protein translation. Splice-site mutations can cause nonsense, missense, or frameshift mutations.

Mutations in coding regions of genes (e.g., exonic mutations) can result in a truncated protein or polypeptide when a mutated messenger RNA (mRNA) is translated into a protein or polypeptide. In an aspect, this disclosure provides a mutation that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids. In an aspect, a non-natural mutation results in a truncation of a polypeptide.

Without being limited by any scientific theory, one way to cause a protein or polypeptide truncation is by the introduction of a premature stop codon in an mRNA transcript of an endogenous gene. In an aspect, this disclosure provides a mutation that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.”

In an aspect, a mutation provided herein comprises a null mutation. As used herein, a “null mutation” refers to a mutation that confers a complete loss-of-function for a protein encoded by a gene comprising the mutation, or, alternatively, a mutation that confers a complete loss-of-function for a small RNA encoded by a genomic locus. A null mutation can cause lack of mRNA transcript production, a lack of small RNA transcript production, a lack of protein function, or a combination thereof.

A mutation provided herein can be positioned in any part of an endogenous gene. In an aspect, a mutation provided herein is positioned within an exon of an endogenous gene. In another aspect, a mutation provided herein is positioned within an intron of an endogenous gene. In a further aspect, a mutation provided herein is positioned within a 5′-UTR of an endogenous gene. In still another aspect, a mutation provided herein is positioned within a 3′-UTR of an endogenous gene. In yet another aspect, a mutation provided herein is positioned within a promoter of an endogenous gene. In yet another aspect, a mutation provided herein is positioned within a terminator of an endogenous gene. In an aspect, a non-natural mutation provided herein comprises a mutation in a sequence region selected from the group consisting of a promoter, a 5′-UTR, a 3′-UTR, an exon, an intron, and a terminator.

The screening and selection of mutagenized tobacco plants can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

In an aspect, a non-natural mutation comprises an insertion or deletion of at least 1 nucleotide in a nucleic acid molecule as compared to a wildtype nucleic acid molecule. In an aspect, a non-natural mutation comprises an insertion or deletion of at least 2 nucleotides in a nucleic acid molecule as compared to a wildtype nucleic acid molecule. In an aspect, a non-natural mutation comprises an insertion or deletion of at least 3 nucleotides in a nucleic acid molecule as compared to a wildtype nucleic acid molecule. In an aspect, a non-natural mutation comprises an insertion or deletion of at least 4 nucleotides in a nucleic acid molecule as compared to a wildtype nucleic acid molecule. In an aspect, a non-natural mutation comprises an insertion or deletion of at least 5 nucleotides in a nucleic acid molecule as compared to a wildtype nucleic acid molecule. In an aspect, a non-natural mutation comprises an insertion or deletion of at least 6 nucleotides in a nucleic acid molecule as compared to a wildtype nucleic acid molecule. In an aspect, a non-natural mutation comprises an insertion or deletion of at least 7 nucleotides in a nucleic acid molecule as compared to a wildtype nucleic acid molecule. In an aspect, a non-natural mutation comprises an insertion or deletion of at least 8 nucleotides in a nucleic acid molecule as compared to a wildtype nucleic acid molecule. In an aspect, a non-natural mutation comprises an insertion or deletion of at least 9 nucleotides in a nucleic acid molecule as compared to a wildtype nucleic acid molecule. In an aspect, a non-natural mutation comprises an insertion or deletion of at least 10 nucleotides in a nucleic acid molecule as compared to a wildtype nucleic acid molecule. In an aspect, a non-natural mutation comprises an insertion or deletion of at least 15 nucleotides in a nucleic acid molecule as compared to a wildtype nucleic acid molecule. In an aspect, a non-natural mutation comprises an insertion or deletion of at least 20 nucleotides in a nucleic acid molecule as compared to a wildtype nucleic acid molecule. In an aspect, a non-natural mutation comprises an insertion or deletion of at least 30 nucleotides in a nucleic acid molecule as compared to a wildtype nucleic acid molecule. In an aspect, a non-natural mutation comprises an insertion or deletion of at least 40 nucleotides in a nucleic acid molecule as compared to a wildtype nucleic acid molecule. In an aspect, a non-natural mutation comprises an insertion or deletion of at least 50 nucleotides in a nucleic acid molecule as compared to a wildtype nucleic acid molecule. In an aspect, a non-natural mutation comprises an insertion or deletion of at least 75 nucleotides in a nucleic acid molecule as compared to a wildtype nucleic acid molecule. In an aspect, a non-natural mutation comprises an insertion or deletion of at least 100 nucleotides in a nucleic acid molecule as compared to a wildtype nucleic acid molecule. In an aspect, a non-natural mutation comprises an insertion or deletion of at least 250 nucleotides in a nucleic acid molecule as compared to a wildtype nucleic acid molecule. In an aspect, a non-natural mutation comprises an insertion or deletion of at least 500 nucleotides in a nucleic acid molecule as compared to a wildtype nucleic acid molecule. In an aspect, a non-natural mutation comprises an insertion or deletion of at least 1000 nucleotides in a nucleic acid molecule as compared to a wildtype nucleic acid molecule.

In an aspect, a non-natural mutation comprises an insertion or deletion of between 1 nucleotide and 1000 nucleotides in a nucleic acid molecule as compared to a wildtype nucleic acid molecule. In an aspect, a non-natural mutation comprises an insertion or deletion of between 1 nucleotide and 750 nucleotides in a nucleic acid molecule as compared to a wildtype nucleic acid molecule. In an aspect, a non-natural mutation comprises an insertion or deletion of between 1 nucleotide and 500 nucleotides in a nucleic acid molecule as compared to a wildtype nucleic acid molecule. In an aspect, a non-natural mutation comprises an insertion or deletion of between 1 nucleotide and 250 nucleotides in a nucleic acid molecule as compared to a wildtype nucleic acid molecule. In an aspect, a non-natural mutation comprises an insertion or deletion of between 1 nucleotide and 100 nucleotides in a nucleic acid molecule as compared to a wildtype nucleic acid molecule. In an aspect, a non-natural mutation comprises an insertion or deletion of between 1 nucleotide and 75 nucleotides in a nucleic acid molecule as compared to a wildtype nucleic acid molecule. In an aspect, a non-natural mutation comprises an insertion or deletion of between 1 nucleotide and 50 nucleotides in a nucleic acid molecule as compared to a wildtype nucleic acid molecule. In an aspect, a non-natural mutation comprises an insertion or deletion of between 1 nucleotide and 25 nucleotides in a nucleic acid molecule as compared to a wildtype nucleic acid molecule. In an aspect, a non-natural mutation comprises an insertion or deletion of between 1 nucleotide and 10 nucleotides in a nucleic acid molecule as compared to a wildtype nucleic acid molecule. In an aspect, a non-natural mutation comprises an insertion or deletion of between 1 nucleotide and 5 nucleotides in a nucleic acid molecule as compared to a wildtype nucleic acid molecule.

In an aspect, a non-natural mutation comprises an insertion or deletion of at least 1 amino acid residue in an amino acid sequence as compared to a wildtype amino acid sequence. In an aspect, a non-natural mutation comprises an insertion or deletion of at least 2 amino acid residues in an amino acid sequence as compared to a wildtype amino acid sequence. In an aspect, a non-natural mutation comprises an insertion or deletion of at least 3 amino acid residues in an amino acid sequence as compared to a wildtype amino acid sequence. In an aspect, a non-natural mutation comprises an insertion or deletion of at least 4 amino acid residues in an amino acid sequence as compared to a wildtype amino acid sequence. In an aspect, a non-natural mutation comprises an insertion or deletion of at least 5 amino acid residues in an amino acid sequence as compared to a wildtype amino acid sequence. In an aspect, a non-natural mutation comprises an insertion or deletion of at least 6 amino acid residues in an amino acid sequence as compared to a wildtype amino acid sequence. In an aspect, a non-natural mutation comprises an insertion or deletion of at least 7 amino acid residues in an amino acid sequence as compared to a wildtype amino acid sequence. In an aspect, a non-natural mutation comprises an insertion or deletion of at least 8 amino acid residues in an amino acid sequence as compared to a wildtype amino acid sequence. In an aspect, a non-natural mutation comprises an insertion or deletion of at least 9 amino acid residues in an amino acid sequence as compared to a wildtype amino acid sequence. In an aspect, a non-natural mutation comprises an insertion or deletion of at least 10 amino acid residues in an amino acid sequence as compared to a wildtype amino acid sequence. In an aspect, a non-natural mutation comprises an insertion or deletion of at least 15 amino acid residues in an amino acid sequence as compared to a wildtype amino acid sequence. In an aspect, a non-natural mutation comprises an insertion or deletion of at least 20 amino acid residues in an amino acid sequence as compared to a wildtype amino acid sequence. In an aspect, a non-natural mutation comprises an insertion or deletion of at least 30 amino acid residues in an amino acid sequence as compared to a wildtype amino acid sequence. In an aspect, a non-natural mutation comprises an insertion or deletion of at least 40 amino acid residues in an amino acid sequence as compared to a wildtype amino acid sequence. In an aspect, a non-natural mutation comprises an insertion or deletion of at least 50 amino acid residues in an amino acid sequence as compared to a wildtype amino acid sequence. In an aspect, a non-natural mutation comprises an insertion or deletion of at least 75 amino acid residues in an amino acid sequence as compared to a wildtype amino acid sequence. In an aspect, a non-natural mutation comprises an insertion or deletion of at least 100 amino acid residues in an amino acid sequence as compared to a wildtype amino acid sequence. In an aspect, a non-natural mutation comprises an insertion or deletion of at least 250 amino acid residues in an amino acid sequence as compared to a wildtype amino acid sequence. In an aspect, a non-natural mutation comprises an insertion or deletion of at least 500 amino acid residues in an amino acid sequence as compared to a wildtype amino acid sequence. In an aspect, a non-natural mutation comprises an insertion or deletion of at least 1000 amino acid residues in an amino acid sequence as compared to a wildtype amino acid sequence.

In an aspect, a non-natural mutation comprises an insertion or deletion of between 1 amino acid residue and 1000 amino acid residues in an amino acid sequence as compared to a wildtype amino acid sequence. In an aspect, a non-natural mutation comprises an insertion or deletion of between 1 amino acid residue and 750 amino acid residues in an amino acid sequence as compared to a wildtype amino acid sequence. In an aspect, a non-natural mutation comprises an insertion or deletion of between 1 amino acid residue and 500 amino acid residues in an amino acid sequence as compared to a wildtype amino acid sequence. In an aspect, a non-natural mutation comprises an insertion or deletion of between 1 amino acid residue and 250 amino acid residues in an amino acid sequence as compared to a wildtype amino acid sequence. In an aspect, a non-natural mutation comprises an insertion or deletion of between 1 amino acid residue and 100 amino acid residues in an amino acid sequence as compared to a wildtype amino acid sequence. In an aspect, a non-natural mutation comprises an insertion or deletion of between 1 amino acid residue and 75 amino acid residues in an amino acid sequence as compared to a wildtype amino acid sequence. In an aspect, a non-natural mutation comprises an insertion or deletion of between 1 amino acid residue and 50 amino acid residues in an amino acid sequence as compared to a wildtype amino acid sequence. In an aspect, a non-natural mutation comprises an insertion or deletion of between 1 amino acid residue and 25 amino acid residues in an amino acid sequence as compared to a wildtype amino acid sequence. In an aspect, a non-natural mutation comprises an insertion or deletion of between 1 amino acid residue and 10 amino acid residues in an amino acid sequence as compared to a wildtype amino acid sequence. In an aspect, a non-natural mutation comprises an insertion or deletion of between 1 amino acid residue and 5 amino acid residues in an amino acid sequence as compared to a wildtype amino acid sequence.

### Nucleic Acids and Amino Acids

The terms “coding nucleic acid sequence” and “coding sequence” are used interchangeably herein. The terms “genomic nucleic acid sequence” and “genomic sequence” are used interchangeably herein. Coding nucleic acid sequences provided herein include SEQ ID NOs: 1 to 20. Endogenous nucleic acid sequences provided herein include SEQ ID NOs: 41 to 60.

As used herein, an “endogenous” nucleic acid sequence refers to a nucleic acid sequence that occurs naturally in the genome of an organism. Endogenous nucleic acid sequences do not include heterologous sequences inserted into a genome via deliberate human intervention. Similarly, endogenous amino acid sequences are sequences that exist naturally via translation of an endogenous nucleic acid molecule. In an aspect, a nucleic acid sequence provided herein is an endogenous nucleic acid sequence.

As used herein, “heterologous” refers to a sequence (nucleic acid or amino acid) that originates from a foreign species, or, if from the same species, is substantially modified from its native form in composition and/or genomic locus by deliberate human intervention. The term also is applicable to nucleic acid constructs, also referred to herein as “polynucleotide constructs” or “nucleotide constructs.” In this manner, a “heterologous” nucleic acid construct is intended to mean a construct that originates from a foreign species, or, if from the same species, is substantially modified from its native form in composition and/or genomic locus by deliberate human intervention. Heterologous nucleic acid constructs include, but are not limited to, recombinant nucleotide constructs that have been introduced into a plant or plant part thereof, for example, via transformation methods or subsequent breeding of a transgenic plant with another plant of interest.

As used herein, a “gene” refers to a polynucleotide that can produce a functional unit (e.g., without being limiting, for example, a protein, or a small RNA molecule). A gene can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a small RNA molecule or a precursor thereof. In another aspect, a gene encodes a protein.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

Without being limiting, two aliphatic (e.g., glycine, alanine, valine, leucine, isoleucine) amino acid residues can be substituted for each other in a conservative substitution; two hydroxyl (e.g., serine, cysteine, threonine, methionine) amino acid residues can be substituted for each other in a conservative substitution; two aromatic (e.g., phenylalanine, tyrosine, tryptophan) amino acid residues can be substituted for each other in a conservative substitution; two basic (e.g., histidine, lysine, arginine) amino acid residues can be substituted for each other in a conservative substitution; and two acid (e.g., aspartate, glutamate, asparagine, glutamine) amino acid residues can be substituted for each other in a conservative substitution.

For optimal alignment of sequences to calculate their percent identity, various pair-wise or multiple sequence alignment algorithms and programs are known in the art, such as ClustalW or Basic Local Alignment Search Tool® (BLAST™), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31:3497-3500 (2003); Thompson et al., “Clustal W: Improving the sensitivity of progressive multiple sequence alignment through sequence weighting, position-specific gap penalties and weight matrix choice,” Nucleic Acids Research 22:4673-4680 (1994); Larkin M A et al., “Clustal W and Clustal X version 2.0,” Bioinformatics 23:2947-48 (2007); and Altschul et al. “Basic local alignment search tool.” J. Mol. Biol. 215:403-410 (1990), the entire contents and disclosures of which are incorporated herein by reference.

The terms “percent complementarity” or “percent complementary” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded. A nucleic acid molecule can encode a polypeptide or a small RNA.

As used herein, a “recombinant nucleic acid” refers to a nucleic acid molecule formed by laboratory methods of genetic recombination, such as, without being limiting, molecular cloning. Similarly, a “recombinant DNA construct” refers to a DNA molecule formed by laboratory methods of genetic recombination.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

In one aspect, this disclosure provides methods of detecting recombinant nucleic acids and polypeptides in plant cells. Without being limiting, nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

In an aspect, an oligonucleotide probe is a TaqMan™ probe. TaqMan™ probes are often used to increase the specificity of quantitative PCR. TaqMan™ probes rely on the 5′ to 3′ exonuclease activity of Taq polymerase to cleave a dual-labeled probe during hybridization to the complementary target sequence and fluorophore-based detection. As in other quantitative PCR methods, the resulting fluorescence signal permits quantitative measurements of the accumulation of the product during the exponential stages of the PCR; however, the TaqMan™ probe significantly increases the specificity of the detection.

In an aspect, an oligonucleotide probe comprises at nucleic acid sequence at least 80% identical to a nucleic acid sequence selected from the group consisting of SEQ ID NOs: 1 to 20 and 41 to 60. In an aspect, an oligonucleotide probe comprises at nucleic acid sequence at least 85% identical to a nucleic acid sequence selected from the group consisting of SEQ ID NOs: 1 to 20 and 41 to 60. In an aspect, an oligonucleotide probe comprises at nucleic acid sequence at least 90% identical to a nucleic acid sequence selected from the group consisting of SEQ ID NOs: 1 to 20 and 41 to 60. In an aspect, an oligonucleotide probe comprises at nucleic acid sequence at least 91% identical to a nucleic acid sequence selected from the group consisting of SEQ ID NOs: 1 to 20 and 41 to 60. In an aspect, an oligonucleotide probe comprises at nucleic acid sequence at least 92% identical to a nucleic acid sequence selected from the group consisting of SEQ ID NOs: 1 to 20 and 41 to 60. In an aspect, an oligonucleotide probe comprises at nucleic acid sequence at least 93% identical to a nucleic acid sequence selected from the group consisting of SEQ ID NOs: 1 to 20 and 41 to 60. In an aspect, an oligonucleotide probe comprises at nucleic acid sequence at least 94% identical to a nucleic acid sequence selected from the group consisting of SEQ ID NOs: 1 to 20 and 41 to 60. In an aspect, an oligonucleotide probe comprises at nucleic acid sequence at least 95% identical to a nucleic acid sequence selected from the group consisting of SEQ ID NOs: 1 to 20 and 41 to 60. In an aspect, an oligonucleotide probe comprises at nucleic acid sequence at least 96% identical to a nucleic acid sequence selected from the group consisting of SEQ ID NOs: 1 to 20 and 41 to 60. In an aspect, an oligonucleotide probe comprises at nucleic acid sequence at least 97% identical to a nucleic acid sequence selected from the group consisting of SEQ ID NOs: 1 to 20 and 41 to 60. In an aspect, an oligonucleotide probe comprises at nucleic acid sequence at least 98% identical to a nucleic acid sequence selected from the group consisting of SEQ ID NOs: 1 to 20 and 41 to 60. In an aspect, an oligonucleotide probe comprises at nucleic acid sequence at least 99% identical to a nucleic acid sequence selected from the group consisting of SEQ ID NOs: 1 to 20 and 41 to 60. In another aspect, an oligonucleotide probe comprises at nucleic acid sequence at 100% identical to a nucleic acid sequence selected from the group consisting of SEQ ID NOs: 1 to 20 and 41 to 60.

In an aspect, an oligonucleotide probe is adjacent to a polymorphic nucleotide position of one or more marker loci. As used herein, “adjacent” refers to a distance of between 0 nucleotides to 50 nucleotides from the closest end (3′ or 5′) of the oligonucleotide probe and the polymorphic nucleotide position. As used herein, a “polymorphic nucleotide position” refers to a difference (e.g., insertion, deletion, substitution) between two or more alleles of a given marker locus. A polymorphic nucleotide position can be found by generating a pairwise comparison between allele sequences. For example, if a first allele comprises the nucleotide sequence ATTTG and a second allele comprises the nucleotide sequence TTTTG, the first nucleotide would be the “polymorphic nucleotide position.”

In an aspect, a nucleic acid sequence provided herein is at least 70% identical or complementary to a sequence selected from the group consisting of SEQ ID NOs: 1 to 20 and 41 to 60. In another aspect, a nucleic acid sequence provided herein is at least 75% identical or complementary to a sequence selected from the group consisting of SEQ ID NOs: 1 to 20 and 41 to 60. In another aspect, a nucleic acid sequence provided herein is at least 80% identical or complementary to a sequence selected from the group consisting of SEQ ID NOs: 1 to 20 and 41 to 60. In another aspect, a nucleic acid sequence provided herein is at least 85% identical or complementary to a sequence selected from the group consisting of SEQ ID NOs: 1 to 20 and 41 to 60. In another aspect, a nucleic acid sequence provided herein is at least 88% identical or complementary to a sequence selected from the group consisting of SEQ ID NOs: 1 to 20 and 41 to 60. In another aspect, a nucleic acid sequence provided herein is at least 90% identical or complementary to a sequence selected from the group consisting of SEQ ID NOs: 1 to 20 and 41 to 60. In another aspect, a nucleic acid sequence provided herein is at least 91% identical or complementary to a sequence selected from the group consisting of SEQ ID NOs: 1 to 20 and 41 to 60. In another aspect, a nucleic acid sequence provided herein is at least 92% identical or complementary to a sequence selected from the group consisting of SEQ ID NOs: 1 to 20 and 41 to 60. In another aspect, a nucleic acid sequence provided herein is at least 93% identical or complementary to a sequence selected from the group consisting of SEQ ID NOs: 1 to 20 and 41 to 60. In another aspect, a nucleic acid sequence provided herein is at least 94% identical or complementary to a sequence selected from the group consisting of SEQ ID NOs: 1 to 20 and 41 to 60. In another aspect, a nucleic acid sequence provided herein is at least 95% identical or complementary to a sequence selected from the group consisting of SEQ ID NOs: 1 to 20 and 41 to 60. In another aspect, a nucleic acid sequence provided herein is at least 96% identical or complementary to a sequence selected from the group consisting of SEQ ID NOs: 1 to 20 and 41 to 60. In another aspect, a nucleic acid sequence provided herein is at least 97% identical or complementary to a sequence selected from the group consisting of SEQ ID NOs: 1 to 20 and 41 to 60. In another aspect, a nucleic acid sequence provided herein is at least 98% identical or complementary to a sequence selected from the group consisting of SEQ ID NOs: 1 to 20 and 41 to 60. In another aspect, a nucleic acid sequence provided herein is at least 99% identical or complementary to a sequence selected from the group consisting of SEQ ID NOs: 1 to 20 and 41 to 60. In another aspect, a nucleic acid sequence provided herein is 100% identical or complementary to a sequence selected from the group consisting of SEQ ID NOs: 1 to 20 and 41 to 60.

In an aspect, an endogenous nucleic acid sequence provided herein is at least 70% identical or complementary to a sequence selected from the group consisting of SEQ ID NOs: 1 to 20 and 41 to 60. In another aspect, an endogenous nucleic acid sequence provided herein is at least 75% identical or complementary to a sequence selected from the group consisting of SEQ ID NOs: 1 to 20 and 41 to 60. In another aspect, an endogenous nucleic acid sequence provided herein is at least 80% identical or complementary to a sequence selected from the group consisting of SEQ ID NOs: 1 to 20 and 41 to 60. In another aspect, an endogenous nucleic acid sequence provided herein is at least 85% identical or complementary to a sequence selected from the group consisting of SEQ ID NOs: 1 to 20 and 41 to 60. In another aspect, an endogenous nucleic acid sequence provided herein is at least 88% identical or complementary to a sequence selected from the group consisting of SEQ ID NOs: 1 to 20 and 41 to 60. In another aspect, an endogenous nucleic acid sequence provided herein is at least 90% identical or complementary to a sequence selected from the group consisting of SEQ ID NOs: 1 to 20 and 41 to 60. In another aspect, an endogenous nucleic acid sequence provided herein is at least 91% identical or complementary to a sequence selected from the group consisting of SEQ ID NOs: 1 to 20 and 41 to 60. In another aspect, an endogenous nucleic acid sequence provided herein is at least 92% identical or complementary to a sequence selected from the group consisting of SEQ ID NOs: 1 to 20 and 41 to 60. In another aspect, an endogenous nucleic acid sequence provided herein is at least 93% identical or complementary to a sequence selected from the group consisting of SEQ ID NOs: 1 to 20 and 41 to 60. In another aspect, an endogenous nucleic acid sequence provided herein is at least 94% identical or complementary to a sequence selected from the group consisting of SEQ ID NOs: 1 to 20 and 41 to 60. In another aspect, an endogenous nucleic acid sequence provided herein is at least 95% identical or complementary to a sequence selected from the group consisting of SEQ ID NOs: 1 to 20 and 41 to 60. In another aspect, an endogenous nucleic acid sequence provided herein is at least 96% identical or complementary to a sequence selected from the group consisting of SEQ ID NOs: 1 to 20 and 41 to 60. In another aspect, an endogenous nucleic acid sequence provided herein is at least 97% identical or complementary to a sequence selected from the group consisting of SEQ ID NOs: 1 to 20 and 41 to 60. In another aspect, an endogenous nucleic acid sequence provided herein is at least 98% identical or complementary to a sequence selected from the group consisting of SEQ ID NOs: 1 to 20 and 41 to 60. In another aspect, an endogenous nucleic acid sequence provided herein is at least 99% identical or complementary to a sequence selected from the group consisting of SEQ ID NOs: 1 to 20 and 41 to 60. In another aspect, an endogenous nucleic acid sequence provided herein is 100% identical or complementary to a sequence selected from the group consisting of SEQ ID NOs: 1 to 20 and 41 to 60.

As used herein, the term “polypeptide” refers to a chain of at least two covalently linked amino acids. Polypeptides can be encoded by polynucleotides provided herein. Proteins provided herein can be encoded by nucleic acid molecules provided herein. Proteins can comprise polypeptides provided herein. As used herein, a “protein” refers to a chain of amino acid residues that is capable of providing structure or enzymatic activity to a cell.

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

Detection (e.g., of an amplification product, of a hybridization complex, of a polypeptide) can be accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

In an aspect, an amino acid sequence provided herein is at least 70% identical or similar to an amino acid sequence selected from the group consisting of SEQ ID NOs: 21 to 40. In another aspect, an amino acid sequence provided herein is at least 75% identical or similar to an amino acid sequence selected from the group consisting of SEQ ID NOs: 21 to 40. In another aspect, an amino acid sequence provided herein is at least 80% identical or similar to an amino acid sequence selected from the group consisting of SEQ ID NOs: 21 to 40. In another aspect, an amino acid sequence provided herein is at least 85% identical or similar to an amino acid sequence selected from the group consisting of SEQ ID NOs: 21 to 40. In another aspect, an amino acid sequence provided herein is at least 88% identical or similar to an amino acid sequence selected from the group consisting of SEQ ID NOs: 21 to 40. In another aspect, an amino acid sequence provided herein is at least 90% identical or similar to an amino acid sequence selected from the group consisting of SEQ ID NOs: 21 to 40. In another aspect, an amino acid sequence provided herein is at least 91% identical or similar to an amino acid sequence selected from the group consisting of SEQ ID NOs: 21 to 40. In another aspect, an amino acid sequence provided herein is at least 92% identical or similar to an amino acid sequence selected from the group consisting of SEQ ID NOs: 21 to 40. In another aspect, an amino acid sequence provided herein is at least 93% identical or similar to an amino acid sequence selected from the group consisting of SEQ ID NOs: 21 to 40. In another aspect, an amino acid sequence provided herein is at least 94% identical or similar to an amino acid sequence selected from the group consisting of SEQ ID NOs: 21 to 40. In another aspect, an amino acid sequence provided herein is at least 95% identical or similar to an amino acid sequence selected from the group consisting of SEQ ID NOs: 21 to 40. In another aspect, an amino acid sequence provided herein is at least 96% identical or similar to an amino acid sequence selected from the group consisting of SEQ ID NOs: 21 to 40. In another aspect, an amino acid sequence provided herein is at least 97% identical or similar to an amino acid sequence selected from the group consisting of SEQ ID NOs: 21 to 40. In another aspect, an amino acid sequence provided herein is at least 98% identical or similar to an amino acid sequence selected from the group consisting of SEQ ID NOs: 21 to 40. In another aspect, an amino acid sequence provided herein is at least 99% identical or similar to an amino acid sequence selected from the group consisting of SEQ ID NOs: 21 to 40. In another aspect, an amino acid sequence provided herein is 100% identical or similar to an amino acid sequence selected from the group consisting of SEQ ID NOs: 21 to 40.

In an aspect, a nucleic acid sequence provided herein encodes a polypeptide comprising an amino acid sequence at least 70% identical or similar to an amino acid sequence selected from the group consisting of SEQ ID NOs: 21 to 40 In an aspect, a nucleic acid sequence provided herein encodes a polypeptide comprising an amino acid sequence at least 75% identical or similar to an amino acid sequence selected from the group consisting of SEQ ID NOs: 21 to 40. In an aspect, a nucleic acid sequence provided herein encodes a polypeptide comprising an amino acid sequence at least 80% identical or similar to an amino acid sequence selected from the group consisting of SEQ ID NOs: 21 to 40. In an aspect, a nucleic acid sequence provided herein encodes a polypeptide comprising an amino acid sequence at least 85% identical or similar to an amino acid sequence selected from the group consisting of SEQ ID NOs: 21 to 40. In an aspect, a nucleic acid sequence provided herein encodes a polypeptide comprising an amino acid sequence at least 88% identical or similar to an amino acid sequence selected from the group consisting of SEQ ID NOs: 21 to 40. In an aspect, a nucleic acid sequence provided herein encodes a polypeptide comprising an amino acid sequence at least 90% identical or similar to an amino acid sequence selected from the group consisting of SEQ ID NOs: 21 to 40. In an aspect, a nucleic acid sequence provided herein encodes a polypeptide comprising an amino acid sequence at least 91% identical or similar to an amino acid sequence selected from the group consisting of SEQ ID NOs: 21 to 40. In an aspect, a nucleic acid sequence provided herein encodes a polypeptide comprising an amino acid sequence at least 92% identical or similar to an amino acid sequence selected from the group consisting of SEQ ID NOs: 21 to 40. In an aspect, a nucleic acid sequence provided herein encodes a polypeptide comprising an amino acid sequence at least 93% identical or similar to an amino acid sequence selected from the group consisting of SEQ ID NOs: 21 to 40. In an aspect, a nucleic acid sequence provided herein encodes a polypeptide comprising an amino acid sequence at least 94% identical or similar to an amino acid sequence selected from the group consisting of SEQ ID NOs: 21 to 40. In an aspect, a nucleic acid sequence provided herein encodes a polypeptide comprising an amino acid sequence at least 95% identical or similar to an amino acid sequence selected from the group consisting of SEQ ID NOs: 21 to 40. In an aspect, a nucleic acid sequence provided herein encodes a polypeptide comprising an amino acid sequence at least 96% identical or similar to an amino acid sequence selected from the group consisting of SEQ ID NOs: 21 to 40. In an aspect, a nucleic acid sequence provided herein encodes a polypeptide comprising an amino acid sequence at least 97% identical or similar to an amino acid sequence selected from the group consisting of SEQ ID NOs: 21 to 40. In an aspect, a nucleic acid sequence provided herein encodes a polypeptide comprising an amino acid sequence at least 98% identical or similar to an amino acid sequence selected from the group consisting of SEQ ID NOs: 21 to 40. In an aspect, a nucleic acid sequence provided herein encodes a polypeptide comprising an amino acid sequence at least 99% identical or similar to an amino acid sequence selected from the group consisting of SEQ ID NOs: 21 to 40. In an aspect, a nucleic acid sequence provided herein encodes a polypeptide comprising an amino acid sequence 100% identical or similar to an amino acid sequence selected from the group consisting of SEQ ID NOs: 21 to 40.

In an aspect, an endogenous nucleic acid sequence provided herein encodes a polypeptide comprising an amino acid sequence at least 70% identical or similar to an amino acid sequence selected from the group consisting of SEQ ID NOs: 21 to 40. In an aspect, an endogenous nucleic acid sequence provided herein encodes a polypeptide comprising an amino acid sequence at least 75% identical or similar to an amino acid sequence selected from the group consisting of SEQ ID NOs: 21 to 40. In an aspect, an endogenous nucleic acid sequence provided herein encodes a polypeptide comprising an amino acid sequence at least 80% identical or similar to an amino acid sequence selected from the group consisting of SEQ ID NOs: 21 to 40. In an aspect, an endogenous nucleic acid sequence provided herein encodes a polypeptide comprising an amino acid sequence at least 85% identical or similar to an amino acid sequence selected from the group consisting of SEQ ID NOs: 21 to 40. In an aspect, an endogenous nucleic acid sequence provided herein encodes a polypeptide comprising an amino acid sequence at least 88% identical or similar to an amino acid sequence selected from the group consisting of SEQ ID NOs: 21 to 40. In an aspect, an endogenous nucleic acid sequence provided herein encodes a polypeptide comprising an amino acid sequence at least 90% identical or similar to an amino acid sequence selected from the group consisting of SEQ ID NOs: 21 to 40. In an aspect, an endogenous nucleic acid sequence provided herein encodes a polypeptide comprising an amino acid sequence at least 91% identical or similar to an amino acid sequence selected from the group consisting of SEQ ID NOs: 21 to 40. In an aspect, an endogenous nucleic acid sequence provided herein encodes a polypeptide comprising an amino acid sequence at least 92% identical or similar to an amino acid sequence selected from the group consisting of SEQ ID NOs: 21 to 40. In an aspect, an endogenous nucleic acid sequence provided herein encodes a polypeptide comprising an amino acid sequence at least 93% identical or similar to an amino acid sequence selected from the group consisting of SEQ ID NOs: 21 to 40. In an aspect, an endogenous nucleic acid sequence provided herein encodes a polypeptide comprising an amino acid sequence at least 94% identical or similar to an amino acid sequence selected from the group consisting of SEQ ID NOS: 21 to 40. In an aspect, an endogenous nucleic acid sequence provided herein encodes a polypeptide comprising an amino acid sequence at least 95% identical or similar to an amino acid sequence selected from the group consisting of SEQ ID NOs: 21 to 40. In an aspect, an endogenous nucleic acid sequence provided herein encodes a polypeptide comprising an amino acid sequence at least 96% identical or similar to an amino acid sequence selected from the group consisting of SEQ ID NOs: 21 to 40. In an aspect, an endogenous nucleic acid sequence provided herein encodes a polypeptide comprising an amino acid sequence at least 97% identical or similar to an amino acid sequence selected from the group consisting of SEQ ID NOs: 21 to 40. In an aspect, an endogenous nucleic acid sequence provided herein encodes a polypeptide comprising an amino acid sequence at least 98% identical or similar to an amino acid sequence selected from the group consisting of SEQ ID NOs: 21 to 40. In an aspect, an endogenous nucleic acid sequence provided herein encodes a polypeptide comprising an amino acid sequence at least 99% identical or similar to an amino acid sequence selected from the group consisting of SEQ ID NOS: 21 to 40. In an aspect, an endogenous nucleic acid sequence provided herein encodes a polypeptide comprising an amino acid sequence 100% identical or similar to an amino acid sequence selected from the group consisting of SEQ ID NOs: 21 to 40.

### Promoters

As commonly understood in the art, the term “promoter” refers to a DNA sequence that contains an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein.

Promoters that drive expression in all or most tissues of the plant are referred to as “constitutive” promoters. In an aspect, a constitutive promoter is selected from the group consisting of a Cauliflower Mosaic Virus 35S promoter, a ubiquitin promoter, an actin promoter, an opine promoter, and an alcohol dehydrogenase promoter.

Promoters that drive expression during certain periods or stages of development are referred to as “developmental” promoters.

Promoters that drive enhanced expression in certain tissues of an organism relative to other tissues of the organism are referred to as “tissue-preferred” promoters. Thus, a “tissue-preferred” promoter causes relatively higher or preferential expression in a specific tissue(s) of a plant, but with lower levels of expression in other tissue(s) of the plant. As a non-limiting example, a root tissue-preferred promoter exhibits higher activity in root tissue, but may also exhibit activity, albeit at lower levels, in additional tissues such as stem, leaves, and floral tissues. A “tissue-specific” promoter causes expression only in a specific tissue. As a non-limiting example, a root tissue-specific promoter drives expression only in root tissue. In an aspect, a tissue-specific promoter is a root tissue-specific promoter. In another aspect, a tissue-preferred promoter is a root tissue-preferred promoter. In an aspect, a tissue-specific promoter is a leaf tissue-specific promoter. In another aspect, a tissue-preferred promoter is a leaf tissue-preferred promoter. In an aspect, a tissue-specific promoter is a trichome tissue-specific promoter. In another aspect, a tissue-preferred promoter is a trichome tissue-preferred promoter.

In an aspect, a root tissue-preferred promoter is a cassava vein mosaic virus (CsVMV) promoter.

An “inducible” promoter is a promoter that initiates transcription in response to an environmental stimulus such as heat, cold, drought, light, or other stimuli, such as wounding or chemical application.

In an aspect, a promoter provided herein is a constitutive promoter. In another aspect, a promoter provided herein is an inducible promoter. In a further aspect, a promoter provided herein is a developmental promoter. In another aspect, a promoter is a tissue-preferred or tissue-specific promoter. In a further aspect, a promoter is selected from the group consisting of a constitutive promoter, a tissue-preferred promoter, a tissue-specific promoter, and an inducible promoter.

In an aspect, this disclosure provides a heterologous promoter. In another aspect, this disclosure provides a promoter that is operably linked to a heterologous polynucleotide. In another aspect, this disclosure provides a polynucleotide sequence that is operably linked to a heterologous promoter.

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous. In an aspect, a promoter provided herein is operably linked to a heterologous nucleic acid molecule.

In an aspect, an inducible promoter provides trichome specific or preferred expression. In one aspect, a trichome specific or preferred inducible promoter comprises a sequence selected from the group consisting of SEQ ID Nos: 62 to 69 and a functional fragment thereof. In an aspect, a specific or preferred promoter can be an inducible promoter.

In an aspect, an inducible promoter provides root specific or preferred expression. In one aspect, a root specific or preferred inducible promoter comprises a sequence selected from the group consisting of SEQ ID Nos: 70 to 80 and a functional fragment thereof. Table 1 provides a comparison of estimated leaf versus root specific expression level driven by SEQ ID Nos: 70 to 80.

In an aspect, an inducible promoter provides leaf specific or preferred expression. In one aspect, a leaf specific or preferred inducible promoter comprises a sequence selected from the group consisting of SEQ ID Nos: 81 to 90 and a functional fragment thereof. Table 2 provides a comparison of estimated leaf versus root specific expression level driven by SEQ ID Nos: 81 to 90.

TABLE 1
Examples of inducible promoters for topping-responsive root-specific or preferred expression.

| | | | | | | | Root | | | |
|---|---|---|---|---|---|---|---|---|---|---|
| | | | | | | | | | | 3 days |
| | | Leaf | | | | | | | | after |
| | | | | 3 days | 4 weeks | | | | 3 days | topping |
| SEQ | Gene | Layby | Before | after | after | | Layby | Flowering | after | (nitrogen |
| ID NO | Id | stage | Topping | Topping | Topping | Senescence | stage | time | Topping | deficient) |
| SEQ ID | g78655 | 0.0 | 0.0 | 0.0 | 0.0 | 1.1 | 1.7 | 1.0 | 146.0 | 251.0 |
| NO: 1 | | | | | | | | | | |
| SEQ ID | g72021 | 0.0 | 0.0 | 1.7 | 0.7 | 3.3 | 4.3 | 6.9 | 501.7 | 468.7 |
| NO: 2 | | | | | | | | | | |
| SEQ ID | g78252 | 3.2 | 0.0 | 0.0 | 1.5 | 0.0 | 0.9 | 3.0 | 77.3 | 167.8 |
| NO: 3 | | | | | | | | | | |
| SEQ ID | g65720 | 0.0 | 0.0 | 1.7 | 0.0 | 3.8 | 0.9 | 0.0 | 33.5 | 6.0 |
| NO: 4 | | | | | | | | | | |
| SEQ ID | g74108 | 3.2 | 3.3 | 0.0 | 0.7 | 3.6 | 2.6 | 4.0 | 81.6 | 142.9 |
| NO: 5 | | | | | | | | | | |
| SEQ ID | g47466 | 12.7 | 31.6 | 0.0 | 69.6 | 43.2 | 0.0 | 1.0 | 22.3 | 10.6 |
| NO: 6 | | | | | | | | | | |
| SEQ ID | g102868 | 3.2 | 3.3 | 0.0 | 0.0 | 0.2 | 0.9 | 1.0 | 36.9 | 23.4 |
| NO: 7 | | | | | | | | | | |
| SEQ ID | g23057 | 0.0 | 0.0 | 1.7 | 0.0 | 8.7 | 2.6 | 13.8 | 247.4 | 287.2 |
| NO: 8 | | | | | | | | | | |
| SEQ ID | g34684 | 12.7 | 41.6 | 43.0 | 67.4 | 104.0 | 21.5 | 105.7 | 1947.5 | 2177.0 |
| NO: 9 | | | | | | | | | | |
| SEQ ID | g105948 | 15.8 | 3.3 | 0.0 | 8.8 | 11.9 | 16.3 | 14.8 | 78.2 | 768.7 |
| NO: 10 | | | | | | | | | | |
| SEQ ID | g81261 | 145.6 | 68.3 | 72.3 | 90.2 | 73.1 | 44.7 | 115.6 | 2206.9 | 2112.0 |
| NO: 11 | | | | | | | | | | |

TABLE 2
Examples of inducible promoters for topping-responsive
leaf-specific or preferred expression.

| | | | 1 day | 3 days | 1 week | 2 weeks | 3 weeks | |
|---|---|---|---|---|---|---|---|---|
| SEQ | | Before | after | after | after | after | after | Harvest |
| ID NO | Gene | Topping | Topping | Topping | Topping | Topping | Topping | time |
| SEQ ID | g2237 | 20 | 170 | 344 | 305 | 706 | 432 | 1049 |
| NO: 12 | | | | | | | | |
| SEQ ID | g31142 | 41 | 61 | 177 | 480 | 167 | 1530 | 892 |
| NO: 13 | | | | | | | | |
| SEQ ID | g75488 | 41 | 9 | 306 | 534 | 47 | 2297 | 138 |
| NO: 14 | | | | | | | | |
| SEQ ID | g94193 | 119 | 128 | 846 | 1650 | 377 | 4337 | 1546 |
| NO: 15 | | | | | | | | |
| SEQ ID | g34756 | 15 | 86 | 113 | 73 | 22 | 183 | 611 |
| NO: 16 | | | | | | | | |
| SEQ ID | g104299 | 48 | 110 | 166 | 576 | 138 | 1521 | 994 |
| NO: 17 | | | | | | | | |
| SEQ ID | g44810 | 21 | 19 | 99 | 80 | 492 | 140 | 521 |
| NO: 18 | | | | | | | | |
| SEQ ID | g71671 | 15 | 26 | 58 | 210 | 40 | 348 | 299 |
| NO: 19 | | | | | | | | |
| SEQ ID | g29427 | 34 | 34 | 395 | 314 | 474 | 591 | 339 |
| NO: 20 | | | | | | | | |
| SEQ ID | g49024 | 29 | 28 | 229 | 230 | 280 | 382 | 595 |
| NO: 21 | | | | | | | | |

### Small RNA Molecules

In an aspect, a nucleic acid molecule provided herein is a small RNA molecule (also referred to as simply a “small RNA”). In another aspect, a nucleic acid molecule encodes a small RNA molecule.

As used herein, a “small RNA molecule” refers to a non-coding RNA molecule of between 16 nucleotides and 70 nucleotides in length. In an aspect, a small RNA molecule comprises between 16 nucleotides and 40 nucleotides. In another aspect, a small RNA molecule comprises between 16 nucleotides and 30 nucleotides. In another aspect, a small RNA molecule comprises between 18 nucleotides and 50 nucleotides. In another aspect, a small RNA molecule comprises between 18 nucleotides and 40 nucleotides. In another aspect, a small RNA molecule comprises between 18 nucleotides and 30 nucleotides. In another aspect, a small RNA molecule comprises between 18 nucleotides and 25 nucleotides. In another aspect, a small RNA molecule comprises between 20 nucleotides and 28 nucleotides. In another aspect, a small RNA molecule comprises between 20 nucleotides and 24 nucleotides. In another aspect, a small RNA molecule comprises between 21 nucleotides and 23 nucleotides. In another aspect, a small RNA molecule comprises 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides.

In an aspect, a small RNA molecule is selected from the group consisting of a double-stranded RNA, a small interfering RNA (siRNA), a trans-acting siRNA, and a microRNA (miRNA).

miRNAs are generally of between about 19 to about 25 nucleotides (commonly about 20-24 nucleotides in plants), that guide cleavage in trans of target transcripts, negatively regulating the expression of genes involved in various regulation and development pathways. In some cases, miRNAs serve to guide in-phase processing of siRNA primary transcripts.

It is appreciated in the art that, in plants, miRNAs and targeted nucleic acids often do not share perfect complementarity (although miRNAs and targeted nucleic acids can have perfect complementarity). miRNAs and their targets can have several mismatches between them while still enabling the miRNA to reduce the expression and/or function of the target gene. See, for example, Liu et al., Plant Cell, 26:741-753 (2014) and Wang et al., Curr. Opin. Plant Biol., 27:118-124 (2015).

In an aspect, a small RNA molecule comprises 100% complementarity with a nucleic acid molecule comprising a sequence selected from the group consisting of SEQ ID NOs: 1 to 20 and 41 to 60. In an aspect, a small RNA molecule comprises at least 95% complementarity over 21 consecutive nucleotides of a nucleic acid molecule comprising a sequence selected from the group consisting of SEQ ID NOs: 1 to 20 and 41 to 60. In an aspect, a small RNA molecule comprises at least 90% complementarity over 21 consecutive nucleotides of a nucleic acid molecule comprising a sequence selected from the group consisting of SEQ ID NOs: 1 to 20 and 41 to 60. In an aspect, a small RNA molecule comprises at least 85% complementarity over 21 consecutive nucleotides of a nucleic acid molecule comprising a sequence selected from the group consisting of SEQ ID NOs: 1 to 20 and 41 to 60. In an aspect, a small RNA molecule comprises at least 95% complementarity over 20 consecutive nucleotides of a nucleic acid molecule comprising a sequence selected from the group consisting of SEQ ID NOs: 1 to 20 and 41 to 60. In an aspect, a small RNA molecule comprises at least 90% complementarity over 20 consecutive nucleotides of a nucleic acid molecule comprising a sequence selected from the group consisting of SEQ ID NOs: 1 to 20 and 41 to 60. In an aspect, a small RNA molecule comprises at least 85% complementarity over 20 consecutive nucleotides of a nucleic acid molecule comprising a sequence selected from the group consisting of SEQ ID NOs: 1 to 20 and 41 to 60. In an aspect, a small RNA molecule comprises at least 95% complementarity over 19 consecutive nucleotides of a nucleic acid molecule comprising a sequence selected from the group consisting of SEQ ID NOS: 1 to 20 and 41 to 60. In an aspect, a small RNA molecule comprises at least 90% complementarity over 19 consecutive nucleotides of a nucleic acid molecule comprising a sequence selected from the group consisting of SEQ ID NOs: 1 to 20 and 41 to 60. In an aspect, a small RNA molecule comprises at least 85% complementarity over 19 consecutive nucleotides of a nucleic acid molecule comprising a sequence selected from the group consisting of SEQ ID NOs: 1 to 20 and 41 to 60.

Many microRNA genes (MIR genes) have been identified and made publicly available in a database (“miRBase”, available online at microrna[dot]sanger[dot]ac[dot]uk/sequences; also see Griffiths-Jones et al. (2003) Nucleic Acids Res., 31:439-441). MIR genes have been reported to occur in intergenic regions, both isolated and in clusters in the genome, but can also be located entirely or partially within introns of other genes (both protein-coding and non-protein-coding). For a review of miRNA biogenesis, see Kim (2005) Nature Rev. Mol. Cell. Biol., 6:376-385. Transcription of MIR genes can be, at least in some cases, under promotional control of a MIR gene's own promoter. The primary transcript, termed a “pri-miRNA”, can be quite large (several kilobases) and can be polycistronic, containing one or more pre-miRNAs (fold-back structures containing a stem-loop arrangement that is processed to the mature miRNA) as well as the usual 5′ “cap” and polyadenylated tail of an mRNA.

Maturation of a mature miRNA from its corresponding precursors (pri-miRNAs and pre-miRNAs) differs significantly between animals and plants. For example, in plant cells, microRNA precursor molecules are believed to be largely processed to the mature miRNA entirely in the nucleus, whereas in animal cells, the pri-miRNA transcript is processed in the nucleus by the animal-specific enzyme Drosha, followed by export of the pre-miRNA to the cytoplasm where it is further processed to the mature miRNA. Mature miRNAs in plants are typically 21 nucleotides in length.

Transgenic expression of miRNAs (whether a naturally occurring sequence or an artificial sequence) can be employed to regulate expression of the miRNA's target gene or genes. Inclusion of a miRNA recognition site in a transgenically expressed transcript is also useful in regulating expression of the transcript. Recognition sites of miRNAs have been validated in all regions of an mRNA, including the 5′ untranslated region, coding region, and 3′ untranslated region, indicating that the position of the miRNA target site relative to the coding sequence may not necessarily affect suppression. Because miRNAs are important regulatory elements in eukaryotes, transgenic suppression of miRNAs is useful for manipulating biological pathways and responses. Finally, promoters of MIR genes can have very specific expression patterns (e.g., cell-specific, tissue-specific, temporally specific, or inducible), and thus are useful in recombinant constructs to induce such specific transcription of a DNA sequence to which they are operably linked. Various utilities of miRNAs, their precursors, their recognition sites, and their promoters are described in detail in U.S. Patent Application Publication 2006/0200878 A1, incorporated by reference herein. Non-limiting examples of these utilities include: (1) the expression of a native miRNA or miRNA precursor sequence to suppress a target gene; (2) the expression of an artificial miRNA or miRNA precursor sequence to suppress a target gene; (3) expression of a transgene with a miRNA recognition site, where the transgene is suppressed when the mature miRNA is expressed; (4) expression of a transgene driven by a miRNA promoter.

Designing an artificial miRNA sequence can be as simple as substituting sequence that is complementary to the intended target for nucleotides in the miRNA stem region of the miRNA precursor, as demonstrated by Zeng et al. (2002) Mol. Cell, 9:1327-1333. One non-limiting example of a general method for determining nucleotide changes in the native miRNA sequence to produce the engineered miRNA precursor includes the following steps: (a) Selecting a unique target sequence of at least 18 nucleotides specific to the target gene, e.g., by using sequence alignment tools such as BLAST (see, for example, Altschul et al. (1990) J. Mol. Biol., 215:403-410; Altschul et al. (1997) Nucleic Acids Res., 25:3389-3402), for example, of both tobacco cDNA and genomic DNA databases, to identify target transcript orthologues and any potential matches to unrelated genes, thereby avoiding unintentional silencing of non-target sequences; (b) Analyzing the target gene for undesirable sequences (e.g., matches to sequences from non-target species), and score each potential 19-mer segment for GC content, Reynolds score (see Reynolds et al. (2004) Nature Biotechnol., 22:326-330), and functional asymmetry characterized by a negative difference in free energy (“.DELTA . . . DELTA.G” or “ΔΔG”) (see Khvorova et al. (2003) Cell, 115:209-216). Preferably 19-mers are selected that have all or most of the following characteristics: (1) a Reynolds score>4, (2) a GC content between about 40% to about 60%, (3) a negative ΔΔG, (4) a terminal adenosine, (5) lack of a consecutive run of 4 or more of the same nucleotide; (6) a location near the 3′ terminus of the target gene; (7) minimal differences from the miRNA precursor transcript. Positions at every third nucleotide in an siRNA have been reported to be especially important in influencing RNAi efficacy and an algorithm, “siExplorer” is publicly available at rna[dot]chem[dot]t[dot]u-tokyo[dot]ac[dot]jp/siexplorer.htm (see Katoh and Suzuki (2007) Nucleic Acids Res., 10.1093/nar/gk11120); (c) Determining the reverse complement of the selected 19-mers to use in making a modified mature miRNA. The additional nucleotide at position 20 is preferably matched to the selected target sequence, and the nucleotide at position 21 is preferably chosen to cither be unpaired to prevent spreading of silencing on the target transcript or paired to the target sequence to promote spreading of silencing on the target transcript; and (d) transforming the artificial miRNA into a plant.

Without being limited by any scientific theory, it is appreciated in the art that an RNAi knockdown of a candidate gene (e.g., via the use of an artificial miRNA or an siRNA) and a mutation (e.g., missense or nonsense mutations) in the same candidate gene can both cause reduction of expression and/or decreased protein activity and can cause identical or similar phenotypes in plants. See, for example, Agrawal et al., Microbiology and Molecular Biology Reviews, 67:657-685 (2003).

In an aspect, inducing a mutation comprises the use of a prime editor. As used herein, a “prime editor” refers to a Cas nickase fused to an engineered reverse transcriptase. Prime editors can introduce all 12 transition and transversion mutations and small insertions or deletions, as well as combinations thereof. Prime editors use a prime editing guide RNA (pegRNA) that specifies the target site for editing and encodes the desired edit. In an aspect, a pegRNA is provided to a tobacco cell. In an aspect, a pegRNA comprises a nucleic acid sequence that binds to a nucleic acid sequence selected from the group consisting of SEQ ID NOs: 1 to 20 or 41 to 60.

Additional information about base editors and prime editors, and their use in plants, can be found in Molla et al., Nature Plants, 7:1166-1187 (2021). See also Anzalone et al., Nature, 576:149-157 (2019); Komor et al., Nature, 533:420-424 (2016); and Gaudelli et al., Nature, 551:464-471 (2017).

In an aspect, a small RNA provided herein comprises at nucleic acid sequence at least 75% identical or complementary to a nucleic acid sequence selected from the group consisting of SEQ ID NOs: 1 to 20 and 41 to 60. In an aspect, a small RNA provided herein comprises at nucleic acid sequence at least 80% identical or complementary to a nucleic acid sequence selected from the group consisting of SEQ ID NOs: 1 to 20 and 41 to 60. In an aspect, a small RNA provided herein comprises at nucleic acid sequence at least 85% identical or complementary to a nucleic acid sequence selected from the group consisting of SEQ ID NOs: 1 to 20 and 41 to 60. In an aspect, a small RNA provided herein comprises at nucleic acid sequence at least 90% identical or complementary to a nucleic acid sequence selected from the group consisting of SEQ ID NOs: 1 to 20 and 41 to 60. In an aspect, a small RNA provided herein comprises at nucleic acid sequence at least 95% identical or complementary to a nucleic acid sequence selected from the group consisting of SEQ ID NOs: 1 to 20 and 41 to 60. In an aspect, a small RNA provided herein comprises at nucleic acid sequence at least 96% identical or complementary to a nucleic acid sequence selected from the group consisting of SEQ ID NOs: 1 to 20 and 41 to 60. In an aspect, a small RNA provided herein comprises at nucleic acid sequence at least 97% identical or complementary to a nucleic acid sequence selected from the group consisting of SEQ ID NOs: 1 to 20 and 41 to 60. In an aspect, a small RNA provided herein comprises at nucleic acid sequence at least 98% identical or complementary to a nucleic acid sequence selected from the group consisting of SEQ ID NOs: 1 to 20 and 41 to 60. In an aspect, a small RNA provided herein comprises at nucleic acid sequence at least 99% identical or complementary to a nucleic acid sequence selected from the group consisting of SEQ ID NOs: 1 to 20 and 41 to 60. In an aspect, a small RNA provided herein comprises at nucleic acid sequence 100% identical or complementary to a nucleic acid sequence selected from the group consisting of SEQ ID NOs: 1 to 20 and 41 to 60.

In an aspect, a small RNA provided herein comprises at nucleic acid sequence at least 75% complementary to a nucleic acid sequence selected from the group consisting of SEQ ID NOs: 1 to 20 and 41 to 60. In an aspect, a small RNA provided herein comprises at nucleic acid sequence at least 80% complementary to a nucleic acid sequence selected from the group consisting of SEQ ID NOs: 1 to 20 and 41 to 60. In an aspect, a small RNA provided herein comprises at nucleic acid sequence at least 85% identical or complementary to a nucleic acid sequence selected from the group consisting of SEQ ID NOs: 1 to 20 and 41 to 60. In an aspect, a small RNA provided herein comprises at nucleic acid sequence at least 90% identical or complementary to a nucleic acid sequence selected from the group consisting of SEQ ID NOs: 1 to 20 and 41 to 60. In an aspect, a small RNA provided herein comprises at nucleic acid sequence at least 95% identical or complementary to a nucleic acid sequence selected from the group consisting of SEQ ID NOs: 1 to 20 and 41 to 60. In an aspect, a small RNA provided herein comprises at nucleic acid sequence at least 96% identical or complementary to a nucleic acid sequence selected from the group consisting of SEQ ID NOs: 1 to 20 and 41 to 60. In an aspect, a small RNA provided herein comprises at nucleic acid sequence at least 97% identical or complementary to a nucleic acid sequence selected from the group consisting of SEQ ID NOs: 1 to 20 and 41 to 60. In an aspect, a small RNA provided herein comprises at nucleic acid sequence at least 98% identical or complementary to a nucleic acid sequence selected from the group consisting of SEQ ID NOs: 1 to 20 and 41 to 60. In an aspect, a small RNA provided herein comprises at nucleic acid sequence at least 99% identical or complementary to a nucleic acid sequence selected from the group consisting of SEQ ID NOs: 1 to 20 and 41 to 60. In an aspect, a small RNA provided herein comprises at nucleic acid sequence 100% identical or complementary to a nucleic acid sequence selected from the group consisting of SEQ ID NOs: 1 to 20 and 41 to 60.

In an aspect, a small RNA provided herein comprises a nucleic acid sequence at least 88.7% identical or complementary to at least 18 contiguous nucleotides of a nucleic acid sequence selected from the group consisting of SEQ ID NOs: 1 to 20 and 41 to 60. In another aspect, a small RNA provided herein comprises a nucleic acid sequence at least 94.3% identical or complementary to at least 18 contiguous nucleotides of a nucleic acid sequence selected from the group consisting of SEQ ID NOs: 1 to 20 and 41 to 60. In another aspect, a small RNA provided herein comprises a nucleic acid sequence 100% identical or complementary to at least 18 contiguous nucleotides of a nucleic acid sequence selected from the group consisting of SEQ ID NOs: 1 to 20 and 41 to 60. In another aspect, a small RNA provided herein comprises a nucleic acid sequence at least 85% identical or complementary to at least 20 contiguous nucleotides of a nucleic acid sequence selected from the group consisting of SEQ ID NOs: 1 to 20 and 41 to 60. In another aspect, a small RNA provided herein comprises a nucleic acid sequence at least 90% identical or complementary to at least 20 contiguous nucleotides of a nucleic acid sequence selected from the group consisting of SEQ ID NOs: 1 to 20 and 41 to 60. In another aspect, a small RNA provided herein comprises a nucleic acid sequence at least 95% identical or complementary to at least 20 contiguous nucleotides of a nucleic acid sequence selected from the group consisting of SEQ ID NOs: 1 to 20 and 41 to 60. In another aspect, a small RNA provided herein comprises a nucleic acid sequence 100% identical or complementary to at least 20 contiguous nucleotides of a nucleic acid sequence selected from the group consisting of SEQ ID NOs: 1 to 20 and 41 to 60.

In an aspect, a small RNA molecule provided herein is capable of binding to and reducing the expression of a nucleic acid sequence encoding a polypeptide at least 70% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 75% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 80% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 85% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 88% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 90% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 91% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 92% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 93% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 94% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 95% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 96% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 97% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 98% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide at least 99% identical or similar to an amino acid sequence selected from the group consisting of SEQ ID NOs: 21 to 40. In an aspect, a small RNA molecule provided herein is capable of binding to and reducing the expression of a nucleic acid sequence encoding a polypeptide 100% identical or similar to an amino acid sequence selected from the group consisting of SEQ ID NOs: 21 to 40.

In an aspect, a small RNA molecule provided herein is capable of binding to and reducing the expression of a nucleic acid sequence at least 70% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 75% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 80% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 85% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 88% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 90% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 91% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 92% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 93% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 94% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 95% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 96% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 97% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 98% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence at least 99% identical to nucleic acid sequence selected from the group consisting of SEQ ID NOs: 1 to 20 and 41 to 60, or an RNA transcribed therefrom. In an aspect, a small RNA molecule provided herein is capable of binding to and reducing the expression of a nucleic acid sequence 100% identical to nucleic acid sequence selected from the group consisting of SEQ ID NOs: 1 to 20 and 41 to 60, or an RNA transcribed therefrom.

As used herein, “capable of binding to” is synonymous with “capable of hybridizing to.” In an aspect, a first nucleic acid molecule that is capable of binding to a second nucleic acid molecule binds to the second nucleic acid molecule. As used herein, a first nucleic acid molecule can “hybridize” a second nucleic acid molecule via non-covalent interactions (e.g., Watson-Crick base-pairing) in a sequence-specific, antiparallel manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine pairing with thymine, adenine pairing with uracil, and guanine (G) pairing with cytosine (C) [DNA, RNA]. In addition, it is also known in the art that for hybridization between two RNA molecules (e.g., dsRNA), guanine base pairs with uracil. For example, G/U base-pairing is partially responsible for the degeneracy (i.e., redundancy) of the genetic code in the context of tRNA anti-codon base-pairing with codons in mRNA. In the context of this disclosure, a guanine of a protein-binding segment (dsRNA duplex) of a subject DNA-targeting RNA molecule is considered complementary to an uracil, and vice versa. As such, when a G/U base-pair can be made at a given nucleotide position a protein-binding segment (dsRNA duplex) of a subject DNA-targeting RNA molecule, the position is not considered to be noncomplementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer nucleotides) the position of mismatches becomes important (see Sambrook et al.). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST® programs (basic local alignment search tools) and PowerBLAST programs known in the art (see Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

### Reduced Expression/Activity

In an aspect, a small RNA molecule reduces the expression of any nucleic acid sequence to which it is capable of binding. In another aspect, a non-natural mutation provided herein reduces the expression of the mutated nucleic acid sequence as compared to the non-mutated nucleic acid sequence in a control plant grown under similar growth conditions.

Unless specified otherwise, all comparisons to control plants require similar growth conditions or comparable growth conditions for the two plants being compared. As used herein, “grown under comparable conditions,” “similar growth conditions” or “comparable growth conditions” refer to similar environmental conditions and/or agronomic practices for growing and making meaningful comparisons between two or more plant genotypes so that neither environmental conditions nor agronomic practices would contribute to or explain any difference observed between the two or more plant genotypes. Environmental conditions include, for example, light, temperature, water (humidity), and nutrition (e.g., nitrogen and phosphorus). Agronomic practices include, for example, seeding, clipping, undercutting, transplanting, topping, and suckering. See Chapters 4B and 4C of Tobacco, Production, Chemistry and Technology, Davis & Nielsen, eds., Blackwell Publishing, Oxford (1999), pp 70-103.

Reduced expression of an endogenous nucleic acid sequence can be measured using any suitable method known in the art. Non-limiting examples of measuring expression include quantitative reverse transcriptase polymerase chain reaction (qRT-PCR), RNA blot (e.g., a Northern blot), RNA sequencing. Differences in expression can be described as an absolute quantification or a relative quantification. See, for example, Livak and Schmittgen, Methods, 25:402-408 (2001). If an endogenous nucleic acid sequence encodes a protein, changes in expression can be inferred by examining the accumulation of the encoded protein. Non-limiting examples of measuring protein accumulation include Western blots and enzyme-linked immunosorbent assays (ELISAs).

In an aspect, a reduction in expression is measured using qRT-PCR. In another aspect, a reduction in expression is measured using an RNA blot. In another aspect, a reduction in expression is measured using RNA sequencing. In a further aspect, a reduction in expression is measured using a Western blot. In yet a further aspect, a reduction in expression is measured using an ELISA.

In an aspect, a non-natural mutation in a nucleic acid sequence selected from the group consisting of SEQ ID NOs: 1 to 20 and 41 to 60 results in a reduced level of expression of the nucleic acid sequence as compared to the nucleic acid sequence lacking the non-natural mutation in a control plant grown under similar growth conditions. In an aspect, a non-natural mutation in a nucleic acid sequence encoding an amino acid sequence selected from the group consisting of SEQ ID NOs: 21 to 40 results in a reduced level of expression of the nucleic acid sequence as compared to the nucleic acid sequence lacking the non-natural mutation in a control plant grown under similar growth conditions.

In an aspect, a non-natural mutation in a nucleic acid sequence selected from the group consisting of SEQ ID NOs: 1 to 20 and 41 to 60 results in a reduced level of expression of the corresponding amino acid sequence (SEQ ID NOs: 21 to 40) as compared to the nucleic acid sequence lacking the non-natural mutation in a control plant grown under similar growth conditions. In an aspect, a non-natural mutation in a nucleic acid sequence encoding an amino acid sequence selected from the group consisting of SEQ ID NOs: 21 to 40 results in a reduced level of expression of the corresponding amino acid sequence (SEQ ID NOs: 21 to 40) as compared to the nucleic acid sequence lacking the non-natural mutation in a control plant grown under similar growth conditions.

In an aspect, a reduction in expression comprises a reduction of at least 1% as compared to expression in the same tissue of a control plant grown under similar growth conditions. In another aspect, a reduction in expression comprises a reduction of at least 5% as compared to expression in the same tissue of a control plant grown under similar growth conditions. In another aspect, a reduction in expression comprises a reduction of at least 10% as compared to expression in the same tissue of a control plant grown under similar growth conditions. In another aspect, a reduction in expression comprises a reduction of at least 25% as compared to expression in the same tissue of a control plant grown under similar growth conditions. In another aspect, a reduction in expression comprises a reduction of at least 50% as compared to expression in the same tissue of a control plant grown under similar growth conditions. In another aspect, a reduction in expression comprises a reduction of at least 75% as compared to expression in the same tissue of a control plant grown under similar growth conditions. In another aspect, a reduction in expression comprises a reduction of at least 90% as compared to expression in the same tissue of a control plant grown under similar growth conditions. In another aspect, a reduction in expression comprises a reduction of at least 95% as compared to expression in the same tissue of a control plant grown under similar growth conditions.

As used herein, when referring to a protein, “activity” refers to the ability to carry out a protein function, such as, without being limited by any scientific theory, binding nucleic acids, binding other proteins, or enzymatic activity.

In an aspect, a reduction in protein activity comprises a reduction of at least 1% as compared to protein activity in the same tissue of a control plant grown under similar growth conditions. In another aspect, a reduction in protein activity comprises a reduction of at least 5% as compared to protein activity in the same tissue of a control plant grown under similar growth conditions. In another aspect, a reduction in protein activity comprises a reduction of at least 10% as compared to protein activity in the same tissue of a control plant grown under similar growth conditions. In another aspect, a reduction in protein activity comprises a reduction of at least 25% as compared to protein activity in the same tissue of a control plant grown under similar growth conditions. In another aspect, a reduction in protein activity comprises a reduction of at least 50% as compared to protein activity in the same tissue of a control plant grown under similar growth conditions. In another aspect, a reduction in protein activity comprises a reduction of at least 75% as compared to protein activity in the same tissue of a control plant grown under similar growth conditions. In another aspect, a reduction in protein activity comprises a reduction of at least 90% as compared to protein activity in the same tissue of a control plant grown under similar growth conditions. In another aspect, a reduction in protein activity comprises a reduction of at least 95% as compared to protein activity in the same tissue of a control plant grown under similar growth conditions.

In an aspect, a reduction in expression comprises a reduction of between 1% and 99% as compared to expression in the same tissue of a control plant grown under similar growth conditions. In another aspect, a reduction in expression comprises a reduction of between 1% and 90% as compared to expression in the same tissue of a control plant grown under similar growth conditions. In another aspect, a reduction in expression comprises a reduction of between 1% and 75% as compared to expression in the same tissue of a control plant grown under similar growth conditions. In another aspect, a reduction in expression comprises a reduction of between 1% and 50% as compared to expression in the same tissue of a control plant grown under similar growth conditions. In another aspect, a reduction in expression comprises a reduction of between 1% and 25% as compared to expression in the same tissue of a control plant grown under similar growth conditions. In another aspect, a reduction in expression comprises a reduction of between 25% and 90% as compared to expression in the same tissue of a control plant grown under similar growth conditions. In another aspect, a reduction in expression comprises a reduction of between 50% and 90% as compared to expression in the same tissue of a control plant grown under similar growth conditions. In another aspect, a reduction in expression comprises a reduction of between 25% and 75% as compared to expression in the same tissue of a control plant grown under similar growth conditions.

In an aspect, a reduction in protein activity comprises a reduction of between 1% and 99% as compared to protein activity in the same tissue of a control plant grown under similar growth conditions. In another aspect, a reduction in protein activity comprises a reduction of between 1% and 90% as compared to protein activity in the same tissue of a control plant grown under similar growth conditions. In another aspect, a reduction in protein activity comprises a reduction of between 1% and 75% as compared to protein activity in the same tissue of a control plant grown under similar growth conditions. In another aspect, a reduction in protein activity comprises a reduction of between 1% and 50% as compared to protein activity in the same tissue of a control plant grown under similar growth conditions. In another aspect, a reduction in protein activity comprises a reduction of between 1% and 25% as compared to protein activity in the same tissue of a control plant grown under similar growth conditions. In another aspect, a reduction in protein activity comprises a reduction of between 25% and 90% as compared to protein activity in the same tissue of a control plant grown under similar growth conditions. In another aspect, a reduction in protein activity comprises a reduction of between 50% and 90% as compared to protein activity in the same tissue of a control plant grown under similar growth conditions. In another aspect, a reduction in protein activity comprises a reduction of between 25% and 75% as compared to protein activity in the same tissue of a control plant grown under similar growth conditions.

In an aspect, a reduction in expression comprises a statistically significant reduction as compared to expression in the same tissue of a control plant grown under similar growth conditions. One of ordinary skill in the art would recognize that any level of reduction is envisioned, so long as the level of reduction has been determined to be statistically significant using an accepted statistical hypothesis test. As a non-limiting example, a Student's t-test is one statistical hypothesis test that can be used to determine if a reduction in expression between a modified plant and a control plant is statistically significant. As used herein, “statistically significant” refers to a p-value of less than or equal to 0.05.

In an aspect, a non-natural mutation results in a reduced level of activity by a protein or polypeptide encoded by a nucleic acid sequence provided herein as compared to the activity of a control plant grown under similar growth conditions. In another aspect, a non-natural mutation in an endogenous nucleic acid sequence selected from the group consisting of SEQ ID NOs: 1 to 20 and 41 to 60 reduces the level of activity by a protein or polypeptide encoded by the nucleic acid sequence as compared to activity of a protein or polypeptide encoded by the endogenous nucleic acid sequence in a control tobacco plant when grown under similar growth conditions, where the nucleic acid sequence lacks the non-natural mutation in the control tobacco plant. In another aspect, a non-natural mutation in an endogenous nucleic acid sequence, where the endogenous nucleic acid sequence encodes an amino acid sequence selected from the group consisting of SEQ ID NOs: 21 to 40 reduces the level of activity by a protein or polypeptide encoded by the nucleic acid sequence as compared to activity of a protein or polypeptide encoded by the endogenous nucleic acid sequence in a control tobacco plant when grown under similar growth conditions, where the nucleic acid sequence lacks the non-natural mutation in the control tobacco plant.

As used herein, when referring to a protein or polypeptide, “activity” refers to the ability to carry out an enzymatic function.

### Increased Expression/Activity

In an aspect, a non-natural mutation results in increased expression of a nucleic acid sequence. In an aspect, a non-natural mutation results in an increased level of expression of said nucleic acid sequence as compared to expression of said nucleic acid sequence in the same tissue of a control tobacco plant when grown under similar growth conditions, wherein said nucleic acid sequence lacks the at least one non-natural mutation in said control tobacco plant.

In an aspect, an increased level of expression comprises an increase of at least 5% as compared to expression in the same tissue of a control plant grown under similar growth conditions. In another aspect, an increased level of expression comprises an increase of at least 10% as compared to expression in the same tissue of a control plant grown under similar growth conditions. In another aspect, an increased level of expression comprises an increase of at least 25% as compared to expression in the same tissue of a control plant grown under similar growth conditions. In another aspect, an increased level of expression comprises an increase of at least 50% as compared to expression in the same tissue of a control plant grown under similar growth conditions. In another aspect, an increased level of expression comprises an increase of at least 75% as compared to expression in the same tissue of a control plant grown under similar growth conditions. In another aspect, an increased level of expression comprises an increase of at least 100% as compared to expression in the same tissue of a control plant grown under similar growth conditions. In another aspect, an increased level of expression comprises an increase of at least 200% as compared to expression in the same tissue of a control plant grown under similar growth conditions. In another aspect, an increased level of expression comprises an increase of at least 500% as compared to expression in the same tissue of a control plant grown under similar growth conditions.

In an aspect, a non-natural mutation results in an increased level of activity by a protein or polypeptide encoded by said nucleic acid sequence as compared to activity of a protein or polypeptide encoded by said nucleic acid sequence in a control tobacco plant when grown under similar growth conditions, wherein said nucleic acid sequence lacks the at least one non-natural mutation in said control tobacco plant.

### Plants

As used herein, a “control plant” refers to a plant of identical, or nearly identical, genetic makeup as the modified plant being compared, except for a modification (i.e. non-natural mutation in an endogenous gene, small RNA, recombinant nucleic acid construct) provided herein that was introduced to the modified plant. A control plant is grown under the same conditions and at the same time as the comparator plant.

As used herein, a tobacco plant can be from any plant from the Nicotiana genus including, but not limited to Nicotiana tabacum, Nicotiana amplexicaulis PI 271989; Nicotiana benthamiana PI 555478; Nicotiana bigelovii PI 555485; Nicotiana debneyi; Nicotiana excelsior PI 224063; Nicotiana glutinosa PI 555507; Nicotiana goodspeedii PI 241012; Nicotiana gossei PI 230953; Nicotiana hesperis PI 271991; Nicotiana knightiana PI 555527; Nicotiana maritima PI 555535; Nicotiana megalosiphon PI 555536; Nicotiana nudicaulis PI 555540; Nicotiana paniculata PI 555545; Nicotiana plumbaginifolia PI 555548; Nicotiana repanda PI 555552; Nicotiana rustica; Nicotiana suaveolens PI 230960; Nicotiana sylvestris PI 555569; Nicotiana tomentosa PI 266379; Nicotiana tomentosiformis; and Nicotiana trigonophylla PI 555572. In an aspect, a tobacco plant described here is a Nicotiana tabacum plant.

Other plant species suitable for use with the disclosures provided herein may include members of the genera Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea. Additional suitable species may include Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale (tritic wheat times rye), bamboo, Helianthus annuus (sunflower), Carthamus tinctorius (safflower), Jatropha curcas (jatropha), Ricinus communis (castor), Elaeis guineensis (palm), Linum usitatissimum (flax), Brassica juncea, Beta vulgaris (sugarbeet), Manihot esculenta (cassaya), Lycopersicon esculentum (tomato), Lactuca sativa (lettuce), Musyclise alca (banana), Solanum tuberosum (potato), Brassica oleracea (broccoli, cauliflower, Brussels sprouts), Camellia sinensis (tea), Fragaria ananassa (strawberry), Theobroma cacao (cocoa), Coffeycliseca (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Rosa spp. (rose), Dianthus caryophyllus (carnation), Petunia spp. (petunia), Poinsettia pulcherrima (poinsettia), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass) and Phleum pratense (timothy), Panicum virgatum (switchgrass), Sorghuycliseor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), or Pennisetum glaucum (pearl millet).

In an aspect, preferred plants for use with the current disclosure include any plant or subspecies selected form the group consisting of plants genera of tobacco (Nicotiana spp.), cannabis (Cannabis spp.), or tea (Camellia spp). Suitably, the tobacco plant is a Nicotiana tabacum tobacco plant, preferably, a Burley type Nicotiana tabacum tobacco plant. Other plants suitable for use in the present disclosure include, but are not limited to, Camellia sinensis and Cannabis sativa.

In an aspect, tobacco parts provided include, but are not limited to, a leaf, a stem, a root, a trichome, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast. In an aspect, tobacco part provided does not include seed. In an aspect, this disclosure provides tobacco plant cells, tissues, and organs that are not reproductive material and do not mediate the natural reproduction of the plant. In another aspect, this disclosure also provides tobacco plant cells, tissues, and organs that are reproductive material and mediate the natural reproduction of the plant. In another aspect, this disclosure provides tobacco plant cells, tissues, and organs that cannot maintain themselves via photosynthesis. In another aspect, this disclosure provides somatic tobacco plant cells. Somatic cells, contrary to germline cells, do not mediate plant reproduction.

Cells, tissues and organs can be from seed, fruit, leaf, cotyledon, hypocotyl, meristem, embryos, endosperm, root, shoot, stem, trichome, pod, flower, inflorescence, stalk, pedicel, style, stigma, receptacle, petal, sepal, pollen, anther, filament, ovary, ovule, pericarp, phloem, vascular tissue. In another aspect, this disclosure provides a tobacco plant chloroplast. In a further aspect, this disclosure provides epidermal cells, stomata cell, leaf or root hairs, a storage root, or a tuber. In another aspect, this disclosure provides a tobacco protoplast.

Skilled artisans understand that tobacco plants naturally reproduce via seeds, not via asexual reproduction or vegetative propagation. In an aspect, this disclosure provides tobacco endosperm.

This disclosure provides cells from tobacco plants provided herein.

As used herein, a “progeny tobacco plant” or “progeny tobacco seed” can be from any filial generation, e.g., F₁, F₂, F₃, F₄, F₅, F₆, F₇, etc.

In an aspect, a tobacco plant, or part thereof, is of a tobacco variety selected from the group consisting of a flue-cured variety, a bright variety, a Burley variety, a Virginia variety, a Maryland variety, a dark variety, a Galpão variety, an Oriental variety, and a Turkish variety. In one aspect, a modified tobacco plant, or part thereof, provided herein is of a tobacco variety selected from the group consisting of a flue-cured variety, a bright variety, a Burley variety, a Virginia variety, a Maryland variety, a dark variety, a Galpão variety, an Oriental variety, and a Turkish variety.

In an aspect, a tobacco cell is of a tobacco variety selected from the group consisting of a flue cured variety, a bright variety, a Burley variety, a Virginia variety, a Maryland variety, a dark variety, a Galpão variety, an Oriental variety, and a Turkish variety. In an aspect, a modified tobacco cell is of a tobacco variety selected from the group consisting of a flue cured variety, a bright variety, a Burley variety, a Virginia variety, a Maryland variety, a dark variety, a Galpão variety, an Oriental variety, and a Turkish variety.

In an aspect, a tobacco leaf is of a tobacco variety selected from the group consisting of a flue cured variety, a bright variety, a Burley variety, a Virginia variety, a Maryland variety, a dark variety, a Galpão variety, an Oriental variety, and a Turkish variety.

In an aspect, a cured tobacco leaf or plant part is of a tobacco variety selected from the group consisting of a flue cured variety, a bright variety, a Burley variety, a Virginia variety, a Maryland variety, a dark variety, a Galpão variety, an Oriental variety, and a Turkish variety. Skilled artisans further understand that cured tobacco does not constitute a living organism and is not capable of growth or reproduction.

Flue-cured tobaccos (also called “Virginia” or “bright” tobaccos) amount to approximately 40% of world tobacco production. Flue-cured tobaccos are often also referred to as “bright tobacco” because of the golden-yellow to deep-orange color it reaches during curing. Flue-cured tobaccos have a light, bright aroma and taste. Flue-cured tobaccos are generally high in sugar and low in oils. Major flue-cured tobacco growing countries are Argentina, Brazil, China, India, Tanzania and the United States of America. In one aspect, tobacco plants or seeds or modified tobacco plants or seeds provided herein are of a flue-cured tobacco variety selected from the group consisting of the varieties listed in Table 3, and any variety essentially derived from any one of the foregoing varieties. See WO 2004/041006 A1. In a further aspect, modified tobacco plants or seeds provided herein are in a flue-cured variety selected from the group consisting of K326, K346, and NC196.

TABLE 3
Flue-cured Tobacco Varieties.

| | | |
|---|---|---|
| 400 (TC 225) | K 346 | Reams 134 |
| 401 (TC 226) | K 346 (TC 569) | Reams 158 |
| 401 Cherry Red (TC 227) | K 358 | Reams 713 |
| 401 Cherry Red Free (TC 228) | K 394 (TC 321) | Reams 744 |
| Cash (TC 250) | K 399 | Reams M1 |
| Cash (TI 278) | K 399 (TC 322) | RG 11 (TC 600) |
| CC 101 | K 730 | RG 13 (TC 601) |
| CC 1063 | Lonibow (TI 1573) | RG 17 (TC 627) |
| CC 13 | Lonibow (TI 1613) | RG 22 (TC 584) |
| CC 143 | McNair 10 (TC 330) | RG 8 (TC 585) |
| CC 200 | McNair 135 (TC 337) | RG 81 (TC 618) |
| CC 27 | McNair 30 (TC 334) | RG H51 |
| CC 301 | McNair 373 (TC 338) | RG4H 217 |
| CC 33 | McNair 944 (TC 339) | RGH 12 |
| CC 35 | MK94 (TI 1512) | RGH 4 |
| CC 37 | MS K 326 | RGH 51 |
| CC 400 | MS NC 71 | RGH 61 |
| CC 500 | MS NC 72 | SC 58 (TC 400) |
| CC 600 | NC 100 | SC 72 (TC 403) |
| CC 65 | NC 102 | Sp. G-168 |
| CC 67 | NC 1071 (TC 364) | SPEIGHT 168 |
| CC 700 | NC 1125-2 | Speight 168 (TC 633) |
| CC 800 | NC 12 (TC 346) | Speight 172 (TC 634) |
| CC 900 | NC 1226 | Speight 178 |
| Coker 139 (TC 259) | NC 196 | Speight 179 |
| Coker 139 yb1, yb2 | NC 2326 (TC 365) | Speight 190 |
| Coker 140 (TC 260) | NC 27 NF (TC 349) | Speight 196 |
| Coker 176 (TC 262) | NC 291 | SPEIGHT 220 |
| Coker 187 (TC 263) | NC 297 | SPEIGHT 225 |
| Coker 187-Hicks (TC 265) | NC 299 | SPEIGHT 227 |
| Coker 209 (TC 267) | NC 37 NF (TC 350) | SPEIGHT 236 |
| Coker 258 (TC 270) | NC 471 | Speight G-10 (TC 416) |
| Coker 298 (TC 272) | NC 55 | Speight G-102 |
| Coker 316 (TC 273) | NC 567 (TC 362) | Speight G-108 |
| Coker 319 (TC 274) | NC 60 (TC 352) | Speight G-111 |
| Coker 347 (TC 275) | NC 606 | Speight G-117 |
| Coker 371-Gold (TC 276) | NC 6140 | Speight G-126 |
| Coker 411 (TC 277) | NC 71 | Speight G-15 (TC 418) |
| Coker 48 (TC 253) | NC 72 | Speight G-23 |
| Coker 51 (TC 254) | NC 729 (TC 557) | Speight G-28 (TC 420) |
| Coker 86 (TC 256) | NC 810 (TC 659) | Speight G-33 |
| CU 263 (TC 619) | NC 82 (TC 356) | Speight G-41 |
| CU 561 | NC 8640 | Speight G-5 |
| DH95-1562-1 | NC 89 (TC 359) | Speight G-52 |
| Dixie Bright 101 (TC 290) | NC 92 | Speight G-58 |
| Dixie Bright 102 (TC 291) | NC 925 | Speight G-70 |
| Dixie Bright 244 (TC 292) | NC 95 (TC 360) | Speight G-70 (TC 426) |
| Dixie Bright 27 (TC 288) | NC 98 (TC 361) | Speight G-80 (TC 427) |
| Dixie Bright 28 (TC 289) | NC EX 24 | Speight NF3 (TC 629) |
| GF 157 | NC PY 10 (TC 367) | STNCB |
| GF 318 | NC TG 61 | VA 182 |
| GL 26H | Oxford 1 (TC 369) | VA 45 (TC 559) |
| GL 338 | Oxford 1-181 (TC 370) | Vesta 30 (TC 439) |
| GL 350 | Oxford 2 (TC 371) | Vesta 33 (TC 440) |
| GL 368 | Oxford 207 (TC 632) | Vesta 5 (TC 438) |
| GL 395 | Oxford 26 (TC 373) | Vesta 62 (TC 441) |
| GL 600 | Oxford 3 (TC 372) | Virginia (TI 220) |
| GL 737 | Oxford 414 NF | Virginia (TI 273) |
| GL 939 | PD 611 (TC 387) | Virginia (TI 877) |
| GL 939 (TC 628) | PVH 03 | Virginia 115 (TC 444) |
| Hicks (TC 310) | PVH 09 | Virginia 21 (TC 443) |
| Hicks Broadleaf (TC 311) | PVH 1118 | Virginia Bright (TI 964) |
| K 149 (TC 568) | PVH 1452 | Virginia Bright Leaf (TC 446) |
| K 317 | PVH 1600 | Virginia Gold (TC 447) |
| K 326 | PVH 2110 | White Stem Orinoco (TC 451) |
| K 326 (TC 319) | PVH 2275 | |
| K 340 (TC 320) | R 83 (Line 256-1) (TI 1400) | |

Air-cured tobaccos include “Burley,” “Maryland,” and “dark” tobaccos. The common factor linking air-cured tobaccos is that curing occurs primarily without artificial sources of heat and humidity. Burley tobaccos are light to dark brown in color, high in oil, and low in sugar. Burley tobaccos are typically air-cured in barns. Major Burley growing countries include Argentina, Brazil, Italy, Malawi, and the United States of America.

Maryland tobaccos are extremely fluffy, have good burning properties, low nicotine and a neutral aroma. Major Maryland growing countries include the United States of America and Italy.

In one aspect, tobacco plants or seeds or modified tobacco plants or seeds provided herein are of a Burley tobacco variety selected from the group consisting of the tobacco varieties listed in Table 4, and any variety essentially derived from any one of the foregoing varieties. In a further aspect, modified tobacco plants or seeds provided herein are in a Burley variety selected from the group consisting of TN 90, KT 209, KT 206, KT212, and HB 4488.

TABLE 4
Burley Tobacco Varieties.

| | | |
|---|---|---|
| 4407 LC | HB 4108P | KY 54 (TC 71) |
| AA-37-1 | HB 4151P | KY 56 (TC 72) |
| Burley 21 (TC 7) | HB 4192P | KY 56 (TC 72) |
| Burley 49 (TC 10) | HB 4194P | KY 57 (TC 73) |
| Burley 64 (TC 11) | HB 4196 | KY 58 (TC 74) |
| Burley Mammoth KY 16 (TC 12) | HB 4488 | KY 8654 (TC 77) |
| Clay 402 | HB 4488P | KY 8959 |
| Clay 403 | HB04P | KY 9 (TC 54) |
| Clay 502 | HB 4488 LC | KY 907 LC |
| Clays 403 | HIB 21 | KY 908 (TC 630) |
| GR 10 (TC 19) | HPB 21 | NBH 98 (Screened) |
| GR 10 (TC 19) | HY 403 | NC 1206 |
| GR 10A (TC 20) | Hybrid 403 LC | NC 129 |
| GR 13 (TC 21) | Hybrid 404 LC | NC 2000 LC |
| GR 14 (TC 22) | Hybrid 501 LC | NC 2002 LC |
| GR 149 LC | KDH-959 (TC 576) | NC 3 LC |
| GR 153 | KDH-960 (TC 577) | NC 5 LC |
| GR 17 (TC 23) | KT 200 LC | NC 6 LC |
| GR 17B (TC 24) | KT 204 LC | NC 7 LC |
| GR 18 (TC 25) | KT 206 LC | NC BH 129 LC |
| GR 19 (TC 26) | KT 209 LC | NC03-42-2 |
| GR 2 (TC 15) | KT 210 LC | Newton 98 |
| GR 24 (TC 27) | KT 212 LC | R 610 LC |
| GR 36 (TC 28) | KT 215 LC | R 630 LC |
| GR 38 (TC 29) | KY 1 (TC 52) | R 7-11 |
| GR 38A (TC 30) | KY 10 (TC 55) | R 59-116 LC |
| GR 40 (TC 31) | KY 12 (TC 56) | RG 17 |
| GR 42 (TC 32) | KY 14 (TC 57) | TKF 1801 LC |
| GR 42C (TC 33) | KY 14 × L8 LC | TKF 2002 LC |
| GR 43 (TC 34) | KY 15 (TC 58) | TKF 4024 LC |
| GR 44 (TC 35) | KY 16 (TC 59) | TKF 4028 LC |
| GR 45 (TC 36) | KY 17 (TC 60) | TKF 6400 LC |
| GR 46 (TC 37) | KY 19 (TC 61) | TKF 7002 LC |
| GR 48 (TC 38) | KY 21 (TC 62) | TKS 2002 LC |
| GR 5 (TC 16) | KY 22 (TC 63) | TN 86 (TC 82) |
| GR 53 (TC 39) | KY 24 (TC 64) | TN 90 LC |
| GR 6 (TC 17) | KY 26 (TC 65) | TN 97 Hybrid LC |
| GR 9 (TC 18) | KY 33 (TC 66) | TN 97 LC |
| GR139 NS | KY 34 (TC 67) | VA 116 |
| GR139 S | KY 35 (TC 68) | VA 119 |
| HB 04P | KY 41A (TC 69) | Virgin A Mutante (TI 1406) |
| HB 04P LC | KY 5 (TC 53) | Virginia 509 (TC 84) |
| HB 3307P LC | KY 52 (TC 70) | |

In another aspect, tobacco plants or seeds or modified tobacco plants or seeds provided herein are of a Maryland tobacco variety selected from the group consisting of the tobacco varieties listed in Table 5, and any variety essentially derived from any one of the foregoing varieties.

TABLE 5
Maryland Tobacco Varieties.

Maryland 10 (TC 498)
Maryland 14 D2 (TC 499)
Maryland 201 (TC 503)
Maryland 21 (TC 500)
Maryland 341 (TC 504)
Maryland 40
Maryland 402
Maryland 59 (TC 501)
Maryland 601
Maryland 609 (TC 505)
Maryland 64 (TC 502)
Maryland 872 (TC 506)
Maryland Mammoth (TC 507)

Dark air-cured tobaccos are distinguished from other tobacco types primarily by its curing process, which gives dark air-cured tobacco its medium-brown to dark-brown color and a distinct aroma. Dark air-cured tobaccos are mainly used in the production of chewing tobacco and snuff. In one aspect, modified tobacco plants or seeds provided herein are of a dark air-cured tobacco variety selected from the group consisting of Sumatra, Jatim, Dominican Cubano, Besuki, One sucker, Green River, Virginia sun-cured, and Paraguayan Passado, and any variety essentially derived from any one of the foregoing varieties.

Dark fire-cured tobaccos are generally cured with low-burning wood fires on the floors of closed curing barns. Dark fire-cured tobaccos are typically used for making pipe blends, cigarettes, chewing tobacco, snuff, and strong-tasting cigars. Major growing regions for dark fire-cured tobaccos are Tennessee, Kentucky, and Virginia in the United States of America. In one aspect, tobacco plants or seeds or modified tobacco plants or seeds provided herein are of a dark fire-cured tobacco variety selected from the group consisting of the tobacco varieties listed in Table 6, and any variety essentially derived from any one of the foregoing varieties.

TABLE 6
Dark Tobacco Varieties.

| | | |
|---|---|---|
| Black Mammoth (TC 461) | KY 171 (TC 475) | PD 7309 LC |
| Black Mammoth Small Stalk (TC 641) | KY 171 LC | PD 7312 LC |
| Certified Madole (TC 463) | KY 171 NS | PD 7318 LC |
| D-534-A-1 (TC 464) | KY 180 (TC 573) | PD 7319 LC |
| DAC ULT 302 | KY 190 (TC 574) | Petico M PG04 |
| DAC ULT 303 | Little Crittenden | PY KY 160 (TC 612) |
| DAC ULT 306 | Little Crittenden (TC 476) | PY KY 171 (TC 613) |
| DAC ULT 308 | Little Crittenden LC | Shirey |
| | (certified) | |
| DAC ULT 312 | Little Crittenden PhPh | TI 1372 |
| DF 300 (TC 465) | Lizard Tail Turtle Foot | TN D94 |
| DF 485 (TC 466) | Madole (TC 478) | TN D94 (TC 621) |
| DF 516 (TC 467) | Madole (TC 479) | TN D950 |
| DF 911 (TC 468) | MS KY 171 | TN D950 (PhPh) |
| DT 508 | MS NL Madole LC | TN D950 |
| DT 518 (Screened) | MS TN D950 LC | TN D950 (TC 622) |
| DT 538 LC | Nance (TC 616) | TR Madole (TC 486) |
| DT 592 | Narrow Leaf Madole LC | VA 309 |
| | (certified) | |
| Improved Madole (TC 471) | Neal Smith Madole (TC 646) | VA 309 (TC 560) |
| Jernigan's Madole (TC 472) | Newtons VH Madole | VA 309 LC (certified) |
| KT 14LC | NL Madole | VA 310 (TC 487) |
| KT D17LC | NL Madole (PhPh) | VA 331 (TC 592) |
| KT D4 LC | NL Madole (TC 484) | VA 355 (TC 638) |
| KT D6 LC | NL Madole LC | VA 359 |
| KT D8 LC | NL Madole LC (PhPh) | VA 359 (Screened) |
| KY 153 (TC 216) | NL Madole NS | VA 359 (TC 639) |
| KY 157 (TC 217) | One Sucker (TC 224) | VA 359 LC (certified) |
| KY 160 | OS 400 | VA 403 (TC 580) |
| KY 160 (TC 218) | PD 302H | VA 405 (TC 581) |
| KY 163 (TC 219) | PD 312H | VA 409 (TC 562) |
| KY 165 (TC 220) | PD 318H | VA 510 (TC 572) |
| KY 170 (TC 474) | PD 7302 LC | |
| KY 171 (PhPh) | PD 7305 | |

Oriental tobaccos are also referred to as Greek, aroma and Turkish tobaccos due to the fact that they are typically grown in eastern Mediterranean regions such as Turkey, Greece, Bulgaria, Macedonia, Syria, Lebanon, Italy, and Romania. The small plant size, small leaf size, and unique aroma properties of Oriental tobacco varieties are a result of their adaptation to the poor soil and stressful climatic conditions in which they have been developed. In one aspect, tobacco plants or seeds or modified tobacco plants or seeds provided herein are of an Oriental tobacco variety selected from the group consisting of the tobacco varieties listed in Table 7, and any variety essentially derived from any one of the foregoing varieties.

TABLE 7
Oriental Tobacco Varieties.

| | | |
|---|---|---|
| Bafra (TI 1641) | Edirne (TI 1671) | Samsun (TC 536) |
| Bahce (TI 1730) | Ege (TI 1642) | Samsun 959 (TI 1570) |
| Bahia (TI 1416) | Ege-64 (TI 1672) | Samsun Evkaf (TI 1723) |
| Bahia (TI 1455) | Izmir (Akhisar) (TI 1729) | Samsun Holmes NN (TC 540) |
| Baiano (TI 128) | Izmir (Gavurkoy) (TI 1727) | Samsun Maden (TI 1647) |
| Basma | Izmir Ege 64 | Samsun NO 15 (TC 541) |
| Basma (TI 1666) | Izmir-Incekara (TI 1674) | Samsun-BLK SHK Tol (TC 542) |
| Basma Drama | Izmir-Ozbas (TI 1675) | Samsun-Canik (TI 1678) |
| Basma Hybrid (PhPh) | Jaka Dzebel (TI 1326) | Samsun-Maden (TI 1679) |
| Basma Zihna I | Kaba-Kulak | Saribaptar 407 - Izmir Region |
| Bitlis (TI 1667) | Kagoshima Maruba (TI 158) | Smyrna (TC 543) |
| Bitlis (TI 1725) | Katerini | Smyrna No. 23 (TC 545) |
| Bubalovac (TI 1282) | Katerini S53 | Smyrna No. 9 (TC 544) |
| Bursa (TI 1650) | Krumovgrad 58 | Smyrna-Blk Shk Tol (TC 546) |
| Bursa (TI 1668) | MS Basma | Trabzon (TI 1649) |
| Canik (TI 1644) | MS Katerini S53 | Trabzon (TI 1682) |
| Djebel 174 (TI 1492) | Nevrokop 1146 | Trapezund 161 (TI 1407) |
| Djebel 359 (TI 1493) | Ozbas (TI 1645) | Turkish (TC 548) |
| Djebel 81 | Perustitza (TI 980) | Turkish Angshit (TI 90) |
| Dubec 566 (TI 1409) | Prilep (TI 1291) | Turkish Samsum (TI 92) |
| Dubec 7 (TI 1410) | Prilep (TI 1325) | Turkish Tropizoid (TI 93) |
| Dubek 566 (TI 1567) | Prilep 12-2/1 | Turkish Varotic (TI 89) |
| Duzce (TI 1670) | Prilep 23 | Xanthi (TI 1662) |

In an aspect, tobacco plants or seeds or modified tobacco plants or seeds provided herein are of an cigar tobacco variety selected from the group consisting of the tobacco varieties listed in Table 8, and any variety essentially derived from any one of the foregoing varieties.

TABLE 8
Cigar Tobacco Varieties.

| | | |
|---|---|---|
| Bahai (TI 62) | Castillo Negro, Blanco, Pina (TI 449) | Enshu (TI 1586) |
| Beinhart 1000 | Caujaro (TI 893) | Florida 301 |
| Beinhart 1000 (TI 1562) | Chocoa (TI 289) | Florida 301 (TC 195) |
| Beinhart 1000-1 (TI 1561) | Chocoa (TI 313) | PA Broadleaf (TC 119) |
| Bergerac C | Connecticut 15 (TC 183) | Pennsylvania Broadleaf |
| Bergerac C (TI 1529) | Connecticut Broadleaf | Pennsylvania Broadleaf (TC 119) |
| Big Cuban (TI 1565) | Connecticut Broadleaf (TC 186) | Petite Havana SR1 |
| Castillo Negro, Blanco, | Connecticut Shade (TC 188) | Petite Havana SR1 (TC 105) |
| Pina (TI 448) | | |
| Castillo Negro, Blanco, | Criollo, Colorado (TI 1093) | |
| Pina (TI 448A) | | |

In an aspect, tobacco plants or seeds or modified tobacco plants or seeds provided herein are of a tobacco variety selected from the group consisting of the tobacco varieties listed in Table 9, and any variety essentially derived from any one of the foregoing varieties.

TABLE 9
Other Tobacco Varieties.

Chocoa (TI 319)
Hoja Parada (TI 1089)
Hoja Parado (Galpão) (TI 1068)
Perique (St. James Parrish)
Perique (TC 556)
Perique (TI 1374)
Sylvestris (TI 984)
TI 179

In an aspect, a tobacco plant, or part thereof, is from a variety selected from the group consisting of the tobacco varieties listed in Tables 3 to 9. In another aspect, a tobacco plant, or part thereof, is from a variety listed in Table 3. In another aspect, a tobacco plant, or part thereof, is from a variety listed in Table 4. In another aspect, a tobacco plant, or part thereof, is from a variety listed in Table 5. In another aspect, a tobacco plant, or part thereof, is from a variety listed in Table 6. In another aspect, a tobacco plant, or part thereof, is from a variety listed in Table 7. In another aspect, a tobacco plant, or part thereof, is from a variety listed in Table 8. In another aspect, a tobacco plant, or part thereof, is from a variety listed in Table 9.

In an aspect, a modified tobacco plant, or part thereof, is from a variety selected from the group consisting of the tobacco varieties listed in Tables 3 to 9. In an aspect, a modified tobacco plant, or part thereof, is from a variety listed in Table 3. In another aspect, a modified tobacco plant, or part thereof, is from a variety listed in Table 4. In another aspect, a modified tobacco plant, or part thereof, is from a variety listed in Table 5. In another aspect, a modified tobacco plant, or part thereof, is from a variety listed in Table 6. In another aspect, a modified tobacco plant, or part thereof, is from a variety listed in Table 7. In another aspect, a modified tobacco plant, or part thereof, is from a variety listed in Table 8. In another aspect, a modified tobacco plant, or part thereof, is from a variety listed in Table 9.

In an aspect, a tobacco seed is from a variety selected from the group consisting of the tobacco varieties listed in Tables 3 to 9. In another aspect, a tobacco seed is from a variety listed in Table 3. In another aspect, a tobacco seed is from a variety listed in Table 4. In another aspect, a tobacco seed is from a variety listed in Table 5. In another aspect, a tobacco seed is from a variety listed in Table 6. In another aspect, a tobacco seed is from a variety listed in Table 7. In another aspect, a tobacco seed is from a variety listed in Table 8. In another aspect, a tobacco seed is from a variety listed in Table 9.

In an aspect, a tobacco cell is from a variety selected from the group consisting of the tobacco varieties listed in Tables 3 to 9. In another aspect, a tobacco cell is from a variety listed in Table 3. In another aspect, a tobacco cell is from a variety listed in Table 4. In another aspect, a tobacco cell is from a variety listed in Table 5. In another aspect, a tobacco cell is from a variety listed in Table 6. In another aspect, a tobacco cell is from a variety listed in Table 7. In another aspect, a tobacco cell is from a variety listed in Table 8. In another aspect, a tobacco cell is from a variety listed in Table 9.

All foregoing mentioned specific varieties of flue-cured, dark air-cured, Burley, Maryland, dark fire-cured, cigar, or Oriental type are listed only for exemplary purposes. Any additional flue-cured, dark air-cured, Burley, Maryland, dark fire-cured, cigar, or Oriental varieties are also contemplated in the present application.

As used herein, a “Secretor” [S] is a plant with a phenotype comprising at least secreting glandular trichomes.

As used herein, a “non-secretor” [NS] is a plant with a phenotype comprising non-secreting glandular trichomes.

In an aspect, a tobacco plant or variety provided herein is an inbred tobacco plant or variety. As used herein, an “inbred” tobacco variety is a variety that has been bred for genetic homogeneity.

As used herein, a “hybrid” is created by crossing two plants from different varieties or species, such that the progeny comprises genetic material from each parent. Skilled artisans recognize that higher order hybrids can be generated as well. For example, a first hybrid can be made by crossing Variety C with Variety D to create a C×D hybrid, and a second hybrid can be made by crossing Variety E with Variety F to create an E×F hybrid. The first and second hybrids can be further crossed to create the higher order hybrid (C×D)×(E×F) comprising genetic information from all four parent varieties. In an aspect, a modified tobacco plant provided herein is a hybrid tobacco plant. In another aspect, a modified tobacco seed provided herein is a hybrid tobacco seed. In an aspect, a tobacco plant or variety provided herein is a hybrid tobacco plant or variety. In another aspect, a modified tobacco plant provided herein is a hybrid tobacco plant.

In an aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a non-natural mutation in an endogenous nucleic acid sequence, where the endogenous nucleic acid sequence encodes a polypeptide comprising an amino acid sequence at least 80% identical or similar to an amino acid sequence selected from the group consisting of SEQ ID NOs: 21 to 40, where the non-natural mutation is not present in the endogenous nucleic acid sequence in a control tobacco plant of the first tobacco variety; and (b) selecting for at least one progeny tobacco seed, or a plant germinated therefrom, wherein the at least one tobacco seed or plant germinated therefrom comprises the non-natural mutation. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a non-natural mutation in an endogenous nucleic acid sequence at least 80% identical to a nucleic acid sequence selected from the group consisting of SEQ ID NOs: 41 to 60, where the non-natural mutation is not present in the endogenous nucleic acid sequence in a control tobacco plant of the first tobacco variety; and (b) selecting for at least one progeny tobacco seed, or a plant germinated therefrom, wherein the at least one tobacco seed or plant germinated therefrom comprises the non-natural mutation. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a non-natural mutation in an endogenous nucleic acid sequence at least 80% identical to a nucleic acid sequence selected from the group consisting of SEQ ID NOs: 41 to 60, where the non-natural mutation is not present in the endogenous nucleic acid sequence in a control tobacco plant of the first tobacco variety; and (b) selecting for at least one progeny tobacco seed, or a plant germinated therefrom, wherein the at least one tobacco seed or plant germinated therefrom comprises the non-natural mutation. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a non-natural mutation in an endogenous nucleic acid sequence selected from the group consisting of SEQ ID NOs: 41 to 60, where the non-natural mutation is not present in the endogenous nucleic acid sequence in a control tobacco plant of the first tobacco variety; and (b) selecting for at least one progeny tobacco seed, or a plant germinated therefrom, wherein the at least one tobacco seed or plant germinated therefrom comprises the non-natural mutation. In an aspect, in any of the foregoing methods, the first tobacco variety and second tobacco variety are the same tobacco variety. In another aspect, in any of the foregoing methods, the first tobacco variety and second tobacco variety are two different tobacco varieties.

In an aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40, were the recombinant DNA construct is not present in a control tobacco plant of the first tobacco variety; and (b) selecting for at least one progeny tobacco seed, or a plant germinated therefrom, wherein the at least one progeny tobacco seed or plant germinated therefrom comprises the recombinant DNA construct. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence encoding at least one small RNA molecule capable of binding to and reducing the expression of an endogenous nucleic acid sequence at least 80% identical or similar to a nucleic acid sequence selected from the group consisting of SEQ ID NOs: 1 to 20, were the recombinant DNA construct is not present in a control tobacco plant of the first tobacco variety; and (b) selecting for at least one progeny tobacco seed, or a plant germinated therefrom, wherein the at least one progeny tobacco seed or plant germinated therefrom comprises the recombinant DNA construct. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence encoding at least one small RNA molecule capable of binding to and reducing the expression of an endogenous nucleic acid sequence at least 80% identical or similar to a nucleic acid sequence selected from the group consisting of SEQ ID NOs: 41 to 60, were the recombinant DNA construct is not present in a control tobacco plant of the first tobacco variety; and (b) selecting for at least one progeny tobacco seed, or a plant germinated therefrom, where the at least one progeny tobacco seed or plant germinated therefrom comprises the recombinant DNA construct. In an aspect, in any of the foregoing methods, the first tobacco variety and second tobacco variety are the same tobacco variety. In another aspect, in any of the foregoing methods, the first tobacco variety and second tobacco variety are two different tobacco varieties. In a further aspect, a recombinant DNA construct of each of the above aspects comprises a promoter sequence selected from the group consisting of SEQ ID NOs: 62 to 90.

In an aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOS: 21 to 40, where the recombinant DNA construct is not present in a control tobacco plant of the first tobacco variety; and (b) selecting for at least one progeny tobacco seed, or a plant germinated therefrom, where the at least one progeny tobacco seed or plant germinated therefrom comprises the recombinant DNA construct. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence at least 80% identical to a nucleic acid sequence selected from the group consisting of SEQ ID NOs: 1 to 20, where the recombinant DNA construct is not present in a control tobacco plant of the first tobacco variety; and (b) selecting for at least one progeny tobacco seed, or a plant germinated therefrom, where the at least one progeny tobacco seed or plant germinated therefrom comprises the recombinant DNA construct. In another aspect, this disclosure provides a method for producing a modified tobacco plant comprising: (a) crossing at least one tobacco plant of a first tobacco variety with at least one tobacco plant of a second tobacco variety to produce at least one progeny tobacco seed, where the at least one tobacco plant of the first tobacco variety comprises a recombinant DNA construct, where the recombinant DNA construct comprises a heterologous promoter operably linked to a nucleic acid sequence at least 80% identical to a nucleic acid sequence selected from the group consisting of SEQ ID NOS: 1 to 20, where the recombinant DNA construct is not present in a control tobacco plant of the first tobacco variety; and (b) selecting for at least one progeny tobacco seed, or a plant germinated therefrom, where the at least one progeny tobacco seed or plant germinated therefrom comprises the recombinant DNA construct. In an aspect, in any of the foregoing methods, the first tobacco variety and second tobacco variety are the same tobacco variety. In another aspect, in any of the foregoing methods, the first tobacco variety and second tobacco variety are two different tobacco varieties. In a further aspect, a recombinant DNA construct of each of the above aspects comprises a promoter sequence selected from the group consisting of SEQ ID NOs: 62 to 90.

As used herein, the term “crossing” refers to the deliberate mating of two plants. In an aspect, crossing comprises pollination and/or fertilization of a first tobacco plant by a second tobacco plant. The two tobacco plants being crossed can be distantly related, closely related, or identical. In an aspect, the two tobacco plants being crossed are both modified tobacco plants. In an aspect, the two tobacco plants being crossed are of the same tobacco variety. In an aspect, the two tobacco plants being crossed are of two different tobacco varieties. In an aspect, one of the two tobacco plants being crossed is male sterile. In an aspect, one of the two tobacco plants being crossed is female sterile. In an aspect, at least one of the two tobacco plants being crossed is a hybrid tobacco plant. In an aspect, at least one of the two tobacco plants being crossed is a modified tobacco plant.

In an aspect, a tobacco plant or variety provided herein is male sterile. In another aspect, a tobacco plant or variety provided herein is cytoplasmic male sterile (CMS). In an aspect, a modified tobacco plant or variety provided herein is male sterile. In another aspect, a modified tobacco plant or variety provided herein is cytoplasmic male sterile (CMS). Male sterile tobacco plants can be produced by any method known in the art. Methods of producing male sterile tobacco are described in Wernsman, E. A., and Rufty, R. C. 1987. Chapter Seventeen. Tobacco. Pages 669-698 In: Cultivar Development. Crop Species. W. H. Fehr (ed.), MacMillan Publishing Go., Inc., New York, N.Y. 761 pp.

In another aspect, a tobacco plant or variety provided herein is female sterile. In another aspect, a modified tobacco plant or variety provided herein is female sterile. As a non-limiting example, female sterile plants can be made by mutating the STIG1 gene. See, for example, Goldman et al. 1994, EMBO Journal 13:2976-2984. In an aspect, a modified tobacco plant provided herein is female sterile.

In an aspect, a modified tobacco plant described herein is a low-alkaloid variety or low-alkaloid plant. As a non-limiting example, LA Burley 21 (LA BU21) is a low-alkaloid variety of tobacco. LA BU21 is produced by incorporation of a low alkaloid gene(s) from a Cuban cigar variety into Burley 21 through several backcrosses. It has approximately 0.2% total alkaloids (dry weight) compared to the about 3.5% (dry weight) of its parent, Burley 21. LA BU21 has a leaf grade well below commercially acceptable standards. LA BU21 also exhibits other unfavorable leaf phenotypes characterized by lower yields, delayed ripening and senescence, higher susceptibility to insect herbivory, and poor end-product quality after curing. LA BU21 leaves further exhibit traits such as higher polyamine content, higher chlorophyll content and more mesophyll cells per unit leaf area. See US2019/0271000 for more characterization of LA BU21 leaf phenotypes.

In an aspect, the present disclosure provides tobacco plants or parts thereof, exhibiting a comparable insect herbivory susceptibility relative to a comparable leaf of a control plant not comprising the same mutation or transgene. In one aspect, a comparable insect herbivory susceptibility is within 20%, 17.5%, 15%, 12.5%, 10%, 7.5%, 5%, 2.5%, or 1% of the level in a comparable leaf of a control plant not comprising the same mutation or transgene. In an aspect, a comparable insect herbivory susceptibility is between 0.5% and 1%, between 1% and 2%, between 2% and 3%, between 3% and 4%, between 4% and 5%, between 5% and 6%, between 6% and 7%, between 7% and 8%, between 8% and 9%, between 9% and 10%, between 11% and 12%, between 12% and 13%, between 13% and 14%, between 14% and 15%, between 15% and 16%, between 16% and 17%, between 17% and 18%, between 18% and 19%, or between 19% and 20% of the level in a comparable leaf of a control plant not comprising the same mutation or transgene. In a further aspect, a comparable insect herbivory susceptibility is between 0.5% and 5%, between 5% and 10%, or between 10% and 20% of the level in a comparable leaf of a control plant not comprising the same mutation or transgene.

Insect herbivory susceptibility level can be assayed by methods known in the art, for example, in an insect feeding assay. In short, a quarter inch layer of 0.7% agar in water is added to a 100 mm Petri dish and allowed to solidify. Leaf discs are cut from the petri dish lid, placed in the plates and pushed gently into the agar. Leaf discs are taken from plants at the 4-5 leaf stage. Discs were taken from lamina only to exclude major midribs. A single disc is taken from each of the four largest leaves of the plant generating 4 replicates per plant. Four plants are sampled for a total of 16 biological replicates test line. A single budworm (e.g., Heliothis sp., Helicoverpa sp.) at the second instar stage is added to the leaf and allowed to feed for 48 hours at ambient temperature. After 48 hours the budworm larvae are weighed and final larval weights are recorded.

In an aspect, a tobacco plant, or part thereof, comprises relative to a control tobacco plant: a first genome modification providing a lower level of nicotine or total alkaloid (e.g., in or targeting one or more NIC1, NIC2, QPT, PMT, ADC, AO, or ODC genes), and a second genome modification providing a comparable level of one or more traits selected from the group consisting of total leaf polyamine level, total root polyamine level, total leaf chlorophyll level, mesophyll cell number per leaf area unit, and leaf epidermal cell size; and where the control plant does not have both the first and the second genome modifications. In one aspect, a tobacco plant, or part thereof, comprises relative to a control tobacco plant: a first genome modification providing a lower level of nicotine or total alkaloid (e.g., in or targeting one or more NIC1, NIC2, QPT, PMT, ADC, AO, or ODC genes), and a second genome modification providing a comparable level of total leaf polyamine level, where the control plant does not have both the first and the second genome modifications. In an aspect, a tobacco plant, or part thereof, comprises relative to a control tobacco plant: a first genome modification providing a lower level of nicotine or total alkaloid (e.g., in or targeting one or more NIC1, NIC2, QPT, PMT, ADC, AO, or ODC genes), and a second genome modification providing a comparable level of total root polyamine level, where the control plant does not have both the first and the second genome modifications. In one aspect, a tobacco plant, or part thereof, comprises relative to a control tobacco plant: a first genome modification providing a lower level of nicotine or total alkaloid (e.g., in or targeting one or more NIC1, NIC2, QPT, PMT, ADC, AO, or ODC genes), and a second genome modification providing a comparable level of total leaf chlorophyll level, where the control plant does not have both the first and the second genome modifications. In an aspect, a tobacco plant, or part thereof, comprises relative to a control tobacco plant: a first genome modification providing a lower level of nicotine or total alkaloid (e.g., in or targeting one or more NIC1, NIC2, QPT, PMT, ADC, AO, or ODC genes), and a second genome modification providing a comparable level of mesophyll cell number per leaf area unit, where the control plant does not have both the first and the second genome modifications. In one aspect, a tobacco plant, or part thereof, comprises relative to a control tobacco plant: a first genome modification providing a lower level of nicotine or total alkaloid (e.g., in or targeting one or more NIC1, NIC2, QPT, PMT, ADC, AO, or ODC genes), and a second genome modification providing a comparable level of leaf epidermal cell size, where the control plant does not have both the first and the second genome modifications. In an aspect, a second genome modification is in or targeting an NIC1, NIC2, QPT, PMT, ADC, AO, or ODC gene.

In an aspect, a first genome modification, a second genome modification, or both comprise a transgene, a mutation, or both. In one aspect, a genome modification, a second genome modification, or both comprise a transgene. In an aspect, a first genome modification, a second genome modification, or both comprise a mutation. In one aspect, a first genome modification, a second genome modification, or both are not transgene-based. In an aspect, a first genome modification, a second genome modification, or both are not mutation-based.

In an aspect, tobacco plants provided herein comprise a reduced amount of total conjugated polyamines in leaves relative to the control tobacco plant. In one aspect, tobacco plants provided herein comprise a reduced amount of total conjugated polyamines in roots relative to the control tobacco plant. Used here, conjugated polyamines include, but are not limited to, soluble conjugated polyamines such as phenolamides containing a backbone consisting of a free polyamine (e.g., putrescine, spermine, and/or spermidine) conjugated with one or more phenylpropanoids such as ferulic, caffeic and courmaric acids. Conjugated polyamines also include, but are not limited to, insoluble conjugated polyamines incorporated into structural polymers such as lignin. In an aspect, tobacco plants provided herein comprise a reduced amount of total free polyamines (e.g., putrescine, spermine, and spermidine) in leaves relative to the control tobacco plant. In one aspect, tobacco plants provided herein comprise a reduced amount of total conjugated polyamines in roots relative to the control tobacco plant. In an aspect, tobacco plants provided herein comprise a reduced amount of total conjugated form of one or more polyamines selected from the group consisting of putrescine, spermidine and spermine in leaves relative to the control tobacco plant. In one aspect, tobacco plants provided herein comprise a reduced amount of total conjugated form of one or more polyamines selected from the group consisting of putrescine, spermidine and spermine in roots relative to the control tobacco plant. In an aspect, tobacco plants provided herein comprise a reduced amount of total free form of one or more polyamines selected from the group consisting of putrescine, spermidine and spermine in leaves relative to the control tobacco plant. In one aspect, tobacco plants provided herein comprise a reduced amount of total conjugated form of one or more polyamines selected from the group consisting of putrescine, spermidine and spermine in roots relative to the control tobacco plant.

In an aspect, a characteristic or a trait of a tobacco plant described here are measured at a time selected from the group consisting of immediately before flowering, at topping, 1 week-post-topping (WPT), 2 WPT, 3 WPT, 4 WPT, 5 WPT, 6 WPT, 7 WPT, 8 WPT, and at harvest. In one aspect, tobacco plants provided herein comprising a first and a second genome modification are capable of producing a leaf with a leaf grade comparable to that of a leaf from a control plant. In an aspect, tobacco plants provided herein comprising a first and a second genome modification have a total leaf yield comparable to a control plant.

In one aspect, a tobacco plant of the present disclosure comprises a nic/mutation, a nic2 mutation, or both.

In an aspect, a modified tobacco plant provided herein further comprises a transgene or mutation directly suppressing the expression or activity of one or more, two or more, three or more, four or more, five or more, six or more, seven or more, eight or more, nine or more, ten or more, eleven or more, twelve or more, thirteen or more, fourteen or more, fifteen or more, sixteen or more, or seventeen or more genes or loci encoding a protein selected from the group consisting of agmatine deiminase (AIC), arginase, diamine oxidase, methylputrescine oxidase (MPO), NADH dehydrogenase, phosphoribosylanthranilate isomerase (PRAI), putrescine N-methyltransferase (PMT), quinolate phosphoribosyl transferase (QPT), S-adenosyl-methionine synthetase (SAMS), A622, NBB1, berberine bridge enzyme-like (BBL), MYC2, Nic1_ERF, Nic2_ERF, ethylene response factor (ERF) transcription factor, nicotine uptake permease (NUP), and MATE transporter. See Dewey and Xie, Molecular genetics of alkaloid biosynthesis in Nicotiana tabacum, Phytochemistry 94 (2013) 10-27.

In an aspect, a modified tobacco plant provided herein further comprises a mutation in an ERF gene of Nic2 locus (Nic2_ERF). In an aspect, a modified tobacco plant provided herein further comprises one or more mutations in one or more, two or more, three or more, four or more, five or more, six or more, seven or more, eight or more, nine or more, or all ten genes selected from the group consisting of ERF32, ERF34, ERF39, ERF189, ERF115, ERF221, ERF104, ERF179, ERF17, and ERF168. See Shoji et al., Plant Cell, (10):3390-409 (2010); and Kajikawa et al., Plant physiol. 2017, 174:999-1011. In one aspect, a modified tobacco plant provided herein further comprises one or more mutations in ERF189, ERF115, or both. In an aspect, a modified tobacco plant provided herein further comprises one or more transgenes targeting and suppressing a gene encoding one or more, two or more, three or more, four or more, five or more, six or more, seven or more, eight or more, nine or more, or all ten proteins selected from the group consisting of ERF32, ERF34, ERF39, ERF189, ERF115, ERF221, ERF104, ERF179, ERF 17, and ERF168.

In an aspect, a modified tobacco plant provided herein further comprises a mutation in an ERF gene of Nic1 locus (Nic1_ERF) (or Nic1b locus as in WO/2019/140297). See also 5 WO/2018/237107. In an aspect, a modified tobacco plant provided herein further comprises one or more mutations in two or more, three or more, four or more, five or more, six or more, or seven or more genes selected from the group consisting of ERF101, ERF110, ERFnew, ERF199, ERF19, ERF130, ERF16, ERF29, ERF210, and ERF91L2. See WO/2019/140297 and Kajikawa et al., Plant physiol. 2017, 174:999-1011. In an aspect, a modified tobacco plant provided herein further comprises one or more mutations in one or more, two or more, three or more, four or more, five or more, or all six genes selected from the group consisting of ERFnew, ERF199, ERF19, ERF29, ERF210, and ERF91L2. In an aspect, a modified tobacco plant provided herein further comprises one or more transgenes targeting and suppressing a gene encoding one or more, two or more, three or more, four or more, five or more, six or more, or seven or more genes selected from the group consisting of ERF101, ERF110, ERFnew, ERF199, ERF19, ERF130, ERF16, ERF29, ERF210, and ERF91L2.

In an aspect, a modified tobacco plant provided herein further comprise a first genetic modification comprising a mutation in a gene or locus encoding a protein selected from the group consisting of aspartate oxidase, agmatine deiminase (AIC), arginase, diamine oxidase, arginine decarboxylase (ADC), methylputrescine oxidase (MPO), NADH dehydrogenase, ornithine decarboxylase (ODC), phosphoribosylanthranilate isomerase (PRAI), putrescine N-methyltransferase (PMT), quinolate phosphoribosyl transferase (QPT), and S-adenosyl-methionine synthetase (SAMS), A622, NBB1, BBL, MYC2, Nic1_ERF, Nic2_ERF, ethylene response factor (ERF) transcription factor, nicotine uptake permease (NUP), and MATE transporter, and further comprises a second genetic modification targeting one or more amino acid sequences at least 80% identical or similar to an amino acid sequence selected from the group consisting of SEQ ID NOs: 117-174. In one aspect, a modified tobacco plant provided herein comprises a first genetic modification comprises a transgene targeting and suppressing a gene or locus encoding a protein selected from the group consisting of aspartate oxidase, agmatine deiminase (AIC), arginase, diamine oxidase, arginine decarboxylase (ADC), methylputrescine oxidase (MPO), NADH dehydrogenase, ornithine decarboxylase (ODC), phosphoribosylanthranilate isomerase (PRAI), putrescine N-methyltransferase (PMT), quinolate phosphoribosyl transferase (QPT), and S-adenosyl-methionine synthetase (SAMS), A622, NBB1, BBL, MYC2, Nic1, Nic2, ethylene response factor (ERF) transcription factor, nicotine uptake permease (NUP), and MATE transporter, and further comprises a second genetic modification targeting one or more amino acid sequences at least 80% identical or similar to an amino acid sequence selected from the group consisting of SEQ ID NOs: 117-174.

### Cured Tobacco/Tobacco Products

In an aspect, this disclosure provides a method comprising preparing a tobacco product using cured tobacco material from a modified tobacco plant, where the modified tobacco plant comprises a non-natural mutation in an endogenous nucleic acid sequence encoding a polypeptide comprising an amino acid sequence at least 80% identical or similar to an amino acid sequence selected from the group consisting of SEQ ID NOs: 21 to 40. In another aspect, this disclosure provides a method comprising preparing a tobacco product using cured tobacco material from a modified tobacco plant, where the modified tobacco plant comprises a non-natural mutation in an endogenous nucleic acid sequence at least 80% identical to a nucleic acid sequence selected from the group consisting of SEQ ID NOs: 41 to 60. In another aspect, this disclosure provides a method comprising preparing a tobacco product using cured tobacco material from a modified tobacco plant, where the modified tobacco plant comprises a non-natural mutation in an endogenous nucleic acid sequence at least 80% identical to a nucleic acid sequence selected from the group consisting of SEQ ID NOs: 41 to 60.

In an aspect, this disclosure provides a method comprising preparing a tobacco product using cured tobacco material from a modified tobacco plant, where the modified tobacco plant comprises a recombinant DNA construct, where the recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40. In another aspect, this disclosure provides a method comprising preparing a tobacco product using cured tobacco material from a modified tobacco plant, where the modified tobacco plant comprises a recombinant DNA construct, and where the recombinant DNA construct comprises a heterologous promoter operably linked to a nucleic acid sequence encoding at least one small RNA molecule capable of binding to and reducing the expression of an endogenous nucleic acid sequence at least 80% identical to a nucleic acid sequence selected from the group consisting of SEQ ID NOs: 1 to 20 and 41 to 60. In another aspect, this disclosure provides a method comprising preparing a tobacco product using cured tobacco material from a modified tobacco plant, where the modified tobacco plant comprises a recombinant DNA construct, and where the recombinant DNA construct comprises a heterologous promoter operably linked to a nucleic acid sequence encoding at least one small RNA molecule capable of binding to and reducing the expression of an endogenous nucleic acid sequence at least 80% identical to a nucleic acid sequence selected from the group consisting of SEQ ID NOs: 1 to 20 and 41 to 60.

In an aspect, this disclosure provides a method comprising preparing a tobacco plant using cured tobacco material from a modified tobacco plant, where the modified tobacco plant comprises a recombinant DNA construct, and where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40. In another aspect, this disclosure provides a method comprising preparing a tobacco plant using cured tobacco material from a modified tobacco plant, where the modified tobacco plant comprises a recombinant DNA construct, and where the recombinant DNA construct comprises a heterologous promoter operably linked to a nucleic acid sequence at least 80% identical to a nucleic acid sequence selected from the group consisting of SEQ ID NOs: 1 to 20 and 41 to 60. In another aspect, this disclosure provides a method comprising preparing a tobacco plant using cured tobacco material from a modified tobacco plant, where the modified tobacco plant comprises a recombinant DNA construct, and where the recombinant DNA construct comprises a heterologous promoter operably linked to a nucleic acid sequence at least 80% identical to a nucleic acid sequence selected from the group consisting of SEQ ID NOs: 1 to 20 and 41 to 60.

“Curing” is the aging process that reduces moisture and brings about the destruction of chlorophyll giving tobacco leaves a golden color and by which starch is converted to sugar. Cured tobacco therefore has a higher reducing sugar content and a lower starch content compared to harvested green leaf. In one aspect, tobacco plants or plant components provided herein can be cured using conventional means, e.g., flue-cured, barn-cured, fire-cured, air-cured or sun-cured. See, for example, Tso (1999, Chapter 1 in Tobacco, Production, Chemistry and Technology, Davis & Nielsen, cds., Blackwell Publishing, Oxford) for a description of different types of curing methods. Cured tobacco is usually aged in a wooden drum (e.g., a hogshead) or cardboard cartons in compressed conditions for several years (e.g., two to five years), at a moisture content ranging from 10% to about 25%. See, U.S. Pat. Nos. 4,516,590 and 5,372,149. Cured and aged tobacco then can be further processed. Further processing includes conditioning the tobacco under vacuum with or without the introduction of steam at various temperatures, pasteurization, and fermentation.

Information regarding the harvesting of burley and dark tobacco varieties can be found in the 2019-2020 Burley and Dark Tobacco Production Guide (December 2018) published by the University of Kentucky, The University of Tennessee, Virginia Tech, and North Carolina State University, which is incorporated herein by reference in its entirety.

In an aspect, this disclosure provides cured tobacco material from any tobacco plant, or part thereof, provided herein. In an aspect, this disclosure provides cured tobacco material from any modified tobacco plant, or part thereof, provided herein.

In an aspect, cured tobacco material comprises tobacco material selected from the group selected from cured leaf material, cured stem material, cured bud material, cured flower material, and cured root material. In another aspect, cured tobacco material comprises cured leaf material, cured stem material, or both. In a further aspect, cured tobacco material comprises cured leaf material. In yet another aspect, cured tobacco material comprises cured stem material.

In an aspect, cured tobacco material comprises flue-cured tobacco material. In another aspect, cured tobacco material comprises air-cured tobacco material. In another aspect, cured tobacco material comprises fire-cured tobacco material. In another aspect, cured tobacco material comprises sun-cured tobacco material. In another aspect, cured tobacco material provided herein is selected from the group consisting of air-cured tobacco material, fire-cured tobacco material, sun-cured tobacco material, and flue-cured tobacco material. In another aspect, cured tobacco material is from a tobacco variety selected from the group consisting of a flue-cured variety, a bright variety, a Burley variety, a Virginia variety, a Maryland variety, a dark variety, an Oriental variety, and a Turkish variety.

In an aspect, cured tobacco leaf provided herein is selected from the group consisting of air-cured tobacco leaf, fire-cured tobacco leaf, sun-cured tobacco leaf, and flue-cured tobacco leaf. In an aspect, cured tobacco leaf is from a tobacco variety selected from the group consisting of a flue-cured variety, a bright variety, a Burley variety, a Virginia variety, a Maryland variety, a dark variety, an Oriental variety, and a Turkish variety.

Fermentation typically is characterized by high initial moisture content, heat generation, and a 10 to 20% loss of dry weight. See, for example, U.S. Pat. Nos. 4,528,993, 4,660,577, 4,848,373, 5,372,149; U.S. Publication No. 2005/0178398; and Tso (1999, Chapter 1 in Tobacco, Production, Chemistry and Technology, Davis & Nielsen, eds., Blackwell Publishing, Oxford). Cured, aged, and fermented tobacco can be further processed (e.g., cut, shredded, expanded, or blended). See, for example, U.S. Pat. Nos. 4,528,993; 4,660,577; and 4,987,907. In an aspect, this disclosure provides fermented tobacco material from any tobacco plant, or part thereof, provided herein. In another aspect, this disclosure provides fermented tobacco material from any modified tobacco plant, or part thereof, provided herein.

Tobacco material obtained from the tobacco lines, varieties or hybrids of the present disclosure can be used to make tobacco products. As used herein, “tobacco product” is defined as any product made or derived from tobacco that is intended for human use or consumption. In an aspect, this disclosure provides a tobacco product comprising plant material from a tobacco plant provided herein. In another aspect, this disclosure provides a tobacco product comprising plant material from a modified tobacco plant provided herein. In another aspect, this disclosure provides a tobacco product comprising cured tobacco material. In another aspect, this disclosure provides a tobacco product comprising fermented tobacco material. In another aspect, this disclosure provides a tobacco product comprising a tobacco blend.

Tobacco products include, without limitation, cigarette products (e.g., cigarettes and bidi cigarettes), cigar products (e.g., cigar wrapping tobacco and cigarillos), pipe tobacco products, products derived from tobacco, tobacco-derived nicotine products, smokeless tobacco products (e.g., moist snuff, dry snuff, and chewing tobacco), films, chewables, tabs, shaped parts, gels, consumable units, insoluble matrices, hollow shapes, reconstituted tobacco, expanded tobacco, and the like. See, e.g., U.S. Patent Publication No. US 2006/0191548.

As used herein, “cigarette” refers a tobacco product having a “rod” and “filler”. The cigarette “rod” includes the cigarette paper, filter, plug wrap (used to contain filtration materials), tipping paper that holds the cigarette paper (including the filler) to the filter, and all glues that hold these components together. The “filler” includes (1) all tobaccos, including but not limited to reconstituted and expanded tobacco, (2) non-tobacco substitutes (including but not limited to herbs, non-tobacco plant materials and other spices that may accompany tobaccos rolled within the cigarette paper), (3) casings, (4) flavorings, and (5) all other additives (that are mixed into tobaccos and substitutes and rolled into the cigarette).

In an aspect, a tobacco product comprises reconstituted tobacco. In another aspect, this disclosure provides reconstituted tobacco comprising cured tobacco material. As used herein, “reconstituted tobacco” refers to a part of tobacco filler made from tobacco dust and other tobacco scrap material, processed into sheet form and cut into strips to resemble tobacco. In addition to the cost savings, reconstituted tobacco is very important for its contribution to cigarette taste from processing flavor development using reactions between ammonia and sugars.

In an aspect, a tobacco product comprises expanded tobacco. As used herein, “expanded tobacco” refers to a part of tobacco filler which is processed through expansion of suitable gases so that the tobacco is “puffed” resulting in reduced density and greater filling capacity. It reduces the weight of tobacco used in cigarettes.

Tobacco products derived from plants of the present disclosure also include cigarettes and other smoking articles, particularly those smoking articles including filter elements, where the rod of smokable material includes cured tobacco within a tobacco blend. In an aspect, a tobacco product of the present disclosure is selected from the group consisting of a cigarillo, a non-ventilated recess filter cigarette, a vented recess filter cigarette, a cigar, snuff, pipe tobacco, cigar tobacco, cigarette tobacco, chewing tobacco, leaf tobacco, hookah tobacco, shredded tobacco, and cut tobacco. In another aspect, a tobacco product of the present disclosure is selected from the group consisting of a cigarette, a heated tobacco product, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, pouched chewing tobacco product, gum, a tablet, a lozenge, and a dissolving strip.

In another aspect, a tobacco product of the present disclosure is a smokeless tobacco product. In an aspect, a smokeless tobacco product is selected from the group consisting of loose leaf chewing tobacco, plug chewing tobacco, moist snuff, nasal snuff, dry snuff, and snus.

Smokeless tobacco products are not combusted and include, but not limited to, chewing tobacco, moist smokeless tobacco, snus, and dry snuff. Chewing tobacco is coarsely divided tobacco leaf that is typically packaged in a large pouch-like package and used in a plug or twist. Moist smokeless tobacco is a moist, more finely divided tobacco that is provided in loose form or in pouch form and is typically packaged in round cans and used as a pinch or in a pouch placed between an adult tobacco consumer's check and gum. Snus is a heat-treated smokeless tobacco. Dry snuff is finely ground tobacco that is placed in the mouth or used nasally.

In yet another aspect, a tobacco product of the present disclosure is selected from the group consisting of an electronically heated cigarette, an e-cigarette, an electronic vaporing device.

In an aspect, a tobacco product is a heated tobacco product. As used herein, a “heated tobacco product” is a tobacco product that is heated to a lower temperature (e.g., about 600° C.) than a conventional cigarette. Heated tobacco products generate an aerosol or smoke that can be inhaled upon heating to an appropriate temperature. Heated tobacco products are also referred to as “heat-not-burn” tobacco products. Heated tobacco products are often used in electronic devices that use a battery to heat the heated tobacco product or a lit carbon ember that heats the heated tobacco product. In an aspect, a heated tobacco product is a film. In an aspect, a heated tobacco product does not combust when heated.

In an aspect, a tobacco product comprises a reconstituted tobacco film. In an aspect, a tobacco product comprises a humectant. In an aspect, a humectant is glycerin.

In an aspect, a tobacco product of the present disclosure can be a blended tobacco product.

In an aspect, a tobacco product of the present disclosure can comprise a freeze dried, uncured tobacco leaf or part thereof. In a further aspect, freeze dried, uncured tobacco can be blended with cured tobacco as part of a tobacco product of the present disclosure.

In another aspect, this disclosure provides a tobacco blend comprising cured tobacco material. A tobacco blend can comprise any combination of cured tobacco, uncured tobacco, fermented tobacco, unfermented tobacco, expanded tobacco, and reconstituted tobacco.

In an aspect, a tobacco blend comprises at least 5% cured tobacco by weight. In an aspect, a tobacco blend comprises at least 10% cured tobacco by weight. In an aspect, a tobacco blend comprises at least 15% cured tobacco by weight. In an aspect, a tobacco blend comprises at least 20% cured tobacco by weight. In an aspect, a tobacco blend comprises at least 25% cured tobacco by weight. In an aspect, a tobacco blend comprises at least 30% cured tobacco by weight. In an aspect, a tobacco blend comprises at least 35% cured tobacco by weight. In an aspect, a tobacco blend comprises at least 40% cured tobacco by weight. In an aspect, a tobacco blend comprises at least 45% cured tobacco by weight. In an aspect, a tobacco blend comprises at least 50% cured tobacco by weight. In an aspect, a tobacco blend comprises at least 55% cured tobacco by weight. In an aspect, a tobacco blend comprises at least 60% cured tobacco by weight. In an aspect, a tobacco blend comprises at least 65% cured tobacco by weight. In an aspect, a tobacco blend comprises at least 70% cured tobacco by weight. In an aspect, a tobacco blend comprises at least 75% cured tobacco by weight. In an aspect, a tobacco blend comprises at least 80% cured tobacco by weight. In an aspect, a tobacco blend comprises at least 85% cured tobacco by weight. In an aspect, a tobacco blend comprises at least 90% cured tobacco by weight. In an aspect, a tobacco blend comprises at least 95% cured tobacco by weight.

In an aspect, a tobacco blend comprises at least 5% cured tobacco by volume. In an aspect, a tobacco blend comprises at least 10% cured tobacco by volume. In an aspect, a tobacco blend comprises at least 15% cured tobacco by volume. In an aspect, a tobacco blend comprises at least 20% cured tobacco by volume. In an aspect, a tobacco blend comprises at least 25% cured tobacco by volume. In an aspect, a tobacco blend comprises at least 30% cured tobacco by volume. In an aspect, a tobacco blend comprises at least 35% cured tobacco by volume. In an aspect, a tobacco blend comprises at least 40% cured tobacco by volume. In an aspect, a tobacco blend comprises at least 45% cured tobacco by volume. In an aspect, a tobacco blend comprises at least 50% cured tobacco by volume. In an aspect, a tobacco blend comprises at least 55% cured tobacco by volume. In an aspect, a tobacco blend comprises at least 60% cured tobacco by volume. In an aspect, a tobacco blend comprises at least 65% cured tobacco by volume. In an aspect, a tobacco blend comprises at least 70% cured tobacco by volume. In an aspect, a tobacco blend comprises at least 75% cured tobacco by volume. In an aspect, a tobacco blend comprises at least 80% cured tobacco by volume. In an aspect, a tobacco blend comprises at least 85% cured tobacco by volume. In an aspect, a tobacco blend comprises at least 90% cured tobacco by volume. In an aspect, a tobacco blend comprises at least 95% cured tobacco by volume.

### Transformation

In an aspect, this disclosure provides a method of producing a modified tobacco plant comprising: (a) inducing a non-natural mutation in at least one tobacco cell in an endogenous nucleic acid sequence encoding a polypeptide comprising an amino acid sequence at least 80% identical or similar to an amino acid sequence selected from the group consisting of SEQ ID NOs: 21 to 40; (b) selecting at least one tobacco cell comprising the non-natural mutation from step (a); and (c) regenerating at least one modified tobacco plant from the at least one tobacco cell selected in step (b). In another aspect, this disclosure provides a method of producing a modified tobacco plant comprising: (a) inducing a non-natural mutation in at least one tobacco cell in an endogenous nucleic acid sequence at least 80% identical to a nucleic acid sequence selected from the group consisting of SEQ ID NOs: 41 to 60; (b) selecting at least one tobacco cell comprising the non-natural mutation from step (a); and (c) regenerating at least one modified tobacco plant from the at least one tobacco cell selected in step (b). In another aspect, this disclosure provides a method of producing a modified tobacco plant comprising: (a) inducing a non-natural mutation in at least one tobacco cell in an endogenous nucleic acid sequence at least 80% identical to a nucleic acid sequence selected from the group consisting of SEQ ID NOs: 1 to 20 and 41 to 60; (b) selecting at least one tobacco cell comprising the non-natural mutation from step (a); and (c) regenerating at least one modified tobacco plant from the at least one tobacco cell selected in step (b). In another aspect, this disclosure provides a method of producing a modified tobacco plant comprising: (a) inducing a non-natural mutation in at least one tobacco cell in an endogenous nucleic acid sequence selected from the group consisting of SEQ ID NOs: 1 to 20 and 41 to 60; (b) selecting at least one tobacco cell comprising the non-natural mutation from step (a); and (c) regenerating at least one modified tobacco plant from the at least one tobacco cell selected in step (b). In an aspect, any of the foregoing methods further comprises (d) growing the modified tobacco plant regenerated in step (c). In another aspect, any of the foregoing methods further comprises: (e) crossing the modified tobacco plant grown in step (d) with a second tobacco plant; and (f) obtaining at least one seed from the crossing in step (c).

In an aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21-40; (b) selecting at least one tobacco cell comprising the recombinant DNA construct; and (c) regenerating at least one modified tobacco plant from the at least one tobacco cell selected in step (b). In another aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid encoding at least one small RNA molecule capable of binding to and reducing the expression of an endogenous nucleic acid sequence at least 80% identical to a nucleic acid sequence selected from the group consisting of SEQ ID NOs: 1 to 20 and 41 to 60; (b) selecting at least one tobacco cell comprising the recombinant DNA construct; and (c) regenerating at least one modified tobacco plant from the at least one tobacco cell selected in step (b). In another aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid encoding at least one small RNA molecule capable of binding to and reducing the expression of an endogenous nucleic acid sequence at least 80% identical to a nucleic acid sequence selected from the group consisting of SEQ ID NOs: 1 to 20 and 41 to 60; (b) selecting at least one tobacco cell comprising the recombinant DNA construct; and (c) regenerating at least one modified tobacco plant from the at least one tobacco cell selected in step (b). In an aspect, any of the foregoing methods further comprises (d) growing the modified tobacco plant regenerated in step (c). In another aspect, any of the foregoing methods further comprises: (c) crossing the modified tobacco plant grown in step (d) with a second tobacco plant; and (f) obtaining at least one seed from the crossing in step (c).

In an aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40; (b) selecting at least one tobacco cell comprising the recombinant DNA construct; and (c) regenerating at least one modified tobacco plant from the at least one tobacco cell selected in step (b). In another aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid sequence at least 80% identical to a nucleic acid sequence selected from the group consisting of SEQ ID NOs: 1 to 20 and 41 to 60; (b) selecting at least one tobacco cell comprising the recombinant DNA construct; and (c) regenerating at least one modified tobacco plant from the at least one tobacco cell selected in step (b). In another aspect, this disclosure provides a method of producing a modified tobacco plant comprising: (a) introducing a recombinant DNA construct to at least one tobacco cell, where the recombinant DNA construct comprises a heterologous promoter operably linked to a nucleic acid sequence at least 80% identical to a nucleic acid sequence selected from the group consisting of SEQ ID NOs: 1 to 20 and 41 to 60; (b) selecting at least one tobacco cell comprising the recombinant DNA construct; and (c) regenerating at least one modified tobacco plant from the at least one tobacco cell selected in step (b). In an aspect, any of the foregoing methods further comprises (d) growing the modified tobacco plant regenerated in step (c). In another aspect, any of the foregoing methods further comprises: (c) crossing the modified tobacco plant grown in step (d) with a second tobacco plant; and (f) obtaining at least one seed from the crossing in step (c).

In an aspect, this disclosure provides a method comprising transforming a tobacco cell with a recombinant DNA construct, where the recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 60. In another aspect, this disclosure provides a method comprising transforming a tobacco cell with a recombinant DNA construct, where the recombinant DNA construct comprises a heterologous promoter operably linked to a nucleic acid sequence encoding at least one small RNA molecule capable of binding to and reducing the expression of an endogenous nucleic acid sequence at least 80% identical a nucleic acid sequence selected from the group consisting of SEQ ID NOs: 1 to 20 and 41 to 60. In another aspect, this disclosure provides a method comprising transforming a tobacco cell with a recombinant DNA construct, where the recombinant DNA construct comprises a heterologous promoter operably linked to a nucleic acid sequence encoding at least one small RNA molecule capable of binding to and reducing the expression of an endogenous nucleic acid sequence at least 80% identical a nucleic acid sequence selected from the group consisting of SEQ ID NOs: 1 to 20 and 41 to 60. In another aspect, the foregoing methods further comprise regenerating a modified tobacco plant from the transformed tobacco cell.

In an aspect, this disclosure provides a method comprising transforming a tobacco cell with a recombinant DNA construct, where the recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40. In another aspect, this disclosure provides a method comprising transforming a tobacco cell with a recombinant DNA construct, where the recombinant DNA construct comprises a heterologous promoter operably linked to a nucleic acid sequence at least 80% identical to a nucleic acid sequence selected from the group consisting of SEQ ID NOs: 1 to 20 and 41 to 60. In another aspect, this disclosure provides a method comprising transforming a tobacco cell with a recombinant DNA construct, where the recombinant DNA construct comprises a heterologous promoter operably linked to a nucleic acid sequence at least 80% identical to a nucleic acid sequence selected from the group consisting of SEQ ID NOs: 1 to 20 and 41 to 60. In another aspect, the foregoing methods further comprise regenerating a modified tobacco plant from the transformed tobacco cell.

In one aspect, plants suitable for use in genetic modification include monocotyledonous and dicotyledonous plants and plant cell systems, including species from one of the following families: Acanthaceae, Alliaceae, Alstroemeriaceae, Amaryllidaceae, Apocynaceae, Arecaceac, Asteraceae, Berberidaceae, Bixaceae, Brassicaceac, Bromeliaceac, Cannabaccac, Caryophyllaccac, Cephalotaxaceac, Chenopodiaceae, Colchicaceae, Cucurbitaceac, Dioscoreaceae, Ephedraceae, Erythroxylaceae, Euphorbiaceae, Fabaceae, Lamiaceae, Linaceac, Lycopodiaceae, Malvaceae, Melanthiaceae, Musaceae, Myrtaceae, Nyssaceae, Papaveraceac, Pinaceae, Plantaginaceae, Poaceae, Rosaceae, Rubiaceae, Salicaceac, Sapindaceae, Solanaceae, Taxaceae, Theaceae, or Vitaceae. In another aspect, Suitable species may include members of the genera Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

Numerous methods for introducing a recombinant DNA construct to a plant cell are known in the art, which can be used according to methods of the present application to produce a transgenic plant cell and plant. Any suitable method or technique for transformation of a plant cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants. Other methods for plant transformation, such as microinjection, electroporation, vacuum infiltration, pressure, sonication, silicon carbide fiber agitation, polyethylene glycol (PEG)-mediated transformation, etc., are also known in the art. Transgenic plants produced by these transformation methods can be chimeric or non-chimeric for the transformation event depending on the methods and explants used.

Methods of transforming plant cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Methods of transforming tea plants, (Camellia ssp.) are also known in the art, for example in Mondal et al., Plant Cell Rep. 20:712-720 (2001). Any appropriate method known to those skilled in the art can be used to transform a tobacco cell with any of the nucleic acid molecules provided herein.

In an aspect, a method of introducing a nucleic acid molecule to a tobacco cell comprises Agrobacterium-mediated transformation. In another aspect, a method of introducing a nucleic acid molecule to a cell comprises PEG-mediated transformation. In another aspect, a method of introducing a nucleic acid molecule to a cell comprises biolistic transformation. In another aspect, a method of introducing a nucleic acid molecule to a cell comprises liposome-mediated transfection (lipofection). In another aspect, a method of introducing a nucleic acid molecule to a cell comprises lentiviral transfection.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of WO 91/17424 and WO 91/16024. Delivery can be to cells (e.g. in vitro or ex vivo administration) or target tissues (e.g. in vivo administration).

Any plant cell from which a fertile plant can be regenerated is contemplated as a useful recipient cell for practice of this disclosure. Any tobacco cell from which a fertile tobacco plant can be regenerated is contemplated as a useful recipient cell for practice of this disclosure. Any Cannabis cell from which a fertile Cannabis plant can be regenerated is contemplated as a useful recipient cell for practice of this disclosure. Any Camellia cell from which a fertile Camellia plant can be regenerated is contemplated as a useful recipient cell for practice of this disclosure. In an aspect, a recombinant DNA construct is introduced to a tobacco cell. In an aspect, a recombinant DNA construct is introduced to a tobacco protoplast cell. In another aspect, a recombinant DNA construct is introduced to a tobacco callus cell. In an aspect, a recombinant DNA construct is introduced to a tobacco cell selected from the group consisting of a seed cell, a fruit cell, a leaf cell, a cotyledon cell, a hypocotyl cell, a meristem cell, an embryo cell, an endosperm cell, a root cell, a shoot cell, a stem cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, and a phloem cell.

Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U. S. Patent Application Publication 2004/0216189, all of which are incorporated herein by reference.

### Leaf Grade

As used herein, “USDA leaf grade index” refers to a subdivision of a leaf type according to group, quality, and color. In one aspect, a USDA grade quality score is quantified as a 0-100 numerical representation of the grade as determined by a certified tobacco leaf grader, and is a weighted average of all stalk positions. A higher grade index indicates higher quality. As used below, a “point” refers to each whole number numerical representation of the USDA leaf grade score. For example, the difference between a USDA leaf grade index score of 90 and a score of 85 is 5 points.

Alternatively, leaf grade can be determined via hyper-spectral imaging. See e.g., WO 2011/027315 (published on Mar. 10, 2011, and incorporated by reference in its entirety).

As used herein, a “certified tobacco leaf grader” refers to a person trained to grade tobacco leaves in accordance with USDA Official Standards Grades defined by the United States Department of Agriculture (USDA), Agricultural Marketing Systems as published in 7 CFR § 29. A USDA leaf grade index score may be assigned by an employee, a past employee, or a person otherwise trained to grade tobacco leaves in accordance with USDA Official Standards Grades. Exemplary steps of a standard operation for commercial inspection service begins with a grower delivering tobacco to market after which the tobacco is arranged on flat baskets as lots. Each lot is weighed and then inspected by a certified tobacco leaf grader. After examination, the grader assigns a grade to each lot which becomes a certificate of grade indicating group, quality, and color. The steps for grading experimental lots is similar; however, experimental tobacco is not taken to market or otherwise used for commercial purposes.

Tobacco grades are evaluated based on factors including, but not limited to, the leaf stalk position, leaf size, leaf color, leaf uniformity and integrity, ripeness, texture, elasticity, sheen (related with the intensity and the depth of coloration of the leaf as well as the shine), hygroscopicity (the faculty of the tobacco leaves to absorb and to retain the ambient moisture), and green nuance or cast. Leaf grade can be determined, for example, using an Official Standard Grade published by the Agricultural Marketing Service of the US Department of Agriculture (7 U.S.C. § 511). See, e.g., Official Standard Grades for Burley Tobacco (U.S. Type 31 and Foreign Type 93), effective Nov. 5, 1990 (55 F.R. 40645); Official Standard Grades for Flue-Cured Tobacco (U.S. Types 11, 12, 13, 14 and Foreign Type 92), effective Mar. 27, 1989 (54 F.R. 7925); Official Standard Grades for Pennsylvania Seedleaf Tobacco (U.S. Type 41), effective Jan. 8, 1965 (29 F.R. 16854); Official Standard Grades for Ohio Cigar-Leaf Tobacco (U.S. Types 42, 43, and 44), effective Dec. 8, 1963 (28 F.R. 11719 and 28 F.R. 11926); Official Standard Grades for Wisconsin Cigar-Binder Tobacco (U.S. Types 54 and 55), effective Nov. 20, 1969 (34 F.R. 17061); Official Standard Grades for Wisconsin Cigar-Binder Tobacco (U.S. Types 54 and 55), effective Nov. 20, 1969 (34 F.R. 17061); Official Standard Grades for Georgia and Florida Shade-Grown Cigar-Wrapper Tobacco (U.S. Type 62), Effective April 1971. A USDA grade index value can be determined according to an industry accepted grade index. See, e.g., Bowman et al, Tobacco Science, 32:39-40 (1988); Legacy Tobacco Document Library (Bates Document #523267826-523267833, Jul. 1, 1988, Memorandum on the Proposed Burley Tobacco Grade Index); and Miller et al., 1990, Tobacco Intern., 192:55-57 (all foregoing references are incorporated by reference in their entirety).

Unless specified otherwise, measurements of leaf grade index values, alkaloid, or nicotine levels mentioned herein for a tobacco plant, variety, cultivar, or line refer to average measurements, including, for example, an average of multiple leaves of a single plant or an average measurement from a population of tobacco plants from a single variety, cultivar, or line. A population of tobacco plants or a collection of tobacco leaves for determining an average measurement (e.g., leaf grading or alkaloid or nicotine level) can be of any size, for example, 2, 5, 10, 15, 20, 25, 30, 35, 40, 50, or more. A population of at least 5 or more tobacco plants is used to determine standard deviation. Industry-accepted standard protocols are followed for determining average measurements or grade index values.

As used herein, “USDA graded leaf group”, “leaf group”, or “group” is a division of a type covering closely related grades based on certain characteristics which are related to stalk position, body, or general quality. Group is the first factor of a USDA grade. Group determination is part of the grading procedure and is assigned by a certified tobacco leaf grader.

In an aspect, a modified tobacco plant comprising a non-natural mutation comprises a comparable or higher USDA leaf grade index as compared to a control tobacco plant lacking the non-natural mutation when grown under similar growth conditions. In another aspect, a modified tobacco plant comprising a recombinant DNA construct comprises a comparable or higher USDA leaf grade index as compared to a control tobacco plant lacking the recombinant DNA construct when grown under similar growth conditions.

As used herein, a “comparable” USDA leaf grade index refers to within 15%. For example, if a control plant has a USDA leaf grade index of 100, a comparable USDA leaf grade index would be between 85 and 100.

In an aspect, a modified tobacco plant comprises a USDA leaf grade index at least 1% higher than the USDA leaf grade index of a control tobacco plant when grown under similar growth conditions. In another aspect, a modified tobacco plant comprises a USDA leaf grade index at least 5% higher than the USDA leaf grade index of a control tobacco plant when grown under similar growth conditions. In another aspect, a modified tobacco plant comprises a USDA leaf grade index at least 10% higher than the USDA leaf grade index of a control tobacco plant when grown under similar growth conditions. In another aspect, a modified tobacco plant comprises a USDA leaf grade index at least 20% higher than the USDA leaf grade index of a control tobacco plant when grown under similar growth conditions. In another aspect, a modified tobacco plant comprises a USDA leaf grade index at least 30% higher than the USDA leaf grade index of a control tobacco plant when grown under similar growth conditions. In another aspect, a modified tobacco plant comprises a USDA leaf grade index at least 40% higher than the USDA leaf grade index of a control tobacco plant when grown under similar growth conditions. In another aspect, a modified tobacco plant comprises a USDA leaf grade index at least 50% higher than the USDA leaf grade index of a control tobacco plant when grown under similar growth conditions. In another aspect, a modified tobacco plant comprises a USDA leaf grade index at least 75% higher than the USDA aspect, a modified tobacco plant comprises a USDA leaf grade index at least 100% higher than the USDA leaf grade index of a control tobacco plant when grown under similar growth conditions.

In an aspect, a modified tobacco plant comprises a USDA leaf grade index between 1% higher and 100% higher than the USDA leaf grade index of a control tobacco plant when grown under similar growth conditions. In an aspect, a modified tobacco plant comprises a USDA leaf grade index between 1% higher and 75% higher than the USDA leaf grade index of a control tobacco plant when grown under similar growth conditions. In an aspect, a modified tobacco plant comprises a USDA leaf grade index between 1% higher and 50% higher than the USDA leaf grade index of a control tobacco plant when grown under similar growth conditions. In an aspect, a modified tobacco plant comprises a USDA leaf grade index between 1% higher and 40% higher than the USDA leaf grade index of a control tobacco plant when grown under similar growth conditions. In an aspect, a modified tobacco plant comprises a USDA leaf grade index between 1% higher and 30% higher than the USDA leaf grade index of a control tobacco plant when grown under similar growth conditions. In an aspect, a modified tobacco plant comprises a USDA leaf grade index between 1% higher and 20% higher than the USDA leaf grade index of a control tobacco plant when grown under similar growth conditions. In an aspect, a modified tobacco plant comprises a USDA leaf grade index between 1% higher and 10% higher than the USDA leaf grade index of a control tobacco plant when grown under similar growth conditions. In an aspect, a modified tobacco plant comprises a USDA leaf grade index between 10% higher and 75% higher than the USDA leaf grade index of a control tobacco plant when grown under similar growth conditions. In an aspect, a modified tobacco plant comprises a USDA leaf grade index between 10% higher and 50% higher than the USDA leaf grade index of a control tobacco plant when grown under similar growth conditions. In an aspect, a modified tobacco plant comprises a USDA leaf grade index between 1% higher and 30% higher than the USDA leaf grade index of a control tobacco plant when grown under similar growth conditions.

In an aspect, a modified tobacco plant comprises a USDA leaf grade index at least 1 point higher than the USDA leaf grade index of a control tobacco plant when grown under similar growth conditions. In another aspect, a modified tobacco plant comprises a USDA leaf grade index at least 2 points higher than the USDA leaf grade index of a control tobacco plant when grown under similar growth conditions. In another aspect, a modified tobacco plant comprises a USDA leaf grade index at least 3 points higher than the USDA leaf grade index of a control tobacco plant when grown under similar growth conditions. In another aspect, a modified tobacco plant comprises a USDA leaf grade index at least 4 points higher than the USDA leaf grade index of a control tobacco plant when grown under similar growth conditions. In another aspect, a modified tobacco plant comprises a USDA leaf grade index at least 5 points higher than the USDA leaf grade index of a control tobacco plant when grown under similar growth conditions. In another aspect, a modified tobacco plant comprises a USDA leaf grade index at least 6 points higher than the USDA leaf grade index of a control tobacco plant when grown under similar growth conditions. In another aspect, a modified tobacco plant comprises a USDA leaf grade index at least 7 points higher than the USDA leaf grade index of a control tobacco plant when grown under similar growth conditions. In another aspect, a modified tobacco plant comprises a USDA leaf grade index at least 8 points higher than the USDA leaf grade index of a control tobacco plant when grown under similar growth conditions. In another aspect, a modified tobacco plant comprises a USDA leaf grade index at least 9 points higher than the USDA leaf grade index of a control tobacco plant when grown under similar growth conditions. In another aspect, a modified tobacco plant comprises a USDA leaf grade index at least 10 points higher than the USDA leaf grade index of a control tobacco plant when grown under similar growth conditions. In another aspect, a modified tobacco plant comprises a USDA leaf grade index at least 11 points higher than the USDA leaf grade index of a control tobacco plant when grown under similar growth conditions. In another aspect, a modified tobacco plant comprises a USDA leaf grade index at least 12 points higher than the USDA leaf grade index of a control tobacco plant when grown under similar growth conditions. In another aspect, a modified tobacco plant comprises a USDA leaf grade index at least 13 points higher than the USDA leaf grade index of a control tobacco plant when grown under similar growth conditions. In another aspect, a modified tobacco plant comprises a USDA leaf grade index at least 14 points higher than the USDA leaf grade index of a control tobacco plant when grown under similar growth conditions. In another aspect, a modified tobacco plant comprises a USDA leaf grade index at least 15 points higher than the USDA leaf grade index of a control tobacco plant when grown under similar growth conditions. In another aspect, a modified tobacco plant comprises a USDA leaf grade index at least 16 points higher than the USDA leaf grade index of a control tobacco plant when grown under similar growth conditions. In another aspect, a modified tobacco plant comprises a USDA leaf grade index at least 17 points higher than the USDA leaf grade index of a control tobacco plant when grown under similar growth conditions. In another aspect, a modified tobacco plant comprises a USDA leaf grade index at least 18 points higher than the USDA leaf grade index of a control tobacco plant when grown under similar growth conditions. In another aspect, a modified tobacco plant comprises a USDA leaf grade index at least 19 points higher than the USDA leaf grade index of a control tobacco plant when grown under similar growth conditions. In another aspect, a modified tobacco plant comprises a USDA leaf grade index at least 20 points higher than the USDA leaf grade index of a control tobacco plant when grown under similar growth conditions. In another aspect, a modified tobacco plant comprises a USDA leaf grade index at least 25 points higher than the USDA leaf grade index of a control tobacco plant when grown under similar growth conditions. In another aspect, a modified tobacco plant comprises a USDA leaf grade index at least 30 points higher than the USDA leaf grade index of a control tobacco plant when grown under similar growth conditions. In another aspect, a modified tobacco plant comprises a USDA leaf grade index at least 35 points higher than the USDA leaf grade index of a control tobacco plant when grown under similar growth conditions. In another aspect, a modified tobacco plant comprises a USDA leaf grade index at least 40 points higher than the USDA leaf grade index of a control tobacco plant when grown under similar growth conditions. In another aspect, a modified tobacco plant comprises a USDA leaf grade index at least 50 points higher than the USDA leaf grade index of a control tobacco plant when grown under similar growth conditions.

In an aspect, a modified tobacco plant comprises a USDA leaf grade index between 1 point higher and 100 points higher than the USDA leaf grade index of a control tobacco plant when grown under similar growth conditions. In another aspect, a modified tobacco plant comprises a USDA leaf grade index between 1 point higher and 75 points higher than the USDA leaf grade index of a control tobacco plant when grown under similar growth conditions. In another aspect, a modified tobacco plant comprises a USDA leaf grade index between 1 point higher and 50 points higher than the USDA leaf grade index of a control tobacco plant when grown under similar growth conditions. In another aspect, a modified tobacco plant comprises a USDA leaf grade index between 1 point higher and 25 points higher than the USDA leaf grade index of a control tobacco plant when grown under similar growth conditions. In another aspect, a modified tobacco plant comprises a USDA leaf grade index between 1 point higher and 10 points higher than the USDA aspect, a modified tobacco plant comprises a USDA leaf grade index between 1 point higher and 5 points higher than the USDA leaf grade index of a control tobacco plant when grown under similar growth conditions. In another aspect, a modified tobacco plant comprises a USDA leaf grade index between 10 points higher and 50 points higher than the USDA leaf grade index of a control tobacco plant when grown under similar growth conditions. In another aspect, a modified tobacco plant comprises a USDA leaf grade index between 10 points higher and 25 points higher than the USDA leaf grade index of a control tobacco plant when grown under similar growth conditions.

In an aspect, a comparable USDA leaf grade index between a modified tobacco plant and a control tobacco plant grown under similar growth conditions is within 1 point. In another aspect, a comparable USDA leaf grade index between a modified tobacco plant and a control tobacco plant grown under similar growth conditions is within 2 points. In another aspect, a comparable USDA leaf grade index between a modified tobacco plant and a control tobacco plant grown under similar growth conditions is within 3 points. In another aspect, a comparable USDA leaf grade index between a modified tobacco plant and a control tobacco plant grown under similar growth conditions is within 4 points. In another aspect, a comparable USDA leaf grade index between a modified tobacco plant and a control tobacco plant grown under similar growth conditions is within 5 points. In another aspect, a comparable USDA leaf grade index between a modified tobacco plant and a control tobacco plant grown under similar growth conditions is within 6 points. In another aspect, a comparable USDA leaf grade index between a modified tobacco plant and a control tobacco plant grown under similar growth conditions is within 7 points. In another aspect, a comparable USDA leaf grade index between a modified tobacco plant and a control tobacco plant grown under similar growth conditions is within 8 points. In another aspect, a comparable USDA leaf grade index between a modified tobacco plant and a control tobacco plant grown under similar growth conditions is within 9 points. In another aspect, a comparable USDA leaf grade index between a modified tobacco plant and a control tobacco plant grown under similar growth conditions is within 10 points. In another aspect, a comparable USDA leaf grade index between a modified tobacco plant and a control tobacco plant grown under similar growth conditions is within 11 points. In another aspect, a comparable USDA leaf grade index between a modified tobacco plant and a control tobacco plant grown under similar growth conditions is within 12 points. In another aspect, a comparable USDA leaf grade index between a modified tobacco plant and a control tobacco plant grown under similar growth conditions is within 13 points. In another aspect, a comparable USDA leaf grade index between a modified tobacco plant and a control tobacco plant grown under similar growth conditions is within 14 points. In another aspect, a comparable USDA leaf grade index between a modified tobacco plant and a control tobacco plant grown under similar growth conditions is within 15 points.

### Aroma/Flavor

Unless otherwise noted, when comparing trichome number, trichome metabolite profiles, and/or trichome density between a leaf from a modified tobacco plant and a leaf from a control tobacco plant, it shall be understood that the comparison must be performed between leaves of the same age/growth stage. Any age or growth stage of leaves can be compared, so long as they are the same. For example, a V5 leaf of a modified tobacco plant should be compared to a V5 leaf from a control tobacco plant. As a point of clarification, it would be improper to compare trichome number and/or trichome density between a newly emerged leaf with a mature, senescing leaf for the purposes of this disclosure.

Density of trichomes can be measured by counting the number of trichomes present in a unit of area. Determination of trichome density can be performed using tools including, but not limited to, a stereomicroscope, an image capture device (e.g., a camera), and computer software. See, for example, Mirnezami et al., Appl Plant Sci., 8(7):e11375 (2020). The number of trichomes on a leaf can be measured by counting the total number of trichomes present on an entire leaf, including all surfaces of its petiole and blade (lamina), unless otherwise noted. Determination of the number of trichomes on a leaf can be performed using tools including, but not limited to, a stereomicroscope, an image capture device (e.g., a camera), and computer software. See, for example, Mirnezami et al., Appl Plant Sci., 8(7):e11375 (2020).

In an aspect, at least one leaf of a modified plant comprises a greater average trichome density as compared to a leaf of a control plant grown under comparable conditions. In an aspect, at least one leaf of a modified plant comprises a greater average trichome density on the abaxial side of the leaf as compared to the abaxial side of a leaf of a control plant grown under comparable conditions. In an aspect, at least one leaf of a modified plant comprises a greater average trichome density on the adaxial side of the leaf as compared to the adaxial side of a leaf of a control plant grown under comparable conditions.

In an aspect, at least two leaves of a modified plant comprise a greater average trichome density as compared to two leaves of a control plant grown under comparable conditions. In an aspect, a majority of the leaves of a modified plant comprise a greater average trichome density as compared to the same number of leaves of a control plant grown under comparable conditions. In an aspect, all of the leaves of a modified plant comprise a greater average trichome density as compared to all of the leaves of a control plant grown under comparable conditions.

In an aspect, at least one stem of a modified plant comprises a greater average trichome density as compared to a stem of a control plant grown under comparable conditions. In an aspect, at least one flower of a modified plant comprises a greater average trichome density as compared to a flower of a control plant grown under comparable conditions. In an aspect, at least one root of a modified plant comprises a greater average trichome density as compared to a root of a control plant grown under comparable conditions.

In an aspect, a modified plant comprises a greater average density of glandular trichomes as compared to a control plant. In an aspect, a modified plant comprises a greater average density of non-glandular trichomes as compared to a control plant.

In an aspect, a modified tobacco plant provided herein comprises a similar level of one or more tobacco aroma compounds selected from the group consisting of 3-methylvaleric acid, valeric acid, isovaleric acid, a labdenoid, a cembrenoid, a sugar ester, and a reducing sugar, compared to a control tobacco plant when grown under similar growth conditions. As used herein, a “similar” level refers to within 20%.

As used herein, “tobacco aroma compounds” are compounds associated with the flavor and aroma of tobacco smoke. These compounds include, but are not limited to, 3-methylvaleric acid, valeric acid, isovaleric acid, cembranoid and labdenoid diterpenes, and sugar esters. Concentrations of tobacco aroma compounds can be measured by any known metabolite profiling methods in the art including, without limitation, gas chromatography mass spectrometry (GC-MS), Nuclear Magnetic Resonance Spectroscopy, liquid chromatography-linked mass spectrometry. See The Handbook of Plant Metabolomics, edited by Weckwerth and Kahl, (Wiley-Blackwell) (May 28, 2013).

As used herein, “reducing sugar(s)” are any sugar (monosaccharide or polysaccharide) that has a free or potentially free aldehyde or ketone group. Glucose and fructose act as nicotine buffers in cigarette smoke by reducing smoke pH and effectively reducing the amount of “free” unprotonated nicotine. Reducing sugars balances smoke flavor, for example, by modifying the sensory impact of nicotine and other tobacco alkaloids. An inverse relationship between sugar content and alkaloid content has been reported across tobacco varieties, within the same variety, and within the same plant line caused by planting conditions. Reducing sugar levels can be measured using a segmented-flow colorimetric method developed for analysis of tobacco samples as adapted by Skalar Instrument Co (West Chester, PA) and described by Davis, Tobacco Science 20:139-144 (1976). For example, a sample is dialyzed against a sodium carbonate solution. Copper neocuproin is added to the sample and the solution is heated. The copper neocuproin chelate is reduced in the presence of sugars resulting in a colored complex which is measured at 460 nm.

### Leaf Surface Biochemicals: Terpenes, Alkaloids, Alkaloid Derivatives, Benzenoids, Flavonoids, and Phenylpropanoids

Terpenes are a class of aromatic organic compound produced by plants and some insects. Terpenes are members of a group of volatile unsaturated hydrocarbons found in the essential oils of plants, based on a cyclic molecule having the formula C₁₀H₁₆; and related structures such as, but not limited to, compounds such as sesquiterpene with the formula C₁₅H₂₄, or a simple derivative of such a compound. Terpenes are hydrocarbon molecules that are often used by plants to either directly deter herbivory or to attract predators or parasites of plant herbivores. Non-limiting examples of terpenes include citral, menthol, camphor, salvinorin A, cannabinoids, and curcuminoids.

Several classes of terpenes have been identified in cannabis, including, but not limited to, myrcene, pinene, limonene, caryophyllene, linalool, terpinolene, camphene, terpincol, phellandrene, carene, humulene, pulegone, and geraniol. Myrcene, specifically beta-myrcene, is a monoterpene and the most common terpene produced by cannabis. Pinene is a bicyclic monoterpenoid. Two naturally occurring structural isomers of pinene are known: alpha-pinene and beta-pinene. Limonene is a monocyclic monoterpenoid and one of the two major compounds formed from pinene. Caryophyllene, specifically beta caryophyllene, is a sesquiterpene common to many plants besides cannabis, including cloves, cinnamon leaves, and black pepper. Caryophyllene is the only terpene known to interact with the endocannabinoid system, as beta-caryophyllene is a functional cannabinoid-2 receptor (CB2) agonist. Linalool is a non-cyclic monoterpenoid, a critical precursor in the formation of Vitamin E, and has been isolated from many different plants. Delta-3-carene is a bicyclic monoterpene. Alpha-Terpincol, terpinen-4-ol, and 4-terpineol are three closely related monoterpenoids. Humulene is a sesquiterpene also known as alpha-humulene. Pulegone, a monocyclic monoterpenoid, is a minor component of cannabis.

In an aspect, a terpene is a terpenoid. Terpenoids (also referred to as isoprenoids) are modified terpenes that contain additional functional groups, which often include oxygen. Terpenoids, which can be cyclic or acyclic, vary in size from five-carbon hemiterpenes to long complex molecules containing thousands of isoprene units. Terpenoids are produced through the condensation of five-carbon isoprene units (e.g., dimethylallyl diphosphate (DMAPP) and isopentenyl diphosphate (IPP)), most often by the sequential head-to-tail addition of DMAPP to IPP. The initial cyclization processes are catalyzed by different terpene synthases and enzyme variation leads to variation in monoterpene structure.

Terpenoids are classified according to the number of isoprene units that comprise the parent terpene. A hemiterpenoid comprises one isoprene unit. A monoterpenoid comprises two isoprene units. A sesquiterpenoid comprises three isoprene units. A diterpenoid comprises four isoprene units. A sesterterpenoid comprises five isoprene units. A triterpenoid comprises six isoprene units. A tetraterpenoid comprises eight isoprene units. A polyterpenoid comprises more than eight isoprene units.

In an aspect, a polypeptide is involved in the biosynthesis of at least one terpene. In an aspect, a polypeptide is involved in the biosynthesis of at least one terpenoid. In an aspect, a polypeptide is involved in the biosynthesis of at least one terpenoid selected from the group consisting of a hemiterpenoid, a monoterpenoid, a sesquiterpenoid, a diterpenoid, a sesterterpenoid, a triterpenoid, a tetraterpenoid, and a polyterpenoid. In an aspect, a polypeptide is involved in the biosynthesis of at least one terpene selected from the group consisting of a hemiterpene, a monoterpene, a sesquiterpene, a diterpene, a sesterterpene, a triterpene, a tetraterpene, and a polyterpene.

In an aspect, a tobacco plant of instant disclosure comprises an increase level of one or more terpenes selected from the group consisting of 3-hydroxy-3-methylglutarate, mevalonate, cis-trans-farnesol, carotene diol (1), carotene diol (2), carotene diol (3), beta-cryptoxanthin, and geranyllinalool. In an aspect, a tobacco plant of instant disclosure comprises an increase level of one or more, two or more, three or more, four or more, five or more, six or more, seven or more, or eight or more terpenes selected from the group consisting of 3-hydroxy-3-methylglutarate, mevalonate, cis-trans-farnesol, carotene diol (1), carotene diol (2), carotene diol (3), beta-cryptoxanthin, and geranyllinalool. In an aspect, a tobacco plant of instant disclosure comprises an decreased level of one or more terpenes selected from the group consisting of 3-hydroxy-3-methylglutarate, mevalonate, cis-trans-farnesol, carotene diol (1), carotene diol (2), carotene diol (3), beta-cryptoxanthin, and geranyllinalool. In an aspect, a tobacco plant of instant disclosure comprises an decreased level of one or more, two or more, three or more, four or more, five or more, six or more, seven or more, or eight or more terpenes selected from the group consisting of 3-hydroxy-3-methylglutarate, mevalonate, cis-trans-farnesol, carotene diol (1), carotene diol (2), carotene diol (3), beta-cryptoxanthin, and geranyllinalool.

In an aspect, a tobacco plant of instant disclosure comprises an increase level of one or more alkaloids selected from the group consisting of cotinine, 2-hydroxypyridine, nornicotine, anatabine, nicotine, tyrosol, and salidroside. In an aspect, a tobacco plant of instant disclosure comprises an increase level of one or more, two or more, three or more, four or more, five or more, six or more, or seven or more alkaloids selected from the group consisting of cotinine, 2-hydroxypyridine, nornicotine, anatabine, nicotine, tyrosol, and salidroside. In an aspect, a tobacco plant of instant disclosure comprises an decreased level of one or more alkaloids selected from the group consisting of cotinine, 2-hydroxypyridine, nornicotine, anatabine, nicotine, tyrosol, and salidroside. In an aspect, a tobacco plant of instant disclosure comprises an decreased level of one or more, two or more, three or more, four or more, five or more, six or more, or seven or more alkaloids selected from the group consisting of cotinine, 2-hydroxypyridine, nornicotine, anatabine, nicotine, tyrosol, and salidroside.

In an aspect, a tobacco plant of instant disclosure comprises an increase level of one or more alkaloid derivatives selected from the group consisting of 3-pyridylacetate, nicotine-N-oxide, norcotinine, 6-hydroxynicotine (1), and 6-hydroxynicotine (2). In an aspect, a tobacco plant of instant disclosure comprises an increase level of one or more, two or more, three or more, four or more, or five or more alkaloid derivatives selected from the group consisting of 3-pyridylacetate, nicotine-N-oxide, norcotinine, 6-hydroxynicotine (1), and 6-hydroxynicotine (2). In an aspect, a tobacco plant of instant disclosure comprises an decreased level of one or more alkaloid derivatives selected from the group consisting of 3-pyridylacetate, nicotine-N-oxide, norcotinine, 6-hydroxynicotine (1), and 6-hydroxynicotine (2). In an aspect, a tobacco plant of instant disclosure comprises an decreased level of one or more, two or more, three or more, four or more, or five or more alkaloid derivatives selected from the group consisting of 3-pyridylacetate, nicotine-N-oxide, norcotinine, 6-hydroxynicotine (1), and 6-hydroxynicotine (2).

In an aspect, a tobacco plant of instant disclosure comprises an increase level of one or more Benzenoids selected from the group consisting of 2,4,6-trihydroxybenzoate, 2,6-dihydroxybenzoic acid, 3,4-dihydroxybenzoate, 4-hydroxybenzoate, benzoate, gentisic acid-5-glucoside, hydroquinone beta-glucopyranoside, protocatechuic acid-3-glucoside, salicylate, salicyluric glucuronide, and salicylate-glucoside. In an aspect, a tobacco plant of instant disclosure comprises an increase level of one or more, two or more, three or more, four or more, five or more, six or more, seven or more, eight or more, nine or more, ten or more, or eleven or more Benzenoids selected from the group consisting of 2,4,6-trihydroxybenzoate, 2,6-dihydroxybenzoic acid, 3,4-dihydroxybenzoate, 4-hydroxybenzoate, benzoate, gentisic acid-5-glucoside, hydroquinone beta-glucopyranoside, protocatechuic acid-3-glucoside, salicylate, salicyluric glucuronide, and salicylate-glucoside. In an aspect, a tobacco plant of instant disclosure comprises an decreased level of one or more Benzenoids selected from the group consisting of 2,4,6-trihydroxybenzoate, 2,6-dihydroxybenzoic acid, 3,4-dihydroxybenzoate, 4-hydroxybenzoate, benzoate, gentisic acid-5-glucoside, hydroquinone beta-glucopyranoside, protocatechuic acid-3-glucoside, salicylate, salicyluric glucuronide, and salicylate-glucoside. In an aspect, a tobacco plant of instant disclosure comprises an decreased level of one or more, two or more, three or more, four or more, five or more, six or more, seven or more, eight or more, nine or more, ten or more, or eleven or more Benzenoids selected from the group consisting of 2,4,6-trihydroxybenzoate, 2,6-dihydroxybenzoic acid, 3,4-dihydroxybenzoate, 4-hydroxybenzoate, benzoate, gentisic acid-5-glucoside, hydroquinone beta-glucopyranoside, protocatechuic acid-3-glucoside, salicylate, salicyluric glucuronide, and salicylate-glucoside.

In an aspect, a tobacco plant of instant disclosure comprises an increase level of one or more Flavonoids selected from the group consisting of dihydrokaempferol, eriodictyol, dihydroquercetin, naringenin, quercetin, quercetin 3-glucoside, rutin (quercetin 3-rutinoside), 3-methoxyapigenin, kaempferol 3-O-glucoside/galactoside, and isorhamnetin 3-rutinoside. In an aspect, a tobacco plant of instant disclosure comprises an increase level of one or more, two or more, three or more, four or more, five or more, six or more, seven or more, eight or more, nine or more, or ten or more Flavonoids selected from the group consisting of dihydrokaempferol, eriodictyol, dihydroquercetin, naringenin, quercetin, quercetin 3-glucoside, rutin (quercetin 3-rutinoside), 3-methoxyapigenin, kaempferol 3-O-glucoside/galactoside, and isorhamnetin 3-rutinoside. In an aspect, a tobacco plant of instant disclosure comprises an decreased level of one or more Flavonoids selected from the group consisting of dihydrokaempferol, eriodictyol, dihydroquercetin, naringenin, quercetin, quercetin 3-glucoside, rutin (quercetin 3-rutinoside), 3-methoxyapigenin, kaempferol 3-O-glucoside/galactoside, and isorhamnetin 3-rutinoside. In an aspect, a tobacco plant of instant disclosure comprises an decreased level of one or more, two or more, three or more, four or more, five or more, six or more, seven or more, eight or more, nine or more, or ten or more Flavonoids selected from the group consisting of dihydrokaempferol, eriodictyol, dihydroquercetin, naringenin, quercetin, quercetin 3-glucoside, rutin (quercetin 3-rutinoside), 3-methoxyapigenin, kaempferol 3-O-glucoside/galactoside, and isorhamnetin 3-rutinoside.

As used herein, the term “biosynthesis” refers to the production of a complex molecule (e.g., without being limiting, a terpene or terpenoid) within a plant or plant cell. To be “involved” with the biosynthesis of a compound, a polypeptide can directly interact with a substrate during the biosynthesis of the compound, or the polypeptide can affect the expression (positively or negatively) of a polypeptide that directly interacts with a substrate (e.g., a transcription factor that promotes the expression of an enzyme that converts a substrate to a new form or a repressor that inhibits expression of an enzyme that converts a substrate to a new form).

In an aspect, a polypeptide is involved in the biosynthesis of a hemiterpene. In an aspect, a polypeptide is involved in the biosynthesis of a hemiterpenoid. In an aspect, a polypeptide is involved in the biosynthesis of a monoterpene. In an aspect, a polypeptide is involved in the biosynthesis of a monoterpenoid. In an aspect, a polypeptide is involved in the biosynthesis of a sesquiterpene. In an aspect, a polypeptide is involved in the biosynthesis of a sesquiterpenoid. In an aspect, a polypeptide is involved in the biosynthesis of a diterpene. In an aspect, a polypeptide is involved in the biosynthesis of a diterpenoid. In an aspect, a polypeptide is involved in the biosynthesis of a sesterterpene. In an aspect, a polypeptide is involved in the biosynthesis of a sesterterpenoid. In an aspect, a polypeptide is involved in the biosynthesis of a triterpene. In an aspect, a polypeptide is involved in the biosynthesis of a triterpenoid. In an aspect, a polypeptide is involved in the biosynthesis of a tetraterpene. In an aspect, a polypeptide is involved in the biosynthesis of a polyterpenoid. In an aspect, a polypeptide is involved in the biosynthesis of a monoterpene. In an aspect, a polypeptide is involved in the biosynthesis of a polyterpenoid.

Terpene synthase (TPS) genes can be grouped into seven clades: TPS-a, TPS-b, TPS-c, TPS-d, TPS-c/f, TPS-g, and TPS-h. TPS-a, TPS-b, and TPS-g are restricted to angiosperms, and TPS-d and TPS-h are specific to gymnosperms and the lycopod Selaginella moellendorffii. The TPS-a clade comprises mostly sesquiterpene synthases and diterpene synthases, while the TPS-b and TPS-g clades comprise mostly monoterpene synthases.

### Terpene Synthase-Like Genes

In an aspect, a polypeptide involved in the biosynthesis of at least one terpenoid is a TPS-a clade member. In an aspect, a polypeptide involved in the biosynthesis of at least one terpenoid is a TPS-b clade member. In an aspect, a polypeptide involved in the biosynthesis of at least one terpenoid is a TPS-c clade member. In an aspect, a polypeptide involved in the biosynthesis of at least one terpenoid is a TPS-e/f clade member. In an aspect, a polypeptide involved in the biosynthesis of at least one terpenoid is a TPS-g clade member. In an aspect, a polypeptide involved in the biosynthesis of at least one terpenoid is a member of a clade selected from the group consisting of TPS-a, TPS-b, TPS-c, TPS-e/f, and TPS-g.

In an aspect, a terpene is menthol. In an aspect, a terpene is menthol or a related compound. In an aspect, a terpene is a labdanoid. In an aspect, a terpene is cembratrienediol. In an aspect, a terpene is levopimaric acid. In an aspect, a terpene is L-leucine. In an aspect, a terpene is neophytadiene. In an aspect, a labdanoid is cis-abienol. In an aspect, a labdanoid is labdane-diol. In an aspect, a terpene is selected from the group consisting of menthol or a related compound, a labdanoid, cembratrienediol, levopimaric acid, and L-leucine. In an aspect, a terpene is selected from the group consisting of menthol or a related compound, a labdanoid, cembratrienediol, levopimaric acid, L-leucine, and neophytadiene. In an aspect, a terpene is selected from the group consisting of menthol, a labdanoid, cembratrienediol, levopimaric acid, and L-leucine. In an aspect, a terpene is selected from the group consisting of menthol, a labdanoid, cembratrienediol, levopimaric acid, L-leucine, and neophytadiene.

In an aspect, a terpene is myrcene. In an aspect, a terpene is pinene. In an aspect, a terpene is limonene. In an aspect, a terpene is caryophyllene. In an aspect, a terpene is linalool. In an aspect, a terpene is terpinolene. In an aspect, a terpene is camphene. In an aspect, a terpene is terpineol. In an aspect, a terpene is phellandrene. In an aspect, a terpene is carene. In an aspect, a terpene is humulene. In an aspect, a terpene is pulegone. In an aspect, a terpene is geraniol.

As used herein, “menthol” refers to the organic compound having a chemical formula of C₁₀H₂₀O and the International Union of Pure and Applied Chemistry (IUPAC) name 5-Methyl-2-(propan-2-yl)cyclohexan-1-ol. Menthol is also referred to as “(−)-Menthol.” Related compounds of menthol include, but are not limited to, (+)-Menthol, (+)-Isomenthol, (+)-Neomenthol, (+)-Neoisomenthol, (−)-Isomenthol, (−)-Neomethol, and (−)-Neoisomenthol. In an aspect, a related compound of menthol is selected from the group consisting of (+)-Menthol, (+)-Isomenthol, (+)-Neomenthol, (+)-Neoisomenthol, (−)-Isomenthol, (−)-Neomethol, and (−)-Neoisomenthol.

As used herein, “neophytadiene” refers to the organic compound having a chemical formula of C₂₀H₃₈ and the IUPAC name of 7,11,15-trimethyl-3-methylidenchexadec-1-ene.

As used herein, “cembratrienediol” refers to the organic compound having a chemical formula of C₂₀H₃₄O₂ and the IUPAC name (1R,3R,4Z,8Z,12S,13Z)-1,5,9-trimethyl-12-propan-2-ylcyclotetradeca-4,8,13-triene-1,3-diol. Cembratrienediol is also referred to as “beta-Cembrenediol.”

As used herein, “levopimaric acid” refers to the organic compound having a chemical formula of C₂₀H₃₀O₂ and the IUPAC name (1R,4aR,4bS,10aR)-1,4a-dimethyl-7-propan-2-yl-2,3,4,4b,5,9,10,10a-octahydrophenanthrene-1-carboxylic acid. Levopimaric acid is also referred to as “L-Pimaric acid.” In an aspect, levopimaric acid is involved in diterpene resin acid production.

As used herein, “L-leucine” refers to the amino acid having the chemical formula C₆H₁₂NO₂ and the IUPAC name (2S)-2-amino-4-methylpentanoic acid.

As used herein, a “labdanoid” refers to a terpenoid derivative of the fundamental parent labdane, a diterpene. A labdane has the chemical formula C₂₀H₃₈ and the IUPAC name (1S,2S,4aS,8aR)-2,5,5,8a-tetramethyl-1-[(3R)-3-methylpentyl]-1,2,3,4,4a,6,7,8-octahydronaphthalene.

A non-limiting example of a labdanoid is cis-abienol. As used herein, “cis-abienol” refers to the organic compound having a chemical formula of C₂₀H₃₄O and the IUPAC name (1R,2R,4aS,8aS)-2,5,5,8a-tetramethyl-1-[(2Z)-3-methylpenta-2,4,-dienyl]-3,4,4a,6,7,8-hexahydro-1H-naphthalen-2-ol.

As used herein, “myrcene” refers to the compound having a chemical formula of C₁₀H₁₆ and the IUPAC name 7-Methyl-3-methylene-octa-1,6-diene. Myrcene, also known as beta-myrcene or alpha-myrcene, belongs to the class of organic compounds known as acyclic monoterpenoids. Thus, myrcene is considered to be an isoprenoid lipid molecule.

As used herein, “pinene” refers to the compound having a chemical formula of C₁₀H₁₆ and the IUPAC name (1S,5S)-2,6,6-Trimethylbicyclo[3.1.1]hept-2-ene ((−)-α-Pinene). (+)-a-Pinene, also known as 2-pinene or acintene a, belongs to the class of organic compounds known as bicyclic monoterpenoids. These are monoterpenoids containing exactly 2 rings, which are fused to each other. Alpha-pinene is a pinene that is bicyclo[3.1.1]hept-2-ene substituted by methyl groups at positions 2, 6 and 6 respectively. It has a role as a plant metabolite.

As used herein, “limonene” refers to the compound having a chemical formula of C₁₀H₁₆ and the IUPAC name 1-methyl-4-(1-methylethenyl)-cyclohexene. Limonene, (+/−)- is a racemic mixture of limonene, a natural cyclic monoterpene.

As used herein, “caryophyllene” or “beta caryophyllene” refers to the organic compound having a chemical formula of C₁₅H₂₄ and the IUPAC name (1R,4E,9S)-4,11,11-trimethyl-8-methylidenebicyclo[7.2.0]undec-4-enc. Beta-Caryophyllene is classified as a sesquiterpenoid. (−)-beta-caryophyllene is a beta-caryophyllene in which the stereocenter adjacent to the exocyclic double bond has S configuration, while the remaining stereocenter has R configuration. It is the most commonly occurring form of beta-caryophyllene, occurring in many essential oils.

As used herein, “linalool” refers to the compound having a chemical formula of C₁₀H₁₈O and the IUPAC name 3,7-dimethylocta-1,6-dien-3-ol. Beta-linalool, also known as 3, 7-Dimethyl-1, 6-octadien-3-ol or 2, 6-dimethylocta-2, 7-dien-6-ol, is classified as an acyclic monoterpenoid.

As used herein, “terpinolene” refers to the organic compound having a chemical formula of C₁₀H₁₆ and the IUPAC name 1-methyl-4-propan-2-ylidenecyclohexene. Terpinolene, also known as alpha-terpinolene or isoterpinene, is classified as a menthane monoterpenoid. These are monoterpenoids with a structure based on an o-, m-, or p-menthane backbone.

As used herein, “camphene” refers to the organic compound having a chemical formula of C₁₀H₁₆ and the IUPAC name 2,2-dimethyl-3-methylidenebicyclo[2.2.1]heptane. Camphene is classified as a bicyclic monoterpenoid, containing two rings which are fused to each other. Camphene is a monoterpene with a bicyclic skeleton that is bicyclo[2.2.1]heptane substituted by geminal methyl groups at position 2 and a methylidene group at position 3.

As used herein, “terpineol” refers to the monoterpene alcohol of which there are four isomers: alpha-terpineol, beta-terpineol, gamma-terpineol, and terpinen-4-ol. Beta-terpineol and gamma-terpineol differ only by the location of the double bond. Terpineol is usually a mixture of these isomers with alpha-terpineol as the major constituent. As used herein, “alpha terpineol” refers to the organic compound having a chemical formula of C₁₀H₁₈O and the IUPAC name 2-(4-methylcyclohex-3-en-1-yl)propan-2-ol.

As used herein, “phellandrene” refers to the cyclic monoterpenes “alpha-phellandrene” or “beta-phellandrene.” As used herein, “alpha-phellandrene” refers to the organic compound having a chemical formula of C₁₀H₁₆ and the IUPAC name 2-methyl-5-propan-2-ylcyclohexa-1,3-diene. Alpha-phellandrene is also known as alpha-phellandrene or dihydro-p-cymene. As used herein, “beta-phellandrene” refers to the organic compound having a chemical formula of C₁₀H₁₆ and the IUPAC name 3-methylidene-6-propan-2-ylcyclohexene. Beta-phellandrene is also known as beta-phellandrene or 2-p-menthadiene.

As used herein, “carene” or “alpha carene” refers to the organic compound having a chemical formula of C₁₀H₁₆ and the IUPAC name 3,7,7-trimethylbicyclo[4.1.0]hept-3-ene. Alpha-Carene, also known as 3-carene or delta-car-3-ene, is classified as a bicyclic monoterpenoid.

As used herein, “humulene” refers to the organic compound having a chemical formula of C₁₅H₂₄ and the IUPAC name (1E,4E,8E)-2,6,6,9-tetramethylcycloundeca-1,4,8-triene.

As used herein, “pulegone” refers to the organic compound having a chemical formula of C₁₀H₁₆O and the IUPAC name (5R)-5-methyl-2-propan-2-ylidenecyclohexan-1-one. (+)-Pulegone, also known as D-pulegone or cis-isopulegone, is classified as a menthane monoterpenoid.

As used herein, “geraniol” refers to the organic compound having a chemical formula of C₁₀H₁₈O and the IUPAC name (2E)-3,7-dimethylocta-2,6-dien-1-ol. Geraniol is an acyclic monoterpenoid, and is also referred to as 2E-Geraniol, (e)-geraniol, or geranyl alcohol.

In an aspect, a polypeptide is geranylgeranyl diphosphate synthase. In an aspect, a polypeptide is 8-hydroxy-copalyl diphosphate synthase. In an aspect, a polypeptide is cis-abienol synthase. In an aspect, a polypeptide is cembratrienol synthase 2a. In an aspect, a polypeptide is levopimaradiene synthase. In an aspect, a polypeptide is 2-isopropylmalate synthetase. In an aspect, a polypeptide is 2-oxoisovalerate dehydrogenase. In an aspect, a polypeptide is geranyl diphosphate synthase. In an aspect, a polypeptide is limonene synthase. In an aspect, a polypeptide is limonene 3-hydroxylase. In an aspect, a polypeptide is isopiperitenol dehydrogenase. In an aspect, a polypeptide is pulegone reductase. In an aspect, a polypeptide is menthofuran synthase. In an aspect, a polypeptide is neomenthol dehydrogenase. In an aspect, a polypeptide is geranylgeranyl pyrophosphate synthase. In an aspect, a polypeptide is kolavenyl diphosphate synthase. In an aspect, a polypeptide is solanesyl diphosphate synthase. In an aspect, a polypeptide is terpene synthase. In an aspect, a polypeptide is 2-alkenal reductase. In an aspect, a polypeptide is germacrene synthase. In an aspect, a polypeptide is cytochrome P450. In an aspect, a polypeptide is a carveol dehydrogenase. In an aspect, a polypeptide is a MYB61 transcription factor.

In an aspect, a polypeptide is selected from the group consisting of geranyl diphosphate synthase (GDP), limonene synthase (LS), limonene 3-hydroxylase (L3OH), isopiperitenol dehydrogenase (IPD), pulegone reductase, menthofuran synthase, and GGPPS neomenthol dehydrogenase (NtNMD).

In an aspect, a polypeptide is selected from the group consisting of geranylgeranyl diphosphate synthase (GGPPS2), 8-hydroxy-copalyl diphosphate synthase, and cis-abienol synthase.

In an aspect, a polypeptide is selected from the group consisting of geranylgeranyl diphosphate synthase, 8-hydroxy-copalyl diphosphate synthase, cis-abicnol synthase, cembratrienol synthase 2a, levopimaradiene synthetase, 2-isopropylmalate synthetase, 2-oxoisovalerate dehydrogenase, geranyl diphosphate synthase, limonene synthase, limonene 3-hydroxylase, isopiperitenol dehydrogenase, pulegone reductase, menthofuran synthase, neomenthol dehydrogenase, phylloplanin, and premnaspirodiene oxygenase.

In an aspect, a polypeptide is selected from the group consisting of geranylgeranyl pyrophosphate synthase, kolavenyl diphosphate synthase, solanesyl diphosphate synthase, terpene synthase, limonene synthase, isopiperitenol dehydrogenase, 2-alkenal reductase, germacrene synthase, 2-isopropylmalate synthase, and MYB61.

In an aspect, a polypeptide is selected from the group consisting of geranylgeranyl diphosphate synthase, 8-hydroxy-copalyl diphosphate synthase, cis-abienol synthase, cembratrienol synthase 2a, levopimaradiene synthetase, 2-isopropylmalate synthetase, 2-oxoisovalerate dehydrogenase, geranyl diphosphate synthase, limonene synthase, limonene 3-hydroxylase, isopiperitenol dehydrogenase, pulegone reductase, menthofuran synthase, neomenthol dehydrogenase, phylloplanin, premnaspirodiene oxygenase, geranylgeranyl pyrophosphate synthase, kolavenyl diphosphate synthase, solanesyl diphosphate synthase, terpene synthase, 2-alkenal reductase, germacrene synthase, and MYB61.

In an aspect, a modified plant, seed, or plant part comprising a recombinant nucleic acid provided herein comprises an increased amount of at least one terpene as compared to a control plant, seed, or plant part lacking the recombinant nucleic acid molecule when grown under similar growth conditions. In an aspect, a modified tobacco or Camelia plant, tobacco or Camelia seed, or tobacco plant part comprising a recombinant nucleic acid provided herein comprises an increased amount of at least one terpene as compared to a control tobacco or Camelia plant, tobacco or Camelia seed, or tobacco or Camelia plant part lacking the recombinant nucleic acid molecule when grown under similar growth conditions.

In an aspect, an increased amount of at least one terpene comprises an increase of at least 0.5%. In an aspect, an increased amount of at least one terpene comprises an increase of at least 1%. In an aspect, an increased amount of at least one terpene comprises an increase of at least 2%. In an aspect, an increased amount of at least one terpene comprises an increase of at least 3%. In an aspect, an increased amount of at least one terpene comprises an increase of at least 4%. In an aspect, an increased amount of at least one terpene comprises an increase of at least 5%. In an aspect, an increased amount of at least one terpene comprises an increase of at least 10%. In an aspect, an increased amount of at least one terpene comprises an increase of at least 12.5%. In an aspect, an increased amount of at least one terpene comprises an increase of at least 15%. In an aspect, an increased amount of at least one terpene comprises an increase of at least 17.5%. In an aspect, an increased amount of at least one terpene comprises an increase of at least 20%. In an aspect, an increased amount of at least one terpene comprises an increase of at least 25%. In an aspect, an increased amount of at least one terpene comprises an increase of at least 30%. In an aspect, an increased amount of at least one terpene comprises an increase of at least 40%. In an aspect, an increased amount of at least one terpene comprises an increase of at least 50%. In an aspect, an increased amount of at least one terpene comprises an increase of at least 60%. In an aspect, an increased amount of at least one terpene comprises an increase of at least 70%. In an aspect, an increased amount of at least one terpene comprises an increase of at least 80%. In an aspect, an increased amount of at least one terpene comprises an increase of at least 90%. In an aspect, an increased amount of at least one terpene comprises an increase of at least 100%. In an aspect, an increased amount of at least one terpene comprises an increase of at least 150%. In an aspect, an increased amount of at least one terpene comprises an increase of at least 200%. In an aspect, an increased amount of at least one terpene comprises an increase of at least 250%. In an aspect, an increased amount of at least one terpene comprises an increase of at least 500%.

In an aspect, an increased amount of at least one terpene comprises an increase of between 0.5% and 500%. In an aspect, an increased amount of at least one terpene comprises an increase of between 0.5% and 250%. In an aspect, an increased amount of at least one terpene comprises an increase of between 0.5% and 100%. In an aspect, an increased amount of at least one terpene comprises an increase of between 0.5% and 75%. In an aspect, an increased amount of at least one terpene comprises an increase of between 0.5% and 50%. In an aspect, an increased amount of at least one terpene comprises an increase of between 0.5% and 25%. In an aspect, an increased amount of at least one terpene comprises an increase of between 0.5% and 10%. In an aspect, an increased amount of at least one terpene comprises an increase of between 0.5% and 5%. In an aspect, an increased amount of at least one terpene comprises an increase of between 0.5% and 500%. In an aspect, an increased amount of at least one terpene comprises an increase of between 5% and 250%. In an aspect, an increased amount of at least one terpene comprises an increase of between 5% and 100%. In an aspect, an increased amount of at least one terpene comprises an increase of between 5% and 50%. In an aspect, an increased amount of at least one terpene comprises an increase of between 25% and 500%. In an aspect, an increased amount of at least one terpene comprises an increase of between 25% and 250%. In an aspect, an increased amount of at least one terpene comprises an increase of between 50% and 100%. In an aspect, an increased amount of at least one terpene comprises an increase of between 100% and 500%.

Terpenes may be extracted using any method known in the art, for example, solvent extraction. See, for example, Jiang et al., Curr Protoc Plant Biol., 1:345-358 (2016). Solventless extraction methods are also known in the art. See, for example, Yang and Xie, Science Direct, 162:135-139 (2006).

The amount of terpenes in a plant can be measured using any method known in the art, including, without being limiting, gas chromatography mass spectrometry (GC-MS), Nuclear Magnetic Resonance Spectroscopy, and liquid chromatography-linked mass spectrometry. See The Handbook of Plant Metabolomics, edited by Weckwerth and Kahl, (Wiley-Blackwell) (May 28, 2013). In an aspect, an amount of at least one terpene refers to the concentration of that terpene in the tissue sampled.

In an aspect, a modified tobacco plant or cannabis plant provided herein further comprises a recombinant nucleic acid molecule comprising a nucleic acid sequence encoding a polypeptide operably linked to a heterologous promoter, where the polypeptide causes an increase in average trichome density as compared to a control tobacco plant or cannabis plant lacking the transgene or modification when grown under similar growth conditions. Non-limiting examples of genes that can be overexpressed to increase average trichome density include NIGIS, NtMYB86, NbGIS, cannabis MYB61, cannabis GIS3, and cannabis non-specific lipid-transfer protein 1-like.

### TSNAs

In an aspect, a modified tobacco plant provided further comprises one or more mutations in one or more loci encoding a nicotine demethylase (e.g., CYP82E4, CYP82E5, CYP82E10) that confer reduced amounts of nornicotine (See U.S. Pat. Nos. 8,319,011; 8,124,851; 9,187,759; 9,228,194; 9,228,195; 9,247,706) compared to a control tobacco plant lacking one or more mutations in one or more loci encoding a nicotine demethylase when grown under similar growth conditions. In an aspect, a modified tobacco plant described further comprises reduced nicotine demethylase activity compared to a control plant when grown and cured under similar growth conditions. In a further aspect, a tobacco plant provided further comprises one or more mutations or transgenes providing an elevated level of one or more antioxidants (See U.S. Patent Application Publication No. 2018/0119163 and WO 2018/067985). In another aspect, a tobacco plant provided further comprises one or more mutations or transgenes providing a reduced level of one or more tobacco-specific nitrosamines (TSNAs). In an aspect, a TSNA is selected from the group consisting of N′-nitrosonornicotine (NNN), 4-methylnitrosoamino-1-(3-pyridyl)-1-butanone (NNK), N′-nitrosoanatabine (NAT), and N′-nitrosoanabasine (NAB).

### TSNA Reduction

“Alkaloids” are complex, nitrogen-containing compounds that naturally occur in plants, and have pharmacological effects in humans and animals. “Nicotine” is the primary natural alkaloid in commercialized cigarette tobacco and accounts for about 90 percent of the alkaloid content in Nicotiana tabacum. Other major alkaloids in tobacco include cotinine, nornicotine, myosmine, nicotyrine, anabasine and anatabine. Minor tobacco alkaloids include nicotine-n-oxide, N-methyl anatabine, N-methyl anabasine, pseudooxynicotine, 2,3 dipyridyl and others.

Modified tobacco plant, or part thereof, comprising a recombinant DNA construct comprising a heterologous promoter operably linked to an Alkaloid levels can be assayed by methods known in the art, for example by quantification based on gas-liquid chromatography, high performance liquid chromatography, radio-immunoassays, and enzyme-linked immunosorbent assays. For example, nicotinic alkaloid levels can be measured by a GC-FID method based on CORESTA Recommended Method No. 7, 1987 and ISO Standards (ISO TC 126N 394 E. See also Hibi et al., Plant Physiology 100:826-35 (1992) for a method using gas-liquid chromatography equipped with a capillary column and an FID detector.

Alternatively, tobacco total alkaloids can be measured using a segmented-flow colorimetric method developed for analysis of tobacco samples as adapted by Skalar Instrument Co. (West Chester, PA) and described by Collins et al., Tobacco Science 13:79-81 (1969). In short, samples of tobacco are dried, ground, and extracted prior to analysis of total alkaloids and reducing sugars. The method then employs an acetic acid/methanol/water extraction and charcoal for decolorization. Determination of total alkaloids was based on the reaction of cyanogen chloride with nicotine alkaloids in the presence of an aromatic amine to form a colored complex which is measured at 460 nm.

In an aspect, the level of total TSNAs or an individual TSNA is measured based on a freeze-dried cured leaf sample using liquid chromatography with tandem mass spectrometry (LC/MS/MS).

As used herein, “marker assay” means a method for detecting a marker at a locus using a method, e.g. measurement of at least one phenotype (such as a visually detectable trait, including red flesh and carliness), restriction fragment length polymorphism (RFLP), single base extension, electrophoresis, sequence alignment, allelic specific oligo-nucleotide hybridization (ASO), random amplified polymorphic DNA (RAPD), microarray-based technologies, PCR-based technologies, and nucleic acid sequencing technologies, etc.

As used herein, “polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism (RFLP), and a tag SNP. A genetic marker, a gene, a DNA-derived sequence, a RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

As used herein, “SNP” or “single nucleotide polymorphism” means a sequence variation that occurs when a single nucleotide (A, T, C, or G) in the genome sequence is altered or variable. “SNP markers” exist when SNPs are mapped to sites on the genome.

As used herein, “germplasm” refers to living sources of genetic material. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed, or tissues from which new plants may be grown, or plant parts, such as leaves, stems, pollen, ovules, or cells that can be cultured into a whole plant.

As used herein, a centimorgan (“cM”) is a unit of measure of recombination frequency and genetic distance between two loci. One cM is equal to a 1% chance that a marker at one genetic locus will be separated from a marker at a second locus due to crossing over in a single generation. Genetic distances can be calculated from experimentally derived recombination values using the Kosambi function (Kosambi, The estimation of map distances from recombination values. Annals of Eugenics, 12:172-75 (1944)).

As used herein, “linked” or “genetically linked” means that the marker or locus is within about 20 cM, 19 cM, 18 cM, 17 cM, 16 cM, 15 CM, 14 cM, 13 cM, 12 cM, 11 cM, 10 cM, 9 cM, 8 CM, 7 CM, 6 CM, 5 CM, 4 CM, 3 CM, 2 cM, 1 cM, 0.5 CM, or less than 0.5 CM of another marker or locus on the same chromosome. For example, 10 cM means that recombination occurs between the marker and the locus with a highly predictable recombination frequency of equal to or less than about 10%.

The genetic linkage of marker molecules can be established by a gene mapping model such as, without limitation, the flanking marker model reported by Lander and Botstein, Genetics, 121:185-199 (1989), and interval mapping, based on maximum likelihood methods described by Lander and Botstein, Genetics, 121:185-199 (1989), and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990). Additional software includes Qgene, Version 2.23 (1996), Department of Plant Breeding and Biometry, 266 Emerson Hall, Cornell University, Ithaca, N.Y.), JoinMap (Kyazma B.V., Wageningen, Netherlands), and mapQTL (Kyazma B.V., Wageningen, Netherlands).

As used herein, the phrase “associated with” or “linked to” refers to a recognizable and/or assayable relationship between two entities. As such, a marker is “associated with” a trait when it is linked to or co-segregates with it and when the presence of the marker is an indicator of whether and/or to what extent the desired trait or trait form will occur in a plant/germplasm comprising the marker. Similarly, a marker is “associated with” or “linked to” an allele when it is linked to or co-segregates with it and when the presence of the marker is an indicator of whether the allele is present in a plant/germplasm comprising the marker. For example, “a marker is linked to red flesh” when that marker is 10 cM or less away from an allele that co-segregates with red flesh.

As used herein, “crossed” or “cross” means to produce progeny via fertilization (e.g. cells, seeds or plants) and includes crosses between plants (sexual) and self-fertilization (selfing).

As used herein, “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. (Ragot et al., Marker-assisted Backcrossing: A Practical Example. Techniques Et Utilisations Des Marqueurs Moleculaires Les Colloques, 72:45-56 (1995); and Openshaw et al., Marker-assisted Selection in Backcross Breeding, in Proceedings Of The Symposium “Analysis of Molecular Marker Data,” pp. 41-43 (1994)). The initial cross gives rise to the F₁ generation. The term “BC₁” refers to the second use of the recurrent parent, “BC₂” refers to the third use of the recurrent parent, and so on. In an aspect, a backcross is performed repeatedly, with a progeny individual of each successive backcross generation being itself backcrossed to the same parental genotype.

As used herein, “genetic element” or “gene” refers to a heritable sequence of DNA, e.g., a genomic sequence, with functional significance. The term “gene” can also be used to refer to, e.g., acDNA or an mRNA encoded by a genomic sequence, as well as to that genomic sequence.

As used herein, “genotype” is the genetic constitution of an individual (or group of individuals) at one or more genetic loci, as contrasted with the observable trait (phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all its genome.

As used herein, a “haplotype” is the genotype of an individual at a plurality of genetic loci. Typically, the genetic loci described by a haplotype are physically and genetically linked, e.g., in the same chromosome interval. Selection based upon a haplotype can be more effective than selection based upon a single marker locus.

A “Quantitative Trait Locus (QTL)” is a chromosomal location that encodes for alleles that affect the expressivity of a phenotype.

As used herein, the term “phenotype” means the detectable characteristics of a cell or organism that can be influenced by gene expression.

As used herein, the term “introgressed,” when used in reference to a genetic locus, refers to a genetic locus that has been introduced into a new genetic background. Introgression of a genetic locus can thus be achieved through plant breeding methods and/or by molecular genetic methods. Such molecular genetic methods include, but are not limited to, various plant transformation techniques or methods that provide for homologous recombination, non-homologous recombination, site-specific recombination, or genomic modifications that provide for locus substitution or locus conversion.

As used herein, “mapping” is the process of defining the linkage relationships of loci through the use of genetic markers, populations segregating for the markers, and standard genetic principles of recombination frequency.

As used herein, “genetic mapping” is the process of defining the linkage relationships of loci through the use of genetic markers, populations segregating for the markers, and standard genetic principles of recombination frequency. A “genetic map location” is a location on a genetic map relative to surrounding genetic markers on the same linkage group where a specified marker can be found within a given species. In contrast, a “physical map” of the genome refers to absolute distances (for example, measured in base pairs or isolated and overlapping contiguous genetic fragments, e.g., contigs). In general, the closer two markers or genomic loci are on the genetic map, the closer they lie to one another on the physical map. A physical map of the genome does not take into account the genetic behavior (e.g., recombination frequencies) between different points on the physical map. A lack of precise proportionality between genetic distances and physical distances can exist due to the fact that the likelihood of genetic recombination is not uniform throughout the genome; some chromosome regions are cross-over “hot spots,” while other regions demonstrate only rare recombination events, if any. Genetic mapping variability can also be observed between different populations of the same crop species. In spite of this variability in the genetic map that may occur between populations, genetic map and marker information derived from one population generally remain useful across multiple populations in identification of plants with desired traits, counter-selection of plants with undesirable traits and in MAS breeding. As one of skill in the art will recognize, recombination frequencies (and as a result, genetic map positions) in any further populations are not static. The genetic distances separating two markers (or a marker and a QTL) can vary depending on how the map positions are determined. For example, variables such as the parental mapping populations used, the software used in marker mapping or QTL mapping, and the parameters input by the user of the mapping software can contribute to the QTL marker genetic map relationships. However, it is not intended that the disclosure be limited to any further mapping populations, use of any further software, or any further set of software parameters to determine linkage of a further marker or chromosome interval with a desired phenotype. It is well within the ability of one of ordinary skill in the art to extrapolate the novel features described herein to any gene pool or population of interest, and using any further software and software parameters. Indeed, observations regarding genetic markers and chromosome intervals in populations in addition to those described herein are readily made using the teaching of the present disclosure.

As used herein, the terms “coupled”, “coupled loci” or “coupling event” refers to a genetic condition in which the alleles of two different loci occur together on one chromosome. In a further aspect, two different loci that are coupled occur within about 20 cM, 19 cM, 18 cM, 17 cM, 16 cM, 15 CM, 14 cM, 13 cM, 12 cM, 11 cM, 10 cM, 9 cM, 8 CM, 7 CM, 6 CM, 5 CM, 4 cM, 3 cM, 2 cM, 1 cM, 0.5 CM, or less than 0.5 cM from each other.

As used herein, “co-segregates” means inherited together during meiosis more often than random assortment would predict. In example, during meiosis in the course of plant breeding, two marker alleles or two phenotypes or a marker allele and a phenotype that are inherited together in the same progeny are said to co-segregate. This typically means that the two marker alleles, two phenotypes, or marker allele and phenotype are linked. For example, two genes coupled on the same chromosome will be inherited together and therefore co-segregate. A marker allele that is linked to a phenotype will tend to co-segregate with that phenotype.

As used herein, “flanking”, “flanking markers”, or an “interval flanked by” is used to describe the boundaries of a chromosome interval or region by designated genetic markers. As used herein, flanking markers designating the boundaries of a given region are included within that region.

As used herein, the term “chromosome interval” designates a contiguous linear span of genomic DNA that resides on a single chromosome.

As used herein, “locus” is a chromosome region where a polymorphic nucleic acid, trait determinant, gene, or marker is located. The loci of this disclosure comprise one or more polymorphisms in a population; e.g., alternative alleles are present in some individuals. A “gene locus” is a specific chromosome location in the genome of a species where a specific gene can be found. A “trait locus” is a specific chromosome location in the genome of a species where a gene conferring a specific trait can be found.

As used herein, “allele” refers to an alternative nucleic acid sequence at a further locus. The length of an allele can be as small as 1 nucleotide base, but is typically larger. For example, a first allele can occur on one chromosome, while a second allele occurs on a second homologous chromosome, e.g., as occurs for different chromosomes of a heterozygous individual, or between different homozygous or heterozygous individuals in a population.

As used herein, the terms “recombinant” or “recombination event” refers to a plant having a new genetic make-up arising as a result of crossing over between homologous chromosomes during meiosis.

As used herein, a “detectable level” refers to a concentration or absolute count of a protein that can be objectively observed using standard techniques in the art. In an aspect, a method of measuring for a detectable level comprises protein sequencing. In an aspect, a method of measuring for a detectable level comprises a Western blot. In an aspect, a method of measuring for a detectable level comprises an ELISA. In an aspect, a method of measuring for a detectable level comprises enzyme kinetics experiments.

Non-limiting examples of plant tissue types include ground tissue, epidermal tissue, and vascular tissue. Further non-limiting examples of plant tissue include leaf tissue, stem tissue, floral tissue, meristem tissue, root tissue, and seed tissue. Additional non-limiting examples of plant tissue types include embryo tissue, endosperm tissue, seed coat tissue, axillary meristem tissue, shoot apical meristem tissue, and root apical meristem tissue.

### Embodiments

Below are non-limiting embodiments of the instant disclosure:

Embodiment 1. A modified tobacco plant, or part thereof, comprising at least one non-natural mutation in an endogenous nucleic acid sequence, wherein the endogenous nucleic acid sequence encodes a polypeptide comprising an amino acid sequence of SEQ ID NOs: 21 to 40.

Embodiment 2. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 21.

Embodiment 3. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 22.

Embodiment 4. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 23.

Embodiment 5. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 24.

Embodiment 6. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 25.

Embodiment 7. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 26.

Embodiment 8. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 27.

Embodiment 9. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 28.

Embodiment 10. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 29.

Embodiment 11. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 30.

Embodiment 12. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 31.

Embodiment 13. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 32.

Embodiment 14. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 33.

Embodiment 15. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 34.

Embodiment 16. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 35.

Embodiment 17. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 36.

Embodiment 18. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 37.

Embodiment 19. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 38.

Embodiment 20. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 39.

Embodiment 21. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80%, 85%, 90%, 91%, 92%, 93%, 94%, 95%, 96%, 97%, 98%, 99%, or 100% identical or similar to an amino acid sequence of SEQ ID NO: 40.

Embodiment 22. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 21.

Embodiment 23. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 22.

Embodiment 24. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 23.

Embodiment 25. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 24.

Embodiment 26. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 25.

Embodiment 27. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 26.

Embodiment 28. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 27.

Embodiment 29. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 28.

Embodiment 30. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 29.

Embodiment 31. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 30.

Embodiment 32. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 31.

Embodiment 33. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 32.

Embodiment 34. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 33.

Embodiment 35. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 34.

Embodiment 36. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 35.

Embodiment 37. A nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 36.

Embodiment 38. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 37.

Embodiment 39. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 38.

Embodiment 40. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 39.

Embodiment 41. A modified tobacco plant, or part thereof, comprising a recombinant DNA construct comprising a heterologous promoter operably linked to a nucleic acid sequence encoding a polypeptide comprising an amino acid sequence at least at least 80%, 85%, 90%, 91%, 92%, 93%, 94%, 95%, 96%, 97%, 98%, 99%, or 100% identical or similar to an amino acid sequence of SEQ ID NO: 40.

Embodiment 42. The modified tobacco plant, or part thereof, of any one of embodiments 1 to 41, wherein a leaf on said modified tobacco plant comprises an equivalent number trichomes as compared to an equivalent leaf of a control tobacco plant.

Embodiment 43. The modified tobacco plant, or part thereof, of any one of embodiments 1 to 41, wherein said modified tobacco plant produces at least one leaf comprising a reduced amount of at least one metabolite as compared to the amount of said metabolite in a control tobacco plant.

Embodiment 44. The modified tobacco plant, or part thereof, of any one of embodiments 1 to 41, wherein said tobacco plant produces at least one leaf comprising a reduced amount of at least one metabolite selected from the group consisting of an alkaloid, alkaloid derivative, Benzenoids, Flavonoids, Phenylpropanoids, and Terpenoids, compared to the amount of said metabolite in a control tobacco plant lacking said recombinant DNA construct when grown under similar growth conditions.

Embodiment 45. The modified tobacco plant, or part thereof, of any one of embodiments 1 to 41, wherein said tobacco plant produces at least one leaf comprising an increased amount of at least one metabolite selected from the group consisting of an alkaloid, alkaloid derivative, Benzenoids, Flavonoids, Phenylpropanoids, and Terpenoids, compared to the amount of said metabolite in a control tobacco plant lacking said recombinant DNA construct when grown under similar growth conditions.

Embodiment 46. The modified tobacco plant, or part thereof, of any one of embodiments 1 to 45, wherein said tobacco plant is homozygous for said at least one non-natural mutation.

Embodiment 47. The modified tobacco plant, or part thereof, of any one of embodiments 1 to 45, wherein said tobacco plant is heterozygous for said at least one non-natural mutation.

Embodiment 48. The modified tobacco plant, or part thereof, of any one of embodiments 43, 44, or 45, wherein said at least one metabolite is selected from the group consisting of an alkaloid, alkaloid derivative, Benzenoids, Flavonoids, Phenylpropanoids, and Terpenoids.

Embodiment 49. The modified tobacco plant, or part thereof, of any one of embodiments 43, 44, or 45, wherein said reduced amount of at least one metabolite comprises a reduction of at least 1%.

Embodiment 50. The modified tobacco plant, or part thereof, of any one of embodiments 43, 44, or 45, wherein said increased amount of at least one metabolite comprises a reduction of at least 1%.

Embodiment 51. The modified tobacco plant, or part thereof, of embodiment 1, wherein said at least one non-natural mutation comprises a mutation selected from the group consisting of an insertion, a deletion, a substitution, a duplication, and an inversion.

Embodiment 52. The modified tobacco plant, or part thereof, of embodiment 1, wherein said at least one non-natural mutation comprises at least one mutation selected from the group consisting of a nonsense mutation, a missense mutation, a frameshift mutation, and a splice-site mutation.

Embodiment 53. The modified tobacco plant, or part thereof, of embodiment 1, wherein said at least one non-natural mutation comprises a null mutation.

Embodiment 54. The modified tobacco plant, or part thereof, of embodiment 1, wherein said at least one non-natural mutation results in a truncation of said polypeptide.

Embodiment 55. The modified tobacco plant, or part thereof, of embodiment 1, wherein said at least one non-natural mutation comprises a mutation in a sequence region selected from the group consisting of a promoter, a 5′-untranslated region (UTR), an exon, an intron, a 3′-UTR, and a terminator.

Embodiment 56. The modified tobacco plant, or part thereof, or part thereof, of embodiment 1, wherein said at least one non-natural mutation results in a reduced level of expression of said nucleic acid sequence as compared to expression of said nucleic acid sequence in the same tissue of a control tobacco plant when grown under similar growth conditions, wherein said nucleic acid sequence lacks the at least one non-natural mutation in said control tobacco plant.

Embodiment 57. The modified tobacco plant, or part thereof, of embodiment 1, wherein said at least one non-natural mutation results in a reduced level of activity by a protein or polypeptide encoded by said nucleic acid sequence as compared to activity of a protein or polypeptide encoded by said nucleic acid sequence in a control tobacco plant when grown under similar growth conditions, wherein said nucleic acid sequence lacks the at least one non-natural mutation in said control tobacco plant.

Embodiment 58. The modified tobacco plant, or part thereof, of any one of embodiments 2 to 41, wherein said promoter comprises a promoter selected from the group consisting of a constitutive promoter, a tissue-preferred promoter, a tissue-specific promoter, and an inducible promoter.

Embodiment 59. The modified tobacco plant, or part thereof, of embodiment 58, wherein said tissue-preferred promoter comprises a leaf-preferred promoter or a trichome-preferred promoter.

Embodiment 60. The modified tobacco plant, or part thereof, of embodiment 58, wherein said tissue-specific promoter comprises a leaf-specific promoter or a trichome-specific promoter.

Embodiment 61. The modified tobacco plant, or part thereof, of embodiment 58, wherein said constitutive promoter is selected from the group consisting of a Cauliflower Mosaic Virus (CaMV) 35S promoter, a ubiquitin promoter, an actin promoter, an opine promoter, and an alcohol dehydrogenase promoter.

Embodiment 62. The modified tobacco plant, or part thereof, of any one of embodiments 2 to 21, wherein the at least one small RNA molecule is selected from the group consisting of a double-stranded RNA, a small interfering RNA (siRNA), a trans-acting siRNA, and a microRNA.

Embodiment 63. The modified tobacco plant, or part thereof, of any one of embodiments 2 to 21, wherein said at least one small RNA molecule comprises between 18 nucleotides and 30 nucleotides.

Embodiment 64. The modified tobacco plant, or part thereof, of any one of embodiments 2 to 21, wherein said at least one small RNA molecule comprises a nucleic acid sequence at least 90% complementary to a sequence selected from the group consisting of SEQ ID NOs: 1 to 20 or 41 to 60.

Embodiment 65. The modified tobacco plant, or part thereof, of any one of embodiments 1 to 41, wherein the modified tobacco plant is of a tobacco variety selected from the group consisting of a flue-cured variety, a bright variety, a Burley variety, a Virginia variety, a Maryland variety, a dark variety, a Galpão variety, an Oriental variety, and a Turkish variety.

Embodiment 66. The modified tobacco plant, or part thereof, of any one of the preceding embodiments, wherein the modified tobacco plant is of a variety selected from the group consisting of the tobacco varieties listed in Tables 2-8.

Embodiment 67. The modified tobacco plant, or part thereof, of any one of the preceding embodiments, wherein the modified tobacco plant is a hybrid.

Embodiment 68. The modified tobacco plant, or part thereof, of any one of the preceding embodiments, wherein the modified tobacco plant is male sterile or cytoplasmically male sterile.

Embodiment 69. The modified tobacco plant, or part thereof, of any one of embodiments 1 to 67, wherein the modified tobacco plant is female sterile.

Embodiment 70. Cured tobacco material from the modified tobacco plant, or part thereof, of any one of the preceding embodiments.

Embodiment 71. The cured tobacco material of embodiment 70, wherein said cured tobacco material comprises cured leaf material, cured stem material, or both.

Embodiment 72. The cured tobacco material of embodiments 70 or 71, wherein said cured tobacco material comprises flue cured tobacco material, air cured tobacco material, fire cured tobacco material, and sun cured tobacco material.

Embodiment 73. A tobacco blend comprising the cured tobacco material of any one of embodiments 70 to 71.

Embodiment 74. The tobacco blend of embodiment 73, wherein said tobacco blend comprises at least 10% cured tobacco by weight.

Embodiment 75. The tobacco blend of embodiment 73, wherein said tobacco blend comprises at least 10% cured tobacco by volume.

Embodiment 76. A tobacco product comprising the tobacco blend of any one of embodiments 73 to 75.

Embodiment 77. A tobacco product comprising the cured tobacco material of any one of embodiments 70 to 72.

Embodiment 78. The tobacco product of embodiment 76 or 77, wherein said tobacco product is selected from the group consisting of a cigarette, heated tobacco product, kretek, bidi cigarette, cigar, cigarillo, non-ventilated cigarette, vented recess filter cigarette, pipe tobacco, snuff, snus, chewing tobacco, moist smokeless tobacco, fine cut chewing tobacco, long cut chewing tobacco, pouched chewing tobacco product, gum, tablet, lozenge, and dissolving strip.

Embodiment 79. The tobacco product of embodiment 76 or 77, wherein said tobacco product is a smokeless tobacco product.

Embodiment 80. The tobacco product of embodiment 79, wherein said smokeless tobacco product is selected from the group consisting of loose leaf chewing tobacco, plug chewing tobacco, moist snuff, nasal snuff, dry snuff, and snus.

Embodiment 81. A reconstituted tobacco comprising the cured tobacco material of any one of embodiments 70 to 72.

Embodiment 82. A method of producing a modified tobacco plant comprising:
inducing a non-natural mutation in at least one tobacco cell in an endogenous nucleic acid sequence encoding a polypeptide comprising an amino acid sequence at least 80% identical or similar to an amino acid sequence selected from the group consisting of SEQ ID NOS: 21 to 40;
selecting at least one tobacco cell comprising said non-natural mutation from step (a); and
regenerating at least one modified tobacco plant from said at least one tobacco cell selected in step (b).

Embodiment 83. A method of producing a modified tobacco plant comprising:
inducing a non-natural mutation in at least one tobacco cell in an endogenous nucleic acid sequence encoding a polypeptide comprising an amino acid sequence at least 85% identical or similar to an amino acid sequence selected from the group consisting of SEQ ID NOS: 21 to 40;
selecting at least one tobacco cell comprising said non-natural mutation from step (a); and
regenerating at least one modified tobacco plant from said at least one tobacco cell selected in step (b).

Embodiment 84. A method of producing a modified tobacco plant comprising:
inducing a non-natural mutation in at least one tobacco cell in an endogenous nucleic acid sequence encoding a polypeptide comprising an amino acid sequence at least 90% identical or similar to an amino acid sequence selected from the group consisting of SEQ ID NOs: 21 to 40;
selecting at least one tobacco cell comprising said non-natural mutation from step (a); and
regenerating at least one modified tobacco plant from said at least one tobacco cell selected in step (b).

Embodiment 85. A method of producing a modified tobacco plant comprising:
inducing a non-natural mutation in at least one tobacco cell in an endogenous nucleic acid sequence encoding a polypeptide comprising an amino acid sequence at least 92.5% identical or similar to an amino acid sequence selected from the group consisting of SEQ ID NOs: 21 to 40;
selecting at least one tobacco cell comprising said non-natural mutation from step (a); and
regenerating at least one modified tobacco plant from said at least one tobacco cell selected in step (b).

Embodiment 86. A method of producing a modified tobacco plant comprising:
inducing a non-natural mutation in at least one tobacco cell in an endogenous nucleic acid sequence encoding a polypeptide comprising an amino acid sequence at least 95% identical or similar to an amino acid sequence selected from the group consisting of SEQ ID NOS: 21 to 40;
selecting at least one tobacco cell comprising said non-natural mutation from step (a); and
regenerating at least one modified tobacco plant from said at least one tobacco cell selected in step (b).

Embodiment 87. A method of producing a modified tobacco plant comprising:
inducing a non-natural mutation in at least one tobacco cell in an endogenous nucleic acid sequence encoding a polypeptide comprising an amino acid sequence at least 97.5% identical or similar to an amino acid sequence selected from the group consisting of SEQ ID NOs: 21 to 40;
selecting at least one tobacco cell comprising said non-natural mutation from step (a); and
regenerating at least one modified tobacco plant from said at least one tobacco cell selected in step (b).

Embodiment 88. A method of producing a modified tobacco plant comprising:
inducing a non-natural mutation in at least one tobacco cell in an endogenous nucleic acid sequence encoding a polypeptide comprising an amino acid sequence identical or similar to an amino acid sequence selected from the group consisting of SEQ ID NOs: 21 to 40;
selecting at least one tobacco cell comprising said non-natural mutation from step (a); and
regenerating at least one modified tobacco plant from said at least one tobacco cell selected in step (b).

Embodiment 89. A method of producing a modified tobacco plant comprising:
introducing a recombinant DNA construct to at least one tobacco cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 90. A method of producing a modified tobacco plant comprising:
introducing a recombinant DNA construct to at least one tobacco cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 85% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 91. A method of producing a modified tobacco plant comprising:
introducing a recombinant DNA construct to at least one tobacco cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 90% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 92. A method of producing a modified tobacco plant comprising:
introducing a recombinant DNA construct to at least one tobacco cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 92.5% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 93. A method of producing a modified tobacco plant comprising:
introducing a recombinant DNA construct to at least one tobacco cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 95% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 94. A method of producing a modified tobacco plant comprising:
introducing a recombinant DNA construct to at least one tobacco cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 97.5% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 95. A method of producing a modified tobacco plant comprising:
introducing a recombinant DNA construct to at least one tobacco cell, wherein said recombinant DNA construct comprises a heterologous promoter operably linked to a nucleic acid encoding at least one small RNA molecule capable of binding to and reducing the expression of an endogenous nucleic acid sequence encoding a polypeptide identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 96. A method of producing a modified tobacco plant comprising:
introducing a recombinant DNA construct to at least one tobacco cell, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 97. A method of producing a modified tobacco plant comprising:
introducing a recombinant DNA construct to at least one tobacco cell, wherein said recombinant DNA construct comprises a heterologous promoter operably linked to a nucleic acid sequence encoding a polypeptide comprising an amino acid sequence at least 85% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 98. A method of producing a modified tobacco plant comprising:
introducing a recombinant DNA construct to at least one tobacco cell, wherein said recombinant DNA construct comprises a heterologous promoter operably linked to a nucleic acid sequence encoding a polypeptide comprising an amino acid sequence at least 90% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 99. A method of producing a modified tobacco plant comprising:
introducing a recombinant DNA construct to at least one tobacco cell, wherein said recombinant DNA construct comprises a heterologous promoter operably linked to a nucleic acid sequence encoding a polypeptide comprising an amino acid sequence at least 92.5% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 100. A method of producing a modified tobacco plant comprising:
introducing a recombinant DNA construct to at least one tobacco cell, wherein said recombinant DNA construct comprises a heterologous promoter operably linked to a nucleic acid sequence encoding a polypeptide comprising an amino acid sequence at least 95% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 101. A method of producing a modified tobacco plant comprising:
introducing a recombinant DNA construct to at least one tobacco cell, wherein said recombinant DNA construct comprises a heterologous promoter operably linked to a nucleic acid sequence encoding a polypeptide comprising an amino acid sequence at least 97.5% identical or similar to an amino acid sequence selected from the group consisting of SEQ ID NOs: 21 to 40;
selecting at least one tobacco cell comprising said recombinant DNA construct; and
regenerating at least one modified tobacco plant from said at least one tobacco cell selected in step (b).

Embodiment 102. A method of producing a modified tobacco plant comprising:
introducing a recombinant DNA construct to at least one tobacco cell, wherein said recombinant DNA construct comprises a heterologous promoter operably linked to a nucleic acid sequence encoding a polypeptide comprising an amino acid sequence identical or similar to an amino acid sequence selected from the group consisting of SEQ ID NOS: 21 to 40;
selecting at least one tobacco cell comprising said recombinant DNA construct; and
regenerating at least one modified tobacco plant from said at least one tobacco cell selected in step (b).

Embodiment 103. The method of any one of embodiments 82 to 102, wherein a leaf on a tobacco plant produced by said method comprises an equivalent number trichomes as compared to an equivalent leaf of a control tobacco plant when grown under similar growth conditions.

Embodiment 104. The method of any one of embodiments 82 to 103, wherein said at least one modified tobacco plant comprises a reduced amount of at least one metabolite as compared to a control tobacco plant when grown under similar growth conditions.

Embodiment 105. The method of any one of embodiments 82 to 103, wherein said at least one modified tobacco plant comprises an increased amount of at least one metabolite as compared to a control tobacco plant when grown under similar growth conditions.

Embodiment 106. The method of any one of embodiments 82 to 95, wherein said endogenous nucleic acid sequence is at least 80% identical to a sequence selected from the group consisting of SEQ ID NOs: 1 to 20 and 41 to 60.

Embodiment 107. The method of any one of embodiments 82 to 95, wherein said endogenous nucleic acid sequence is at least 85% identical to a sequence selected from the group consisting of SEQ ID NOs: 1 to 20 and 41 to 60.

Embodiment 108. The method of any one of embodiments 82 to 95, wherein said endogenous nucleic acid sequence is at least 90% identical to a sequence selected from the group consisting of SEQ ID NOs: 1 to 20 and 41 to 60.

Embodiment 109. The method of any one of embodiments 82 to 95, wherein said endogenous nucleic acid sequence is at least 92.5% identical to a sequence selected from the group consisting of SEQ ID NOs: 1 to 20 and 41 to 60.

Embodiment 110. The method of any one of embodiments 82 to 95, wherein said endogenous nucleic acid sequence is at least 95% identical to a sequence selected from the group consisting of SEQ ID NOs: 1 to 20 and 41 to 60.

Embodiment 111. The method of any one of embodiments 82 to 95, wherein said endogenous nucleic acid sequence is at least 97.5% identical to a sequence selected from the group consisting of SEQ ID NOs: 1 to 20 and 41 to 60.

Embodiment 112. The method of any one of embodiments 82 to 95, wherein said endogenous nucleic acid sequence is identical to a sequence selected from the group consisting of SEQ ID NOs: 1 to 20 and 41 to 60.

Embodiment 113. The method of any one of embodiments 96 to 102, wherein said nucleic acid sequence is at least 80% identical to a sequence selected from the group consisting of SEQ ID NOs: 1 to 20 and 41 to 60.

Embodiment 114. The method of any one of embodiments 96 to 102, wherein said nucleic acid sequence is at least 85% identical to a sequence selected from the group consisting of SEQ ID NOs: 1 to 20 and 41 to 60.

Embodiment 115. The method of any one of embodiments 96 to 102, wherein said nucleic acid sequence is at least 90% identical to a sequence selected from the group consisting of SEQ ID NOs: 1 to 20 and 41 to 60.

Embodiment 116. The method of any one of embodiments 96 to 102, wherein said nucleic acid sequence is at least 92.5% identical to a sequence selected from the group consisting of SEQ ID NOs: 1 to 20 and 41 to 60.

Embodiment 117. The method of any one of embodiments 96 to 102, wherein said nucleic acid sequence is at least 95% identical to a sequence selected from the group consisting of SEQ ID NOs: 1 to 20 and 41 to 60.

Embodiment 118. The method of any one of embodiments 96 to 102, wherein said nucleic acid sequence is at least 97.5% identical to a sequence selected from the group consisting of SEQ ID NOs: 1 to 20 and 41 to 60.

Embodiment 119. The method of any one of embodiments 96 to 102, wherein said nucleic acid sequence is identical to a sequence selected from the group consisting of SEQ ID NOs: 1 to 20 and 41 to 60.

Embodiment 120. The method of embodiment 104 or 105, wherein said at least one metabolite is selected from the group consisting of an alkaloid, alkaloid derivative, Benzenoids, Flavonoids, Phenylpropanoids, and Terpenoids.

Embodiment 121. The method of embodiment 104 or 105, wherein said reduced amount of at least one metabolite comprises a reduction of at least 1%.

Embodiment 122. The method of any one of embodiments 82 to 95, wherein said non-natural mutation comprises a mutation selected from the group consisting of an insertion, a deletion, a substitution, a duplication, and an inversion.

Embodiment 123. The method of any one of embodiments 82 to 95, wherein said non-natural mutation comprises a mutation selected from the group consisting of a nonsense mutation, a missense mutation, a frameshift mutation, and a splice-site mutation.

Embodiment 124. The method of any one of embodiments 82 to 95, wherein said non-natural mutation comprises a null mutation.

The method of any one of embodiments 82 to 95, wherein said non-natural mutation results in a truncation of said polypeptide.

Embodiment 125. The method of any one of embodiments 82 to 95, wherein said non-natural mutation comprises a mutation in a sequence region selected from the group consisting of a promoter, a 5′-untranslated region (UTR), an exon, an intron, a 3′-UTR, and a terminator.

Embodiment 126. The method of any one of embodiments 96 to 102, wherein said inducing comprises the use of an agent selected from the group consisting of: a chemical mutagen, irradiation, a transposon, Agrobacterium, and a nuclease.

Embodiment 127. The method of embodiment 126, wherein said nuclease is selected from the group consisting of a meganuclease, a zinc-finger nuclease, a transcription activator-like effector nuclease, a CRISPR/Cas9 nuclease, a CRISPR/Cpf1 nuclease, a CRISPR/CasX nuclease, a CRISPR/CasY nuclease, a Csm1 nuclease, or any combination thereof.

Embodiment 128. The method of embodiment 126, wherein said chemical mutagen comprises ethyl methanesulfonate.

Embodiment 129. The method of embodiment 126, wherein said irradiation comprises gamma rays, X-rays, ionizing radiation, or fast neutrons.

Embodiment 130. The method of any one of embodiments 89 to 95, wherein said small RNA molecule is selected from the group consisting of a double-stranded RNA, a small interfering RNA (siRNA), a trans-acting siRNA, and a microRNA.

Embodiment 131. The method of any one of embodiments 89 to 95, wherein said at least one small RNA molecule comprises between 18 nucleotides and 30 nucleotides.

Embodiment 132. The method of any one of embodiments 89 to 95, wherein said at least one small RNA molecule comprises a nucleic acid sequence at least 90% complementary to a sequence selected from the group consisting of SEQ ID NOs: 1 to 20 and 41 to 60.

Embodiment 133. The method of any one of embodiments 96 to 102, wherein said promoter comprises a promoter selected from the group consisting of a constitutive promoter, a tissue-preferred promoter, a tissue-specific promoter, and an inducible promoter.

Embodiment 134. The method of embodiment 133, wherein said promoter comprises a sequence selected from the group consisting of SEQ ID Nos: 62 to 90.

Embodiment 135. The method of embodiment 133, wherein said tissue-preferred promoter comprises a root-preferred promoter.

Embodiment 136. The method of embodiment 133, wherein said tissue-specific promoter comprises a root-specific promoter.

Embodiment 137. The method of any one of embodiments 133 to 136, wherein said promoter comprises a sequence selected from the group consisting of SEQ ID Nos: 70 to 80.

Embodiment 138. The method of embodiment 133, wherein said promoter regulates leaf specific or preferred expression.

Embodiment 139. The method of embodiment 133, wherein said inducible promoter comprises a sequence selected from the group consisting of SEQ ID Nos: 81 to 90.

Embodiment 140. The method of embodiment 133, wherein said constitutive promoter is selected from the group consisting of a Cauliflower Mosaic Virus (CaMV) 35S promoter, a ubiquitin promoter, an actin promoter, an opine promoter, and an alcohol dehydrogenase promoter.

Embodiment 141. The method of any one of embodiments 82 to 102, wherein said at least one tobacco cell is a tobacco protoplast cell.

Embodiment 142. The method of any one of embodiments 82 to 102, wherein said at least one tobacco cell is a tobacco callus cell.

Embodiment 143. The method o of any one of embodiments 82 to 102, wherein said at least one tobacco cell is selected from the group consisting of a seed cell, a fruit cell, a leaf cell, a cotyledon cell, a hypocotyl cell, a meristem cell, an embryo cell, an endosperm cell, a root cell, a shoot cell, a stem cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, and a phloem cell.

Embodiment 144. The method of any one of embodiments 82 to 102, wherein said method further comprises:
growing said modified tobacco plant regenerated in step (c).

Embodiment 145. The method of embodiment 144, wherein said method further comprises:
crossing said modified tobacco plant grown in step (d) with a second tobacco plant; and
obtaining at least one seed from said crossing in step (c).

Embodiment 146. The method of any one of embodiments 82 to 95, wherein said at least one non-natural mutation results in a reduced level of expression of said nucleic acid sequence as compared to expression of said nucleic acid sequence in the same tissue of a control tobacco plant when grown under similar growth conditions, wherein said nucleic acid sequence lacks the at least one non-natural mutation in said control tobacco plant.

Embodiment 147. The method of embodiment 146, wherein said reduced level of expression comprises a reduction of at least 5%.

Embodiment 148. The method of any one of embodiments 82 to 95, wherein said at least one non-natural mutation results in an increased level of expression of said nucleic acid sequence as compared to expression of said nucleic acid sequence in the same tissue of a control tobacco plant when grown under similar growth conditions, wherein said nucleic acid sequence lacks the at least one non-natural mutation in said control tobacco plant.

Embodiment 149. The method of embodiment 148, wherein said increased level of expression comprises an increase of at least 5%.

Embodiment 150. The method of any one of embodiments 82 to 95, wherein said at least one non-natural mutation results in a reduced level of activity by a protein or polypeptide encoded by said nucleic acid sequence as compared to activity of a protein or polypeptide encoded by said nucleic acid sequence in a control tobacco plant when grown under similar growth conditions, wherein said nucleic acid sequence lacks the at least one non-natural mutation in said control tobacco plant.

Embodiment 151. The method of any one of embodiments 82 to 95, wherein said at least one non-natural mutation results in an increased level of activity by a protein or polypeptide encoded by said nucleic acid sequence as compared to activity of a protein or polypeptide encoded by said nucleic acid sequence in a control tobacco plant when grown under similar growth conditions, wherein said nucleic acid sequence lacks the at least one non-natural mutation in said control tobacco plant.

Embodiment 152. The method of any one of embodiments 82 to 102, wherein said modified tobacco plant is of a tobacco variety selected from the group consisting of a flue-cured variety, a bright variety, a Burley variety, a Virginia variety, a Maryland variety, a dark variety, a Galpão variety, an Oriental variety, and a Turkish variety.

Embodiment 153. The method of any one of embodiments 82 to 102, wherein said modified tobacco plant is of a variety selected from the group consisting of the varieties listed in Tables 2-8.

Embodiment 154. The method of any one of embodiments 82 to 102, wherein said modified tobacco plant is a hybrid.

Embodiment 155. The method of any one of embodiments 82 to 102, wherein said modified tobacco plant is male sterile or cytoplasmically male sterile.

Embodiment 156. The method of any one of embodiments 82 to 102, wherein said modified tobacco plant is female sterile.

Embodiment 157. The method of any one of embodiments 82 to 102, wherein said modified tobacco plant comprises a comparable or higher USDA leaf grade index as compared to a control tobacco plant when grown under similar growth conditions.

Embodiment 158. A method comprising preparing a tobacco product using cured tobacco material from a modified tobacco plant, wherein said modified tobacco plant comprises a non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 21 to 40.

Embodiment 159. A method comprising preparing a tobacco product using cured tobacco material from a modified tobacco plant, wherein said modified tobacco plant comprises a non-natural mutation in an endogenous nucleic acid sequence, wherein the endogenous nucleic acid sequence encodes a polypeptide comprising an amino acid sequence at least 85% identical or similar to an amino acid sequence selected from the group consisting of SEQ ID NOs: 21 to 40.

Embodiment 160. A method comprising preparing a tobacco product using cured tobacco material from a modified tobacco plant, wherein said modified tobacco plant comprises a non-natural mutation in an endogenous nucleic acid sequence, wherein the endogenous nucleic acid sequence encodes a polypeptide comprising an amino acid sequence at least 90% identical or similar to an amino acid sequence selected from the group consisting of SEQ ID NOs: 21 to 40.

Embodiment 161. A method comprising preparing a tobacco product using cured tobacco material from a modified tobacco plant, wherein said modified tobacco plant comprises a non-natural mutation in an endogenous nucleic acid sequence, wherein the endogenous nucleic acid sequence encodes a polypeptide comprising an amino acid sequence at least 92.5% identical or similar to an amino acid sequence selected from the group consisting of SEQ ID NOs: 21 to 40.

Embodiment 162. A method comprising preparing a tobacco product using cured tobacco material from a modified tobacco plant, wherein said modified tobacco plant comprises a non-natural mutation in an endogenous nucleic acid sequence, wherein the endogenous nucleic acid sequence encodes a polypeptide comprising an amino acid sequence at least 95% identical or similar to an amino acid sequence selected from the group consisting of SEQ ID NOs: 21 to 40.

Embodiment 163. A method comprising preparing a tobacco product using cured tobacco material from a modified tobacco plant, wherein said modified tobacco plant comprises a non-natural mutation in an endogenous nucleic acid sequence, wherein the endogenous nucleic acid sequence encodes a polypeptide comprising an amino acid sequence at least 97.5% identical or similar to an amino acid sequence selected from the group consisting of SEQ ID NOs: 21 to 40.

Embodiment 164. A method comprising preparing a tobacco product using cured tobacco material from a modified tobacco plant, wherein said modified tobacco plant comprises a non-natural mutation in an endogenous nucleic acid sequence, wherein the endogenous nucleic acid sequence encodes a polypeptide comprising an amino acid sequence selected from the group consisting of SEQ ID NOs: 21 to 40.

Embodiment 165. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40.

Embodiment 166. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 85% identical or similar to an amino acid sequence selected from the group consisting of SEQ ID NOs: 21 to 40.

Embodiment 167. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 90% identical or similar to an amino acid sequence selected from the group consisting of SEQ ID NOs: 21 to 40.

Embodiment 168. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 92.5% identical or similar to an amino acid sequence selected from the group consisting of SEQ ID NOs: 21 to 40.

Embodiment 169. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 95% identical or similar to an amino acid sequence selected from the group consisting of SEQ ID NOs: 21 to 40.

Embodiment 170. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 97.5% identical or similar to an amino acid sequence selected from the group consisting of SEQ ID NOs: 21 to 40.

Embodiment 171. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding at least one small RNA molecule capable of binding to and reducing the expression of an endogenous nucleic acid sequence encoding a polypeptide selected from the group consisting of SEQ ID NOs: 21 to 40.

Embodiment 172. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40.

Embodiment 173. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding a polypeptide comprising an amino acid sequence at least 85% identical or similar to an amino acid sequence selected from the group consisting of SEQ ID NOs: 21 to 40.

Embodiment 174. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding a polypeptide comprising an amino acid sequence at least 90% identical or similar to an amino acid sequence selected from the group consisting of SEQ ID NOs: 21 to 40.

Embodiment 175. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding a polypeptide comprising an amino acid sequence at least 92.5% identical or similar to an amino acid sequence selected from the group consisting of SEQ ID NOs: 21 to 40.

Embodiment 176. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding a polypeptide comprising an amino acid sequence at least 95% identical or similar to an amino acid sequence selected from the group consisting of SEQ ID NOs: 21 to 40.

Embodiment 177. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding a polypeptide comprising an amino acid sequence at least 97.5% identical or similar to an amino acid sequence selected from the group consisting of SEQ ID NOs: 21 to 40.

Embodiment 178. A method comprising preparing a tobacco product using cured tobacco material from a modified tobacco plant, wherein said modified tobacco plant comprises a recombinant DNA construct, and wherein said recombinant DNA construct comprises a heterologous promoter operably linked to a nucleic acid sequence encoding a polypeptide comprising an amino acid sequence selected from the group consisting of SEQ ID NOs: 21 to 40.

Embodiment 179. The method of any one of embodiments 158 to 178, wherein said cured tobacco material comprises cured leaf material, cured stem material, or both.

Embodiment 180. The method of any one of embodiments 158 to 178, wherein said cured tobacco material comprises flue-cured tobacco material, air-cured tobacco material, fire-cured tobacco material, and sun-cured tobacco material.

Embodiment 181. The method of any one of embodiments 158 to 178, wherein said tobacco product is selected from the group consisting of a cigarette, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, pouched chewing tobacco product, gum, a tablet, a lozenge, and a dissolving strip.

Embodiment 182. The method of any one of embodiments 158 to 178, wherein said tobacco product is a smokeless tobacco product.

Embodiment 183. The method of embodiment 182, wherein said smokeless tobacco product is selected from the group consisting of loose leaf chewing tobacco, plug chewing tobacco, moist snuff, nasal snuff, dry snuff, and snus.

Embodiment 184. The method of any one of embodiments 158 to 178, wherein said cured tobacco material is of a tobacco variety selected from the group consisting of a flue-cured variety, a bright variety, a Burley variety, a Virginia variety, a Maryland variety, a dark variety, a Galpão variety, an Oriental variety, and a Turkish variety.

Embodiment 185. The method of any one of embodiments 158 to 171, wherein said endogenous nucleic acid sequence comprises a sequence at least 80% identical to a sequence selected from the group consisting of SEQ ID NOs: 1 to 20 and 41 to 60.

Embodiment 186. The method of any one of embodiments 158 to 171, wherein said endogenous nucleic acid sequence comprises a sequence at least 85% identical to a sequence selected from the group consisting of SEQ ID NOs: 1 to 20 and 41 to 60.

Embodiment 187. The method of any one of embodiments 158 to 171, wherein said endogenous nucleic acid sequence comprises a sequence at least 90% identical to a sequence selected from the group consisting of SEQ ID NOs: 1 to 20 and 41 to 60.

Embodiment 188. The method of any one of embodiments 158 to 171, wherein said endogenous nucleic acid sequence comprises a sequence at least 92.5% identical to a sequence selected from the group consisting of SEQ ID NOs: 1 to 20 and 41 to 60.

Embodiment 189. The method of any one of embodiments 158 to 171, wherein said endogenous nucleic acid sequence comprises a sequence at least 95% identical to a sequence selected from the group consisting of SEQ ID NOs: 1 to 20 and 41 to 60.

Embodiment 190. The method of any one of embodiments 158 to 171, wherein said endogenous nucleic acid sequence comprises a sequence at least 97.5% identical to a sequence selected from the group consisting of SEQ ID NOs: 1 to 20 and 41 to 60.

Embodiment 191. The method of any one of embodiments 158 to 171, wherein said endogenous nucleic acid sequence comprises a sequence selected from the group consisting of SEQ ID NOs: 1 to 20 and 41 to 60.

Embodiment 192. The method of any one of embodiments 172 to 178, wherein said nucleic acid sequence comprises a sequence at least 80% identical to a sequence selected from the group consisting of SEQ ID NOs: 1 to 20 and 41 to 60.

Embodiment 193. The method of any one of embodiments 172 to 178, wherein said nucleic acid sequence comprises a sequence at least 85% identical to a sequence selected from the group consisting of SEQ ID NOs: 1 to 20 and 41 to 60.

Embodiment 194. The method of any one of embodiments 172 to 178, wherein said nucleic acid sequence comprises a sequence at least 90% identical to a sequence selected from the group consisting of SEQ ID NOs: 1 to 20 and 41 to 60.

Embodiment 195. The method of any one of embodiments 172 to 178, wherein said nucleic acid sequence comprises a sequence at least 92.5% identical to a sequence selected from the group consisting of SEQ ID NOs: 1 to 20 and 41 to 60.

Embodiment 196. The method of any one of embodiments 172 to 178, wherein said nucleic acid sequence comprises a sequence at least 95% identical to a sequence selected from the group consisting of SEQ ID NOs: 1 to 20 and 41 to 60.

Embodiment 197. The method of any one of embodiments 172 to 178, wherein said nucleic acid sequence comprises a sequence at least 97.5% identical to a sequence selected from the group consisting of SEQ ID NOs: 1 to 20 and 41 to 60.

Embodiment 198. The method of any one of embodiments 172 to 178, wherein said nucleic acid sequence comprises a sequence selected from the group consisting of SEQ ID NOS: 1 to 20 and 41 to 60.

Embodiment 199. A method comprising transforming a tobacco cell with a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40.

Embodiment 200. A method comprising transforming a tobacco cell with a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 85% identical or similar to an amino acid sequence selected from the group consisting of SEQ ID NOs: 21 to 40.

Embodiment 201. A method comprising transforming a tobacco cell with a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 90% identical or similar to an amino acid sequence selected from the group consisting of SEQ ID NOs: 21 to 40.

Embodiment 202. A method comprising transforming a tobacco cell with a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 92.5% identical or similar to an amino acid sequence selected from the group consisting of SEQ ID NOs: 21 to 40.

Embodiment 203. A method comprising transforming a tobacco cell with a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 95% identical or similar to an amino acid sequence selected from the group consisting of SEQ ID NOs: 21 to 40.

Embodiment 204. A method comprising transforming a tobacco cell with a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 97.5% identical or similar to an amino acid sequence selected from the group consisting of SEQ ID NOs: 21 to 40.

Embodiment 205. A method comprising transforming a tobacco cell with a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide selected from the group consisting of SEQ ID NOs: 21 to 40.

Embodiment 206. A method comprising transforming a tobacco cell with a recombinant DNA construct, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40.

Embodiment 207. A method comprising transforming a tobacco cell with a recombinant DNA construct, wherein said recombinant DNA construct comprises a heterologous promoter operably linked to a nucleic acid sequence encoding a polypeptide comprising an amino acid sequence at least 85% identical or similar to an amino acid sequence selected from the group consisting of SEQ ID NOs: 21 to 40.

Embodiment 208. A method comprising transforming a tobacco cell with a recombinant DNA construct, wherein said recombinant DNA construct comprises a heterologous promoter operably linked to a nucleic acid sequence encoding a polypeptide comprising an amino acid sequence at least 90% identical or similar to an amino acid sequence selected from the group consisting of SEQ ID NOs: 21 to 40.

Embodiment 209. A method comprising transforming a tobacco cell with a recombinant DNA construct, wherein said recombinant DNA construct comprises a heterologous promoter operably linked to a nucleic acid sequence encoding a polypeptide comprising an amino acid sequence at least 92.5% identical or similar to an amino acid sequence selected from the group consisting of SEQ ID NOs: 21 to 40.

Embodiment 210. A method comprising transforming a tobacco cell with a recombinant DNA construct, wherein said recombinant DNA construct comprises a heterologous promoter operably linked to a nucleic acid sequence encoding a polypeptide comprising an amino acid sequence at least 95% identical or similar to an amino acid sequence selected from the group consisting of SEQ ID NOs: 21 to 40.

Embodiment 211. A method comprising transforming a tobacco cell with a recombinant DNA construct, wherein said recombinant DNA construct comprises a heterologous promoter operably linked to a nucleic acid sequence encoding a polypeptide comprising an amino acid sequence at least 97.5% identical or similar to an amino acid sequence selected from the group consisting of SEQ ID NOs: 21 to 40.

Embodiment 212. A method comprising transforming a tobacco cell with a recombinant DNA construct, wherein said recombinant DNA construct comprises a heterologous promoter operably linked to a nucleic acid sequence encoding a polypeptide comprising an amino acid sequence at least 100% identical or similar to an amino acid sequence selected from the group consisting of SEQ ID NOs: 21 to 40.

Embodiment 213. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 21 to 40, wherein said non-natural mutation is not present in said endogenous nucleic acid sequence in a control tobacco plant of said first tobacco variety; and
selecting for at least one progeny tobacco seed, or a plant germinated therefrom, wherein said at least one tobacco seed or plant germinated therefrom comprises said non-natural mutation.

Embodiment 214. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a non-natural mutation in an endogenous nucleic acid sequence, wherein the endogenous nucleic acid sequence encodes a polypeptide comprising an amino acid sequence at least 85% identical or similar to an amino acid sequence selected from the group consisting of SEQ ID NOs: 21 to 40, wherein said non-natural mutation is not present in said endogenous nucleic acid sequence in a control tobacco plant of said first tobacco variety; and
selecting for at least one progeny tobacco seed, or a plant germinated therefrom, wherein said at least one tobacco seed or plant germinated therefrom comprises said non-natural mutation.

Embodiment 215. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a non-natural mutation in an endogenous nucleic acid sequence, wherein the endogenous nucleic acid sequence encodes a polypeptide comprising an amino acid sequence at least 90% identical or similar to an amino acid sequence selected from the group consisting of SEQ ID NOs: 21 to 40, wherein said non-natural mutation is not present in said endogenous nucleic acid sequence in a control tobacco plant of said first tobacco variety; and
selecting for at least one progeny tobacco seed, or a plant germinated therefrom, wherein said at least one tobacco seed or plant germinated therefrom comprises said non-natural mutation.

Embodiment 216. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a non-natural mutation in an endogenous nucleic acid sequence, wherein the endogenous nucleic acid sequence encodes a polypeptide comprising an amino acid sequence at least 92.5% identical or similar to an amino acid sequence selected from the group consisting of SEQ ID NOs: 21 to 40, wherein said non-natural mutation is not present in said endogenous nucleic acid sequence in a control tobacco plant of said first tobacco variety; and
selecting for at least one progeny tobacco seed, or a plant germinated therefrom, wherein said at least one tobacco seed or plant germinated therefrom comprises said non-natural mutation.

Embodiment 217. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a non-natural mutation in an endogenous nucleic acid sequence, wherein the endogenous nucleic acid sequence encodes a polypeptide comprising an amino acid sequence at least 95% identical or similar to an amino acid sequence selected from the group consisting of SEQ ID NOs: 21 to 40, wherein said non-natural mutation is not present in said endogenous nucleic acid sequence in a control tobacco plant of said first tobacco variety; and
selecting for at least one progeny tobacco seed, or a plant germinated therefrom, wherein said at least one tobacco seed or plant germinated therefrom comprises said non-natural mutation.

Embodiment 218. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a non-natural mutation in an endogenous nucleic acid sequence, wherein the endogenous nucleic acid sequence encodes a polypeptide comprising an amino acid sequence at least 97.5% identical or similar to an amino acid sequence selected from the group consisting of SEQ ID NOs: 21 to 40, wherein said non-natural mutation is not present in said endogenous nucleic acid sequence in a control tobacco plant of said first tobacco variety; and
selecting for at least one progeny tobacco seed, or a plant germinated therefrom, wherein said at least one tobacco seed or plant germinated therefrom comprises said non-natural mutation.

Embodiment 219. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a non-natural mutation in an endogenous nucleic acid sequence, wherein the endogenous nucleic acid sequence encodes a polypeptide comprising an amino acid sequence selected from the group consisting of SEQ ID NOs: 21 to 40, wherein said non-natural mutation is not present in said endogenous nucleic acid sequence in a control tobacco plant of said first tobacco variety; and
selecting for at least one progeny tobacco seed, or a plant germinated therefrom, wherein said at least one tobacco seed or plant germinated therefrom comprises said non-natural mutation.

Embodiment 220. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40, wherein said recombinant DNA construct is not present in said endogenous nucleic acid sequence in a control tobacco plant of the same variety; and
selecting for at least one progeny tobacco seed, or a plant germinated therefrom, wherein said at least one tobacco seed or plant germinated therefrom comprises said recombinant DNA construct.

Embodiment 221. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 85% identical or similar to an amino acid sequence selected from the group consisting of SEQ ID NOs: 21 to 40, wherein said recombinant DNA construct is not present in said endogenous nucleic acid sequence in a control tobacco plant of the same variety; and
selecting for at least one progeny tobacco seed, or a plant germinated therefrom, wherein said at least one tobacco seed or plant germinated therefrom comprises said recombinant DNA construct.

Embodiment 222. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 90% identical or similar to an amino acid sequence selected from the group consisting of SEQ ID NOs: 21 to 40, wherein said recombinant DNA construct is not present in said endogenous nucleic acid sequence in a control tobacco plant of the same variety; and
selecting for at least one progeny tobacco seed, or a plant germinated therefrom, wherein said at least one tobacco seed or plant germinated therefrom comprises said recombinant DNA construct.

Embodiment 223. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 92.5% identical or similar to an amino acid sequence selected from the group consisting of SEQ ID NOs: 21 to 40, wherein said recombinant DNA construct is not present in said endogenous nucleic acid sequence in a control tobacco plant of the same variety; and
selecting for at least one progeny tobacco seed, or a plant germinated therefrom, wherein said at least one tobacco seed or plant germinated therefrom comprises said recombinant DNA construct.

Embodiment 224. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 95% identical or similar to an amino acid sequence selected from the group consisting of SEQ ID NOs: 21 to 40, wherein said recombinant DNA construct is not present in said endogenous nucleic acid sequence in a control tobacco plant of the same variety; and
selecting for at least one progeny tobacco seed, or a plant germinated therefrom, wherein said at least one tobacco seed or plant germinated therefrom comprises said recombinant DNA construct.

Embodiment 225. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 97.5% identical or similar to an amino acid sequence selected from the group consisting of SEQ ID NOs: 21 to 40, wherein said recombinant DNA construct is not present in said endogenous nucleic acid sequence in a control tobacco plant of the same variety; and
selecting for at least one progeny tobacco seed, or a plant germinated therefrom, wherein said at least one tobacco seed or plant germinated therefrom comprises said recombinant DNA construct.

Embodiment 226. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide sequence selected from the group consisting of SEQ ID NOs: 21 to 40, wherein said recombinant DNA construct is not present in said endogenous nucleic acid sequence in a control tobacco plant of the same variety; and
selecting for at least one progeny tobacco seed, or a plant germinated therefrom, wherein said at least one tobacco seed or plant germinated therefrom comprises said recombinant DNA construct.

Embodiment 227. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40, wherein said recombinant DNA construct is not present in said nucleic acid sequence in a control tobacco plant of the first tobacco variety; and
selecting for at least one progeny tobacco seed, or a plant germinated therefrom, wherein said at least one tobacco seed or plant germinated therefrom comprises said recombinant DNA construct.

Embodiment 228. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding a polypeptide comprising an amino acid sequence at least 85% identical or similar to an amino acid sequence selected from the group consisting of SEQ ID NOs: 21 to 40, wherein said recombinant DNA construct is not present in said nucleic acid sequence in a control tobacco plant of the first tobacco variety; and
selecting for at least one progeny tobacco seed, or a plant germinated therefrom, wherein said at least one tobacco seed or plant germinated therefrom comprises said recombinant DNA construct.

Embodiment 229. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding a polypeptide comprising an amino acid sequence at least 90% identical or similar to an amino acid sequence selected from the group consisting of SEQ ID NOs: 21 to 40, wherein said recombinant DNA construct is not present in said nucleic acid sequence in a control tobacco plant of the first tobacco variety; and
selecting for at least one progeny tobacco seed, or a plant germinated therefrom, wherein said at least one tobacco seed or plant germinated therefrom comprises said recombinant DNA construct.

Embodiment 230. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding a polypeptide comprising an amino acid sequence at least 92.5% identical or similar to an amino acid sequence selected from the group consisting of SEQ ID NOs: 21 to 40, wherein said recombinant DNA construct is not present in said nucleic acid sequence in a control tobacco plant of the first tobacco variety; and
selecting for at least one progeny tobacco seed, or a plant germinated therefrom, wherein said at least one tobacco seed or plant germinated therefrom comprises said recombinant DNA construct.

Embodiment 231. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding a polypeptide comprising an amino acid sequence at least 95% identical or similar to an amino acid sequence selected from the group consisting of SEQ ID NOs: 21 to 40, wherein said recombinant DNA construct is not present in said nucleic acid sequence in a control tobacco plant of the first tobacco variety; and
selecting for at least one progeny tobacco seed, or a plant germinated therefrom, wherein said at least one tobacco seed or plant germinated therefrom comprises said recombinant DNA construct.

Embodiment 232. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding a polypeptide comprising an amino acid sequence at least 97.5% identical or similar to an amino acid sequence selected from the group consisting of SEQ ID NOs: 21 to 40, wherein said recombinant DNA construct is not present in said nucleic acid sequence in a control tobacco plant of the first tobacco variety; and
selecting for at least one progeny tobacco seed, or a plant germinated therefrom, wherein said at least one tobacco seed or plant germinated therefrom comprises said recombinant DNA construct.

Embodiment 233. A method for producing a modified tobacco plant comprising:
crossing at least one tobacco plant of a first tobacco variety with at least one tobacco plant of a second tobacco variety to produce at least one progeny tobacco seed, wherein said at least one tobacco plant of said first tobacco variety comprises a recombinant DNA construct, wherein said recombinant DNA construct comprises a heterologous promoter operably linked to a nucleic acid sequence encoding a polypeptide comprising an amino acid sequence selected from the group consisting of SEQ ID NOs: 21 to 40, wherein said recombinant DNA construct is not present in said nucleic acid sequence in a control tobacco plant of the first tobacco variety; and
selecting for at least one progeny tobacco seed, or a plant germinated therefrom, wherein said at least one tobacco seed or plant germinated therefrom comprises said recombinant DNA construct.

Embodiment 234. The method of any one of embodiments 213 to 233, wherein a leaf on said tobacco plant comprises an equivalent number trichomes as compared to an equivalent leaf of a control tobacco plant.

Embodiment 235. The method of any one of embodiments 213 to 233, wherein said plant germinated in step (b) comprises a reduced amount of at least one metabolite as compared to said control tobacco plant when grown under similar growth conditions.

Embodiment 236. The method of embodiment 235, wherein said at least one metabolite is selected from the group consisting of an alkaloid, alkaloid derivative, Benzenoids, Flavonoids, Phenylpropanoids, and Terpenoids.

Embodiment 237. The method of embodiment 235 or 236, wherein said reduced amount of at least one metabolite comprises a reduction of at least 1%.

Embodiment 238. The method of any one of embodiments 213 to 233, wherein said plant germinated in step (b) comprises an increased amount of at least one metabolite as compared to said control tobacco plant when grown under similar growth conditions.

Embodiment 239. The method of embodiment 238, wherein said at least one metabolite is selected from the group consisting of an alkaloid, alkaloid derivative, Benzenoids, Flavonoids, Phenylpropanoids, and Terpenoids.

Embodiment 240. The method of embodiment 238 or 239, wherein said increased amount of at least one metabolite comprises an increase of at least 1%.

Embodiment 241. The method of any one of embodiments 213 to 233, wherein said endogenous nucleic acid sequence comprises a sequence at least 80% identical to a sequence selected from the group consisting of SEQ ID NOs: 1 to 20 and 41 to 60.

Embodiment 242. The method of any one of embodiments 213 to 233, wherein said endogenous nucleic acid sequence comprises a sequence at least 85% identical to a sequence selected from the group consisting of SEQ ID NOs: 1 to 20 and 41 to 60.

Embodiment 243. The method of any one of embodiments 213 to 233, wherein said endogenous nucleic acid sequence comprises a sequence at least 90% identical to a sequence selected from the group consisting of SEQ ID NOs: 1 to 20 and 41 to 60.

Embodiment 244. The method of any one of embodiments 213 to 233, wherein said endogenous nucleic acid sequence comprises a sequence at least 92.5% identical to a sequence selected from the group consisting of SEQ ID NOs: 1 to 20 and 41 to 60.

Embodiment 245. The method of any one of embodiments 213 to 233, wherein said endogenous nucleic acid sequence comprises a sequence at least 95% identical to a sequence selected from the group consisting of SEQ ID NOs: 1 to 20 and 41 to 60.

Embodiment 246. The method of any one of embodiments 213 to 233, wherein said endogenous nucleic acid sequence comprises a sequence at least 97.5% identical to a sequence selected from the group consisting of SEQ ID NOs: 1 to 20 and 41 to 60.

Embodiment 247. The method of any one of embodiments 213 to 233, wherein said endogenous nucleic acid sequence comprises a sequence selected from the group consisting of SEQ ID NOs: 1 to 20 and 41 to 60.

Embodiment 248. A modified Camellia plant, or part thereof, comprising at least one non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 21 to 40.

Embodiment 249. A modified Camellia plant, or part thereof, comprising at least one non-natural mutation in an endogenous nucleic acid sequence, wherein the endogenous nucleic acid sequence encodes a polypeptide comprising an amino acid sequence at least 85% identical or similar to an amino acid sequence selected from the group consisting of SEQ ID NOs: 21 to 40.

Embodiment 250. A modified Camellia plant, or part thereof, comprising at least one non-natural mutation in an endogenous nucleic acid sequence, wherein the endogenous nucleic acid sequence encodes a polypeptide comprising an amino acid sequence at least 90% identical or similar to an amino acid sequence selected from the group consisting of SEQ ID NOs: 21 to 40.

Embodiment 251. A modified Camellia plant, or part thereof, comprising at least one non-natural mutation in an endogenous nucleic acid sequence, wherein the endogenous nucleic acid sequence encodes a polypeptide comprising an amino acid sequence at least 92.5% identical or similar to an amino acid sequence selected from the group consisting of SEQ ID NOs: 21 to 40.

Embodiment 252. A modified Camellia plant, or part thereof, comprising at least one non-natural mutation in an endogenous nucleic acid sequence, wherein the endogenous nucleic acid sequence encodes a polypeptide comprising an amino acid sequence at least 95% identical or similar to an amino acid sequence selected from the group consisting of SEQ ID NOs: 21 to 40.

Embodiment 253. A modified Camellia plant, or part thereof, comprising at least one non-natural mutation in an endogenous nucleic acid sequence, wherein the endogenous nucleic acid sequence encodes a polypeptide comprising an amino acid sequence at least 97.5% identical or similar to an amino acid sequence selected from the group consisting of SEQ ID NOs: 21 to 40.

Embodiment 254. A modified Camellia plant, or part thereof, comprising at least one non-natural mutation in an endogenous nucleic acid sequence, wherein the endogenous nucleic acid sequence encodes a polypeptide comprising an amino acid sequence selected from the group consisting of SEQ ID NOs: 21 to 40.

Embodiment 255. A modified Camellia plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40.

Embodiment 256. A modified Camellia plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 85% identical or similar to an amino acid sequence selected from the group consisting of SEQ ID NOs: 21 to 40.

Embodiment 257. A modified Camellia plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 90% identical or similar to an amino acid sequence selected from the group consisting of SEQ ID NOs: 21 to 40.

Embodiment 258. A modified Camellia plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 92.5% identical or similar to an amino acid sequence selected from the group consisting of SEQ ID NOs: 21 to 40.

Embodiment 259. A modified Camellia plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 95% identical or similar to an amino acid sequence selected from the group consisting of SEQ ID NOs: 21 to 40.

Embodiment 260. A modified Camellia plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 97.5% identical or similar to an amino acid sequence selected from the group consisting of SEQ ID NOs: 21 to 40.

Embodiment 261. A modified Camellia plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide sequence selected from the group consisting of SEQ ID NOs: 21 to 40.

Embodiment 262. A modified Camellia plant, or part thereof, comprising a recombinant DNA construct comprising a heterologous promoter operably linked to a nucleic acid sequence encoding a polypeptide comprising an amino acid sequence at least 80% identical or similar to an amino acid sequence selected from the group consisting of SEQ ID NOs: 21 to 40.

Embodiment 263. A modified Camellia plant, or part thereof, comprising a recombinant DNA construct comprising a heterologous promoter operably linked to a nucleic acid sequence encoding a polypeptide comprising an amino acid sequence at least 85% identical or similar to an amino acid sequence selected from the group consisting of SEQ ID NOs: 21 to 40.

Embodiment 264 A modified Camellia plant, or part thereof, comprising a recombinant DNA construct comprising a heterologous promoter operably linked to a nucleic acid sequence encoding a polypeptide comprising an amino acid sequence at least 90% identical or similar to an amino acid sequence selected from the group consisting of SEQ ID NOs: 21 to 40.

Embodiment 265. A modified Camellia plant, or part thereof, comprising a recombinant DNA construct comprising a heterologous promoter operably linked to a nucleic acid sequence encoding a polypeptide comprising an amino acid sequence at least 92.5% identical or similar to an amino acid sequence selected from the group consisting of SEQ ID NOs: 21 to 40.

Embodiment 266. A modified Camellia plant, or part thereof, comprising a recombinant DNA construct comprising a heterologous promoter operably linked to a nucleic acid sequence encoding a polypeptide comprising an amino acid sequence at least 95% identical or similar to an amino acid sequence selected from the group consisting of SEQ ID NOs: 21 to 40.

Embodiment 267. A modified Camellia plant, or part thereof, comprising a recombinant DNA construct comprising a heterologous promoter operably linked to a nucleic acid sequence encoding a polypeptide comprising an amino acid sequence at least 97.5% identical or similar to an amino acid sequence selected from the group consisting of SEQ ID NOs: 21 to 40.

Embodiment 268. A modified Camellia plant, or part thereof, comprising a recombinant DNA construct comprising a heterologous promoter operably linked to a nucleic acid sequence encoding a polypeptide comprising an amino acid sequence selected from the group consisting of SEQ ID NOs: 21 to 40.

Embodiment 269. Cured leaf material from the modified Camellia plant, or part thereof, of any one of embodiments 248 to 268.

Embodiment 270. A method of producing a modified Camellia plant comprising:
(a) inducing a non-natural mutation in at least one Camellia cell in an endogenous nucleic acid sequence encoding a polypeptide comprising an amino acid sequence at least 80% identical or similar to an amino acid sequence selected from the group consisting of SEQ ID NOs: 21 to 40;
(b) selecting at least one Camellia cell comprising said non-natural mutation from step (a); and
(c) regenerating at least one modified tobacco plant from said at least one Camellia cell selected in step (b).

Embodiment 271. A method of producing a modified Camellia plant comprising:
(a) inducing a non-natural mutation in at least one Camellia cell in an endogenous nucleic acid sequence encoding a polypeptide comprising an amino acid sequence at least 85% identical or similar to an amino acid sequence selected from the group consisting of SEQ ID NOs: 21 to 40;
(b) selecting at least one Camellia cell comprising said non-natural mutation from step (a); and
(c) regenerating at least one modified tobacco plant from said at least one Camellia cell selected in step (b).

Embodiment 272. A method of producing a modified Camellia plant comprising:
(a) inducing a non-natural mutation in at least one Camellia cell in an endogenous nucleic acid sequence encoding a polypeptide comprising an amino acid sequence at least 90% identical or similar to an amino acid sequence selected from the group consisting of SEQ ID NOs: 21 to 40;
(b) selecting at least one Camellia cell comprising said non-natural mutation from step (a); and
(c) regenerating at least one modified tobacco plant from said at least one Camellia cell selected in step (b).

Embodiment 273. A method of producing a modified Camellia plant comprising:
(a) inducing a non-natural mutation in at least one Camellia cell in an endogenous nucleic acid sequence encoding a polypeptide comprising an amino acid sequence at least 92.5% identical or similar to an amino acid sequence selected from the group consisting of SEQ ID NOs: 21 to 40;
(b) selecting at least one Camellia cell comprising said non-natural mutation from step (a); and
(c) regenerating at least one modified tobacco plant from said at least one Camellia cell selected in step (b).

Embodiment 274. A method of producing a modified Camellia plant comprising:
(a) inducing a non-natural mutation in at least one Camellia cell in an endogenous nucleic acid sequence encoding a polypeptide comprising an amino acid sequence at least 95% identical or similar to an amino acid sequence selected from the group consisting of SEQ ID NOs: 21 to 40;
(b) selecting at least one Camellia cell comprising said non-natural mutation from step (a); and
(c) regenerating at least one modified tobacco plant from said at least one Camellia cell selected in step (b).

Embodiment 275. A method of producing a modified Camellia plant comprising:
(a) inducing a non-natural mutation in at least one Camellia cell in an endogenous nucleic acid sequence encoding a polypeptide comprising an amino acid sequence at least 97.5% identical or similar to an amino acid sequence selected from the group consisting of SEQ ID NOs: 21 to 40;
(b) selecting at least one Camellia cell comprising said non-natural mutation from step (a); and
(c) regenerating at least one modified tobacco plant from said at least one Camellia cell selected in step (b).

Embodiment 276. A method of producing a modified Camellia plant comprising:
(a) inducing a non-natural mutation in at least one Camellia cell in an endogenous nucleic acid sequence encoding a polypeptide comprising an amino acid sequence selected from the group consisting of SEQ ID NOs: 21 to 40;
(b) selecting at least one Camellia cell comprising said non-natural mutation from step (a); and
(c) regenerating at least one modified tobacco plant from said at least one Camellia cell selected in step (b).

Embodiment 277. A method of producing a modified Camellia plant comprising:
(a) introducing a recombinant DNA construct to at least one Camellia cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 80% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 278. A method of producing a modified Camellia plant comprising:
(a) introducing a recombinant DNA construct to at least one Camellia cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 85% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 279. A method of producing a modified Camellia plant comprising:
(a) introducing a recombinant DNA construct to at least one Camellia cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 90% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 280. A method of producing a modified Camellia plant comprising:
(a) introducing a recombinant DNA construct to at least one Camellia cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 92.5% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 281. A method of producing a modified Camellia plant comprising:
(a) introducing a recombinant DNA construct to at least one Camellia cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 95% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 282. A method of producing a modified Camellia plant comprising:
(a) introducing a recombinant DNA construct to at least one Camellia cell, wherein said recombinant DNA construct comprises a heterologous promoter operably linked to a nucleic acid encoding at least one small RNA molecule capable of binding to and reducing the expression of an endogenous nucleic acid sequence encoding a polypeptide at least 97.5% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 283. A method of producing a modified Camellia plant comprising:
(a) introducing a recombinant DNA construct to at least one Camellia cell, wherein said recombinant DNA construct comprises a heterologous promoter operably linked to a nucleic acid encoding at least one small RNA molecule capable of binding to and reducing the expression of an endogenous nucleic acid sequence encoding a polypeptide sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 284. A method of producing a modified Camellia plant comprising:
(a) introducing a recombinant DNA construct to at least one Camellia cell, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 285. A method of producing a modified Camellia plant comprising:
(a) introducing a recombinant DNA construct to at least one Camellia cell, wherein said recombinant DNA construct comprises a heterologous promoter operably linked to a nucleic acid sequence encoding a polypeptide comprising an amino acid sequence at least 85% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 286. A method of producing a modified Camellia plant comprising:
(a) introducing a recombinant DNA construct to at least one Camellia cell, wherein said recombinant DNA construct comprises a heterologous promoter operably linked to a nucleic acid sequence encoding a polypeptide comprising an amino acid sequence at least 90% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 287. A method of producing a modified Camellia plant comprising:
(a) introducing a recombinant DNA construct to at least one Camellia cell, wherein said recombinant DNA construct comprises a heterologous promoter operably linked to a nucleic acid sequence encoding a polypeptide comprising an amino acid sequence at least 92.5% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 288. A method of producing a modified Camellia plant comprising:
(a) introducing a recombinant DNA construct to at least one Camellia cell, wherein said recombinant DNA construct comprises a heterologous promoter operably linked to a nucleic acid sequence encoding a polypeptide comprising an amino acid sequence at least 95% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 289. A method of producing a modified Camellia plant comprising:
(a) introducing a recombinant DNA construct to at least one Camellia cell, wherein said recombinant DNA construct comprises a heterologous promoter operably linked to a nucleic acid sequence encoding a polypeptide comprising an amino acid sequence at least 97.5% identical or similar to an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 290. A method of producing a modified Camellia plant comprising:
(a) introducing a recombinant DNA construct to at least one Camellia cell, wherein said recombinant DNA construct comprises a heterologous promoter operably linked to a nucleic acid sequence encoding a polypeptide comprising an amino acid sequence selected from the group consisting of SEQ ID NOs: 21 to 40;
(b) selecting at least one Camellia cell comprising said recombinant DNA construct; and
(c) regenerating at least one modified Camellia plant from said at least one tobacco cell selected in step (b).

Embodiment 291. Cured leaf material from the modified Camellia plant, or part thereof, of any one of embodiments 270 to 290.

Embodiment 292. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a non-natural mutation in an endogenous nucleic acid sequence, wherein the endogenous nucleic acid sequence encodes a polypeptide comprising an amino acid sequence at least 80% identical or similar to an amino acid sequence selected from the group consisting of SEQ ID NOs: 21 to 40, wherein said non-natural mutation is not present in said endogenous nucleic acid sequence in a control Camellia plant of said first Camellia variety; and
(b) selecting for at least one progeny Camellia seed, or a plant germinated therefrom, wherein said at least one Camellia seed or plant germinated therefrom comprises said non-natural mutation.

Embodiment 293. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a non-natural mutation in an endogenous nucleic acid sequence, wherein the endogenous nucleic acid sequence encodes a polypeptide comprising an amino acid sequence at least 85% identical or similar to an amino acid sequence selected from the group consisting of SEQ ID NOs: 21 to 40, wherein said non-natural mutation is not present in said endogenous nucleic acid sequence in a control Camellia plant of said first Camellia variety; and
(b) selecting for at least one progeny Camellia seed, or a plant germinated therefrom, wherein said at least one Camellia seed or plant germinated therefrom comprises said non-natural mutation.

Embodiment 294. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a non-natural mutation in an endogenous nucleic acid sequence, wherein the endogenous nucleic acid sequence encodes a polypeptide comprising an amino acid sequence at least 90% identical or similar to an amino acid sequence selected from the group consisting of SEQ ID NOs: 21 to 40, wherein said non-natural mutation is not present in said endogenous nucleic acid sequence in a control Camellia plant of said first Camellia variety; and
(b) selecting for at least one progeny Camellia seed, or a plant germinated therefrom, wherein said at least one Camellia seed or plant germinated therefrom comprises said non-natural mutation.

Embodiment 295. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a non-natural mutation in an endogenous nucleic acid sequence, wherein the endogenous nucleic acid sequence encodes a polypeptide comprising an amino acid sequence at least 92.5% identical or similar to an amino acid sequence selected from the group consisting of SEQ ID NOs: 21 to 40, wherein said non-natural mutation is not present in said endogenous nucleic acid sequence in a control Camellia plant of said first Camellia variety; and
(b) selecting for at least one progeny Camellia seed, or a plant germinated therefrom, wherein said at least one Camellia seed or plant germinated therefrom comprises said non-natural mutation.

Embodiment 296. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a non-natural mutation in an endogenous nucleic acid sequence, wherein the endogenous nucleic acid sequence encodes a polypeptide comprising an amino acid sequence at least 95% identical or similar to an amino acid sequence selected from the group consisting of SEQ ID NOs: 21 to 40, wherein said non-natural mutation is not present in said endogenous nucleic acid sequence in a control Camellia plant of said first Camellia variety; and
(b) selecting for at least one progeny Camellia seed, or a plant germinated therefrom, wherein said at least one Camellia seed or plant germinated therefrom comprises said non-natural mutation.

Embodiment 297. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a non-natural mutation in an endogenous nucleic acid sequence, wherein the endogenous nucleic acid sequence encodes a polypeptide comprising an amino acid sequence at least 97.5% identical or similar to an amino acid sequence selected from the group consisting of SEQ ID NOs: 21 to 40, wherein said non-natural mutation is not present in said endogenous nucleic acid sequence in a control Camellia plant of said first Camellia variety; and
(b) selecting for at least one progeny Camellia seed, or a plant germinated therefrom, wherein said at least one Camellia seed or plant germinated therefrom comprises said non-natural mutation.

Embodiment 298. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a non-natural mutation in an endogenous nucleic acid sequence, wherein the endogenous nucleic acid sequence encodes a polypeptide comprising an amino acid sequence selected from the group consisting of SEQ ID NOs: 21 to 40, wherein said non-natural mutation is not present in said endogenous nucleic acid sequence in a control Camellia plant of said first Camellia variety; and
(b) selecting for at least one progeny Camellia seed, or a plant germinated therefrom, wherein said at least one Camellia seed or plant germinated therefrom comprises said non-natural mutation.

Embodiment 299. A method for producing a modified Camellia plant comprising:
(a) crossing at least one Camellia plant of a first Camellia variety with at least one tobacco plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 80% identical or similar to an amino acid sequence selected from the group consisting of SEQ ID NOS: 21 to 40, wherein said recombinant DNA construct is not present in said endogenous nucleic acid sequence in a control Camellia plant of the same variety; and
(b) selecting for at least one progeny Camellia seed, or a plant germinated therefrom, wherein said at least one Camellia seed or plant germinated therefrom comprises said recombinant DNA construct.

Embodiment 300. A method for producing a modified Camellia plant comprising:
(a) crossing at least one Camellia plant of a first Camellia variety with at least one tobacco plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 85% identical or similar to an amino acid sequence selected from the group consisting of SEQ ID NOs: 21 to 40, wherein said recombinant DNA construct is not present in said endogenous nucleic acid sequence in a control Camellia plant of the same variety; and
(b) selecting for at least one progeny Camellia seed, or a plant germinated therefrom, wherein said at least one Camellia seed or plant germinated therefrom comprises said recombinant DNA construct.

Embodiment 301. A method for producing a modified Camellia plant comprising:
(a) crossing at least one Camellia plant of a first Camellia variety with at least one tobacco plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 90% identical or similar to an amino acid sequence selected from the group consisting of SEQ ID NOS: 21 to 40, wherein said recombinant DNA construct is not present in said endogenous nucleic acid sequence in a control Camellia plant of the same variety; and
(b) selecting for at least one progeny Camellia seed, or a plant germinated therefrom, wherein said at least one Camellia seed or plant germinated therefrom comprises said recombinant DNA construct.

Embodiment 302. A method for producing a modified Camellia plant comprising:
(a) crossing at least one Camellia plant of a first Camellia variety with at least one tobacco plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 92.5% identical or similar to an amino acid sequence selected from the group consisting of SEQ ID NOs: 21 to 40, wherein said recombinant DNA construct is not present in said endogenous nucleic acid sequence in a control Camellia plant of the same variety; and
(b) selecting for at least one progeny Camellia seed, or a plant germinated therefrom, wherein said at least one Camellia seed or plant germinated therefrom comprises said recombinant DNA construct.

Embodiment 303. A method for producing a modified Camellia plant comprising:
(a) crossing at least one Camellia plant of a first Camellia variety with at least one tobacco plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 95% identical or similar to an amino acid sequence selected from the group consisting of SEQ ID NOS:

21 to 40, wherein said recombinant DNA construct is not present in said endogenous nucleic acid sequence in a control Camellia plant of the same variety; and
(b) selecting for at least one progeny Camellia seed, or a plant germinated therefrom, wherein said at least one Camellia seed or plant germinated therefrom comprises said recombinant DNA construct.

Embodiment 304. A method for producing a modified Camellia plant comprising:
(a) crossing at least one Camellia plant of a first Camellia variety with at least one tobacco plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide at least 97.5% identical or similar to an amino acid sequence selected from the group consisting of SEQ ID NOS: 21 to 40, wherein said recombinant DNA construct is not present in said endogenous nucleic acid sequence in a control Camellia plant of the same variety; and
(b) selecting for at least one progeny Camellia seed, or a plant germinated therefrom, wherein said at least one Camellia seed or plant germinated therefrom comprises said recombinant DNA construct.

Embodiment 305. A method for producing a modified Camellia plant comprising:
(a) crossing at least one Camellia plant of a first Camellia variety with at least one tobacco plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding at least one small RNA molecule capable of binding to and reducing the expression of an endogenous nucleic acid sequence encoding a polypeptide sequence selected from the group consisting of SEQ ID NOs: 21 to 40, wherein said recombinant DNA construct is not present in said endogenous nucleic acid sequence in a control Camellia plant of the same variety; and
(b) selecting for at least one progeny Camellia seed, or a plant germinated therefrom, wherein said at least one Camellia seed or plant germinated therefrom comprises said recombinant DNA construct.

Embodiment 306. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding a polypeptide comprising an amino acid sequence at least 80% identical or similar to an amino acid sequence selected from the group consisting of SEQ ID NOs: 21 to 40, wherein said recombinant DNA construct is not present in said nucleic acid sequence in a control tobacco plant of the first Camellia variety; and
(b) selecting for at least one progeny tobacco seed, or a plant germinated therefrom, wherein said at least one tobacco seed or plant germinated therefrom comprises said recombinant DNA construct.

Embodiment 307. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding a polypeptide comprising an amino acid sequence at least 85% identical or similar to an amino acid sequence selected from the group consisting of SEQ ID NOs: 21 to 40, wherein said recombinant DNA construct is not present in said nucleic acid sequence in a control tobacco plant of the first Camellia variety; and
(b) selecting for at least one progeny tobacco seed, or a plant germinated therefrom, wherein said at least one tobacco seed or plant germinated therefrom comprises said recombinant DNA construct.

Embodiment 308. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding a polypeptide comprising an amino acid sequence at least 90% identical or similar to an amino acid sequence selected from the group consisting of SEQ ID NOs: 21 to 40, wherein said recombinant DNA construct is not present in said nucleic acid sequence in a control tobacco plant of the first Camellia variety; and
(b) selecting for at least one progeny tobacco seed, or a plant germinated therefrom, wherein said at least one tobacco seed or plant germinated therefrom comprises said recombinant DNA construct.

Embodiment 309. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding a polypeptide comprising an amino acid sequence at least 92.5% identical or similar to an amino acid sequence selected from the group consisting of SEQ ID NOs: 21 to 40, wherein said recombinant DNA construct is not present in said nucleic acid sequence in a control tobacco plant of the first Camellia variety; and
(b) selecting for at least one progeny tobacco seed, or a plant germinated therefrom, wherein said at least one tobacco seed or plant germinated therefrom comprises said recombinant DNA construct.

Embodiment 310. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding a polypeptide comprising an amino acid sequence at least 95% identical or similar to an amino acid sequence selected from the group consisting of SEQ ID NOs: 21 to 40, wherein said recombinant DNA construct is not present in said nucleic acid sequence in a control tobacco plant of the first Camellia variety; and
(b) selecting for at least one progeny tobacco seed, or a plant germinated therefrom, wherein said at least one tobacco seed or plant germinated therefrom comprises said recombinant DNA construct.

Embodiment 311. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding a polypeptide comprising an amino acid sequence at least 97.5% identical or similar to an amino acid sequence selected from the group consisting of SEQ ID NOs: 21 to 40, wherein said recombinant DNA construct is not present in said nucleic acid sequence in a control tobacco plant of the first Camellia variety; and
(b) selecting for at least one progeny tobacco seed, or a plant germinated therefrom, wherein said at least one tobacco seed or plant germinated therefrom comprises said recombinant DNA construct.

Embodiment 312. A method for producing a modified Camellia plant comprising:
(a) crossing at least one Camellia plant of a first Camellia variety with at least one Camellia plant of a second Camellia variety to produce at least one progeny Camellia seed, wherein said at least one Camellia plant of said first Camellia variety comprises a recombinant DNA construct, wherein said recombinant DNA construct comprises a heterologous promoter operably linked to a nucleic acid sequence encoding a polypeptide comprising an amino acid sequence selected from the group consisting of SEQ ID NOs: 21 to 40, wherein said recombinant DNA construct is not present in said nucleic acid sequence in a control tobacco plant of the first Camellia variety; and
(b) selecting for at least one progeny tobacco seed, or a plant germinated therefrom, wherein said at least one tobacco seed or plant germinated therefrom comprises said recombinant DNA construct.

Embodiment 313. Cured leaf material from the modified Camellia plant, or part thereof, of any one of embodiments 292 to 312.

Having now generally described the disclosure, the same will be more readily understood through reference to the following examples that are provided by way of illustration, and are not intended to be limiting of the present disclosure, unless specified.

### Example 1. Phenotypic Evaluation of Secretor and Non-Secretor Lines

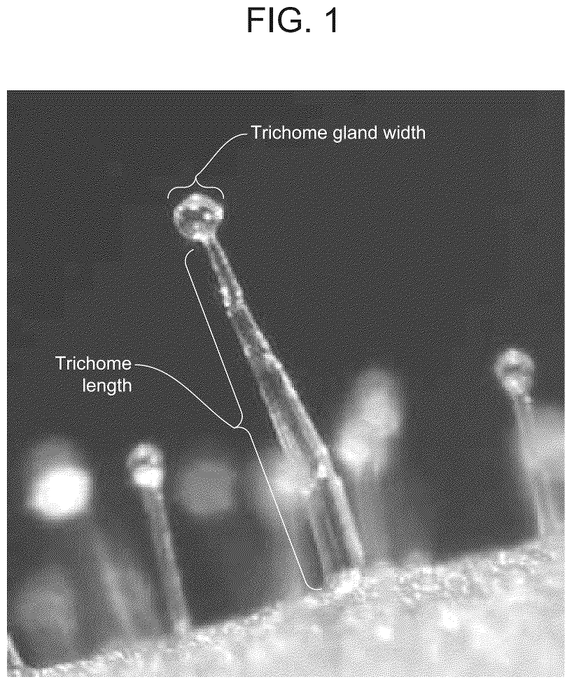

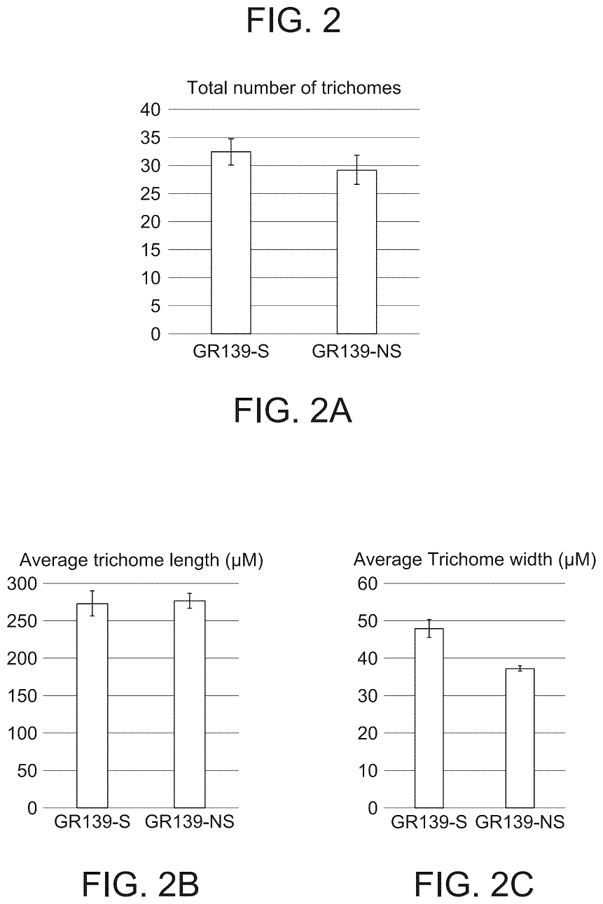

Known and suspected secretor and non-secretor tobacco lines are evaluated phenotypically. The tobacco plants are grown in 10 inch pots. At flowering time, 5 uniform plants per line were selected and the 10th leaf from the bottom of each plant was chosen. The middle portion of the lamina where the veins were minimal was cut using a scalpel and observed with a microscope in a glass Petri plate. Trichome number, length, and gland width was measured on a VHX-7000 Series fully-automated digital microscope system (Keyence Company, Osaka, Japan). The number of trichomes within a 1 mm radius (an area of 3.14 mm²) were analyzed, the length of trichome and diameter of the gland were measured. The data from 5 plants is averaged. See FIGS. 1 and 2A to 2C.

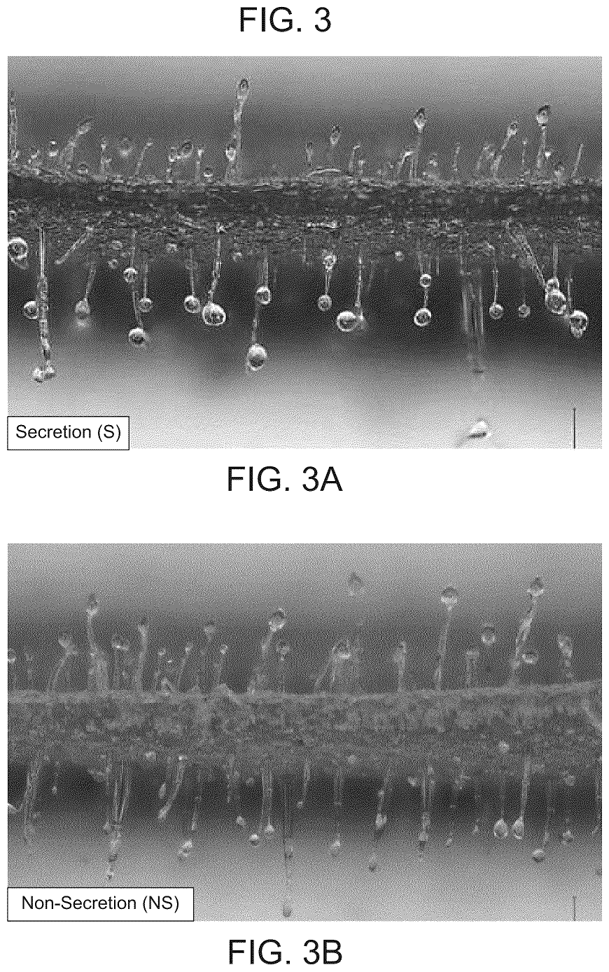

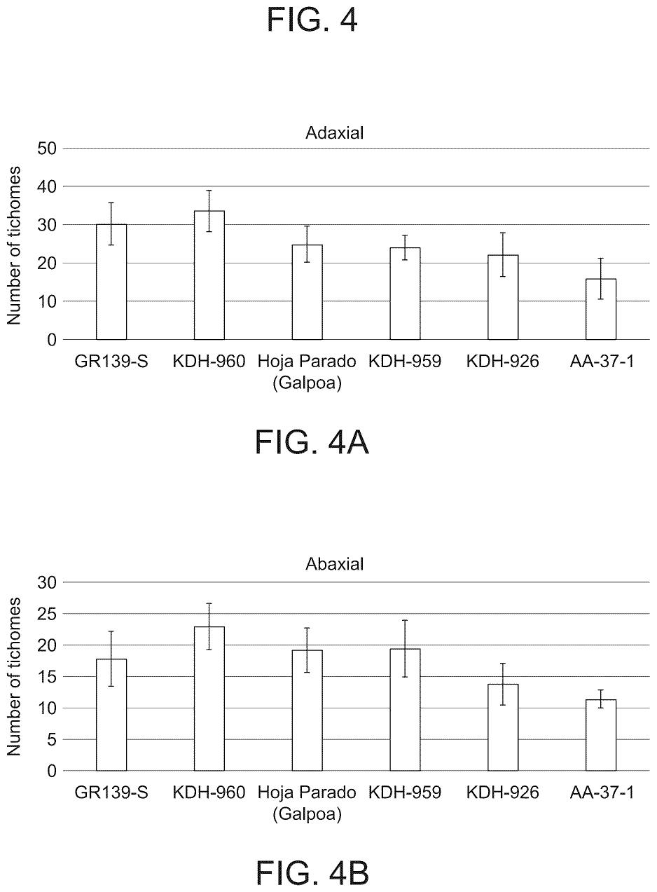

Four secretor(S) lines, KDH-960, KDH-926, GR139S, and TI 1068 (Hoja Parado) are tested. Two non-secreting (NS) lines, Gen09 (also known as GR-193NS) and KDH-959, are tested. One novel line is also analyzed (AA-37-1). Trichome density is measured on the 5th leaf from the top at topping and two weeks after topping of the plant. See FIGS. 3A and B. Trichome number is counted on both the adaxial and abaxial surface of the leaves. Five plants are evaluated and the data is presented in FIG. 4 with error bars showing standard deviation.

GR139 S and GR 139 NS are sister lines derived from TI 1406 with S phenotype denotation signifying secreting trichomes while the NS phenotype denotation signifies non-secreting glandular trichomes. The KDH lines are a series of double haploids that have varying leaf surface phenotypes from secretion to leaf surface compound makeup and trichome density. These DH lines were derived from TI 1068. These lines are described in detail in, for example, M. T. Nielsen, G. A Jones and G. B Collins 1982; Inheritance Pattern for Secreting and Nonsecreting Glandular Trichomes in Tobacco, Crop Science Vol. 22, September-October 1982, p. 1051-1053; J. C Johnson, M. T. Nielsen and G. B Collins 1988. Inheritance of Glandular Trichomes in Tobacco, Crop Science Vol. 28, No. 2 March-April 1988; M. T. Nielsen, C. P. Akers, U. E Jarlfors, G. J. Wagner, and S. Berger 1991, Comparative Ultrastructural Features of Secreting and Nonsecreting Glandular Trichomes of Two Genotypes of Nicotiana Tabacum, BOT. GAZ. 152 (1): 13-22. 1991; M. T. Nielsen and R. F Severson 1990. Variation of Flavor Components on Leaf Surfaces of Tobacco Genotypes Differing in Trichome Density, J. Agric. Food Chem. 1990, 38, 467-471; M. T. Nielsen and R. F Severson 1992. Inheritance of a Diterpene Constituent in Tobacco trichome Exudate, Crop Science, Vol. 32, No. 5:1148-1150, 1992; and M. T Nielsen 1991. Altering Flavor and Aroma Constituents of Burley Tobacco. Tob. Sci. 35:69-73, 1991.

Multiple lines are investigated and are classed into different groups depending on high or low secretion status, trichome structure, and normal or divergent chemistry. Class I includes non-secretor varieties. Class II includes heavy secreting varieties. Class III contained all the normal secreting varieties even though they exhibit differences in leaf surface chemistry based on previous literature. Class IV consisted of varieties which have divergent trichome structures along with the different leaf surface chemistry attributes. See Table 10.

TABLE 10
Selected tobacco varieties classed according to
secretion and leaf surface chemistry attributes.

| Line | Code | PCA Class | Notes |
|---|---|---|---|
| KDH-960 | V1 | Class II | High |
| (TC 577) | | | Secretion |
| TI 1269 | V10 | Class IV | Divergent trichome structure |
| TI 1223 | V11 | Class IV | Divergent |
| | | | trichome |
| | | | structure |
| KDH-959 | V2 | Class III | Normal secretion - |
| (TC 576) | | | Divergent Chemistry |
| KDH-926 | V3 | Class III | Normal Secretion - |
| (TC 575) | | | Divergent Chemistry |
| GR139 S | V4 | Class III | Normal Secretion - |
| | | | Normal Chemistry |
| GR139 NS | V5 | Class I | Low |
| | | | secretion |
| AA-37-1 | V6 | Class III | Normal secretion, |
| | | | Divergent chemistry |
| Hoja | V7 | Class III | Normal secretion, |
| Parado | | | Divergent chemistry |
| (Galpão) | | | |
| (TI 1068) | | | |
| Lonibow | V8 | Class IV | Divergent |
| (TI 1573) | | | trichome structure |
| Lonibow | V9 | Class IV | Divergent trichome |
| (TI 1613) | | | structure |

### Example 2. RNA Sequencing

RNA is isolated from various tobacco leaves at two time points: before topping (6-8 leaf stage), and at flowering. Three technical replicates from three biological replicates are collected for each time point. The isolated RNA is sequenced using Illumina® technology. The secretor and non-secretor lines that were phenotypically screened in Example 1 are evaluated here.

Sequenced reads are assessed for quality, and then mapped to a proprietary tobacco reference genome using CLC Genomics Workbench version 11.0.1 software (QIAGEN) using default parameter settings. A read count matrix including all samples is generating by aggregating the raw counts of the mapped reads for each gene in each sample against a total of 98,752 tobacco genes using the Expression Browser Tool in CLC Genomics Workbench.

RNA sequences are mapped to 98,752 tobacco genes. Genes that do not exhibit expression are removed from further consideration. For the tobacco genes that exhibit expression, gene expression values are normalized by library size in terms of reads mapped per kilobase per million reads (RPKM). Two different pairwise comparisons were made to determine the most relevant genes. GR139S, a secreting line, vs. GR139NS, a non-secreting line, and KDH-960, a secreting line, vs KDH-959. Differentially expressed genes were determined based on a FDR p-value less than 0.001. In order to reduce noise from tobacco genes with consistent expression across both time points, or that have consistently low expression, tobacco genes that exhibit a fold change in expression across all samples of less than two log are removed. The comparison of KDH-960 vs KDH-959 identified 2775 genes that were differentially expressed. The comparison of GR139S vs GR139NS identified 2501 genes that were differentially expressed. Common genes (578 genes) were then manually curated and a top 20 gene list was determined. Selection was based on highest fold change, either up or down, first and then gene annotations were used that may link to known metabolic pathways that may be involved.

### Example 3. Candidate Gene Expression

The top 20 candidate genes are identified due to differential expression patterns from the RNAseq experiments described in Example 2. A list of the top 20 differentially expressed candidate genes is provided in Table 11.

TABLE 11
Top 20 differentially expressed candidates, gene ID numbers, Automated Human Readable
Descriptions (AHRD) annotation, chromosome location, SEQ ID NO of genomic DNA (gDNA)
sequence, SEQ ID NO of cDNA sequence, and SEQ ID NO of amino acid (AA) sequence.

| | | | SEQ ID NOs. | | |
|---|---|---|---|---|---|
| Gene ID | AHRD annotation | Chr. # | gDNA | cDNA | AA |
| g82261 | Isoleucine -- tRNA ligase | 21 | 47 | 7 | 27 |
| g82257 | ATP-dependent dethiobiotin synthetase BioD | 21 | 46 | 6 | 26 |
| g82264 | Transport inhibitor response 1 protein | 21 | 48 | 8 | 28 |
| g82272 | 4-coumarate CoA ligase | 21 | 53 | 13 | 33 |
| g82265 | Transport inhibitor response 1-like protein | 21 | 49 | 9 | 29 |
| g82280 | Cystinosin-like protein | 21 | 56 | 16 | 36 |
| g82270 | Protoporphyrinogen oxidase | 21 | 52 | 12 | 32 |
| g82300 | ADP-ribosylation factor, putative | 21 | 58 | 18 | 38 |
| g82269 | Glucose-1-phosphate cytidylyltransferase | 21 | 51 | 11 | 31 |
| g82274 | Ankyrin repeat-containing protein, putative | 21 | 55 | 15 | 35 |
| g82284 | Acyl-CoA N-acyltransferase with | 21 | 57 | 17 | 37 |
| | RING/FYVE/PHD-type zinc finger protein, | | | | |
| | putative | | | | |
| g82303 | AT-rich interactive domain-containing protein 2 | 21 | 59 | 19 | 39 |
| g82268 | nucleolar protein gar2-related | 21 | 50 | 10 | 30 |
| g82273 | Emb\|CAB62340.1 | 21 | 54 | 14 | 34 |
| g58504 | Math domain-containing protein | 14 | 41 | 1 | 21 |
| g58509 | Protein transport protein sec23, putative | 14 | 43 | 3 | 23 |
| g58563 | Cembratrienol synthase 3 | 14 | 45 | 5 | 25 |
| g58508 | Magnesium transporter MRS2-like protein | 14 | 42 | 2 | 22 |
| g58533 | Cembratrienol synthase 2a | 14 | 44 | 4 | 24 |
| g1641 | Cembratrienol synthase 3 | 0 | 60 | 20 | 40 |

To validate the RNA sequencing data from Example 1, RNA is extracted from leaf samples at the layby stage, and cDNA transcripts are generated from the RNA samples, using methods standard in the art. The resulting cDNA is used to measure the relative expression of each candidate gene with quantitative RT-PCR (qRT-PCR) using gene-specific primers. Relative expression of the candidate genes is tested for the secretor backgrounds KDH960, KDH926 and GR139S. The non-secretor line tested is GR193NS. Testing lines Galpão and AA-37-1 are also analyzed and KDH959 is the wild-type control.

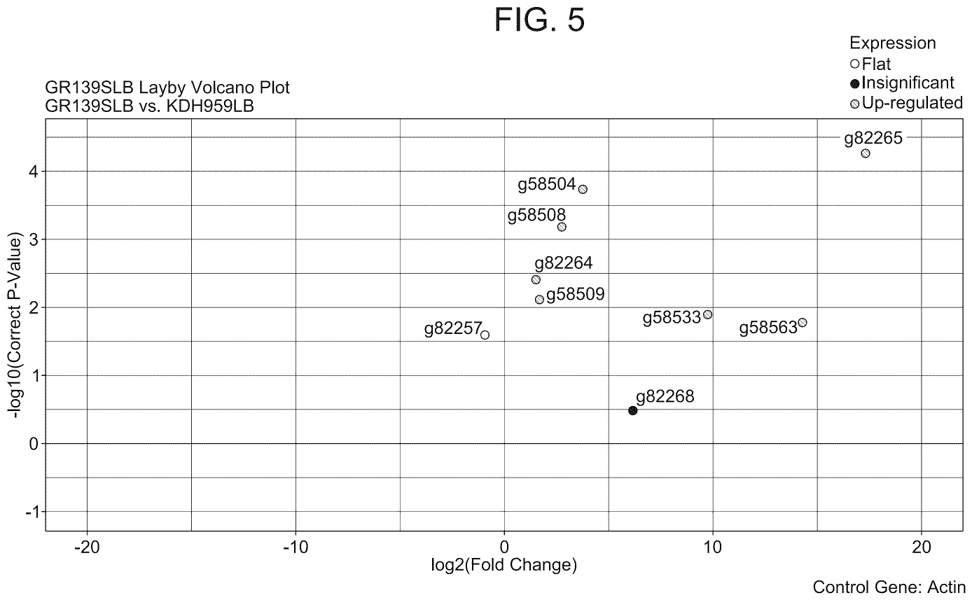

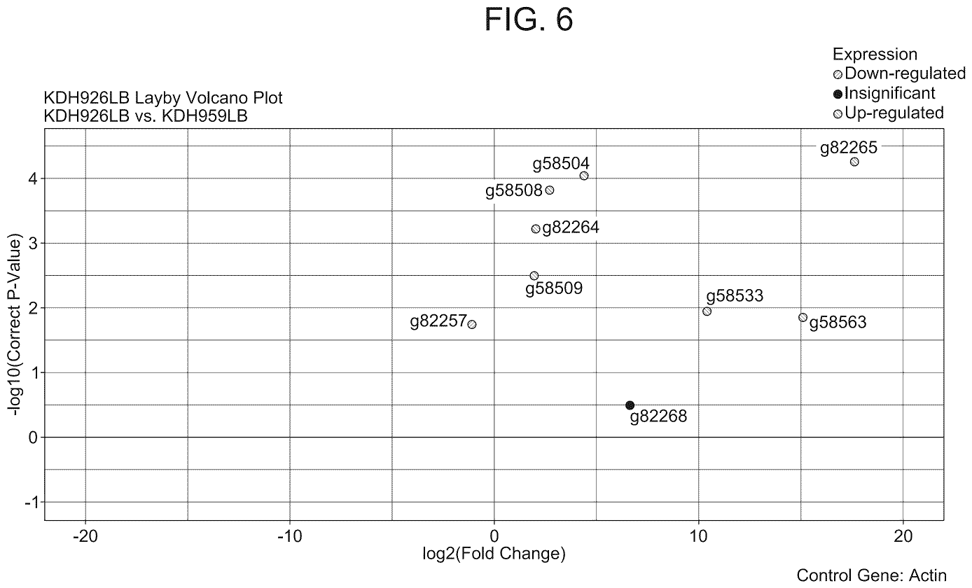

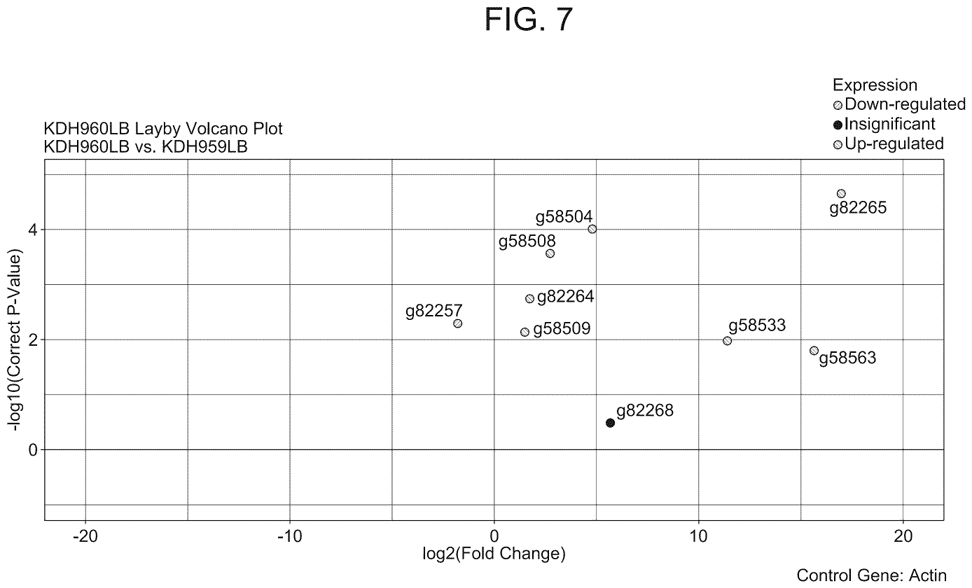

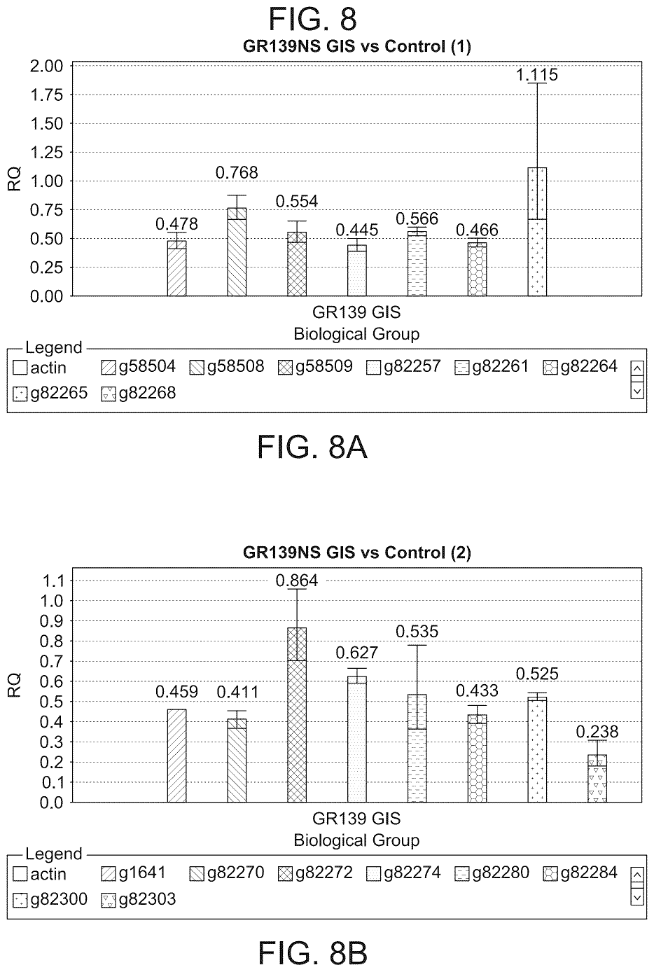

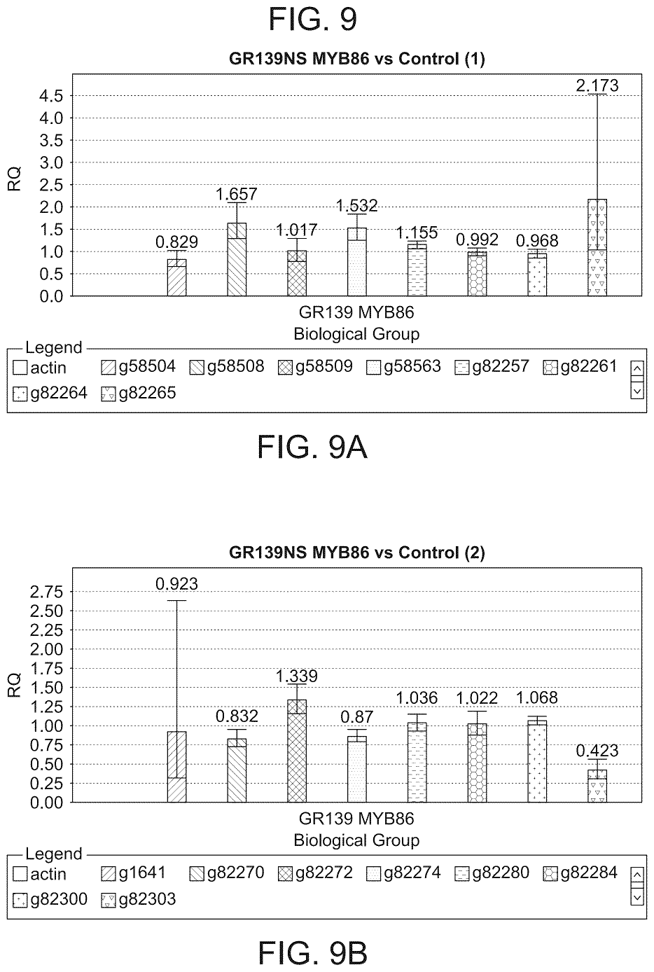

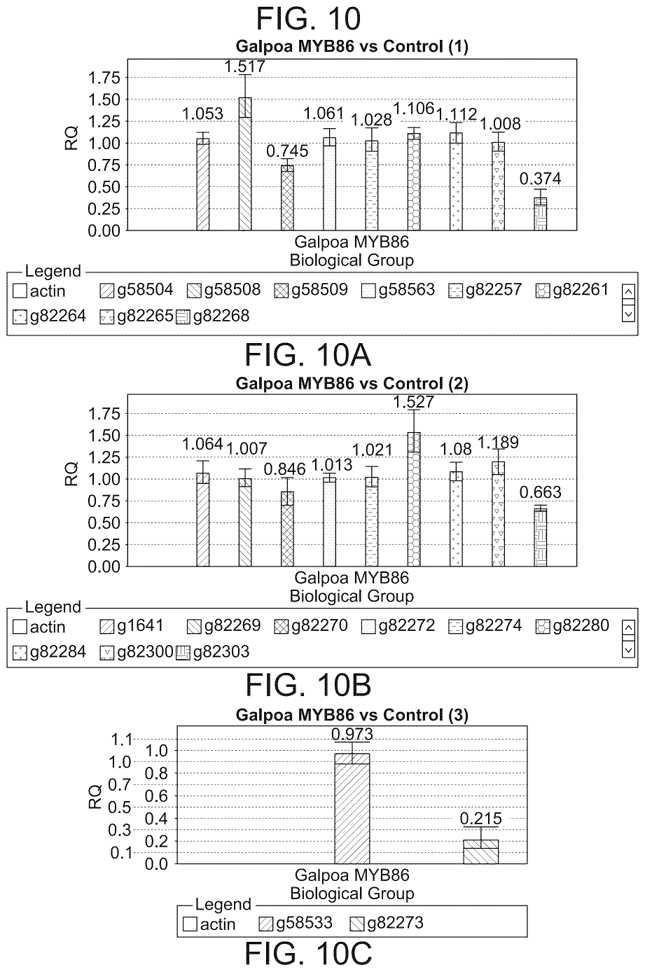

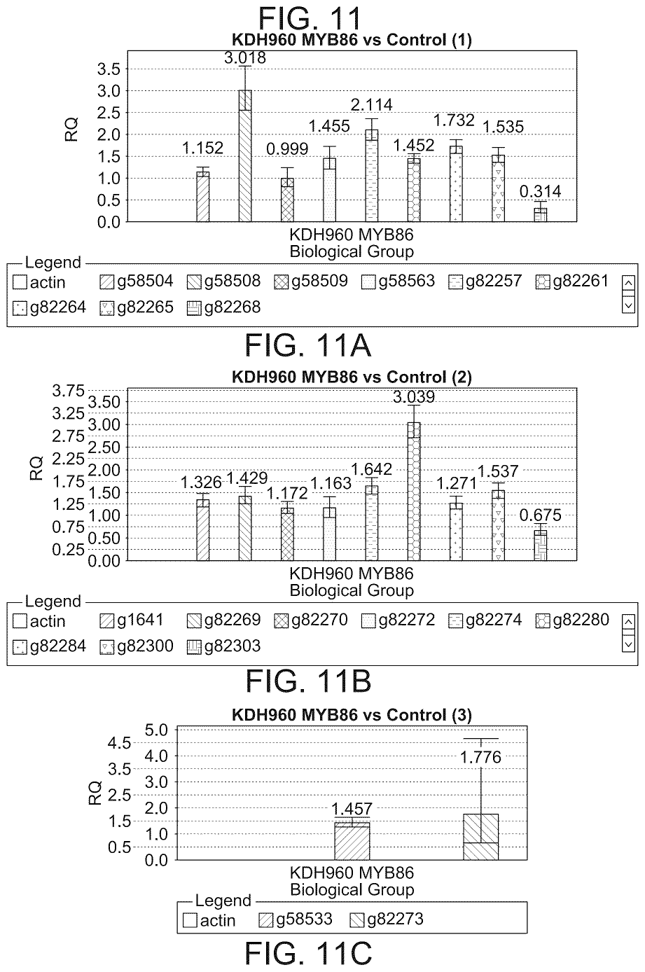

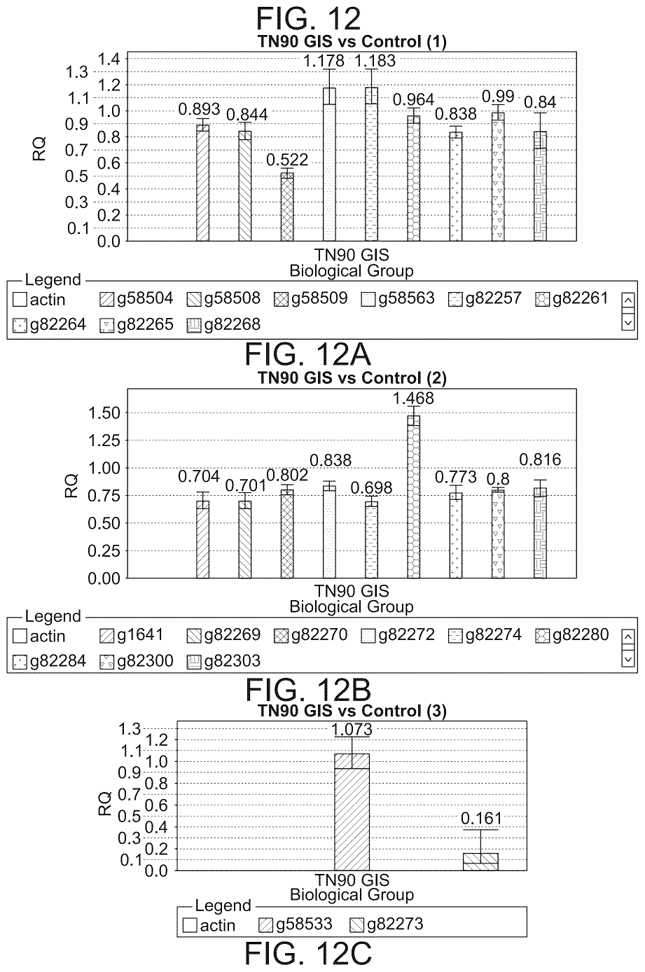

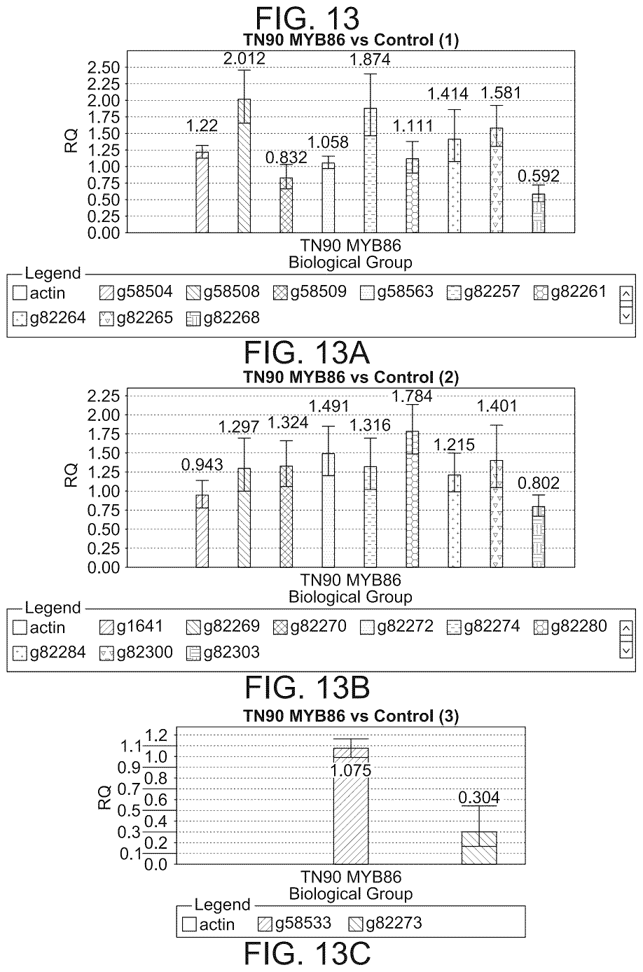

Relative expression of each candidate gene is measured and displayed as fold change, as compared to a control plant. Relative gene expression for selected candidate genes is various tested tobacco lines is presented in Table 12. Volcano plots are presented indicating the fold change for exemplary secretor lines GR139S, KDH926, and KDH960 compared to the non-secretor line KDH959. See FIGS. 5 to 7. Candidate genes showing greater than 1.5-fold expression changes are shown in Table 13. Here, fold changes are measured in GR139NS, GR139NS MYB86, Galpão MYB86, KDH960 MYB86, TN90 GIS, and TN90 MYB86. GR139NS MYB86, Galpão MYB86, KDH960 MYB86, and N90 MYB86 plants express NtMYB86 from a CaMV 35S promoter. TN90 GIS express NtGIS from a CaMV 35S promoter. Relative gene expression for candidate genes in the various Non-secretor and secretor lines, as compared to appropriate controls are shown in FIGS. 8 to 13.

TABLE 12
Summary of relative gene expression, detected by qPCR, for selected candidates genes at
Layby stage for the secretor lines: KDH960, KDH926, GR139s, non-secretor line: GR139NS,
and novel test lines: Galpão and AA-37-1, baseline is KDH959 (non-secretor).

| | Variety | | | | | |
|---|---|---|---|---|---|---|
| Gene ID | KDH960 | KDH926 | GR139S | GR193NS | Galpão | AA-37-1 |
| g58504 | 28.711 | 22.034 | 12.973 | 1.460 | 11.794 | 3.023 |
| g58508 | 6.504 | 6.587 | 6.017 | 0.944 | 19.156 | 2.639 |
| g58509 | 2.565 | 3.465 | 2.915 | 0.722 | 5.024 | 1.455 |
| g58563 | 42,964.008 | 30,688.191 | 16,184.739 | 0.241 | 12,764.680 | 56.375 |
| g82257 | 0.175 | 0.370 | 0.486 | 6.206 | 4.497 | 4.792 |
| g82261 | 1.019 | 1.176 | 0.700 | N/A | 0.096 | N/A |
| g82264 | 3.321 | 4.090 | 2.623 | 4.375 | 17.610 | 5.781 |
| g82265 | 120,087.650 | 192,321.900 | 159,944.360 | 26.199 | 715,654.600 | 11,352.516 |
| g82268 | 50.343 | 100.408 | 74.940 | 1.721 | 11.941 | 11.099 |

TABLE 13
Summary of relative gene expression, detected by qPCR, for selected
candidates genes at Layby stage for the non-secretor GR139NS
compared to MYB86 and GIS overexpressing lines. Fold change greater
than 1.5x as compared to a TN90 control plant are shown.

| | | GR139NS | Galpão | KDH960 | TN90 | TN90 |
|---|---|---|---|---|---|---|
| Gene ID | GR139NS | MYB86 | MYB86 | MYB86 | GIS | MYB86 |
| g58563 | | | 3.248 | 5.517 | | |
| g82268 | 2.337 | | | | | |
| g82257 | | 1.718 | | 1.789 | | 1.874 |
| g58504 | | | | 1.693 | | |
| g58508 | | | | 3.873 | | 2.012 |
| g82264 | | | | 1.656 | | |
| g82265 | | 1.873 | | 2.005 | | 1.581 |
| g82300 | | | | 1.649 | | |
| g1641 | | | 2.594 | 3.594 | | |
| g82272 | | | | 1.581 | | |
| g82274 | | | | 1.579 | | |
| g82280 | | | | 2.128 | | 1.784 |
| g82284 | | | | 1.705 | | |
| g58533 | | | 3.433 | 5.665 | | |
| g82273 | | | 2.049 | 6.363 | | |

### Example 4. Transformation and Regeneration of Modified Tobacco Plants

An ALCS1 expression vector is used as a backbone to generate multiple transformation vectors comprising recombinant DNA constructs (SEQ ID NO: 61). The expression vector contains a Cassava Vein Mosaic Virus (CsVMV) promoter, a NOS terminator, and a cassette comprising a kanamycin selection marker (NPT II) operably linked to an Actin2 promoter and a NOS terminator. Nucleic acid vectors comprising transgenes of interest (e.g., SEQ ID NOs: 1 to 20) are introduced into tobacco leaf discs via Agrobacterium transformation with Agrobacterium strain LBA4404. See, for example, Mayo et al., 2006, Nat. Protoc. 1:1105-11 and Horsch et al., 1985, Science 227:1229-1231. Candidate genes are inserted into the Xbal.BamH restriction sites using standard recombination.

KDH959 and TN90 tobacco plants are grown in Magenta™ GA-7 boxes and leaf discs are cut and placed into Petri plates. Agrobacterium tumefaciens cells comprising a transformation vector are collected by centrifuging a 20 mL cell suspension in a 50 mL centrifuge tube at 3500 RPM for 10 minutes. The supernatant is removed and the Agrobacterium tumefaciens cell pellet is re-suspended in 40 mL liquid re-suspension medium. Tobacco leaves, avoiding the midrib, are cut into eight 0.6 cm discs with a #15 razor blade and placed upside down in a Petri plate. A thin layer of Murashige & Skoog (MS) with B5 vitamin liquid re-suspension medium is added to the Petri plate and the leaf discs are poked uniformly with a fine point needle. About 25 mL of the Agrobacterium tumefaciens suspension is added to the Petri plate and the leaf discs are incubated in the suspension for 10 minutes.

Leaf discs are transferred to co-cultivation Petri plates (½ MS medium) and discs are placed upside down in contact with filter paper overlaid on the co-cultivation TOM medium (MS medium with 20 g/L sucrose; 1 mg/L indole-3-acetic acid; and 2.5 mg/L 6-benzyl aminopurine (BAP)). The Petri plate is sealed with parafilm prior to incubation in dim light (60-80 mE/ms) with 18 hours on, 6 hours off photoperiods at 24° C. for three days. After incubation, leaf discs are transferred to regeneration/selection TOM K medium Petri plates (TOM medium plus 300 mg/L kanamycin). Leaf discs are sub-cultured bi-weekly to fresh TOM K medium in dim light with 18 hours on, 6 hours off photoperiods at 24° C. until shoots become excisable. Shoots from leaves are removed with forceps and inserted in MS basal medium with 100 mg/L kanamycin. Shoots on MS basal medium with 100 mg/L kanamycin are incubated at 24° C. with 18 hours on, 6 hours off photoperiods with high intensity lighting (6080 mE/ms) to induce rooting.

When plantlets comprising both shoots and roots grow large enough (e.g., over half the height of a Magenta™ GA-7 box), they are transferred to soil for acclimatization. Once established, seedlings are transferred to a greenhouse for further growth, breeding, and analysis.

### Example 5. Measuring Metabolite Levels in Tobacco

Tobacco varieties are grown and leaves are harvested in order to measure metabolite levels. Samples are prepared using the automated MicroLab STAR® system from Hamilton Company (Reno, NV). Samples are extracted with methanol under vigorous shaking for 2 min (Glen Mills, Inc., GenoGrinder 2000, Clifton, NJ) to precipitate protein and dissociate small molecules bound to protein or trapped in the precipitated protein matrix, followed by centrifugation to recover chemically diverse metabolites. The resulting extract is analyzed using 4 methods: two separate reverse phase (RP)/UPLC-MS/MS methods using positive ion mode electrospray ionization (ESI), one by RP/UPLC-MS/MS using negative ion mode ESI, and one for analysis by HILIC/UPLC-MS/MS using negative ion mode ESI. Proprietary software was used to match ions to an in-house library of standards for metabolite identification and for metabolite quantitation by peak area integration.

### Example 6. Metabolome Analysis of Secretor and Non-Secretor Lines

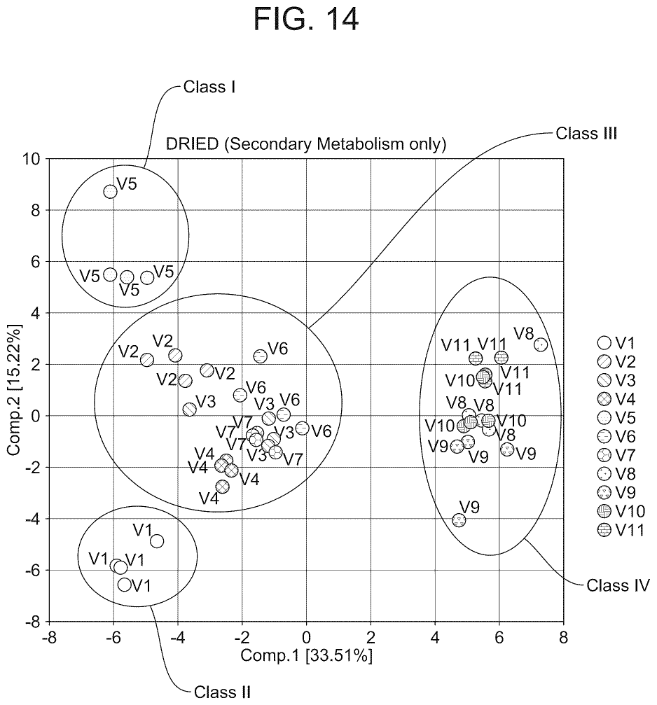

Each of the secretor and non-secretor lines evaluated in Examples 1 to 4 are grown and analyzed for metabolite composition according to the methods of Example 5. The eleven varieties tested are shown in Table 10 above. A total of 759 biochemicals are identified in this screen: 581 compounds of known identity (named biochemicals) and 178 compounds of unknown structural identity. A principal component analysis (PCA) plot is calculated using only compounds associated with the following secondary metabolism: Alkaloids and alkaloid derivatives, Benzenoids, Flavonoids, Phenylpropanoids, and Terpenoids. See FIG. 14. The resulting PCA illustrates the dramatic first component distinction between Class IV varieties (8 through 11) and the remaining varieties owing exclusively to variation in secondary metabolism. It also highlights the differentiation of several other varieties, especially Class I (variety 5) and Class II (variety 1) along Component 2. Class III did show a broader distribution than the other classes. Varieties 3, 6, and 7 were relatively similar to each other, while variety 4 trended toward the variety 1 cluster and variety 2 trended toward the variety 5 cluster.

### Example 7. Overexpression of Candidate Genes in Tobacco Plants

Separate transformation vectors comprising one each of SEQ ID NOs: 1 to 20 under the control of a CsVMV promoter are constructed using the vector described in Example 4. The vectors are used to generate modified tobacco plants as described in Example 4. Control modified tobacco plants are transformed with an “empty” vector that lacks the overexpressed gene (termed “vector controls” or “VC”). Non-secretor tobacco variety KDH959 is transformed using standard Agrobacterium transformation methods known in the art and described above. Transformed plantlets are regenerated and grown using standard procedures. PCR and qPCR are used to identify putative transformants.

### Example 8: Metabolome Analysis in Transformed Tobacco Overexpressing Candidate Genes

Transgenic tobacco plants overexpressing candidate genes are generated according to Example 7. Metabolome analysis is conducted on these plants according to Example 5. Transgenic overexpression plants demonstrate increased and decreased amounts of metabolites selected from the group consisting of Alkaloids and Alkaloid derivatives, Benzenoids, Flavonoids, Phenylpropanoids, and Terpenoids. Specific metabolites in each of these groups are listed in Table 14.

### Example 9. Knockdown of Candidate Genes Using Small RNA Molecules in Tobacco

Knockdown of the candidate genes identified in Table 11 is conducted to test for their effect on the levels of secretion of various metabolites involved in flavor and aroma of cured tobacco.

Separate transformation vectors comprising an artificial miRNA designed to reduce the transcription or translation of one each of SEQ ID NOs: 1 to 21 and 41 to 60 driven by CsVMV are constructed using the vector described in Example 4. Tobacco lines expressing these RNAi constructs can be collectively referred to as “knockdown lines.”

The vectors are used to generate modified tobacco plants as described in Example 4. The miRNA vectors are transformed in Izmir variety tobacco plants. Control modified tobacco plants are transformed with an “empty” vector that lacks the miRNA constructs (referred to as “vector controls” or “VC”). Metabolite measurements and phenotypic analysis of trichome composition are carried out in each line. Knockdown plants demonstrate increased and decreased amounts of metabolites selected from the group consisting of Alkaloids and Alkaloid derivatives, Benzenoids, Flavonoids, Phenylpropanoids, and Terpenoids. Knockdown plants demonstrate increased and decreased amounts of metabolites identified in Table 14. Metabolites higher in high secretor plants (class II plants) are shown in Table 15. Metabolites higher in low secretor plants (class I plants) are shown in Table 16.

### Example 10. Generating Mutations in Candidate Genes

Mutations are produced in each of the genes identified in Table 10 by specifically editing SEQ ID NOs: 1 to 20 and 41 to 60, separately and individually, in the tobacco genome. Tobacco protoplasts are transfected using polyethylene glycol (PEG) with plasmids encoding a CRISPR protein or a CRISPR protein and specific guide RNA (gRNA) targeting individual genes at desired positions. These mutations are produced in TN90, NLM, and K326 tobacco varieties.

Transfected protoplasts are then immobilized in 1% agarose beads and subjected to tissue culture. When calli grow to approximately 1 millimeter in diameter, they are spread on TOM2 plates. Calli are screened for mutations (e.g., insertions or deletions (indels)) at the target positions using fragment analysis. Candidates, showing target position size shifts compared to wildtype control, are selected for further culture and the consequent shoots are tested by fragment analysis again to confirm the presence of mutations.

Modified tobacco plants (TO generation) are grown as described in Example 4. Then, plants are topped, and alkaloid levels are measured as described in Example 5. Modified tobacco plants demonstrate increased and decreased amounts of metabolites selected from the group consisting of Alkaloids and Alkaloid derivatives, Benzenoids, Flavonoids, Phenylpropanoids, and Terpenoids. Modified tobacco plants demonstrate increased and decreased amounts of metabolites identified in Table 14.

TABLE 14
Metabolite Classes and Metabolites examined
in tobacco plants of this disclosure.

| Metabolite Class | Metabolite |
|---|---|
| Alkaloids | cotinine |
| | 2-hydroxypyridine |
| | nornicotine |
| | anatabine |
| | nicotine |
| | tyrosol |
| | salidroside |
| Alkaloid derivative | 3-pyridylacetate |
| | nicotine N-oxide |
| | norcotinine |
| | 6-hydroxynicotine (1) |
| | 6-hydroxynicotine (2) |
| Benzenoids | 2,4,6-trihydroxybenzoate |
| | 2,6-dihydroxybenzoic acid |
| | 3,4-dihydroxybenzoate |
| | 4-hydroxybenzoate |
| | benzoate |
| | gentisic acid-5-glucoside |
| | hydroquinone beta-glucopyranoside |
| | protocatechuic acid-3-glucoside |
| | salicylate |
| | salicyluric glucuronide* |
| | salicylate-glucoside |
| Flavonoids | dihydrokaempferol |
| | eriodictyol |
| | dihydroquercetin |
| | naringenin |
| | quercetin |
| | quercetin 3-glucoside |
| | rutin (quercetin 3-rutinoside) |
| | 3-methoxyapigenin |
| | kaempferol 3-O-glucoside/galactoside |
| | isorhamnetin 3-rutinoside |
| Phenylpropanoids | aesculetin |
| | chlorogenate |
| | 4-hydroxycinnamate |
| | cryptochlorogenic acid |
| | lariciresinol 4-O-glucoside |
| | liriodendrin |
| | caffeate |
| | vanillate |
| | coumaroylquinate (2) |
| | coumaroylquinate (4) |
| | coumaroylquinate (5) |
| | coumaroylquinate (3) |
| | feruloylquinate (1) |
| | feruloylquinate (2) |
| | feruloylquinate (3) |
| | feruloylquinate (4) |
| | feruloylquinate (5) |
| | melilotoside (1) |
| | scopoletin |
| Terpenoids | 3-hydroxy-3-methylglutarate |
| | mevalonate |
| | cis-trans-farnesol |
| | carotene diol (1) |
| | carotene diol (2) |
| | carotene diol (3) |
| | beta-cryptoxanthin |
| | geranyllinalool |

TABLE 15
Metabolites higher in class II (high secretors). Terpenoids and
Flavonoids are higher in the heavy secreter class. Fold change
is calculated by the normalized mean value of the compound detected
in Class I divided by the normalized mean value detected in
Class II. All values have a p-value less than 0.05.

| | | Fold change (Non Secreter |
|---|---|---|
| Class | Metabolite | vs Heavy secreter) |
| Terpenoids | cis-trans-farnesol | 0 |
| | carotene diol (1) | 0.77 |
| | carotene diol (2) | 0.68 |
| | beta-cryptoxanthin | 0.27 |
| | geranyllinalool | 0.02 |
| Flavonoids | dihydrokaempferol | 0.08 |
| | eriodictyol | 0.06 |
| | dihydroquercetin | 0.1 |
| | naringenin | 0.15 |
| | quercetin | 0.15 |
| | quercetin 3-glucoside | 0.39 |
| | rutin (quercetin 3-rutinoside) | 0.37 |
| | 3-methoxyapigenin | 0.2 |
| | kaempferol 3-O- | 0.69 |
| | glucoside/galactoside | |

TABLE 16
Metabolites higher in Class I (low secretors). Benzenoids
are higher in the low secreter class. Fold change is calculated
by the normalized mean value of the compound detected in
Class I divided by the normalized mean value detected in
Class II. All values have a p-value less than 0.05.

| | | Fold change (Non Secreter |
|---|---|---|
| Class | Metabolite | vs Heavy secreter) |
| Benzenoids | 2,6-dihydroxybenzoic acid | 3.28 |
| | 3,4-dihydroxybenzoate | 2.21 |
| | 4-hydroxybenzoate | 1.3 |
| | benzoate | 1.27 |
| | gentisic acid-5-glucoside | 4.45 |
| | hydroquinone beta- | 1.24 |
| | glucopyranoside | |
| | protocatechuic acid-3- | 4.28 |
| | glucoside | |
| | salicylate | 2.96 |
| | salicyluric glucuronide* | 3.07 |
| | salicylate-glucoside | 7.34 |

1. A modified tobacco plant, or part thereof, comprising at least one non-natural mutation in an endogenous nucleic acid sequence, wherein the endogenous nucleic acid sequence encodes a polypeptide comprising an amino acid sequence at least 95% identical or similar to an amino acid sequence selected from the group consisting of SEQ ID NOs: 21 to 40.

2. The modified tobacco plant, or part thereof, of claim 1, wherein the modified tobacco plant produces at least one leaf comprising a reduced amount of at least one metabolite as compared to the amount of the at least one metabolite in a control tobacco plant lacking the at least one non-natural mutation in the endogenous nucleic acid sequence when grown under similar growth conditions.

3. The modified tobacco plant, or part thereof, of claim 1, wherein the modified tobacco plant is of a tobacco variety selected from the group consisting of a flue-cured variety, a bright variety, a Burley variety, a Virginia variety, a Maryland variety, a dark variety, a Galpão variety, an Oriental variety, and a Turkish variety.

4. Cured tobacco material from the modified tobacco plant, or part thereof, of claim 1.

5. The cured tobacco material of claim 4, wherein the cured tobacco material comprises flue-cured tobacco material, air-cured tobacco material, fire-cured tobacco material, or sun-cured tobacco material.

6. A tobacco product comprising the cured tobacco material of claim 5.

7. The tobacco product of claim 6, wherein the tobacco product is a smokeless tobacco product.

8. A modified tobacco plant, or part thereof, comprising a recombinant DNA construct comprising a heterologous promoter operably linked to a nucleic acid encoding at least one small RNA molecule capable of binding to and reducing the expression of an endogenous nucleic acid sequence encoding a polypeptide at least 95% identical or similar to an amino acid sequence selected from the group consisting of SEQ ID NOs: 21 to 40.

9. The modified tobacco plant, or part thereof, of claim 8, wherein the modified tobacco plant produces at least one leaf comprising a reduced amount of at least one metabolite as compared to the amount of the at least one metabolite in a control tobacco plant lacking the recombinant DNA construct when grown under similar growth conditions.

10. The modified tobacco plant, or part thereof, of claim 8, wherein the modified tobacco plant is of a tobacco variety selected from the group consisting of a flue-cured variety, a bright variety, a Burley variety, a Virginia variety, a Maryland variety, a dark variety, a Galpão variety, an Oriental variety, and a Turkish variety.

11. Cured tobacco material from the modified tobacco plant, or part thereof, of claim 8.

12. The cured tobacco material of claim 11, wherein the cured tobacco material comprises flue-cured tobacco material, air-cured tobacco material, fire-cured tobacco material, or sun-cured tobacco material.

13. A tobacco product comprising the cured tobacco material of claim 12.

14. The tobacco product of claim 12, wherein the tobacco product is a smokeless tobacco product.

15. A modified tobacco plant, or part thereof, comprising a recombinant DNA construct comprising a heterologous promoter operably linked to a nucleic acid sequence encoding a polypeptide comprising an amino acid sequence at least 95% identical or similar to an amino acid sequence selected from the group consisting of SEQ ID NOs: 21 to 40.

16. The modified tobacco plant, or part thereof, of claim 15, wherein the modified tobacco plant produces at least one leaf comprising an increased amount of at least one metabolite as compared to the amount of the at least one metabolite in a control tobacco plant lacking the recombinant DNA construct when grown under similar growth conditions.

17. The modified tobacco plant, or part thereof, of claim 15, wherein the modified tobacco plant is of a tobacco variety selected from the group consisting of a flue-cured variety, a bright variety, a Burley variety, a Virginia variety, a Maryland variety, a dark variety, a Galpão variety, an Oriental variety, and a Turkish variety.

18. Cured tobacco material from the modified tobacco plant, or part thereof, of claim 15.

19. The cured tobacco material of claim 18, wherein the cured tobacco material comprises flue-cured tobacco material, air-cured tobacco material, fire-cured tobacco material, or sun-cured tobacco material.

20. A tobacco product comprising the cured tobacco material of claim 18.

21. The tobacco product of claim 20, wherein the tobacco product is a smokeless tobacco product.


SEQUENCE LISTING (90 sequences)

SEQ ID NO: 1 - DNA, 3429 bp, synthetic construct
  source 1..3429: mol_type = other DNA
atggcggcttcaagtgcacatgaggaggcagggacaggaaggtcttatgagggattgtcgagcgggcagcagcaatgcagtgaagcgttggcggaatggaggtcatccgagcaggtagaaaatggaactccatcaacttcgcctccttactgggattgtgatgatgacgacgatggtggccctaaaccttctgagctatatgggaaatatacatggaagattgataagttttcgcaaattaacaagagagaacttcgcagtaatgcatttgatgttggcggctacaaatggtacattctgatttatcctcaggggtgtgatgtttgcaaccatctctctttgtttctttgtgtggctaatcatgacaagctccttccaggttggggtcattttgctcaattcactattgctgtggtgaacaaagacccgaagaagtccaagtattcggatacattacatcgtttctggaagaaagagcatgattgggggtggaaaaaatttatggagttgtcgaaagttttagatggttttgttgatgctgatacgctcataataaaagctcaagttcaagtaatcagggagaaaccagaacatccctttcgttgccttgaccgtcaatatcgaagggagcttgttagggtatatttgactaatgtggaacaaatctgccgccgtttcgtggaagagcgacgagtaaagcttgggaagttgatagaggatagagctcgatggtcaagcttctgtgctttttggctgggaatggaccaaaattctaggtgcgacatgtccaaggagagatcagattcaattttaaaagtggttgtcaagaactttttcatagagaaggaggttacttctactcttgttatggactccttgtacagcgggcttaagtctcttgaaggccagataatgggcaagaaaggtaaagcaaaatattcggatgcagatgaacaactggttcctattgttcgtatggagaaaaacatgtttgtgttggtggatgatgtcttgctattgcttgagagtgctgcattggagccattgcctccaaaggatgagaaaggccctcaaaacagaacaaaggatggcacttctggagatgagtttaacaaagatacaattgagcgtgatgagaggcgtttaactgaattaggtcgtcgaaccattgaaatatttgtcctagcacagattttcagtaaaatagaggttgcgtatcaggaggctgttgctttaaagaggcaagaagagctcatccgtgaagaggaagctgcttggctggcagaaactgaacagaaagctaaacgagcatctggaaaggaaaaaaagtccaagaaaaagcaggctaagcagaagcggaataatcacaaagtaaaggataagggtgttgatgaaaaacctggatccatggagctatataaaattgatcaggatgggcctattggtgaaggaaacgagtacataaatgaggagcgagaagcagtacttggaaaaccagatatactggaagctgtatctgatgtttctgattcaattgattgtgtcccagaggttattaatcctgactctgaagacagagatgcaagtcctgtcaattgggatactgacagctcagaagttcatccttccacggaaactagctgcagtgggttaagtgacctttcagctgtgcaaaatgggttagctggaaggagaagtccttccgtcatggatgatagttcatcgacgtgttccacagattcgattccttccgtggtttcgaatgggccttgcagatggacttcggctaaccataaaaatcagaaatcacctagcagagggaagaaccaaagaaacaagtcaacgtctaatgcggctgattgggctagtgaaacactaagtcagccattagatgctgtttcagacttaggacagctaagtgatagaagttgcggggcacctggatctgagtcgcattctactgtgctttcgtcgaatgagcagcaaaaggtgaagaaggaagtagtagctttgcagcagagaaaggccgagacagagagaccttcaatggagaagccgagtgtcaagtcttctcggagaagccctccaaaagatgcaggctctgctgttcagcagaagtcacagacgaagatctctgtcacaagtgatcctgctttggttaaaagatcctctacagatggtcccaagctagctgataaatcagctctagtgtctaattcatctgaaactgcggtattgaaagctgatccgcataaagctgtagagccaagggtcaaggataagccctcagtgcagcccatttgtattatgactggggagtcctcgtctcaacaagtaacagtctcttctacaacagaaaatttcaaatggcaacaagtgcccgccgtgtccagaccattgtgtgatcctttagttcctggacctaggcctgctgcccctgttgtttccatggttcagacaacaccatcactggctcgttcagtgagtgcagctggccgattgggtcccgatccttctccagcaacacacagctatcttactcagtcctaccgaaatgcaattatgggtggtcctgtttctggaagtccggctagttttagtcaacctcattctccaatttcagcagttaacctatcacattcgtactctcaacaacaacctccactggtttctggggcatcatttttacctcatggtttggaaagagcagaaccttgctcgatgagaccaagtttttcatatggaatggtgaataatggcagcttgcagaatggactgcaatgggagtgtccccaaagggacagtaacaggagtatatctcaacatcatccttctgcatcaaatggaattaggaactttgatatgttcaagactgtaaacagtagaacacatgatcacgttcctgattctctggcttgcacgtctggacgccagtcccagagtgtatcgcctgatgaatttcctcatcttgatattattaacgacttactgaacgatgatcatgggattggaagggcttctatacccgacttgggctttcaaagttttagcagcggatcacagcatctaaatcatgtgttcacattcccaggtgatattggcaccccaactgacctaggtgcctcatcaagctcttgtaggtttgaacgaacacgaagtcatcatgacatgttccagcataactattctggaggcctttttgatagtgctagtgatatgattctgcagcccgatccacgatttatgaatggacatcatattgatgggttagttcctaaccagtggcaaatgataggttctgatccgtcatttcttgggatgaggaatgtgaatattgatggtagtcacccgtatcccctccctgactattcaaacatggcgtgtggtgtaaatgggtacggattatttaggccttcgaatggcctttga

SEQ ID NO: 2 - DNA, 1182 bp, synthetic construct
  source 1..1182: mol_type = other DNA
atggtgatgggtcgagaagtggtgccggtgatggatccgagtcaagtagtcgggttgaagaaaaaaggtaccgggtcacggagttggcttctgatggatgcttccggtttggagactgttttagatgttgataagtatgcgatcatgcatcgcgtgcaaattcacgctcgtgatcttcgcattatcgatccgctactatcttacccttccactattctggggcgtgaaagagctattgttctcaatttggagcatatcaaggctattattacatctgaagaggttcttcttcgagatccattggacgaaaatgttactcctgttgttgaggaacttagaaggcggctgaaaccaatgaatgcaaatcgtgaagatcagagagaggaaaaagatatgaatgttcagcatgatattgaaacaactgaagaagatgagtctccatttgaattccgagctctagaggtcgctttggaagccatctgtagttaccttgctgctcgcacattagaattggaaactgctgtttaccctgctttagacatgctaacatcaaagattagtagccgtaatctggatagagtgcggaaattaaagagtcaaatgacacggttgactgctcgcgtacaaaaggtgagagatgagcttgagcaacttctggatgatgatggtgatatggctgacctttatttgtcaagaaaattggctggtgcatcacctgtaagtggatcaggtgcagccagttggtttcttgcctcacctaccattggctcaaagatatccagggctagtagagctagtattgcaacatttcggggggatgagaatgatgttgaagagcttgaaatgttattggaggcttacttcgggcaaattgatagcacatttaataaactaattacgctacgagaatatattgataacacagaagattacataaatattcagctggacaatcatcgaaatcagctcattcagttagaactttttctcagttccggaactgtgtgtttgtctatctactctttgattgctgcgatttttgggatgaacatcccatattcttggaataaagatcatggttacatgtttaaatgggtggtaatcattagtgctatcttctctgccgtgcttttcatatttataatctcttacgctcgtttcaaagggcttgttgggtcatga

SEQ ID NO: 3 - DNA, 2325 bp, synthetic construct
  source 1..2325: mol_type = other DNA
atggatttcgtggaactggaggcaattgaaggtctccggtggtcatggaattcatggccaatttcaaaatccgaatcttctgccctaattatccctttatcagttatgtgcaatccgttaatgcaatttaacgagctccctatacttaattacgatcctgttatttgctctcaatgcggcgccgttttaaacccttacgcccgtgttgattacgtgtcgcgaatctggaattgcccgttttgttaccggaaaaacccgtttccgagatcgtacctcggaattaacgaaaataatgtgccagctgagctttttccgacttatagtactgttgaatatcacttgggtaaaaagggtttgacccaaaacccgaattccagctctaattttggaaatgtgtcgatgttatcgaaaatgccttcattttcgagcttggattgggcgggttatgggcttggcccggcatttgtgtttgtggttgatgcctgtactagtgatgaggagcttagggtggtgaaaaatgagctacttcatgttattgctcaattgcctgaaactgcattggttgggttggtggtgtttgattcaatggttagagttcatgatctcgggtttggagagtgctgtagggttgtgatgttccacggtgagcgtgagctttcttctgagcagaccaagcaactcctgggaattcatcatgtgaagtaccaagcaggaaaggtacctatggctccaaaacagggatttctgataccactatcagaaggggagttcagcatcacatcagcaattgaagatatacaatcatcagcacaggttatgcaagggcatagaccttttcgagctacaggagtagcagtatcagttgctgtcggactactagaaggatgcttggtctctgcaggttcacgtatcatgatctttacctcagggccagcaacaattggcccagggatgatcgtaagctctgagcttggcaatgccattagaaatcatcgagacatcggtaatggtcatgctgcttactacaagaagtccagtgaattctataagcagatatcaagaagattgtctaattcatccattgcacttgatctctttgcttgttcattggatcaagttggagcagctgagcttagagcttcagttgaaagctcaggagggttcatgatgttaacagagtcatttgactcggaccagttcagaaagtgtttacgtcacatatttagccatgacgaggctggtaatttgaaaatgtgccttgatgcaactatagagatagtaacaacaaaggatgttaaaatttgtggggctcttggtccatgtgtttctctgcagaagaagaatggttcagccagtcacaaggaaattggcgagggaggtacctatacgtggaaattgggggcacttactaacaaaacgtgcattgccttcttttttgaagtaggggatgaacagaaagcccagcctagctctgcatttttcatacagttcataacacactatagatatggaaacacgggaattcgaaagagggtgaccactgctgcaagaaggtgggttgggaaccactctccggaaattgctgctggctttgatcaagaagcagcagcttcagtgatggctaggcttgccattcatagagcagagagcaatttttctcaagatgttgtgcggtggctagataaatgtttgatacgttttgcttcaaagtatggggattatatccaggaagacccctcatcattccgtcttgcaactaatttatcccttttcccacagttcatgtattatttaaggaggtctcagtttattgatgtcttcaacagcactccagacgagactgcttttttccggctgttgttgaaccgtgagggggtagttggctcacttattatggtccagccaaccctcttccagtattcattcgagggtccccctatccccgttcttttggacatctgctccgtctctccagatgttatcttgctctttgactcctacttctatgtggttattcattatggctctaagattgtacaatggcgtaaacttggctatgacaaggatccaagccacgatagtttccgcaagctattagaagctcccgagattgatgcagagcaactggtttcggaaaggattcctgtgccaaagctgataaaatgtgagcaacatagtagtcaggctaggtttctccttgccaagttgaatccatcggttacacacaactcaacatacacagaaggcaaagagcttatatttacggacgaactgagtctacaagtttttattgagcacttgcaagctttagcagtgcagggttga

SEQ ID NO: 4 - DNA, 1797 bp, synthetic construct
  source 1..1797: mol_type = other DNA
atgagtcaatcaatttctccattaatctgttctcactttgcgaaatttcagtcgaatatttggagatgcaatacttctcaactcagagttatacactcatcatatgcctcttttggagggagaagaaaagagagagtaagaagaatgaatcgagcaatggatctttcttcaagctctcgtcatttggcagattttccctcaacaatttggggtgaccattttctctcctacaattctgaaataacagaaattactacccaagagaaaaatgaacatgaaatgctaaaagaaatagttcggaaaatgttggtagaaactccagataatagtacacaaaaactagtcttgattgacacaattcaaagattgggattagcatatcatttcaatgatgagattgaaaactccattcaaaacatctttaatttgtctcaaaatagtgaagatgacgatgaacacaacctttatgttgctgctcttcgttttcgacttgcgaggcaacaaggatattacatgtcttcagatgtgttcaagcaattcactaaccatgacggaaaattcaaggaaaatcatactaatgatgttcaaggattattgagtttgtatgaagcagcacatatgagagtgcacgacgaggaaattctagaagaagctcttatctttaccacgactcatctcgagtccgtgatcccgaatttgagcaactcgcttaaggtacaagttactgaagccttaagccatcctattcgcaaagctataccaagggtgggagcaaggaaatacatacacatatatgaaaacattggaacacataatgatttacttttgaaatttgcaaagttggacttcaacatgttacaaaagcttcatcgaaaagagcttaacgagctaacaagctggtggaaagatttggatcgtgcaaacaaatttccatatgcaaaggacagattagtagaagcttacttttggacggtgggaatatattttgaacctcaatatagtcgttcaagaagtttggtaacaaaagtagtcaaaatgaactccattattgatgacacttatgatgcttatgcaacttttgatgagcttgtgcttttcacggatgcgatccaaagatgggacgaaggtgccatggatttattaccgacatatctgagacctatttatcaaggccttctcgacgttttcaatgaaatggaagaagtattggccaaagaaggtaaagcagatcacatctactatgcgaaaaaagagatgaaaaaggtggcggaagtctattttaaggaagctgaatggttgaatgctaactacattccaaaatgcgaggagtatatgaaaaatggacttgtaagctctaccggtccgatgtatggaataatttctttggttgttatggaggaaattataacaaaagaggcttttgaatggttgacaaatgaacctttgattcttcgagctgcatcaacaatttgtagattaatggatgatatggctgatcatgaagttgaacaacaaagaggacatgttgcttcatttgttgagtgctacatgaaagaatatggagtttcaaagcaagaagcatatgttgagatgcggaaaaaaatcacaaatgcgtggaaagatataaataaggaactcttgcgccctactgcagtaccaatgtttatcctcgaacgatctttaaatttttcaagattggccgatacatttttgaaagatgatgatggatacacaaatcccaaatccaaagttaaagacttgattgcttcgttgtttgtcgaatctgtcgacatatga

SEQ ID NO: 5 - DNA, 1275 bp, synthetic construct
  source 1..1275: mol_type = other DNA
atgttggtagaaactccagataatagtacacaaaaacttgtcttgattgacacaattcaaagattgggattagcatatcatttcaatgatgagattgaaaactccattcaaaacatcttttatttgtcccaaaatagtgaagaagacgatgaacacaacctttatgttgttgctcttcgttttcgacttgcgaggcaacaaggaaattacatgtcttcagatgtgttcaagcaattcactaaccatgacggaaaattcaaggaaaatcatactaatgatgttcgaggattactgagtttgtatgaagcagcacatatgagcgtgcacgacgaggaaattctagaagaatctcttatctttaccaccactcatctcgagtccatgatcccgaatttgagtaactcgcttaaggtacaagttactgaagccttaagccagcctattcgcaaaactataccaagggtgggagcaaggaaatacatatacatttatgaaaatattgaagcacataatgatttgcttttgaaatttgcaaagttggactttaacatgttacaaaagcttcaccggaaagagcttaacgagctaacaagatgggacataggtgccatggattcattaccgacatatctgagacctatttatcaaggccttctcgacgttttcaatgaaatggaagaagtattggccaaagaaggtaaagcagatcgcatctactatgcgaaaaaagagatgaaaaagttggtggcaacctattttaaggaagctgaatggttgaatgctaactacattccaaaatgtgaggagtatatgaaaaatggacttgtaagctgtacgggtatgatgtatggaacaatttctttggttgttatggaagaatttataacaaaagagacttttgaatggttggcaaatgaacctttgattcttcgagcttcatcaacaatttgcagattaatggacgatattgccgatcacgaagttgaacaacaaagaggacatgttgcttcatttgtggagtgctacatgaaagaatatggagtttcaaagcaagaagcatatgttgagatgtggaaaaacattacaaatgcgtggaaatgcataaataaggaactccttcgccctactgcagtaccaatgtttgtccttgaacgaactttaaattatacaagattggttgatacatttttcaaagatgatgatggatacacaaatcccaaatccaaagttaaagacctaattactttgttgtttgtcgaatctatcgacatataa

SEQ ID NO: 6 - DNA, 537 bp, synthetic construct
  source 1..537: mol_type = other DNA
atggcagcctcagaaatgctatcgaaggagcaattgcttcatctttttgatcgcttcgcgttcctcacctcccaacctgatgtcaagaaaaggattgctgatgctgtcagcgacaagcaggaggctgttgctgttaccactgccattcaagaggagatattttccgagatgggaattgatccaagatttggtcttgcttgcttggggaaaataaacatgacctatgagagtgatcgagacctgatgattcgtttttatgaatttgttgcaaaagaagaaatggcttgtgaagaggccgagcttggtcctgacgggtttgctgaaagattaaagatgcaacaaaatttacaagagcagcaactggagatgctgaagtacatgcgcaactttcacatggatgatcagtctgcaatacttgagaagattcaccagcagttggagaaggccaactttgacactgaagcttctgtgctgacagtggagcagatgcaggatgttgttcgacggagagtatcacctttgttccagcccagataa

SEQ ID NO: 7 - DNA, 219 bp, synthetic construct
  source 1..219: mol_type = other DNA
atggcgttaacgaactttatactaacggtggtcggagtgagcgcggcgatattattgctgaggagcgatgtcaaacagtctgcttccatcttcaagcgtaacgttcgtcaaattcgccattggctcgaggaagagtcttcctccgccgccaaggaattggagaaggcaaaaccaaaggagatacctcacaaagacattcctaaggaggataagcattag

SEQ ID NO: 8 - DNA, 1740 bp, synthetic construct
  source 1..1740: mol_type = other DNA
atgtgtttgtgtcaatgtatgatgaattacttccctgaggaagtgttggagcatgtcttcaatttcttgacttcccacagggaccgaaatgcggtctctttggtctgtaattcttggtatagagttgagaggttcagtagagagaaggtgtttgtagggaattgttacgcggtaagccccgagagagtgattgctaggtttccgaggcttaggtcgcttaccttgaaagggaaaccccattttgcggatttcaatttggtccctcatgattggggaggttatgttgacccttggattgaggcgatggctgcgagtggtgtgaacttggaggagcttcgtttgaagaggatggtggtttcggatgagagccttgaactactctcgcgatcctttcctaatttcaagtctttgattttggttagctgtgaaggtttctccactgatggtcttgctgctattgcttccaattgcaggtttttaagggagttggacttgcaggaaaacgaagtcgaagatcgcagaggacagtggcttagttgtttttcagacaactgcactactcttgtctctttgaattttgcttgcctcaaaggagaagttaacttggcagctcttgagaggctagtggctagatgtccaaacctaagaagtctgagattaaatcatactgtgcctcttgatgctcttcaaaggattttaattaaagctcctcacttagtggacttggggacagggagttttatcagtgatccagtttctgagacttacaacaaactaaagaatgctctgaagaggtgtacttctattaggagcttgtcggggttcttggagattgctcctcgctgcctgcctgctatttatccaatttgcctgaatttgtcgtccctgaacttgagctatgcacctggaatctacagtgctgaacttataaagctgattcgtttctgcggaaaactagagcgcctatggattctggatactattggagataaagggctaggtgttgtggcttctacttgtaaagaattgcaggaattaagggtcttcccatctgaccttcatggatttggccatgctgctgtaactgaagagggcttggttgcaatatcagccggttgtccaaagctcaactcattgttgtatttctgccagcagatgactaatgctgcacttataactgttgccaaaaactgcccaaactttattcgtttcagattgtgcactcttaacccaactgtgccagatgcagttaccatgcagccactagacgaaggttttggggcaattgtacagtcgtgcaaaggcctcaagcggctgtcagtctctggtcttctaactgatcaggttttcctttacataggaatgtatgctgagcagcttgagatgctctccatagcctttgctggagatagtgacaaggggatgctatatgtgttgaacgggtgcaagaaaatgaaaaagcttgagatcagggatagcccttttggtaatgtggcacttctggcggacgtggggaagtatgagacaatgcgatccctttggatgtcgtcctgccaagtgactcttggagcatgcaaggctgttgctcagaagatgccgaggctcaatgtggagatcatcagcgagaacgatcagatagacacttgctctgatgacaggcaaaaggtcgagaagatttacttgtatcgaacattggttggtcccaggaaggactccccagatttcgtccggacgttgtag

SEQ ID NO: 9 - DNA, 1032 bp, synthetic construct
  source 1..1032: mol_type = other DNA
atgtgtttgtgtcagtgtatgatgaattacttcccagaggaagtgttggagcatgtcttcaatttcttgacttcccacagggaccgaaatgcggtctctttggtctgtaattcttggtatagagttgagaggttcagtagagagaaggtgtttgtagggaattgttacgcggtaagccccgagagagtgattgctaggtttccgaggcttaggtcgcttaccttgaaagggaaaccccattttgcggatttcaatttggtccctcatgattggggaggttatgttgacccttggattgaggcgatggctgcgagtggtgtgaacttggaggagcttcgtttgaagaggatggtggtttcggatgagagccttgaactactctcgcgatcctttcctaatttcaagtctttgattttggttagctgtgaaggtttctccactgatggtcttgctgctattgcttccaattgcaggtttttaagggagttggacttgcaggaaaacgaagtcgaagatcgcagaggacagtggcttagttgtttttcagacaactgcactactcttgtctctttgaattttgcttgcctcaaaggagaagttaacttggcagctcttgagaggctagtggctagatgtccaaacctaagaagtctgagattaaatcatactgtgcctcttgatgctcttcaaaggattttaattaaagctcctcacttagtggacttggggacagggagttttatcagtgatccagtttctgagacttacaacaaactaaagaatgctctgaagaggtgtacttctattaggagcttgtcggggttcttggagattgctcctcgctgcctgcctgctatttatccaatttgcctgaatttgtcgtccctgaacttgagctatgcacctggaatctacagtgctgaacttataaagctgattcgtttctgcggaaaactagagcgcctatgggttgatatttttgccccccttttaattatttccttgttgctttattttcctacaattgtttga

SEQ ID NO: 10 - DNA, 3201 bp, synthetic construct
  source 1..3201: mol_type = other DNA
atggttttgggattgaagggcagaactaggaatagcccctcagttcaagtcgaatatctaatccatattaaggagattaaaccttggccaccgtcgcattcattgagaacgctccgtgctgttctaatcgaatgggaacacgaagataagcactctggatctactaatcaagttgttccatcgtcgggaactggttctggtattggtgacgggagaattgaattcaatgagtcatttagacttcccgtgacactactaagagaaacttcgcttaaaggtggagacggaaacgcgttccaaaagaattgcgtagaattccacttgtatgaaccccggcgtgataagaccgtgaaaggtcaacttttaggaactgcgatcattgacttggcagattatggtgttgttagagagagtttgagcatatgtccccctattaactgcaagcgaacttataggaacacagcacagcctcttcttttcctgaaaattcagataggtgagaggagtcgtgtgaggtcatccttgagggacaagttaaaaagagaagcttcaatggaccgaaatggttctctttctagattactgagtgaagaatatgctgacgaagctgagtttgcttcatacactgatgatgatgatgatgatgtttcctcgcatttatcagtgcctgtttcttcttcaactaacgaatcaaactatggttcaccccctcaagaagaggatagatccgagggagtaaaaagtagccctcgacgagatgaagacgaaaatgtccttgattataagaaaaggcttgcaaatatggacgagacgccagagacaaaatctctacctagtttgaatggaagtttgtcgcactcctcaactgatttgtcctctgatctggcttggatttcaaagaaaattggtggttgtagtagcattcagtattcgacatcaaatgtgagtgatataactgaggagacacagaatgcctgcatgataatcaaacaggacaagcaagtacaatgtatggaacaaataccggtcaatggcgaaagtgttaatgaacaatccagtcggcagaactcagatccagctgaaaaagcctgtcctattccgcatatcacagatgataacaataactttgtgagcacagttagtaatttctccgatagtgaaattgaagagaacacttccactccacctgtaaattcgctgtgtgatgatgcaagagctgcggttaccaaaaatggttctgttgatggtgaaaacagtgagggttatcagcaaaatgaacaaggaaaagaacctatggaagatgaagggcaatgcaaaaacgatgagtcaatcagttgctattcagaagaggatactgtaaaacatgatttgaaggaaactcatgcaatttctgcttacagggacagttccggagcaaagagtagtacccccgataatgagatattaaagcatgtaatgtcagtcaggtcatcaccagaatcaaataggggtgacggatctgtcggaagtaatcagcttttagtacaagacactctgaaaagtgctcgaggtttttcaagcaatgagagaaaagatcaaaaagcgagtccaagggacacaacaaacattcttttggaaagcaaaatccataagttggagcaaagagtaaagatgcttgaaggagaattgagagaagctgctgctattgaagttggcctttactcagttgttgctgagcatggatgttccatgaataaggtccatgctccggcaagacgcctgtccaggttctatctacatgcatgtaaagaaacctccgtattgaaaaggggaagtgctgctaaaagtgctatttctggaatatatttgattgcaaaagcatgtggacatgatgttgctaggttaactttctggctatcaaattcagtggtgctgagagcaactataaccaaattccatgggcagcagcagttacctctttctactgaaactatgcttggaaattctgttgtcacggacaagaagaagttttccccacttaagtgggaatctcactctagcaacggtgtagaagatgatatttgtgatagtttaggcaattggaaggaccctgttacatttataagagcacttgaaaaagcagaggcttggatattctcccgcatcattgaatctatttggtggcagactctcattccacatatgcagtctggtgctgcaaccgcaatctgtaatagcatgggttccgagataaacaatgtttgcagtaagacgtctagttcaggtgctgaagagcatgtaaatttttccttggatctttggaaaaaagccttgaaagatgcctgtgataggatttgccctgttcgagctggcggacacgaatgtggctgtttgcgtttgctctctaaattgataatggaacaatgtgtggctagattggatgtagctatgttcaatgctatccttcgagagtctgctgatgagatgccttcagatcctatatcagatcccatcagtgatgccgaggtgcttcccattccagctggaaaagctagctttggggcaggtgcacaactgaaaaatgcgatagggaattggtctagatggctcactgaccttgttgggaactcactacacgatgaaaatagagcagataatgatgatgatgggagtgaagaatatgatacatcttccaaatctttctatctcctcgacgcactgagtgatcttatgatgcttccaaaggatatgctgttaagtaggacaataagaaaagaggtctgtcccacatttggtccaattataataagaagggttcttaatgtttttgtcgcggatgagttttgccctgatccaatacctgaatgtgtacttgaagctcttaacacagaggatccttttgacgcggaagaagattcagtcatgagctacccctgcacagcagctcctatagcatataagccaccttcaacagcttcagtggatagtctattaggtgacattagtagccattctaagctgagacggatcggatcttcagtgctcaagaagtcatacacaagcgatgatgagctcgatcaaatggatatgaacttcatcatttctgagggcattgaaacttcaccacttgcaaaatccagtaggatattgaaaggtaatgttgatggaaatgctgtgaggtatcaactccttagggaggtatggataaagagtgaattatga

SEQ ID NO: 11 - DNA, 354 bp, synthetic construct
  source 1..354: mol_type = other DNA
atgcaattgacaaaatggagaaaaatcttcctggattattctatgcaggtaaccacagggggggattgtcagttggcaaagcattatcttctggatgcaatgcagctgatcttgttatatcatatcttgaatccgtctcaactgactccaaaagacattgctgaaatctattttctcatgcagctttctagattgctaaaatcgcttaactttatcgcacaaggtgcaactttgacatgtagcggaactttgacatgtagcccacgacacagctcaagccgcaaatcagtccttcacggggagttgcatttctgcaaaaggctccccagacatggtgacacacttgttctatag

SEQ ID NO: 12 - DNA, 1705 bp, synthetic construct
  source 1..1705: mol_type = other DNA
atggctccttctgccggagaagataaacacagttctgcgaagagagtcgcagtcattggtgcaggcgtcagtgggcttgctgcagcatacaagttgaaaatccatggcttgaatgtgacagtatttgaagcagaagggaaagctggagggaagttacgtagcgtgagccaagatggcctgatatgggatgaaggggcaaatactatgactgaaagtgaaggtgatgttacatttttgattgattctcttggactccgagaaaagcaacaatttccactttcacaaaacaagcgctacattgccagaaatggtactcctgtactgttaccttcaaatccaattgatctgatcaaaagcaattttctttccactggatcaaagcttcagatgcttctggaaccaatattatggaagaataaaaagctctcccaggtgtctgactcacatgaaagtgtcagtggattcttccagcgtcattttggaaaggaggttgttgactatctaattgacccttttgttgctggaacgtgtggtggtgatcctgactcgctttcaatgcaccattcatttccagagttgtggaatttagagaaaaggtttggctcagtcatacttggagctattcgatctaagttatcccctaaaaatgaaaagaagcaagggccacccaaaacttcagcaaataagaagcgccagcggggatctttttcctttttgggcggaatgcaaacacttactgatgcaatatgcaaagatctcagagaagatgaacttagactaaactctagagttctggaattatcttgtagctgtactgaggactctgcgatagatagctggtcaattatttctgcctctccacacaaaaggcaatcagaagaagaatcatttgatgctgtaattatgacggccccactctgtgatgttaagagtatgaagattgctaagagaggaaatccatttctactcaactttattcctgaggttgattatgtaccgctatctgttgttataaccacatttaagagggaaaacgtaaagtatccccttgagggttttggggttcttgtaccttccaaggagcaacaacatggtctcaagacactaggcaccctcttctcttctatgatgtttccagatcgggcaccaaacaatgtttatctctatactacttttgttggtggaagccgaaatagagaacttgcaaaagcctcaaggactgagctgaaagagatagtaacttctgaccttaagcagctgttgggtgctgagggagagccaacatatgtgaatcatctatactggagtaaagcatttccattgtacgggcataactatgattcagtcctagatgcaattgacaaaatggagaaaaatcttcctggattattctatgcaggtaaccacagggggggattgtcagttggcaaagcattatcttctggatgcaatgcagctgatcttgttatatcatatcttgaatccgtctcaactgactccaaaagacattgctgaaatctattttctcatgcagctttctagattgctaaaatcgcttaactttatcgcacaaggtgcaactttgacatgtagcggaactttgacatgtagcccacgacacagctcaagccgcaaatcagtccttcacggggagttgcatttctgcaaaaggctccccagacatggtgacacacttgttctatag

SEQ ID NO: 13 - DNA, 1632 bp, synthetic construct
  source 1..1632: mol_type = other DNA
atggatactacaaattcatcctcctccgacgacccaaacagcggattctgttcaataaccaaaactttccacagtctccggccacctctcttacttcctccagaagcttctcctctttccgctgcttcttacgcactctctctccaacaatgccccgattccaccgccgccgccttcatcgactccgccaccggccgccgcttaaccttctccgaatttcgtacacgtgtcatttccctagcttcctcccttcaaaacacattccgcttctccaaaaacgatgtcgcatttatcctttcaccgaattcaactcacgttccggttctctactttgcactactctccctcggcgttatagtctctcccgctaatcctatgagtacggaatccgagctgtttaaacaaattaagttaacgaatcctgtaatcgcttttgctacgtcgaggaacttccagaagcttcctaagttgaaacacggaacagttcttattgacttaccggagtttgaatcgatgatgacgaatacggagttgaaattcgagcctgtggaagtgaatcaatcggatttggcggcgattatgtattcgtcggggactacaggggaggtaaaaggagtgaagttgagtcatcggaattttattgcaattattgctaatttttatgctcagagacaagagcggacgtcgccggtgccggtgatgttgtatacggtaccgttgtttcatgtgttcggtttacactatgcgttgaaatcggtagcgttgagtgagactgtagttgtgatggagagatttgatttgaggaaaatgttgaacgcggtggaggagtttaaggtgacgcaattggtggtggctccgccggttgttgtggcaatggctaaaggaagtgttaccagtggctatgatttgagttcgttggtagcggttggtagtggaggtgctccgcttggcaaagatgttatccaagcattttcatacaagtttccaaatacaatattatttcaaggttatggactaactgagacaagcggggcagcatttcgagctgcgactacagaagaaatgcttcgtggggattcagtgggaaggctaatagcaaatactgaagcaaaaattgtagatccagaaactggcattgctctgcctcccggtgaacaaggagagctctggattaaaggtccaaccattatgcaaggttatgtgggtgatccgaagacaacttctgaaactctggtgccgggtggatggttgaggactggtgatctttgttatattgaccatcatggttacctttttgttgtagataggctgaaggagctaatcaaatataaaggataccaggttgctcctgctgaacttgaacaacttcttctgtctcacccagatatagtagatgctgctgtgataccataccctgatgaagaagctggtcaagtgcccatggcatttgttgtgagacgtacccaaagctctccagacaaagaacaagtgattgattttatttcaaaacaggttgctccatataagaaaataaggcgtgtagcatttattagctccataccgaagaatccatctgggaagattttgagaaaagaattaagaaggatccatataccagaatctaagttgtaa

SEQ ID NO: 14 - DNA, 882 bp, synthetic construct
  source 1..882: mol_type = other DNA
atggatgttgagaatagtgggccctcctatggatgccacgttggaccacaagataggatgataatgaaccggataatgttaagattccggccgattgcaccgaaaccggtcgccgacggttcaggtccgggttctaaaccggagaatgaaaaggcagaaccggttagcagacgaagagggaagagaaagtacgttagagttaagaaaaataggaagtgtaagaatagtgcagagaaagaagaaagaaaagatggatctgataataatggatctattgtaacccttcagttacttccggagagtagcagcagtgttacaaacccgacagataacgtttcttttcaaaaagaaatctctttctctgagctagatcaaccccaatggatgagttttcatgaatatgataataatggtatatccgccttcagttcaacggatcggttagatctgaaggtgacaacgcagacaccaagaatggtagagtcttgggtgatggtggatagaataacaaacatatttgtggatagtgaaggcttaggaagtacggacatggagaaaatgaagaatctggagacggacacgtatccagggctcgtgtcggatagctcagatagggtgcggtgggtgaatccggcgtaccggagaatggtgaatcctctagaagacggaggagtgacggcggagatggtggtgaagatggttgtgaaggagaaaataagagaggaagaattacctgcagcttttgcatgcattgtgaggttagtgtatacatggaagaataagaaaaactcacgaacaatgccttgtgatgtgtggaaaatggagtttggagggtttgcatggaggtttgattctaatgctgcacttagcttgggtcgttaa

SEQ ID NO: 15 - DNA, 2055 bp, synthetic construct
  source 1..2055: mol_type = other DNA
atgctgaagtctttttcgggtcttgatattcttgccattgccatgcctccaacgtatttacctcttcggtgggagagtactggggaccaatggtggtatgctgcaccaattgattgggcagctgcaaatggtcactatgatttggtacgagagcttcttcgcctagacggcaatcacgttatcaagctcacttcattacgtaggataagaaggcttgagtccgtttgggatgatgaagaacagtttgatgatgtagctagatgccggtcacaagttgcaaagaagttgcttcttgaatgtgagaacaagaaagggaaaaattccctccttaaagctggatatggtggatggcttttgtatactgctgcctctgctggagacttggattttgttcgacaattgctcgaaaaagactcccttttggtatttggagaaggggagtatggtgttactgacatattatatgctgctgctaggagtaaaagttgtgatgtgtttaaggttttgtttgattttgctgtatcaccgaggtttatagcgcgtggtggtagagggtcggaggaacagaatggggacattccgtctgcttataagtgggaaatgatgaatagagctattcatgcggctgctagaggaggcaatgtgaagatgctgaaggagcttattgctgactgttctgatgatatattggcttacagagacattcaaggtgcaactctcttacatacggcagctggcaaaggccaggtcgaggttgttaaatatcttctgaaaagttctgatattatcaactctgtagacaatcaaggcaatactgcattacacgtggctgcttcgaggggccaattagctgttgttgaagctctcatcgttgcttcaccttcattggtcaattcaagaaacaatgctggagatacatttcttcatgttgccattactggtttccaaaacccttatttccgcagattggatcgtcaaattgatctcatgaaacaattagtttgtggaaaatttttcaacattgaggaaattgttaacgctgaaaacaatgacggtagaactgctcttcatttagctgtcattggaaatattcattctgaacttgtggaattacttatgaccgtccgctctattaatgtcaatactcgtgataaggatggaatgacaccactcgatatcctaaggcaacgaccacgttctgcttcctccgagctacttacaagacagttgatctctgctgggggaatttttagtcatcgcgattattctgcaaggagagttgttgcatcccagttgaagatgcaaaacataagtagcagtcctggaacttcttttagaatctctgacaccgaaatattcttatacacgggcattgaacatgcaccagaggacagtaaaaaagctgagattagcaacacaagtgagccgagtcatcgatacatgagtcctgatagctattgttctagaaatgacaagaaatctggttccgcagatcatgcagctgaaaaactgaaacgcttcttccattggccaaaaattaaaaaaagagatactaagaaggtcaagatattggttgatcaatgttctgcaagtaattcggacgtggcaccagtccctcttcgagaaagatactccaagccttcatcattctcaagccacaaacggacgctctctgctagtagtaacattccaagtccaacagcaaagaagaagtttgcttctgggatagtaaacggtgtcatgcaagccataccacatctcagcctccctcgtagatcacctcgtgctagttcattctcgatttcatcattgtcttcacgcagttctttggacaaacaaaaagcaatcgtgattgagactgagctcgctggaccttcttgttctaatcaggtagacggtgtatcatcagactcgactcacaaacaaagtgcaggacataaaaggtcggtgaaccaatatttgtgttttggtgcctcaggacaacctgtcaaggcctcttcaagtgggatgcagccatatgaactttatgagcggtctgttctatccacggcttga

SEQ ID NO: 16 - DNA, 825 bp, synthetic construct
  source 1..825: mol_type = other DNA
atggcggcgtggaattcaattcctctggaagttctgtacaatactttaggatggattgcttttttctcttggtccattagtttttaccctcaagtcatcttgaatttccgacgcaaaagtgtggtgggattgaatttcgatttcgtggtattgaatttgaccaagcactcatcgtatctcatttataatgcttccatgttctttagttctgctgttcagatgcagtatcgtcagaaatatggctataatgagatgatacctgtggctgccaatgatgttgctttctcagtccatgctgttctattgacagcatttaccctgtttcagattgctatttatgatcgtggaaaccagaaggttgcaaagacttctatagcgatcgtgtctgttgcttggttaactgttgcagtttgtgtgttcgtagctctccccagccattcttggctttggctggtctcttgcttcaacacgttgcaggttgttatgacagtgattaaatacattcctcaggctattatgaactttcagcggaagagtactattggatttagtattggcaacattttgctggacttgcttggaggtttgacgaattatggacagatggcagtgcaatccatagatcaaaattcctgggtgaacttttatggaaacataggcaagactttattatcactgatatcaatattctttgacattctcttcattttgcaacattatgtgctctatcctgcttggaagactgcaacttctcgagatgttgatgtgataggcaagaaaccattgcttaaatcttccgatcatgaaaacaaagaagatgtataa

SEQ ID NO: 17 - DNA, 3684 bp, synthetic construct
  source 1..3684: mol_type = other DNA
atggaggatagtgtgagatcaggagggggtgtattgaagaaaaaaagctcttctggttgtttaattattaaaaagaaagatgacaaaatggtgaagaagaggccaaggtttgttatgaatgatcacgtgtcgaatgatgagttatcggagtcaattcggaggaaaaatggtcaattttttagtaatggttatggaaaaagtggtgttagagaaggagaatttggtaggaatgtgaatttgcttgatcttaatgagtacgaagaatgtgatgtgaaaaggcaattgaatgggtatagttatgagatatcagagtcaattcggagaaaaactggtcaaaattttcgtaatggttctgttgtttatggaaaaagttgtgttagagatggagaatttggtaggaatgcggctttgatagatttaaacgagtacgaagaacgtgatgtgaaaaggcaattggatgggtgtggtgatgagatatcagagtcaattcggagaaaaaatggtcaaaaattccgtaatggttctgttgtttatggaaaaagttgtgttagagatggagaatttgttaggaatgcggatttgcttgatttaaatgagtacgaagaatgtgatgtgaaaaggcaattgaatgggtacagtgatgtcagatcgaacatgattgaaagaagagggagttgtagagagttcgggagtgggtctgggagtgtgatggctgagaagaggaagctttcatgtatggatagttcaagtagtttttcaggtagtaggatgaagggcgatgacaatggctttacgagaaggtatgatttgttagagggtgaagtgcgtatgcccatttcattgaccagagaagccactcatgaagcaatcagattgcaagggaaaaatggggtattaaaggttatgttgaataagaagaagaagattgacttaatgcacaaggattttgatcctgcggaaatagaaaatagaaaaggttctcagtcggcagatgttgtcaagaggaatttactagtccatccgtccttttactcaggttctaaacacccggaaaagcaacctttgtctgttaaaacagagaaaaatgagctgaaactggaaaaacctttgttgggtaaaatcatccgttcagtagcctctgaaaaagatgagactgatacgtcacttaagttggcaccgcctagttcagtgcctgctagctcagcaatgggggtgctaaaggaagaaagcaggtccttagcatctgaagatgttattcctgctaaaagcaaagacggaaaagtgaaacgaggtgggagcactgagaagcagcagttgcgagagcgaataaggggaatgctccttgaagctggatggaccattgattatagaccaagaaagaatagagactatctggatgcagtgtacactaatcctagtggaacatcctattggtcaataattaaggcctatgaggcatttcaaaaacgatctgaggtagattcaggtaaaagtaaacctgatggcagctcttgttcatatgctccaatatcagatgatttaattaataaacttaccaggcaaacacggaaaaagattgagaaagaaatgaaaaagaagagaaaagatgatggcaagagagaagatcgtaaaaagacttctgtgagggagtcttcgttgggcacagacactcttcagcatgaggagaggctcggtagctatataaagaagaaagataagttgctgcaaggcaaattacatgcaatagaccgggagaatggggataattcaagtgacaatttgaatgtaaggagacttaaacaggatacagttgcaaaatcatctggtggagttgcttctaattcaatacatggaagaaaaagtaaattaattgggcgatgtacattgctggtccgtcattctgacaagaggggaagttcagatagtgatggctatgttccatgtactggaaaaagaaccttattgtcctggttgattgactccgggacattgaagttgggacagaaggttcaatatgtgaaccgtagaaggactagagtaaaactagaaggttgggtcacacaagatggtgtccactgtggttgctgtagtaaaatccttccagtttcaaaatttgagctccatgctggaagtaaacggcatcaaccatttcaaaacatagttctagagttcggaggtaccttactagagtgcttaattgacacgtggaatcggcaaaaagaatctgatcaccgggatttctacagtataggcattgatggtgatgatcccgaagatgatgcttgtggtatctgtggtgatggtggggaccttatctgttgtgatggttgtccatcaactttccatcagaactgtctgggcataaaaatgcttcctccaggacattggcattgtccaaattgcacttgcaaattttgtggggctgcttgtgaatttcctgaggaggctagtgagagaactgtcaatgaaattctttcttgcagcctctgtgagaaaaaatatcacaaatcatgcagtttggagacggatgctttatctgctatatccaatgatcaatctgcatcattttgtgggcagaaatgccgggagctgtatgaccatttacagaatattcttggtgtcaaacatgaacttgaagcaggattctcatggtccctgattcaacgaacagatttggactcagatatttctcgttgcccctttcctcaacgggtggagtgcaattctaagctagctgttgcggtggctgttattgatgaatgtttcttgccctttgttgacaggagaagtgggataaatattattcataacgtactctacaacactggctcaaatttaagtcggctgaatttccgtggcttctacactgctcttctggaaaggggtgacgagataatatcggttgcatctataaggatccgtggaactcaacttgcggaaatgcctttcattgggacccggaatatatataggcggcaagggatgtgccgtcgactgttgtcagctattgagacagttctctccactttaaaagttgagaagttaatcattcctgcgatatctgagcatccgcataactggaccaaggtttttggatttgaggaacttgaggaatcaaacaagcaagaaatgaaatccatcaatatgttggtatttcccgggacagacatgttacaaaagaagatgttgatggaagacatgcaagaggcttgtgatttacaacatagtcatcctccgccacctgctttggttgaaaaagctgatcaggaatcatctatcaggcatgatcgacatctgcatgatggcgtatgtgtgagcatagtggaaaaaactgatgatgggtttggtcccatggattctggctctccagcttctgctgttcatttaagtgctagcactgtggttagagcacaaggtgattgttgcgagtctgatatccaaatctccagtacggaggcagagaagaattttactgaatcagccacaaatttaatgctctcttctccatctggcgcaagtaacggcagccctgatactgaagatgctgcactaggtcctgcaaatattgctgtcaactctgctgaagtttcttccaggagcacatcggtagaaaacagattgaagaagcttctgatagttctgccactaataacgatcagaaagatatttgtgtggtttgcgtag

SEQ ID NO: 18 - DNA, 552 bp, synthetic construct
  source 1..552: mol_type = other DNA
atgggaggtttcatatcgagattctggttcatgatgtttccagcaaaagagtataagatcgttgtagttggattggataacgctggtaaaacgacgacgctttacaagttgcatttaggagaggtggttactacaaatcctactgttggtagtaatgttgaggagcttgtttataagaacatacgatttgaggtctgggatctaggtgggcaagaaagactgaggacatcatgggcaacttactatcgcggaactcatgctgttatcgtagttatcgatagtactgacagagcccggatttccattatgaaggacgagttgttcaggttgctcccacacgaggatctgcaaagtgcggttctactagtctttgcgaacaaacaggaccttaaagatgccatgacccctgcagagataaccgatgcgctttcccttcatagcatcaagaaccatgattggcatattcaagcctgcagtgccttgactggtgacggtttatacgatggtttagggtggattgcacagcgagttcctggtaaatcaccaacctag

SEQ ID NO: 19 - DNA, 1386 bp, synthetic construct
  source 1..1386: mol_type = other DNA
atgcagaagagacagagcggaggcggagcagccggtggctccgccactccggtagcaaagagaggccgtccggtcggcagtggaggcagcaacgctgccgctgcatctgctgcctctagtgctgccgccgactcggtagctcctccaacactcctcggcccttcccttcatgttccctatgccttcgctgaacagaataataaaaggattgtcctggcacttcaaagtggattgaagagtgagctgacgtgggcattaaacactctcactttgctatcatttaaagagaaagatgaagtccggaaagatgcaacccctcttgcaaaaattcctgggctgcttgatgctctacttcaagttatagatgactggcgtgatatagccttaccaagggtgcttgtgaaggcaccacgtgttcggcttttgggtgcaaattcagctgttactggatttggaaatgaatatgaggccttgaactcaaataatcccttaccccatcctagtgctggatctggatcttccaaagaggcatctgttcataagagtacttccaagactcgccctggagcctggagttttgaagaagatggcctatttaatttggatgaggaggggcgggctgagaagcagctgtgtgctgttgctgcttcaaacattatccgcaacttctctaacatgccagataatgaagtcatcatggcgcagcataggcattgcttggaaaccatatttcaatgtcttgaagattatgttacagaggatgaggaacttgtcacaaatgccctcgagactgtagtgaatttggcgctgttgctgaatcttcggatctttagctcctcaaaaccttcttttatcaaaatgacggagaaacgtgcagtccaggctctcatgggaatgctgggatctgccgtgaaggcctggcacgctggcgctgctgaattaattggtcgtcttataataaatcccgacaatgaacctttcctacttccttttgcttcacagatatataaacgtttagtcgatataatgagcctaccacctactgatgctcaagcagctgctgtaggtgcattatataaccttacagaaataaatatggacagtcgtttgaagctcgccagtgaacgatgggcaattgatcgactgttgaaagtgattaagacaccacatccaacaccagaagtttgcaggaaagcagcaactattttggaaagcctcgtgttggagccacagaacaagcccctcttactggtgtatgaaaatgcttttgcagagatgctctttggggatgcaagatactcggacatatttgcaaggatattatatgaactaacatcacggccaagcaacaaagtcgcatcagcttgtggaatctggggcatgtaa

SEQ ID NO: 20 - DNA, 1002 bp, synthetic construct
  source 1..1002: mol_type = other DNA
atgttggtagaaactctagataatagtacacaaaaactagtcttgattgacacaattcaaagattgggattagcatatcatttcaattatgagattgaaaactccattcaaaacatcttttatttgtctcaaaatagtgaagataacgatgaacacaacctttatgtggctgctcttcgttttcgacttgcgaggcaacaaggaaattacatgtcttcagatgtgttcaagcaattcactaaccatgacagaaaattcaaggaaaatcatactaatgatgttcaaggattactgagtttgtatgaagcagcacatacgagagtgcacgacgaggaaattctagaagaagctcttatatttaccccaactcatctcgagtccgtgatcccgaatttgagtaacgcgcttaagctggtggaaagatttggattgcgcaaacaaatttccatatgcaaaggacagattagtagaagcttacttttggacggtgggaatatatttgaacctcaatatggtcgttcaagaagtatgataacaaaagtagtcaaaatgaactccattattgatgacacttatgatgcttatgcaacttttgatgagcttgtgcttttcacggatgcgatccaaagatgggacgaaggtgccatggattcgttaccgacatatctgagacctatttatcaaggccttctcgacgttttcaatgaaatggaagaagtattggccaaagaagcctattataaggaagctgaatggttgaatgctaactacattccaaaatgtgaggagtatatgaaaaatggagttgtaacctctaccggtacgatgtatggaataatttctttggttgttatggaggaaattataacaaaagaggcttttgaatggttggcaaatgaacctttgattcttcgagctgcatcaacaatctgtagattaatggatgatatggctgatcatgaagttgtttacatcacacccattgggtgcgacccttcctaa

SEQ ID NO: 21 - protein, 1142 aa, synthetic construct
MAASSAHEEAGTGRSYEGLSSGQQQCSEALAEWRSSEQVENGTPSTSPPYWDCDDDDDGGPKPSELYGKYTWKIDKFSQINKRELRSNAFDVGGYKWYILIYPQGCDVCNHLSLFLCVANHDKLLPGWGHFAQFTIAVVNKDPKKSKYSDTLHRFWKKEHDWGWKKFMELSKVLDGFVDADTLIIKAQVQVIREKPEHPFRCLDRQYRRELVRVYLTNVEQICRRFVEERRVKLGKLIEDRARWSSFCAFWLGMDQNSRCDMSKERSDSILKVVVKNFFIEKEVTSTLVMDSLYSGLKSLEGQIMGKKGKAKYSDADEQLVPIVRMEKNMFVLVDDVLLLLESAALEPLPPKDEKGPQNRTKDGTSGDEFNKDTIERDERRLTELGRRTIEIFVLAQIFSKIEVAYQEAVALKRQEELIREEEAAWLAETEQKAKRASGKEKKSKKKQAKQKRNNHKVKDKGVDEKPGSMELYKIDQDGPIGEGNEYINEEREAVLGKPDILEAVSDVSDSIDCVPEVINPDSEDRDASPVNWDTDSSEVHPSTETSCSGLSDLSAVQNGLAGRRSPSVMDDSSSTCSTDSIPSVVSNGPCRWTSANHKNQKSPSRGKNQRNKSTSNAADWASETLSQPLDAVSDLGQLSDRSCGAPGSESHSTVLSSNEQQKVKKEVVALQQRKAETERPSMEKPSVKSSRRSPPKDAGSAVQQKSQTKISVTSDPALVKRSSTDGPKLADKSALVSNSSETAVLKADPHKAVEPRVKDKPSVQPICIMTGESSSQQVTVSSTTENFKWQQVPAVSRPLCDPLVPGPRPAAPVVSMVQTTPSLARSVSAAGRLGPDPSPATHSYLTQSYRNAIMGGPVSGSPASFSQPHSPISAVNLSHSYSQQQPPLVSGASFLPHGLERAEPCSMRPSFSYGMVNNGSLQNGLQWECPQRDSNRSISQHHPSASNGIRNFDMFKTVNSRTHDHVPDSLACTSGRQSQSVSPDEFPHLDIINDLLNDDHGIGRASIPDLGFQSFSSGSQHLNHVFTFPGDIGTPTDLGASSSSCRFERTRSHHDMFQHNYSGGLFDSASDMILQPDPRFMNGHHIDGLVPNQWQMIGSDPSFLGMRNVNIDGSHPYPLPDYSNMACGVNGYGLFRPSNGL

SEQ ID NO: 22 - protein, 393 aa, synthetic construct
MVMGREVVPVMDPSQVVGLKKKGTGSRSWLLMDASGLETVLDVDKYAIMHRVQIHARDLRIIDPLLSYPSTILGRERAIVLNLEHIKAIITSEEVLLRDPLDENVTPVVEELRRRLKPMNANREDQREEKDMNVQHDIETTEEDESPFEFRALEVALEAICSYLAARTLELETAVYPALDMLTSKISSRNLDRVRKLKSQMTRLTARVQKVRDELEQLLDDDGDMADLYLSRKLAGASPVSGSGAASWFLASPTIGSKISRASRASIATFRGDENDVEELEMLLEAYFGQIDSTFNKLITLREYIDNTEDYINIQLDNHRNQLIQLELFLSSGTVCLSIYSLIAAIFGMNIPYSWNKDHGYMFKWVVIISAIFSAVLFIFIISYARFKGLVGS

SEQ ID NO: 23 - protein, 774 aa, synthetic construct
MDFVELEAIEGLRWSWNSWPISKSESSALIIPLSVMCNPLMQFNELPILNYDPVICSQCGAVLNPYARVDYVSRIWNCPFCYRKNPFPRSYLGINENNVPAELFPTYSTVEYHLGKKGLTQNPNSSSNFGNVSMLSKMPSFSSLDWAGYGLGPAFVFVVDACTSDEELRVVKNELLHVIAQLPETALVGLVVFDSMVRVHDLGFGECCRVVMFHGERELSSEQTKQLLGIHHVKYQAGKVPMAPKQGFLIPLSEGEFSITSAIEDIQSSAQVMQGHRPFRATGVAVSVAVGLLEGCLVSAGSRIMIFTSGPATIGPGMIVSSELGNAIRNHRDIGNGHAAYYKKSSEFYKQISRRLSNSSIALDLFACSLDQVGAAELRASVESSGGFMMLTESFDSDQFRKCLRHIFSHDEAGNLKMCLDATIEIVTTKDVKICGALGPCVSLQKKNGSASHKEIGEGGTYTWKLGALTNKTCIAFFFEVGDEQKAQPSSAFFIQFITHYRYGNTGIRKRVTTAARRWVGNHSPEIAAGFDQEAAASVMARLAIHRAESNFSQDVVRWLDKCLIRFASKYGDYIQEDPSSFRLATNLSLFPQFMYYLRRSQFIDVFNSTPDETAFFRLLLNREGVVGSLIMVQPTLFQYSFEGPPIPVLLDICSVSPDVILLFDSYFYVVIHYGSKIVQWRKLGYDKDPSHDSFRKLLEAPEIDAEQLVSERIPVPKLIKCEQHSSQARFLLAKLNPSVTHNSTYTEGKELIFTDELSLQVFIEHLQALAVQG

SEQ ID NO: 24 - protein, 598 aa, synthetic construct
MSQSISPLICSHFAKFQSNIWRCNTSQLRVIHSSYASFGGRRKERVRRMNRAMDLSSSSRHLADFPSTIWGDHFLSYNSEITEITTQEKNEHEMLKEIVRKMLVETPDNSTQKLVLIDTIQRLGLAYHFNDEIENSIQNIFNLSQNSEDDDEHNLYVAALRFRLARQQGYYMSSDVFKQFTNHDGKFKENHTNDVQGLLSLYEAAHMRVHDEEILEEALIFTTTHLESVIPNLSNSLKVQVTEALSHPIRKAIPRVGARKYIHIYENIGTHNDLLLKFAKLDFNMLQKLHRKELNELTSWWKDLDRANKFPYAKDRLVEAYFWTVGIYFEPQYSRSRSLVTKVVKMNSIIDDTYDAYATFDELVLFTDAIQRWDEGAMDLLPTYLRPIYQGLLDVFNEMEEVLAKEGKADHIYYAKKEMKKVAEVYFKEAEWLNANYIPKCEEYMKNGLVSSTGPMYGIISLVVMEEIITKEAFEWLTNEPLILRAASTICRLMDDMADHEVEQQRGHVASFVECYMKEYGVSKQEAYVEMRKKITNAWKDINKELLRPTAVPMFILERSLNFSRLADTFLKDDDGYTNPKSKVKDLIASLFVESVDI

SEQ ID NO: 25 - protein, 424 aa, synthetic construct
MLVETPDNSTQKLVLIDTIQRLGLAYHFNDEIENSIQNIFYLSQNSEEDDEHNLYVVALRFRLARQQGNYMSSDVFKQFTNHDGKFKENHTNDVRGLLSLYEAAHMSVHDEEILEESLIFTTTHLESMIPNLSNSLKVQVTEALSQPIRKTIPRVGARKYIYIYENIEAHNDLLLKFAKLDFNMLQKLHRKELNELTRWDIGAMDSLPTYLRPIYQGLLDVFNEMEEVLAKEGKADRIYYAKKEMKKLVATYFKEAEWLNANYIPKCEEYMKNGLVSCTGMMYGTISLVVMEEFITKETFEWLANEPLILRASSTICRLMDDIADHEVEQQRGHVASFVECYMKEYGVSKQEAYVEMWKNITNAWKCINKELLRPTAVPMFVLERTLNYTRLVDTFFKDDDGYTNPKSKVKDLITLLFVESIDI

SEQ ID NO: 26 - protein, 178 aa, synthetic construct
MAASEMLSKEQLLHLFDRFAFLTSQPDVKKRIADAVSDKQEAVAVTTAIQEEIFSEMGIDPRFGLACLGKINMTYESDRDLMIRFYEFVAKEEMACEEAELGPDGFAERLKMQQNLQEQQLEMLKYMRNFHMDDQSAILEKIHQQLEKANFDTEASVLTVEQMQDVVRRRVSPLFQPR

SEQ ID NO: 27 - protein, 72 aa, synthetic construct
MALTNFILTVVGVSAAILLLRSDVKQSASIFKRNVRQIRHWLEEESSSAAKELEKAKPKEIPHKDIPKEDKH

SEQ ID NO: 28 - protein, 194 aa, synthetic construct
MTNAALITVAKNCPNFIRFRLCTLNPTVPDAVTMQPLDEGFGAIVQSCKGLKRLSVSGLLTDQVFLYIGMYAEQLEMLSIAFAGDSDKGMLYVLNGCKKMKKLEIRDSPFGNVALLADVGKYETMRSLWMSSCQVTLGACKAVAQKMPRLNVEIISENDQIDTCSDDRQKVEKIYLYRTLVGPRKDSPDFVRTL

SEQ ID NO: 29 - protein, 343 aa, synthetic construct
MCLCQCMMNYFPEEVLEHVFNFLTSHRDRNAVSLVCNSWYRVERFSREKVFVGNCYAVSPERVIARFPRLRSLTLKGKPHFADFNLVPHDWGGYVDPWIEAMAASGVNLEELRLKRMVVSDESLELLSRSFPNFKSLILVSCEGFSTDGLAAIASNCRFLRELDLQENEVEDRRGQWLSCFSDNCTTLVSLNFACLKGEVNLAALERLVARCPNLRSLRLNHTVPLDALQRILIKAPHLVDLGTGSFISDPVSETYNKLKNALKRCTSIRSLSGFLEIAPRCLPAIYPICLNLSSLNLSYAPGIYSAELIKLIRFCGKLERLWVDIFAPLLIISLLLYFPTIV

SEQ ID NO: 30 - protein, 1066 aa, synthetic construct
MVLGLKGRTRNSPSVQVEYLIHIKEIKPWPPSHSLRTLRAVLIEWEHEDKHSGSTNQVVPSSGTGSGIGDGRIEFNESFRLPVTLLRETSLKGGDGNAFQKNCVEFHLYEPRRDKTVKGQLLGTAIIDLADYGVVRESLSICPPINCKRTYRNTAQPLLFLKIQIGERSRVRSSLRDKLKREASMDRNGSLSRLLSEEYADEAEFASYTDDDDDDVSSHLSVPVSSSTNESNYGSPPQEEDRSEGVKSSPRRDEDENVLDYKKRLANMDETPETKSLPSLNGSLSHSSTDLSSDLAWISKKIGGCSSIQYSTSNVSDITEETQNACMIIKQDKQVQCMEQIPVNGESVNEQSSRQNSDPAEKACPIPHITDDNNNFVSTVSNFSDSEIEENTSTPPVNSLCDDARAAVTKNGSVDGENSEGYQQNEQGKEPMEDEGQCKNDESISCYSEEDTVKHDLKETHAISAYRDSSGAKSSTPDNEILKHVMSVRSSPESNRGDGSVGSNQLLVQDTLKSARGFSSNERKDQKASPRDTTNILLESKIHKLEQRVKMLEGELREAAAIEVGLYSVVAEHGCSMNKVHAPARRLSRFYLHACKETSVLKRGSAAKSAISGIYLIAKACGHDVARLTFWLSNSVVLRATITKFHGQQQLPLSTETMLGNSVVTDKKKFSPLKWESHSSNGVEDDICDSLGNWKDPVTFIRALEKAEAWIFSRIIESIWWQTLIPHMQSGAATAICNSMGSEINNVCSKTSSSGAEEHVNFSLDLWKKALKDACDRICPVRAGGHECGCLRLLSKLIMEQCVARLDVAMFNAILRESADEMPSDPISDPISDAEVLPIPAGKASFGAGAQLKNAIGNWSRWLTDLVGNSLHDENRADNDDDGSEEYDTSSKSFYLLDALSDLMMLPKDMLLSRTIRKEVCPTFGPIIIRRVLNVFVADEFCPDPIPECVLEALNTEDPFDAEEDSVMSYPCTAAPIAYKPPSTASVDSLLGDISSHSKLRRIGSSVLKKSYTSDDELDQMDMNFIISEGIETSPLAKSSRILKGNVDGNAVRYQLLREVWIKSEL

SEQ ID NO: 31 - protein, 117 aa, synthetic construct
MQLTKWRKIFLDYSMQVTTGGDCQLAKHYLLDAMQLILLYHILNPSQLTPKDIAEIYFLMQLSRLLKSLNFIAQGATLTCSGTLTCSPRHSSSRKSVLHGELHFCKRLPRHGDTLVL

SEQ ID NO: 32 - protein, 435 aa, synthetic construct
MAPSAGEDKHSSAKRVAVIGAGVSGLAAAYKLKIHGLNVTVFEAEGKAGGKLRSVSQDGLIWDEGANTMTESEGDVTFLIDSLGLREKQQFPLSQNKRYIARNGTPVLLPSNPIDLIKSNFLSTGSKLQMLLEPILWKNKKLSQVSDSHESVSGFFQRHFGKEVVDYLIDPFVAGMCGGDPDSLSMHHSFPELWNLEKRFGSVILGAIRSKLSPKNEKKQGPPKTSANKKRQRGSFSFLGGMQTLTDAICKDLREDELRLNSRVLELSCSCTEDSAIDSWSIISASPHKRQSEEESFDAVIMTAPLCDVKSMKIAKRGNPFLLNFIPEVDYVPLSVVITTFKRENVKYPLEGFGVLVPSKEQQHGLKTLGTLFSSMMFPDRAPNNVYLYTTFVGGSRNRELAKASRTELKEIVTSDLKQLLGAEGEPTYVKYVNS

SEQ ID NO: 33 - protein, 543 aa, synthetic construct
MDTTNSSSSDDPNSGFCSITKTFHSLRPPLLLPPEASPLSAASYALSLQQCPDSTAAAFIDSATGRRLTFSEFRTRVISLASSLQNTFRFSKNDVAFILSPNSTHVPVLYFALLSLGVIVSPANPMSTESELFKQIKLTNPVIAFATSRNFQKLPKLKHGTVLIDLPEFESMMTNTELKFEPVEVNQSDLAAIMYSSGTTGEVKGVKLSHRNFIAIIANFYAQRQERTSPVPVMLYTVPLFHVFGLHYALKSVALSETVVVMERFDLRKMLNAVEEFKVTQLVVAPPVVVAMAKGSVTSGYDLSSLVAVGSGGAPLGKDVIQAFSYKFPNTILFQGYGLTETSGAAFRAATTEEMLRGDSVGRLIANTEAKIVDPETGIALPPGEQGELWIKGPTIMQGYVGDPKTTSETLVPGGWLRTGDLCYIDHHGYLFVVDRLKELIKYKGYQVAPAELEQLLLSHPDIVDAAVIPYPDEEAGQVPMAFVVRRTQSSPDKEQVIDFISKQVAPYKKIRRVAFISSIPKNPSGKILRKELRRIHIPESKL

SEQ ID NO: 34 - protein, 293 aa, synthetic construct
MDVENSGPSYGCHVGPQDRMIMNRIMLRFRPIAPKPVADGSGPGSKPENEKAEPVSRRRGKRKYVRVKKNRKCKNSAEKEERKDGSDNNGSIVTLQLLPESSSSVTNPTDNVSFQKEISFSELDQPQWMSFHEYDNNGISAFSSTDRLDLKVTTQTPRMVESWVMVDRITNIFVDSEGLGSTDMEKMKNLETDTYPGLVSDSSDRVRWVNPAYRRMVNPLEDGGVTAEMVVKMVVKEKIREEELPAAFACIVRLVYTWKNKKNSRTMPCDVWKMEFGGFAWRFDSNAALSLGR

SEQ ID NO: 35 - protein, 684 aa, synthetic construct
MLKSFSGLDILAIAMPPTYLPLRWESTGDQWWYAAPIDWAAANGHYDLVRELLRLDGNHVIKLTSLRRIRRLESVWDDEEQFDDVARCRSQVAKKLLLECENKKGKNSLLKAGYGGWLLYTAASAGDLDFVRQLLEKDSLLVFGEGEYGVTDILYAAARSKSCDVFKVLFDFAVSPRFIARGGRGSEEQNGDIPSAYKWEMMNRAIHAAARGGNVKMLKELIADCSDDILAYRDIQGATLLHTAAGKGQVEVVKYLLKSSDIINSVDNQGNTALHVAASRGQLAVVEALIVASPSLVNSRNNAGDTFLHVAITGFQNPYFRRLDRQIDLMKQLVCGKFFNIEEIVNAENNDGRTALHLAVIGNIHSELVELLMTVRSINVNTRDKDGMTPLDILRQRPRSASSELLTRQLISAGGIFSHRDYSARRVVASQLKMQNISSSPGTSFRISDTEIFLYTGIEHAPEDSKKAEISNTSEPSHRYMSPDSYCSRNDKKSGSADHAAEKLKRFFHWPKIKKRDTKKVKILVDQCSASNSDVAPVPLRERYSKPSSFSSHKRTLSASSNIPSPTAKKKFASGIVNGVMQAIPHLSLPRRSPRASSFSISSLSSRSSLDKQKAIVIETELAGPSCSNQVDGVSSDSTHKQSAGHKRSVNQYLCFGASGQPVKASSSGMQPYELYERSVLSTA

SEQ ID NO: 36 - protein, 274 aa, synthetic construct
MAAWNSIPLEVLYNTLGWIAFFSWSISFYPQVILNFRRKSVVGLNFDFVVLNLTKHSSYLIYNASMFFSSAVQMQYRQKYGYNEMIPVAANDVAFSVHAVLLTAFTLFQIAIYDRGNQKVAKTSIAIVSVAWLTVAVCVFVALPSHSWLWLVSCFNTLQVVMTVIKYIPQAIMNFQRKSTIGFSIGNILLDLLGGLTNYGQMAVQSIDQNSWVNFYGNIGKTLLSLISIFFDILFILQHYVLYPAWKTATSRDVDVIGKKPLLKSSDHENKEDV

SEQ ID NO: 37 - protein, 1227 aa, synthetic construct
MEDSVRSGGGVLKKKSSSGCLIIKKKDDKMVKKRPRFVMNDHVSNDELSESIRRKNGQFFSNGYGKSGVREGEFGRNVNLLDLNEYEECDVKRQLNGYSYEISESIRRKTGQNFRNGSVVYGKSCVRDGEFGRNAALIDLNEYEERDVKRQLDGCGDEISESIRRKNGQKFRNGSVVYGKSCVRDGEFVRNADLLDLNEYEECDVKRQLNGYSDVRSNMIERRGSCREFGSGSGSVMAEKRKLSCMDSSSSFSGSRMKGDDNGFTRRYDLLEGEVRMPISLTREATHEAIRLQGKNGVLKVMLNKKKKIDLMHKDFDPAEIENRKGSQSADVVKRNLLVHPSFYSGSKHPEKQPLSVKTEKNELKLEKPLLGKIIRSVASEKDETDTSLKLAPPSSVPASSAMGVLKEESRSLASEDVIPAKSKDGKVKRGGSTEKQQLRERIRGMLLEAGWTIDYRPRKNRDYLDAVYTNPSGTSYWSIIKAYEAFQKRSEVDSGKSKPDGSSCSYAPISDDLINKLTRQTRKKIEKEMKKKRKDDGKREDRKKTSVRESSLGTDTLQHEERLGSYIKKKDKLLQGKLHAIDRENGDNSSDNLNVRRLKQDTVAKSSGGVASNSIHGRKSKLIGRCTLLVRHSDKRGSSDSDGYVPCTGKRTLLSWLIDSGTLKLGQKVQYVNRRRTRVKLEGWVTQDGVHCGCCSKILPVSKFELHAGSKRHQPFQNIVLEFGGTLLECLIDTWNRQKESDHRDFYSIGIDGDDPEDDACGICGDGGDLICCDGCPSTFHQNCLGIKMLPPGHWHCPNCTCKFCGAACEFPEEASERTVNEILSCSLCEKKYHKSCSLETDALSAISNDQSASFCGQKCRELYDHLQNILGVKHELEAGFSWSLIQRTDLDSDISRCPFPQRVECNSKLAVAVAVIDECFLPFVDRRSGINIIHNVLYNTGSNLSRLNFRGFYTALLERGDEIISVASIRIRGTQLAEMPFIGTRNIYRRQGMCRRLLSAIETVLSTLKVEKLIIPAISEHPHNWTKVFGFEELEESNKQEMKSINMLVFPGTDMLQKKMLMEDMQEACDLQHSHPPPPALVEKADQESSIRHDRHLHDGVCVSIVEKTDDGFGPMDSGSPASAVHLSASTVVRAQGDCCESDIQISSTEAEKNFTESATNLMLSSPSGASNGSPDTEDAALGPANIAVNSAEVSSRSTSVENRLKKLLIVLPLITIRKIFVWFA

SEQ ID NO: 38 - protein, 183 aa, synthetic construct
MGGFISRFWFMMFPAKEYKIVVVGLDNAGKTTTLYKLHLGEVVTTNPTVGSNVEELVYKNIRFEVWDLGGQERLRTSWATYYRGTHAVIVVIDSTDRARISIMKDELFRLLPHEDLQSAVLLVFANKQDLKDAMTPAEITDALSLHSIKNHDWHIQACSALTGDGLYDGLGWIAQRVPGKSPT

SEQ ID NO: 39 - protein, 461 aa, synthetic construct
MQKRQSGGGAAGGSATPVAKRGRPVGSGGSNAAAASAASSAAADSVAPPTLLGPSLHVPYAFAEQNNKRIVLALQSGLKSELTWALNTLTLLSFKEKDEVRKDATPLAKIPGLLDALLQVIDDWRDIALPRVLVKAPRVRLLGANSAVTGFGNEYEALNSNNPLPHPSAGSGSSKEASVHKSTSKTRPGAWSFEEDGLFNLDEEGRAEKQLCAVAASNIIRNFSNMPDNEVIMAQHRHCLETIFQCLEDYVTEDEELVTNALETVVNLALLLNLRIFSSSKPSFIKMTEKRAVQALMGMLGSAVKAWHAGAAELIGRLIINPDNEPFLLPFASQIYKRLVDIMSLPPTDAQAAAVGALYNLTEINMDSRLKLASERWAIDRLLKVIKTPHPTPEVCRKAATILESLVLEPQNKPLLLVYENAFAEMLFGDARYSDIFARILYELTSRPSNKVASACGIWGM

SEQ ID NO: 40 - protein, 333 aa, synthetic construct
MLVETLDNSTQKLVLIDTIQRLGLAYHFNYEIENSIQNIFYLSQNSEDNDEHNLYVAALRFRLARQQGNYMSSDVFKQFTNHDRKFKENHTNDVQGLLSLYEAAHTRVHDEEILEEALIFTPTHLESVIPNLSNALKLVERFGLRKQISICKGQISRSLLLDGGNIFEPQYGRSRSMITKVVKMNSIIDDTYDAYATFDELVLFTDAIQRWDEGAMDSLPTYLRPIYQGLLDVFNEMEEVLAKEAYYKEAEWLNANYIPKCEEYMKNGVVTSTGTMYGIISLVVMEEIITKEAFEWLANEPLILRAASTICRLMDDMADHEVVYITPIGCDPS

SEQ ID NO: 41 - DNA, 11742 bp, synthetic construct
  source 1..11742: mol_type = other DNA
atggcggcttcaagtgcacatgaggaggcagggacaggaaggtcttatgagggattgtcgagcgggcagcagcaatgcagtgaagcgttggcggaatggaggtcatccgagcaggtagaaaatggaactccatcaacttcgcctccttactgggattgtgatgatgacgacgatggtggtatggtgctttcactgctttgggtctccaattctttttaccaattcattcaaatttatgtttgatagtctatgactttgtgatattttatttcttagtatttccttcagtcttttaaggtattgtggtttctttatgatgaatacttggtacacgatgttttcgctttctttaggattttctttctatgctaagagtgatatgctgtgtaggagtctcactgttgattatggagggctggacttatctgcgtatatttatgtaaagttatctagattgccagaattccttctttttggatgaatatttctttcctctctttggtgtaggaggctagtataccattttctttgagagtttcttttagaaagtgttttaattagttctttgtattttccttgtctttagtttccttgtaaaatttccttctccctcccccctcccccccaagttaatcagtcaaagtagcttctaatattctcaaatcttcttttactgtcccttttctagtccctaatgcaaaaagtggacatcattactgtatttcatatacaaggtctatgtgattttgtaatcatggacactatttctgctatcttacatataagcagtgatgttaggtggattgtacaatatacattgatgggtgatttcgtccatggtagatccagtatactctggttttaaagctactttctttcacaattctctgcctccgctccgttgttttaccctttcctaatttttctagttcttcacttttcagctctagtgaaattgagacaactcttttgtcctaattacatagttgttgaaaaaagctattgcagtctagttgtatcttttacttagaagatatcatagtggctattaccttttaaatcaatataacttaaaggatatcacagattcatagtgtctgcttacttgcttagcactatcccagcttcctgattttgataataaataaggatataaagttagaagatttagtttaagtgatagatcgaaattgcaaaataatgcgccttttgcctattgggttgagatatgatgcttgagttataattgagaattttctttttgcatggaactctgtgtcattcttattcagttatacctttggtttttattcagttatcctcttttattgtcttcaggccctaaaccttctgagctatatgggaaatatacatggaagattgataagttttcgcaaattaacaagagagaacttcgcagtaatgcatttgatgttggcggctacaaatggtatgatgttcttgtgaagaattgttttgtttaactttaagctcattttaaaactgcttttagaatcttaatagtaagcctccatgtgcaaccctatattattagttagttcttgagattagattaagaaaggcggcttgtatctttatattgatagaagagtggttattttatattaactagaataacaatgcatccatttatttacaatgataatgacactagctcagaggcggatttaggatatggagatggtgggtgtcataattttctttaatatacaccttgtaatgactaatatagagtttgttaggaacatatattcggttgggttccgtctcttttgagtttgttcgagtcaacttagtaggtgttttttatttgcatatatgaagattacatctcaaatgtctaagacaaacgtaattagaattttatgccggaatcatacctttaattaacatcacaagtaaagtcaacacttccactaatggaatttttttttgtatatttaaaaaataatttgcacaagtagaataacatcgtcaataagaaaattacaccaattaaacaagaaaaataaaagaacattttcaattggtaaactacgcccccataagtaaatgtagaattagatgatatataaatttcgtaaaatcaaaatcataattttcatgttttttctaagcaatatttacaaaatttcatcacaatttgatagtaaagagatttaaattgaacttaacatccatagaatagcacaattttttatgatatactccctccgtttcaatttatgtgaacctgtttgactggcacggagttaagaaaaaaatgaagacttttggaatttgtggtcctaaacaagtccaaatggggcccaaagtatttgtatggttataaaagcttctcattaagggtagagttgtaactttaagctaaattgttaccaaatttagaaagggttcattctttttggaacggaccaaaaaggaaataggttcacataaactggaacggagggagtaataaatagaagaaattatgaaaaattaaaacggatcataatctaaaatttccattgagacccaagtctttcgattttgaaagaagaaacttcgcaaaattgagactaaaatatatgcttatatttatgggattttaaaattttggagtttctttttgagtgttttcgctagaagtcacagaaaaactaatactagaataaaggaaagagaaatataaaaaagaataaaaaggcaagcaggaaattgggagataactgaaaagggaaacgactatgacaaaaggaaatgaaaagcacaaaaaagggagccaaataaagtttaacataaattcgaacttgggtcacacaagaaaagcgttcaaaaaggtctcccaactatggcatctttgtttagtttgcaacaacgggtggcagaatcatatatttaacctagtttaagaaaatttcaatataggtacataataattttatcaacctagcgggtgccataccatccccatcctaaggggtggatccgctcctgcactacctgtatcgtattcttcctatttctaatgctatattaacttatattaccatttgttaggctatgttgagtcttcttatttccaatgccacatgaattgccaatatgctttccaccaccattcttcggattggagcatataatacaagcgtttacttttatgcaccaagcttgttataaataaataccgacttctaggatgtctcataaggatgttttgctgtttgttcatttgaacaaacggactaagtgctaatatggcacaaccataagaggagtaggggagacaaggttaggcactgggtagcgcagtgcatctgtctcaggtacagaggtgacgaggggtgttaacctggccacccaatcctaacaatataatagagaagtaaagattgtaaatacgggttccgtacttcaagtaggcaataaagaatgtccaatgcatatttagcagactataacagctgcctctcagctgcctcattgtttccgggaagtaaactaggaatggttaccagtaatatgattataagtactagaaccaattatccaaagatttctattacttttgtgagataaaaatgtagagtcaccaatacgtactgtagtggaggtgaaaggactgagcttgcttggatgcgttttgtttgaaggtactcttggtaatttttgattagtctttgagacattttgaagactgttaagattgtttagtttctactagactgagaggaaacgggacgtgttgataaggtcaggattctgttagactggatgacagacctgtgatactctgattgacttgataatagactaagagaaagacttgaaacaagagaattggctaagaaaatgactcgtgacttactcgagtggtttgtcacccggtaactataatttacttatgtaaaaagttattacaaattgtattaacaggaacaacaattaatcctcttatatcaaatgctatactacctattttcattttgtgttatatttacttttctaacttataatgctacattaactcttcttattttgtagttcttcattaagtgctagtatcattttaacacaagctatgttagtaggatgaacataagtaaaagaggagaattgagagttattttcgtggtttggttggaaaaatagttgaggtgcatgataactccctacgtcacctttctccaattcttgggaaacttaaatggtaatcacaaaatcgtttttactacgtactgaacaaaaagtaaactctcttactttcctctagaaatgaatggttaatgggtgacgtaaggaaagtaatgattaagtcctattcctgttcccttcctcttgtccattgctgaaccaaataaacttgtagcttagttcgaggcagatgcatggctgactagctggcacaacagttatattcttgttgttttgatgtagaggttggtgatagtaattgttaatttggagtttgggttatcaaggttgctactcttgttatcatagtggaagttccatgggggcatgcatttgcagcaacatctgctgtatcatcatatgctcgttacctattgttgaatggaaattaccagttgatgaattaggaattgactgacaaatgtttgctccaactgttgcgacaggtacattctgatttatcctcaggggtgtgatgtttgcaaccatctctctttgtttctttgtgtggctaatcatgacaagctccttccaggtgtggtaatgttgatagttcatcacaagctatctagaaattgatgtgttagctatgattatgatgttttgttcctattttggtaagaacaggttggggtcattttgctcaattcactattgctgtggtgaacaaagacccgaagaagtccaagtattcgggttggtttgttgggtctgttctttttgcaatgaatttattggttccgttcgtgctcacattaatagacagttgcaaatttttgcagatacattacatcgtttctggaagaaagagcatgattgggggtggaaaaaatttatggagttgtcgaaagttttagatggttttgttgatgctgatacgctcataataaaagctcaagttcaagtaatcaggtcttttgcaagccattcctatttgagactttttagatgaacgcttgcagaaatgcgtataccctttgttcaaatgcatataccctttgttttaccttaaagaggtttagcagatgcactaatagcatgcttcttccgccttttttgtggtaaatgtcaaagtgatatctgatgtatgcttgtgtatggggtgtaaaaggggcagctagttgaccttttcttttaacttttgcctaataaaagagcagcaaatcggttagagatgtggacatgccgagcatggcatatgacctgagatggacatactgacatgaaaactaaacatcattgaattgagttttgcaaaatttctattttttatagcttatatggggattaatctaataaaaagttatacatgcacactatatatatgatacaaatattaaccaggcaattcagcgatctttttaactcgacatatcatccttgtgttcttttatccttgtaatattttactattatttattgacttgcatatcatagggagaaaatatagaacagaaaattgtaggaggaagtgtcttcaaatattgttgaggacattttgtatggtggtcaaacagttaatgaagaattttggagtatgacaggaatttgatgcatttcttgagtgtttttaaaagtgcctttcaacatgaacatgagtgcagtgcccttgttacatattgtgaagtttgaaccagttagcaacttagaccataatagaccaaggcaacaagatgtcacaattggaaggctctatttactttacataaatcttcaatcgaacttttagtttagcttgaggattgtagcagttcttttttgaattattctttctgagaatagaaagtactgtcctttatagtattttccagtaagttgctcttaagcagcccccttcatgcttgaatgggtctcaaacagtgattgttggtttatactgctatgatccttgcaatgctaaactgctatgatctctgtttaatgtctgtagggagaaaccagaacatccctttcgttgccttgaccgtcaatatcgaagggagcttgttagggtatatttgactaatgtggaacaaatctgccgccgtttcgtggaagagcgacgagtaaagcttgggaagttgatagaggatagagctcgatggtcaaggtagggatgacgatattcttacttttggccatgttttaggggctttcagtttccatagggcactatcgtctactttggtttgtgctctcttttgtgtctagtctagttgggttgtttgtggcttgtgactcatggttaatggttttatgttgaataaactgtgttcctcatctccttaggagaaatgcttaagattttgatgtcagtatttcatttgtcaaatttctcagcattttcacctaaaggactacttgtgatgaaatttcagcttctgtgctttttggctgggaatggaccaaaattctaggtgcgacatgtccaaggagagatcagattcaattttaaaagtggttgtcaagaactttttcatagagaaggaggttacttctactcttgttatggactccttgtacagcgggcttaagtctcttgaaggccagataatgggcaagaaaggtaaagcaaaatattcggatgcagatgaacaactggttcctattgttcgtatggagaaaaacatgtttgtgttggtggatgatgtcttgctattgcttgagagtgctgcattggagccattgcctccaaaggatgagaaaggccctcaaaacagaacaaaggtgagtgacataatgccattgttgaagctatatgccagatatattctcatggttatagcttgagttgtgcagggctactgcaaaaacagaccccccccccccccccccccccgcgcgcgcgcgcgatattggcattcacaattgataagaatgttacccaacaattgactggaggtcaacatttaaatttaagatgtctctttctggcaatgttcaatgctatcagactgctcgtattaatcacgcgtgatagtagattaagattctcttatttattgtgtgttcttggatttgtttgttgcttatttatgtatttagtttggccctgttatttaggagtctatcaaattccaataagtaacagtatcattgtgtgatcgttgacaagcagttacatgtagtccttgatctgtttggacacatgttaagctcgtttcctcgataaacttctgcgtttcagcaagaataaaaatacaccttttgccagtggtaactgtcaactggagtgcttttctatttaggaatttatatttgagagaattttatggcattgggccagtgaagaattttatggtgtctaaaaggagaattttagaatcctcctttttttacctattataacaccatccccacaccccccccccccccccccccaaaaaaaaaaaaaaaacaacaacacacattttgcaagctatgtgtgaactttgtggctcatgttctgcatgcttgcactttatctatctttttagcagataacatgtatccataccccatagacacttgcttattgtaccattgttttttgtttcaggatggcacttctggagatgagtttaacaaagatacaattgagcgtgatgagaggcgtttaactgaattaggtcgtcgaaccattgaaatatttgtcctagcacagattttcaggtaaaaactcttttgatcaattatttgcgcattttagtcacttaattgtgatataatatataaactgctggttaggtataaaagtttctctttggccttgctatttaaatggttagaacgtatagattctgttgctttttggttcgtgtttttgcatggaggccatttcatgtttggaggtctgacccatcttcatgatctagcttggaatacttcaagtctaggttgtaattttctcttgaatcctatttgtctaaacttgagatgtattgaggagttctcttaattctagtttagcaattatacattaattccaagtttagtatattttgtgacgttctgtaatatcatctcatgcacaacttatgtagtgtacactagttggttaaatatacttctcttattttgtggcaaaagttaatacgtatgtcgtccagtccacctatttgttggattgaagttaatttggtttggcctcacggttggataaggtgcgcttccagcaaaacaaaatatcttttccttttggaagtacaatttcagaatcaaatttgctccaaaccaatccagaccttagtagccctcatacagttatcttttacttcaaccacaggaggtccaggagtagacttctcttgaatccattcttacttctgtcagctggatcacctaagtacaacctaaaggttttacctcatgtttctatgcatgctgatgtcacttgattggtagaaaataaaggaaaccagtagttggtgcttcgtttctatataggatcaggtaaaggggaagctcatgttgtatgacacgtcactcaaacactcatacgtgcaggtattatgcacttatctttaatgaccttgatttaatgttaattgattcttctgctatgctggaatgttcgttaaactaacataaatagttaagagtggcatcagtaactgcattttcctcaatgtgaggtctcgtactttcttaaatagcggcagtttgttattcttgcaattttaattaacaatgactttttcatttatcttctgtagtaaaatagaggttgcgtatcaggaggctgttgctttaaagaggcaagaagagctcatccgtgaagaggaagctgcttggctggcagaaactgaacagaaagctaaacgagcatctggaaaggaaaaaaagtccaagaaaaagcaggtattgcattttttttatgtatgcaggacaagaaacaagctttggtattatctatgaattagggcctttgctttgaggacagatacatttatcgtgcttgctttgtatgtcaggctaagcagaagcggaataatcacaaagtaaaggataagggtgttgatgaaaaacctggatccatggagctatataaaattgatcaggatgggcctattggtgaaggaaacgagtacataaatgaggagcgagaagcagtacttggaaaaccagatatactggaagctgtatctgatgtttctgattcaattgattgtgtcccagaggttattaatcctgactctgaagacagagatgcaagtcctgtcaattgggatactgacagctcagaagttcatccttccacggaaactagctgcagtgggttaagtgacctttcagctgtgcaaaatgggttagctggaaggagaagtccttccgtcatggatgatagttcatcgacgtgttccacagattcgattccttccgtggtttcgaatgggccttgcagatggacttcggctaaccataaaaatcagaaatcacctagcaggtaaccttctttatgttttttttggggggggggggggggggggagggggagcatagtctaaatatatttgaaatgttggttgtattctaaaattgtggtgcttcaactattcagagggaagaaccaaagaaacaagtcaacgtctaatgcggctgattgggctagtgaaacactaagtcagccattagatgctgtttcagacttaggacagctaagtgatagaagttgcggggcacctggatctgagtcgcattctactgtgctttcgtcgaatgagcagcaaaaggtgaagaaggttattatcttccctgcactccctcttttccttctgaagaaccaactggaatcgtcataaaatacttttacctaagagagaccacctaatgttgatgaatttttttttatttttggttcttttctctctctttcttatccctcattatttcaccacattgccatcataaaacctcaagttcatggtaaacaaaaaacgaaatgcagggaaagaaaaatctcacggagacttagtttcgtgttcttactgttcatgtaggcaattagtattctcgaaatgccggtaaattgcctctatttgttaagatgcgtatgcctttccaatgagtttattgggaaatgtcttttcttagacactgtgattctgtttgtctccagttaataccaatttcatgctactttcttactctgagcaggaagtagtagctttgcagcagagaaaggccgagacagagagaccttcaatggagaagccgagtgtcaagtcttctcggagaagccctccaaaagatgcaggctctgctgttcagcagaagtcacagacgaagatctctgtcacaagtgatcctgctttggttaaaagatcctctacagatggtcccaagctagctgataaatcagctctagtgtctaattcatctgaaactgcggtattgaaagctgatccgcataaagctgtagagccaagggtcaaggataagccctcagtgcagcccatttgtattatgactggggagtcctcgtctcaacaagtaacagtctcttctacaacagaaaatttcaaatggcaacaagtgcccgccgtgtccagaccattgtgtgatcctttagttcctggacctaggcctgctgcccctgttgtttccatggttcagacaacaccatcactggctcgttcagtgagtgcagctggccgattgggtcccgatccttctccagcaacacacagctatcttactcagtcctaccgaaatgcaattatgggtggtcctgtttctggaagtccggctagttttagtcaacctcattctccaatttcagcagttaacctatcacattcgtactctcaacaacaacctccactggtttctggggcatcatttttacctcatggtttggaaagagcagaaccttgctcgatgagaccaagtttttcatatggaatggtgaataatggcagcttgcagaatggactgcaatgggagtgtccccaaagggacagtaacaggagtatatctcaacatcatccttctgcatcaaatggaattaggaactttgatatgttcaagactgtaaacagtagaacacatgatcacgttcctgattctctggcttgcacgtctggacgccagtcccagagtgtatcgcctgatgaatttcctcatcttgatattattaacgacttactgaacgatgatcatgggattggaagggcttctatacccgacttgggctttcaaagttttagcagcggatcacagcatctaaatcatgtgttcacattcccaggtgatattggcaccccaactgacctaggtgcctcatcaagctcttgtaggtttgaacgaacacgaagtcatcatgacatgttccagcataactattctggaggcctttttgatagtgctagtgatatgattctgcagcccgatccacgatttatgaatggacatcatattgatgggttagttcctaaccagtggcaaatgataggttctgatccgtcatttcttgggatgaggaatgtgaatattgatggtagtcacccgtatcccctccctgactattcaaacatggcgtgtggtgtaaatgggtacggattatttaggccttcgaatggcctttga

SEQ ID NO: 42 - DNA, 8237 bp, synthetic construct
  source 1..8237: mol_type = other DNA
tcatgacccaacaagccctttgaaacgagcgtaagagattataaatatgaaaagcacggcagagaagatagcactaatgattaccacctgcacagcattaaaccatccatcaaccaaataatgtgtgtttcccatcaggtatagtactcacacaatcatattaacgacagccaccacaaagtgtcaaacttacccatttaaacatgtaaccatgatctttattccaagaatatgggatgttcatcccaaaaatcgcagcaatcaaagagtagatagacaaacacacagttccggaactgagaaaaagttctaactgcagccaacatgacaaaagcacatatgagcatagagacaactctaatcgtgagattcaacgagattgaaaagagatgcaatgaaagataggtaggggatttagatatttccaaaggagagcacctgaatgagctgatttcgatgattgtccagctgaaataagcagcataaggtttacatcattacgtattcagatgaatcacatcaaactgaaaattatcttggcattttaactacctgaatatttatgtaatcttctgtgttatcaatatattctcgtagctgcaaattaaaatagacaaatgttaactggaaaatgaagataaagaacactctaacatgtcactcagcactgaaaatttccaaagaacaaaatctaaaagatcactgcatttccacccatctagtagacatgtcagaaaggccattattaggaacaaatgctaagaagtacaggaaactgaagagagaggcatagtgttgaatgaagggattcaatgaaagcaacaaacactagtccacatagagatcccattactcggacgatggaaggtctcctccctattcacaagaatcacaaccctctcacaattacttcatttttcaacatttgcttaaatcagaataatttgacctgatttttgttgtgtatctcaagactctttgtatcttactcatgaaagaaaaggtaaaagaaagcaataacaattcagggttcataaagaaggaatatctatatctaaacataaggagacttgattgctccctccgacaatctgcactattcaatcgttaacaatggaacttcagaggcaagcatactactaccactaaagactcttatagccatctgaaataatgtggtatctctactatataagcacattgaccaaatctaaagaagactgtcaaacataaaatccacaaaaacacacaagggactaaaggttctaaaaggtttcgccagcttaatatatccaattccacaagagggcaaaacttgcataccgtaattagtttattaaatgtgctatcaatttgcccgaagtaagcctgcaagaaagaaaaacatttcttgtaagtagggctcagaagcatgctttcttttttcaacttctccttctcttaggtataacaaatgatgccattagaagaagtttacctccaataacatttcaagctcttcaacatcattctcatccccccgaaatgttgcaatactagctctactagccctggatatctttgagccaatggtaggtgaggcaagaaaccaactggctgcacctgatccacttacaggtgatgcaccagccaattttcttgacaaataaaggtcagccatatcaccatcatcatccagaagttgctcaagctcatctctcacctgcaattaagtacataatgaagatgatgaatccgtggttgtttaaacaaactgtgtggtaatgatgatgcaaggagcgcaataatccaactgcaataaaatcattccattacagatattattgttaataagagtagaaatcacggggcacgcgtaagttgaagctattcacatagtatatcaaaatggagtggagccccaaggctttgcttcaggcaaaaaaagtacagcacgagaggtgttgtcagcgcatttcgagtttaaaaaatttagtggtaacaccaggtctggcatttcagcagagaagagtgacgaatcccgttatccaccaagttttgaggatgaaacagcagatttcttggtcatcaaaacaatataccaaaatggagaaaaaggatgtatgtttcacataatgggtcacgattctattatctacatgttaatcatgtaggaaaatttaacttcttcttgctaaatttaagatgattaccaagttattatttccccccttttcctggtgcggaggtggcagcttctagagaatgatctatctccaagaaatttagatatcccagcagcatatgtttatatgaggctggattcagcgatcatgaagagacatttttaatgaaataaagctatattataaaatgtctcttttaacaaaatatcctcaaaaatccgaagagaaacaagttcaaaggatttaaatattgttcagttagttaaaacacactaataaaaatgaaaacgtaaataagaaagttaacgtcaatgtcacttaaataataggtaagacataaccttttgtacgcgagcagtcaaccgtgtcatttgactctttaatttccgcactctatccagattacggctactaatctgcattaaacattatttgggaaaaattaccccatgaaagacagataaaactctagcaattaaccttctaaagattgcagacgcagagacatcaacatatatgtgcatgcaggagattaagggcgaggatcaagaaaagggaaccacgtaacgaaaagaatctcaagattcttaacatgaaaagaaacatttcgaactttcaaatgcagtacaaaatcaaacttgttgatgttagtagctagcatctaagctatcccatgggatccaaacaaaactttacaaggacaaaccacaggaaacggacacaatgtccaagatgccaaaaaacagcagaagggactgaaataccaaaaactaagaaaacaatatatattctactttttttataagaagaaattatatgcattcgtccaatagggaacgatatttgagcaagaaagagctcttttggtaagtttcgtgaaataccttcaattgaaatacacagaggcgagtaaggggttttttttgtgtgtgtgtgtgtggggggggggggggggggttaaaataaaagaagacgccttactagtctgtgatgaggtagtatggcctgtcctaattgacatcctattatcctttaagaagtaaatatgacaatgttttactccaaaagcaatcaaaagttttctgtgatagaacatgcatatgaaggatacccattctataattaacaatagcggaaagaagtaagtaatgaaacttttctatgggcatagttcaatcgttcctcatttcaaaagaagtagaagagagtccaaagaatatttcatatggcaaggaatttacaaaaataaaaataactacaatttctactgtataaaaaggaattacctttatgttatttactgttaataataaagtgttatctagggtagagacagtccaacactttatggtgtctcgttttctttaccttataattcctagttgaattttatggtgtcttgttttcttgagaagataacaatggtcaaaatcaaaaagaaagggtaacaaaaagaaaaagaaaaagcagacaaagaataggatcttggttcacatagacaactaaaaaacttgaaagctgaaatagaaagtttccagcatacacatgcaaaaatacaacaacaaccgcgcggtgaaatcccaaaagtgcggtatacacatgtacaaatagacatgtaaattatcttttcgttaatgtgtgtgctgtgaaaagttagggattgtgagtcatcaataaaaatataactggaaatataggataaacaaggaaaaaaaagaaaagataagaatatttactgaaggattttccctctagggatggaagacctatgtcctttagcatactgtaatgcaaatgtaacaatgtaaacaaacactaaaccgattaaaatttcatacacagacctttgatgttagcatgtctaaagcagggtaaacagcagtttccaattctaatgtgcgagcagcaaggtaactacagatggcttccaaagcgacctctagagctcggaattcaaatggagactctgcattgtgtttggcacaagaaaaagagtcagcatacttacgaaaagaacatgaataatatacaaagaaatgagaagcatgagctcgagaatcgaatatgagaaattcacaagtacagaaaattgagaatgataagacgaaaaccatcttcttcagttgtttcaatatcatgctgaacattcatatctttttcctctctctgatcttcacgatttgcattcattggtttcagccgccttctaagttcctcaacaacaggagtaacattttcgtccaatggatctcgaagaagaacctgtccattagaaaacttagaaatagtcttatatgtggggctgatttaataaacaaaagttactcaatgtttatatctatttttatgaagatagtaaagatgtatgtatgcgcctcaccctttttacaagagtatgcattctcctcaccaaaaaataaaaaaattgggagccaaggaagttggatcaaatcagaatggcaggaccgaatctgactcattttgtctaattacagagattaattttgtttatcccaaatagatttcactcatgtggctaaaaattaaaggccagtgtactcattcctgttgagttgaattcagaaaaatatagtacaagcaggaattctagtcctacttgggatcaaggttaatctagattaagatgcgaaaaaaaaaaactcagttctcatctttcccggattgaccaagacctgaattaagattaatatagaatcactcagttcaaagtatgtttagatccgaaagtagcaagtagggttctaaagttgactgaagttgcttccaccaaatatagattagggagctagcctaatcaatatgtaagtatcataaaaagtgttcagaacctaaaggatttaactttaaaacttaataatccattagaatcgacctttaatatttcaaagcagactttttgttgaatatctacttctctaattaaatgttggaaagtctactttgcgggtcttgaaaaggaaagctaagctgtcatactttttcagggtttagtggaacatgagataaaaatcatttttctaggaataactctattggtattcctctatggtggaccggaccagcatgtatatgaccaaattctggaaatgaaaaaagaggacatttggaaagaaataacagaaatggatgaaatagttttgagctcaaatgaacattttaatcaatttcagactttgaccaagattcaagtagggattattttagttgcagcacaaaatcccaactgtctctctgagttttggaaagcatagattgtcccaaacccatgtaaacaaagatggttgcagcaagatgctggccagtacaaaaataaaagtctacttatgatgaattatctgagctccctgtgtgtgagcttccaaacacatagcccatcctaaaatccatataaaagatgagggttgctgtgtgttaatgaatccaattgtgatgaatttcatgaattctgaattaactggaatatcggcatccaggtttagtgttagatgagcaacaggttccataacattgtggggcctggaaggacaaagtataagcaagggttcctacaccatgaatgactgtgggtgaagaagtgatgctacaaaaaaagggctccacatcttattggacatatgagataatgaaactaccacaagagagtcatattcgagtagatagtttcgacataggctcgttgataggaaagtaaccaaaattagtggatgccttaaaataggaggagagactaagttgacaaggacaactttacaatcttcttaccgcaatgatatacaacatttccgttacactcgtaaaattaggtctaataaagtaaagatgccataaaaatatgagggttagagagcatgtggtctagatggtataaaaaataccgatagtatcctatagagatttacaagttaagaccagtcaaggtgatattctcaatgaaagatcacaaagacttaaaacacattcagaagctaaatcctgctatttctacaaaatgcttgtaatagcatcctgctatcctttttcctataatagcatatgtagaatacagttacacgtccttgtattattttttaaaagtacatgtcctagtattattgaacgggacaagaggaaaacggaaaaggtagcatttttttacttcaacacaaataaaagtgagcctctccatctggacgataaaagataatacacaaattgtcgttatctagtctttgtccccttgtgatgtcccaactcctaacttgaaatgatgagagtcaaacataagatttcgcaacacacaacaattgctatcgtctcagtaatgagaaactgctggcacaactatcattcagcaaattgatggtatactaaaacagttaaacctcaagtgaattggcaagatcggtgactgcatcctcacttcgaaaacaaacaaaatgtagttccatagactccataaatacaattattagaaatttaactaactttgtaaaagagaagtgcagaaaacaagaagctttaccaaaaagaacataaattacctcttcagatgtaataatagccttgatatgctgcccttaatcacaatagaataaaagatgcaacaatcagtaaacaagacacttgaaaatgaaaaatgaaagtcaaagttaaaatgtcaaccaaaaagcaaactgggaaaaagcagctatgaattggcttccatcttttaccacaaaggagaagaaaaaaattaattagttaatttactatagttatttatggaaacaaaaaggaaacaaatttcaaaaacttttgcatattatctcctaatacagagtgcacattccaacaactatggctcagtgacaaaattgaggatggctttacaattctcaatattcattttgctgcatttcaacccgtttcattccaacaacaactaaacctcagtctcaaacaagttcaggtcagctaatcactgcccacattactcaatttaaatcatcgcataccaatattatgcaaatacaaataaaaagtgttagaagctctgtatattttctaaatgtataaatctccgaaaggtaaaaatactctataactagaggtaagttcaaatccaagtagaggcaaaaaaaaaaaaacactagatgatttcattccatctgcctaaaccttggtaggctgtgttatccaatacctgtgcggatggaaggtaccagctactcactggaatataacccgagcaccgccgtcataaaaaaaatactcaataattcgagataagttaacatcccatcggaagccaaaaatactaggatatttcttctcatctgcctaagtcttggtaggcatactttctcgctatttgtgctgctggtaggagataatagacatccaatggaatatacctcaagccagcaaggacaccaccattataataaaatactcaataattcaggattcaagcaacaacaacaactattgtgtctcagtctcaaacaaattcgggtcagctatacgaatcttcactaaccacattactccatttaaatctatctcatgccaatattatgaaatgtaaataaaaagtacttcgtttgtcccagtttatatgtcatagttgaaatttcgagagtcaattccttagttttgacaggtgaattcggacatagaattttagaaaaaattaaataaaatttacatgtttgtaaactacataaaaagtttataagtaataataattaacaattcaaactattttgccacataaattgggatggagggagtattaggcgaataattcgagattacgaataagaaaaaaaaacaaaccacctccaaattgagaacaatagctctttcacgccccagaatagtggaagggtaagatagtagcggatcgataatgcgaagatcacgagcgtgaatttgcacgcgatgcatgatcgcatacttatcaacatctaaaacagtctccaaaccggaagcatccatcagaagccaactccgtgacccggtaccttttttcttcaacccgactacttgactcggatccatcaccggcaccacttctcgacccatcaccat

SEQ ID NO: 43 - DNA, 5299 bp, synthetic construct
  source 1..5299: mol_type = other DNA
tcaaccctgcactgctaaagcttgcaagtgctcaataaaaacttgtagactcagttcgtccgtaaatataagctctttgccttctgtgtatgttgagttgtgtgtaaccgatggattcaacttggcaaggagaaacctagcctgactactatgttgctcacattttatcagctttggcacaggaatcctttccgaaaccagttgctctgcatcaatctcgggagcttctaatagcttgcggaaactatcgtggcttggatccttgtcatagccaagtttacgccattgtacaatcttagagccataatgaataaccacatagaagtaggagtcaaagagcaagataacatctggagagacggagcagatgtccaaaagaacggggatagggggaccctcgaatgaatactggaagagggttggctggaccataataagtgagccaactaccccctcacggttcaacaacagccggaaaaaagcagtctcgtctggagtgctgttgaagacatcaataaactgagacctccttaaataatacatgaactgtgggaaaagggataaattagttgcaagacggaatgatgaggggtcttcctggatataatccccatactttgaagcaaaacgtatcaaacatttatctagccaccgcacaacatcttgagaaaaattgctctctgctctatgaatggcaagcctagccatcactgaagctgctgcttcttgatcaaagccagcagcaatttccggagagtggttcccaacccaccttcttgcagcagtggtcaccctctttcgaattcccgtgtttccatatctatagtgtgttatgaactgtatgaaaaatgcagagctaggctgggctttctgttcatcccctacttcaaaaaagaaggcaatgcacgttttgttagtaagtgcccccaatttccacgtataggtacctccctcgccaatttccttgtgactggctgaaccattcttcttctgcagagaaacacatggaccaagagccccacaaattttaacatcctttgttgttactatctctatagttgcatcaaggcacattttcaaattaccagcctcgtcatggctaaatatgtgacgtaaacactttctgaactggtccgagtcaaatgactctgttaacatcatgaaccctcctgagctttcaactgaagctctaagctcagctgctccaacttgatccaatgaacaagcaaagagatcaagtgcaatggatgaattagacaatcttcttgatatctgcttatagaattcactggacttcttgtagtaagcagcatgaccattaccgatgtctcgatgatttctaatggcattgccaagctcagagcttacgatcatccctgggccaattgttgctggccctgaggtaaagatcatgatacgtgaacctgcagagaccaagcatccttctagtagtccgacagcaactgatactgctactcctgtagctcgaaaaggtctatgcccttgcataacctgtgctgatgattgtatatcttcaattgctgatgtgatgctgaactccccttctgatagtggtatcagaaatccctgttttggagccataggtacctttcctgcttggtacttcacatgatgaattcccaggagttgcttggtctgatgatacagaaaaacaaagggaaaatggttaaaatttaatgcacaacaaatttctagaaattgcggtttgggcacttatcttctttgcaattaattctattttttttttgtgtgtggggggggggggggggggagggggggtgcttgaagctgacaatttaaagctattacttttttttatggttcagatattgatcagaaaaattggttatccacgactaaccgctccaaagccctttaattattgtgagcttaggcaacatacaattaaactaagaatttttggcatctaatgccttgaactacataacccctttttttacaagtataatttaattgataatggtaaagacgtgcatgagaggtataccaaaaagtaaagaatctaccgtagatatgtttctcttcaaaagacatacaatcttctatacaaataggaacctcatggttgcacgaaaggtcttttttttaccaggagcaggaggctattcctcgtgtacagaatcaagctttttcccttcaaaagttctcctatttctttctattcatactaccaacataacttctaatagtgcatccttcacccatacccggcataacccattgtatttcgaaccatcttaaaagctccaccacaaacgtgatgctacctcgaaccatcttaaaagctccaccacaaacgtgatgctacctggtaatgcagtaagagattatacatgtcatctccaaattgtttgcacatgatgaagcaatagctagaatatgtaatcctcctcttcctcatgttttcggctgacataatcacccctagccgtcaaccaagcgaagaaaatacatctcaaaactaactaaccccttaaaataacattttttggcagcaaacctttgaacttacaaacaagaagcaactgttccattgcattagttttcactaagcagaattaagtctaagggtgcagtacgaatactgttttcctgttttgttgtctttttactcaactacggaccaacattgctaagcatcaatctagaacttcaaatgcattgtttctccatcttttttcccatcattcggtttttatctttttgtgctttctctttatttcttcaactcctactcttgttaggcattgcttctctggccaattctttttgtcccaatgaagaagaagaaggaaacaatccccaccaccactaccaccaccaaccaacaaaattacaaatataaaaataagagaaaaagaaaaaaaatggataagttgctggaaaacaagtttttgctagcctctgcaggtggggtgtcctgctggctgtcaataaaagatgactggaagagatatgaaaaagaataaggtggcgccaaggaagagaaagagttgtagggtaatagtaaagaggatgttgaaggagcatatactcattaaagtagggtcgaagtaacataaataaagtgggggaaagcagaaaataacaagaaagcacacgaaatgaaattaggccttggggttagggaaaactatgagtaagtaattctaaagaaaagaagagaaaagaaaaaacataagaaaaaagaaaggggaaaactaatgaagtagctgacttggttctccaaaatggtgagcatgacaagaagaagagtagagaaactggaatctccaataattctctcaaagttgacattgactttagttaagaactgtcggaagggattagttgctaccttttaaatgtttaggtggttatatgtcatgaaagatggtgtatggggtaagctgccaaatttgcaactgttataaggagctctgcagcactttaatgtcccaaaagaatttaattatgttaggtacatattcattagttagcacaaagttttagactagaattaaggcatagatatgttataccgatgagatccctaatcaaaatgacaccaatataaagttgtagaaggagaagctataaatacatctcaatctagtaaatgcatagccttgttggctacaaggctacctaagacaccaaagcacttcctcttaagctaactacctcttgcgtgtctctcatcataacatatattcattcctttcaaatttgaataccattgtttgaccataaattctatgatctgttgatcaaatgcactatacaaattggagcaaacaggcccagctgacgatactcgttataacggcaaatatcatgttctgtatcgataaaaagtgaaccgtatattcatctgtagaaccaggaacttcaaaatggcaacccttcaactttcatttgacacttctttgctgacagaaaaaacagattaaaaaacctaatgcgtagaatttcttattctccttaaaagtagagtgcacatccccaccatacccaaaggttcctcttttttattctctttcacaaattccgagaatcataaaggctcattccttgtatatttcccatttcttattgaaagaaattatcagcgtgtcccctacttcatcaaggttcaatcaaagagctacaactcaggcaagcacgcaacatgcataacagcactaatacaaaaaattctcttgttgctttctgatgcataaaacatttgggaagcttacactttttaaattgtgttatttttgctataatacgtgatatgctatatttcatcagtattatgcgtttcaaaagtagctcactatcgataaattggatatattagtgaattgaaccagatagtacattgaactggataaccccattagcaaagacttggatgatatttcacattcaggatttaagttctatgcactgacaatgtaaagattttttaaaccatcaagtaagattaacctaccataataggtaactctttctttcaagcatgtataataacctaataaacaaagtgtgaacttgttataaccaattatattacagtctttacatagtcagtgcatataactgaaatccattcacattttacctgctcagaagaaagctcacgctcaccgtggaacatcacaaccctacagcactctccaaacccgagatcatgaactctaaccattgaatcaaacaccaccaacccaaccaatgcagtttcaggcaattgagcaataacatgaagtagctcatttttcaccaccctaagctcctcatcactagtacaggcatcaaccacaaacacaaatgccgggccaagcccataacccgcccaatccaagctcgaaaatgaaggcattttcgataacatcgacacatttccaaaattagagctggaattcgggttttgggtcaaaccctttttacccaagtgatattcaacagtactataagtcggaaaaagctcagctggcacattattttcgttaattccgaggtacgatctcggaaacgggtttttccggtaacaaaacgggcaattccagattcgcgacacgtaatcaacacgggcgtaagggtttaaaacggcgccgcattgagagcaaataacaggatcgtaattaagtatagggagctcgttaaattgcattaacggattgcacataactgataaagggataattagggcagaagattcggattttgaaattggccatgaattccatgaccaccggagaccttcaattgcctccagttccacgaaatccat

SEQ ID NO: 44 - DNA, 4497 bp, synthetic construct
  source 1..4497: mol_type = other DNA
atgagtcaatcaatttctccattaatctgttctcactttgcgaaatttcagtcgaatatttggagatgcaatacttctcaactcagagttatacactcatcatatgcctcttttggagggagaagaaaagagagagtaagaagaatgaatcgagcaatggatctttcttcaagctctcgtcatttggcagattttccctcaacaatttggggtgaccattttctctcctacaattctgaaataacagtaagtcacaatcccatgcttatattctaccgtcaaacttctctataacaactccatttgttgcgatcgttttttattgttatagtgaagtgttgttatagagattatatattataatataacatagtaatcggttccgagaaaacttgattctttagtgaatgactgttatatggggatgttatagagaggtctgactgtgttacctaaatgaataatgaagatgttataattttattagagatggatgaagcatttaaacatgaaagattcaagctttaagattggtctttatcgtacttttgaaattatgagtttggaatttaatacttcctccgtctcaaaaaagaatgaatcttttactatatggggtcaaataagattttctttggccatattttttgcaaatgcatttcaaatattttgaatttttaattgttgtgacttacaataccttttatgcagtttctaaatgtgtaaaatatatttcaaaacatttaaagaatatatgttcatcacattaaaaaatattgaacttgactctcatgctccgacaaggtttaaataaattgaaacgaatggaatattactattttgtaaaaacttaggtaggttttcactatatatctaataatgtattcaaaactcataaataatgatcaaaaggagccttgtgaccgggaggtcacgggttcgagccgtggaaacaacctcttgcaaaaatgtaaggtaagactgcatacaatagactcttgtggtccgccccttctccggaccccgcacatagcggaagcttattgcactaggctgccctttttagtgattaacatatgtggatctaggatttaaattttttgggttcaacctttaaggatctgttacaattttagtagaatgttacacataaatttgtgctccgtgaatgtattgagtcagatgaacctggtattatacatgcggatacgctcctgatgctcaatttctgcctgcaggaaattactacccaagagaaaaatgaacatgaaatgctaaaagaaatagttcggaaaatgttggtagaaactccagataatagtacacaaaaactagtcttgattgacacaattcaaagattgggattagcatatcatttcaatgatgagattgaaaactccattcaaaacatctttaatttgtctcaaaatagtgaagatgacgatgaacacaacctttatgttgctgctcttcgttttcgacttgcgaggcaacaaggatattacatgtcttcaggtaccttacatttctgccctttcccgcacagcttcattttttttcgttgttaaaagacagttcggcgcataaaatatctcatgtatacgcagggtcaggacgaaccgcccccaaggggtgtaaagtatgcaacttaccctaatactaaatatctcgtgtatacacagggtcaggacaagtcgcacccaaggggtgtaatgtagacaacttatcctaatgctattagtaactgattttatggctcgaacacataaattataggtcacacagtaacaactttaccgttgctcaaagactcgccttcctctttttttagttatcgcaccttatttgtgcagagaatagcaagtttcgagatctgcttctatatagaagacttctgtattatacttttttattttgtccttctgcttaaaaatagtaaaaaactatagtgtggaaattgtaaatttcttaactagctgtgaaatcaaatagttattataggaatattatttaagactccacttatggaaaaccactgggttgttgttgttattgtcaataataacttggggtacgatttacttctttttccatggcttgtccacgactatatttctattaacaatgttgtgactatgctttctttgagtcgagggtctattgataacaggctctcgatctttacaaggtaaaagtaatgtctgcgtacacactctactccgcagactccacttgtaggatttcactgaatattttttgttgttgttgttgttgtaataacttagggtttaatttcttgatgctaatgaaattcatttctttcaaaatataaacatggtgttcaaccagatgtgttcaagcaattcactaaccatgacggaaaattcaaggaaaatcatactaatgatgttcaaggattattgagtttgtatgaagcagcacatatgagagtgcacgacgaggaaattctagaagaagctcttatctttaccacgactcatctcgagtccgtgatcccgaatttgagcaactcgcttaaggtacaagttactgaagccttaagccatcctattcgcaaagctataccaagggtgggagcaaggaaatacatacacatatatgaaaacattggaacacataatgatttacttttgaaatttgcaaagttggacttcaacatgttacaaaagcttcatcgaaaagagcttaacgagctaacaaggtacatctactattcttgtcatcttcataattatggtacaatcagacctctctataaaatacatcctttataacaacagttcactataacggtcaagttttctttaaaatcaatgttttatgttaccaaattattttgaaagaaatgtgactattatagagaggtttgactgtaactcgcgctaattaataacacctaaagtttaagtatgttaatgctgttatgatatctatagctggtggaaagatttggatcgtgcaaacaaatttccatatgcaaaggacagattagtagaagcttacttttggacggtgggaatatattttgaacctcaatatagtcgttcaagaagtttggtaacaaaagtagtcaaaatgaactccattattgatgacacttatgatgcttatgcaacttttgatgagcttgtgcttttcacggatgcgatccaaaggtaaaattatatataataaaatctttctataacaacgtcatttattctgatatttttttaagatgctatagtgaagtattgttatatagaaatatattatgacaacttagactttgcagatgggacgaaggtgccatggatttattaccgacatatctgagacctatttatcaaggccttctcgacgttttcaatgaaatggaagaagtattggccaaagaaggtaaagcagatcacatctactatgcgaaaaaagaggtaatccttgattaagttacattaattactacttaatagttaattaagtaaaccaagttgtagggaagaatcgcaattttgaactattagtacattttctgttacttttttagatgaaaaaggtggcggaagtctattttaaggaagctgaatggttgaatgctaactacattccaaaatgcgaggagtatatgaaaaatggacttgtaagctctaccggtccgatgtatggaataatttctttggttgttatggaggaaattataacaaaagaggcttttgaatggttgacaaatgaacctttgattcttcgagctgcatcaacaatttgtagattaatggatgatatggctgatcatgaagtaagtataacaatataattttcattttatataacaatagccatcgtaattcgcgaattttgtccctaaatacaatacaaaaacaactacaataacaaaaacaacatatccaatatattcctacagtacgggtctaggaagagagatgtgtacgcagatcttaccctaccttatagaggtagaaatgttgttcccgatagaccctcgactcaaaaaaagcatttctcagtctgatttcgagtctaggtggcacttttgcatgataaaataaatagacatgcttgataaattacaacttcaatgatcacatttacttaaactgaattatggaactttagaaacggctgattaaaatggtaaaatattgtataatattaatgaagaaattgaaatatattatgttgtaggttgaacaacaaagaggacatgttgcttcatttgttgagtgctacatgaaagaatatggagtttcaaagcaagaagcatatgttgagatgcggaaaaaaatcacaaatgcgtggaaagatataaataaggaactcttgcgccctactgcagtaccaatgtttatcctcgaacgatctttaaatttttcaagattggccgatacatttttgaaagatgatgatggatacacaaatcccaaatccaaagttaaagacttgattgcttcgttgtttgtcgaatctgtcgacatatga

SEQ ID NO: 45 - DNA, 3085 bp, synthetic construct
  source 1..3085: mol_type = other DNA
ttatatgtcgatagattcgacaaacaacaaagtaattaggtctttaactttggatttgggatttgtgtatccatcatcatctttgaaaaatgtatcaaccaatcttgtataatttaaagttcgttcaaggacaaacattggtactgcagtagggcgaaggagttccttatttatgcatttccacgcatttgtaatgtttttccacatctcaacatatgcttcttgctttgaaactccatattctttcatgtagcactccacaaatgaagcaacatgtcctctttgttgttcaacctacaacataatatacttcaatatcttcattaatattatacaatattttaccattttaatcagtcttttctaaagtttcataaatcactttaagtaaatgtgctcattgaagttgtaatttatcaagcatgtctatttattatatcatgcaaaagtgccacctagactggaaatcggagtgagaaatatttttttttttttgggtcgagagtctatcggaaactgtttttctacctccacctagttgttgtttttgtattgtatttagggacaaaattcgcgaattaggatgactaattgttgattctggatattatataaaatgaaaattatattgttatacttacttcgtgatcggcaatatcgtccattaatctgcaaattgttgatgaagctcgaagaatcaaaggttcatttgccaaccattcaaaagtctcttttgttataaattcttccataacaaccaaagaaattgttccatacatcatacccgtacagcttacaagtccatttttcatatactcctcacattttggaatgtagttagcattcaaccattcagcttccttaaaataggttgccaccaactttttcatctaaaaaggtaacataaaaagtactaataattcaaaattgtgattcttccttacaacttggtttaattaattaacttattgagtaataattaatgtaacttaatgggttacctcttttttcgcatagtagatgcgatctgctttaccttctttggccaatacttcttccatttcattgaaaacgtcgagaaggccttgataaataggtctcagatatgtcggtaatgaatccatggcacctatgtcccatctgcaaagtctaagctgtcataatatatttctctataacaacattttactatagtagtttaaaaatgtcagaacaaatggcgctgttataaaaaaaatttaatatatataattttacctttggatagcatccgtgaaaagcacaagctcatcaaaagttgcataagcatcataagtgtcatcaatgatggagttagttttgactacttttgttatcattcttcttgaacgactatactgaggttcaaaatatattcccaccatccaaaagtaagcttctaccaatctgtcctttgcatatggaaatttgtttgcaaaatccaaatctttccaccagctatagatatcacaacaatattaacatacttaaactttaggtgttattaattagcgcgagttacagtcaaacatttttataatagcctcgtttgtttcgaaaatttttggctgttataataaagtgttgttatatagaacatgtattataatctaacataaaagattggatccgaagaaaactaggccgttatagtgaagtgttgttataaatgatagtttttatagaaatgtctgactattctataattatgaagatgataagaatggtagatataccttgttagctcgttaagctctttccggtgaagcttttgtaacatgttaaagtccaactttgcaaatttcaaaagcaaatcattatgtgcttcaatattttcataaatgtatatgtatttccttgctcccacccttggtatagttttgcgaataggctggcttaaggcttcagtaacttgtaccttaagcgagttactcaaattcgggatcatggactcgagatgagtggtggtaaagataagagattcttctagaatttcctcgtcgtgcacgctcatatgtgctgcttcatacaaactcagtaatcctcgaacatcattagtatgattttccttgaattttccgtcatggttagtgaattgcttgaacacatctggttgaacaccatgtttatattttgaaagaaatgaatttcgttagcttcaagaaattaaaccctaagttattacaacaacaaccaaaaaacaatccactgaaatcctacaagtggggtctgcggagtatagtgtgtatgcagaccttaactctaccttgtaaaggtagatggcttgttatcaatagacactcgactcaagaaaagcatagtcacaacagtgttaaaagaaatatagttgcggagaagccacagaaaaagaagtaaaccccaagttattattgaaaaaattactagtagtacatatctaccaaaagaacttaaataatattcctataataactatttgatttcacagctagttaagaaatttacaatttccacatttagttttttaagatttttaagtataaggacaaaatcaaaatgtataatacagaagtcttctatatagaagcagatctcgaaactcgctattctctgcacaaataagatgcaataacaaaaggaaggcgagctttggagcaacggtaaagttatcactttgtaatctacaatttacgtgttcgagccataaaatcagttattaatacttgtattaggataagttgcctgcattacaccccttgggtgcggcccttcctgaccctgcgtatacacgagatattttatacaccggaccccttttaacaacgaaaaaaatgaagctgtgcgggaaagggcagaaatataaggtacctgaagacatgtaatttccttgttgcctcgcaagtcgaaaacgaagagcaacaacataaaggttgtgttcatcgtcttcttcactattttgggacaaataaaagatgttttgaatggagttttcaatctcatcattgaaatgatatgctaatcccaatctttgaattgtgtcaatcaagacaagtttttgtgtactattatctggagtttctaccaacat

SEQ ID NO: 46 - DNA, 3972 bp, synthetic construct
  source 1..3972: mol_type = other DNA
atggcagcctcagaaatgctatcgaaggagcaattgcttcatctttttgatcgcttcgcgttcctcacctcccaacctggtacagacctcttcctttcttcaacttcatttgcatctttacgttttctttgcttttttttataatttcttttataactatatgtaagagatttaccatttctccttacttttcttactgctaagaattatttttatttctcaacagatgtcaagaaaaggattgctgatgctgtcagcgacaagcaggtgaaaatttcgtctttgaaaataactgttttttcaacttttttaagtgtaattttatgtttattgaatttatgcatctgtagcagcttgcttttaaagcagttgattccgtgtagctataccaaaggttcttttaagctctattaagcaaatgttgaagcagctaaaagctcaaaaaaaggcttatatgaaagaattttggtaattgtgtgaaagaatactcttcttattcacttttggcatgctgtaaaaaaaattatttccacttggttactttgtggtcgattcttaagaaataaggtttcttagaggttcatctttatgtgaagtcactttatcattcataaagtttttcctcaatattcaaattaactttttattgcaataatttgctattaactttatggatataattataattgggccgtgagactgcagtatgcacgtggagtgtggacgacatgagtcgtgcaaatgtatgataaatgaaaaattataacttattagtttactattttactatactagagtcttttctagttaacaaatggttgatttaatgattgattttacgggaaatgcttgtgggatttactatgagaagattcagtttttgaatttccttttgaaggacctattgaaataaatgagtggacaaattatcacttaagatatgcaataatatttgtttctacatccatgaatttaatcaaactccttttggaaagcatcaaatctgcttctctgcttggaaaaggccataatgatcccctgagcccaagtgtatattccccactcctagggggtaggacgtcatgggacttctgttcatcttgtatgtactacattggcgcatgaactacctatgcactcaaaggaagttcctagttggttttgttcctctcgatgtcctctattggggaatcaaaacctaaatggatcattataaggtgcaacaatgaattgccaatggtgccttaatggaaaatgttagaacaagagtacctcatgtgaactcttaacgaaacaaataccatggcatgtttgttagcaatctgtattcgaaaatatattcaaaaattaaaaaaatgctataaaagcataaatgcagaagaacaaaattataccgagcccgttgaattcacagtgtttccttaaggaactaaatccccacctagtactcgaggtttaggattatttcctcccaggatagaacggattaccctcactggtgtaacggtacttcaaactccagtgacaacgaacgcaaagaacagtagtaaatcacacttactgctttcgtttctattaaaacaatgcagaaagaaggagaaatcagaatttttgtaaggaaaatctaagggagagttcatggtatttatagccaaaaaagttgggttatactgaagagttacaactcttcatgtaaggttgcaacttgcaactcaaaatggctgttttataaaacggccatttatgcaaaccgccaaattcaaattataatgggtaaattaaaacggaggaaaatagaattaccgttatagaaacagtctaatttggattaattaatattgcgttaatattaataaataaatttggtccaataagttaatcaatcaaataatttgatcaaatccaaatccaaagccgagctgagcgacgacgacgacgacgttagacttgccttcttctcaactccttaagtgctagaagaagtgcaattgtatatatacacatcaaaactcttttcctcctccaatatgagacaatatccctttgtcaaagagggaaattcaaaaaatttcattttcccaccatttctcattcactctattttaagctctaagaagcttaaacccaacaatgttggacttgggaggatcatttgtgattggcgtcttatttctgtatcaatacggtaagagttgatttttggagaatcaaaaatgcctttctcatgaatataatagttgtgatctccattgacttgtttaatgtccatatcttccatattctatcaatagttaattttctccatttacggttgtgctttgaaattgaaagatggaacatcgttgtatgcaggaggctgttgctgttaccactgccattcaagaggagatattttccgagatgggaattggtgagctttaaattactcgtgctttacttcaaggacagttcaacttaggcaatttttgagtattaaagagcaagtgctcttttttctgctgtatatcatacgtatgttagtctgtacatatgttcgagcattcgtgcaaagactaagcactttttactctaatctagttgaaccgtgttgcttggtcattatatagtggtccttgtttttcaaaaaagagatgccaaacaccagttttgcagatagagctgggcagaattgcagatctgttacaatgtgattttgcagatgttaatatatacttgtgatgccgatggtatttctgctatttcataaaatggtatatttagcttggaagaagaactatattgcccactttctacaggatgataatcttgtcattttgcttgtttcttagtttaatttgataagaagttagattgagtttctgctctctttttgttattttcattggttcaaaaactaatactaaagtttaccattggtgatatgcggtgtcatctaataaatggggaaaaagcatacctccaatcgttattcatgcctcatatagttatatcataataatactataaccaacgggagatctttatttctctgccgcagatccaagatttggtcttgcttgcttggggaaaataaacatgacctatgagagtgatcgagacctgatgattcgtttttatgaatttgttgcaaagtaagatcagcctattccatctgcgtattgttaagatgaacttccattttgtgttatttcccctttataccttttgaagaaaatttcttggcacagtgttcaataggatattgaggccattcttgctaacaaatccaatacagatgtaatgaatgttgagttctctgaagaaaataacttggattttgttccttctattttactggccatgaagagaagaaatggcttgtgaagaggccgagcttggtcctgacgggtttgctgaaagattaaagatgcaacaaaatttacaagagcaggtaagagttctcttctcttgatgcttaattatgctatagatgtggtaataatagtggaaataaacattttgacagcaactggagatgctgaagtacatgcgcaactttcacatggatgatcagtctgcaatacttgagaaggtaatgatgaaagactacatattaagacgatctccccatgaatgaagatgctggtttcataaatagaacattaaaaagcatgaaatctctgcttgaataagtctagataacatttttagaagttgaagagtacccgaagtaatggggtttgtcgaccacgagcttttgttgtcagagtttacaactccataaaagtgatgaaacttacattagatttcccaatcaattagcaacttattttgcatgttgtaccaatgcttttctaaattgactggcatgtttgacagattcaccagcagttggagaaggccaactttgacactgaagcttctgtgctgacagtggagcagatgcaggatgttgttcgacggagagtatcacctttgttccagcccagataa

SEQ ID NO: 47 - DNA, 1688 bp, synthetic construct
  source 1..1688: mol_type = other DNA
atggcgttaacgaactttatactaacggtggtcggagtgagcgcggcgatattattgctgaggagcgatgtcaaacagtctgcttccatcttcaagcgtaacgttcgtcaaattcgccattggctcgaggaagagtcttcctccgccgccaagtatgcccttccacttcccttttcccttcaatattttttgcttatcgaaagtcctgtgctgtaattctcatcgattgataaaaacgagacctttatacaatggaatcgcaaagaatcagtttacattcccgtaactaactttgattcccagtaaacaaatttccttccaaattgaatcaaccgctaactaactgctttaactacctaatgctgacgtggcatcttcgagcttcttctccagctgctctatcttttattactccttgatccttctcttgcaaataccactgcagctagactgtgctggaattgggctcaacttagctacattgagctgattcagtaactcctctagagatagagccagtgtttgaagtgtatgagttctgaatttgccacggaacccatagctcgtcttagtttcttggtccgcaattaaatatttttacatatttgatggatttctaagtacaaatatagtgtttaagcaaaagtgattgggttcagccgaacccgcacccaatgggctagcttcacctctgcctcctgcatcattatccctcccttgctggcccattggcatcatcacactatgcaatatactggtagcttgaaaacagttattacgaaactaatttgcaaatttgtgtttttggagacgtacaaataaagtatccatgtaatgaacggactgaagttctgtcaatgaagcacgtaaataatgccaaaagtctataaccaccgcttattcgatgtatgtaacacactactcctacatgttatgtctcttaatctgtatcgttctttccaactaagaaaaaagtgcatttttttcttttgttcctgggcaaaataataacttttgggttttttaataggttatctagatgccagtggtattgaatgttcttggtgatttcaaaagcaacagtttgcttctgaagtttgttttgtttctcgcttggtaaagtctaaagagtaatataaaaattagagttctaggcaagtcaagtcaagtaaacttcaggtggtttttcttttgggcagccccttatagtttttgttatcgagatatcttctctttttacattgttcccgaggatgttttggggaatctgctcctgtgctgcaaagactggcagtctggcattacagtttcagttgaggctcatgcttttatagtcaattttttaatatgtaatttatgatgaataatatttgaagtttatcatttaattgtaggtttaggatctttagctttggctttatcaaaataaagatagctagctttgtttcctttcccttctcgttccttagttttctctgaacaagggacatccatccacgtcagattttaaattggtttctcattgtagcttcttctgttagctttttgattttgaaatcctgtagctgtagtgtatatatcgttgtcatgatgttatgaaattccattgatgtttctatagggaattggagaaggcaaaaccaaaggagatacctcacaaagacattcctaaggaggataagcattag

SEQ ID NO: 48 - DNA, 585 bp, synthetic construct
  source 1..585: mol_type = other DNA
ctacaacgtccggacgaaatctggggagtccttcctgggaccaaccaatgttcgatacaagtaaatcttctcgaccttttgcctgtcatcagagcaagtgtctatctgatcgttctcgctgatgatctccacattgagcctcggcatcttctgagcaacagccttgcatgctccaagagtcacttggcaggacgacatccaaagggatcgcattgtctcatacttccccacgtccgccagaagtgccacattaccaaaagggctatccctgatctcaagctttttcattttcttgcacccgttcaacacatatagcatccccttgtcactatctccagcaaaggctatggagagcatctcaagctgctcagcatacattcctatgtaaaggaaaacctgatcagttagaagaccagagactgacagccgcttgaggcctttgcacgactgtacaattgccccaaaaccttcgtctagtggctgcatggtaactgcatctggcacagttgggttaagagtgcacaatctgaaacgaataaagtttgggcagtttttggcaacagttataagtgcagcattagtcat

SEQ ID NO: 49 - DNA, 1758 bp, synthetic construct
  source 1..1758: mol_type = other DNA
tcaaacaattgtaggaaaataaagcaacaaggaaataattaaaaggggggcaaaaatatcaacccataggcgctctagttttccgcagaaacgaatcagctttataagttcagcactgtagattccaggtgcatagctcaagttcagggacgacaaattcaggcaaattggataaatagcaggcaggcagcgaggagcaatctccaagaaccccgacaagctcctaatagaagtacacctcttcagagcattctttagtttgttgtaagtctcagaaactggatcactgataaaactccctgtccccaagtccactaagtgaggagctttaattaaaatcctttgaagagcatcaagaggcacagtatgatttaatctcagacttcttaggtttggacatctagccactagcctctcaagagctgccaagttaacttctcctttgaggcaagcaaaattcaaagagacaagagtagtgcagttgtctgaaaaacaactaagccactgtcctctgcgatcttcgacttcgttttcctgcaagtccaactcccttaaaaacctgcaagaatcagacgaccaatttcagttaattatctgccctatgtatcattcaaatgcatcatagatttgcctttacacaagctcaaaccatccttgcagctcctcacagatgttcttcaccgagacatcacgggtatgaaaatttgtagagaacctgacatatcttacttcatacgcagagtgagctataaagaagcttgcatctttaagagacagatgttcttcaccgagacatcacgggtatgaaaatttgtagagaacctgacatatcttacttcatacgcagagtgagctataaagaagcttgcatctttaagagacagtttgattgcgaaagaacatgacatttcaacatgaactaccatgagccaccaaaaagagaaagatttattctattagaacttattaaagtagaccgacgaacagaacttgtgtaaactgtagactgagtttaacacccaatactaaacaaacgtgcagaactaaactaacattgcaaaacagagaaatatcccaacagcacaattttgtatattgcttggttaagaatgaatacccagagaagccacaaggatgaagaactgcatcaatgtaggaaagcactactactacattaatgcaactcacgaaaagcaaacacacataaccaacgattagacaagaaataataatgcatatatattatattaggctgaaagattcgctgcagcaaaggaatatatacctgcaattggaagcaatagcagcaagaccatcagtggagaaaccttcacagctaaccaaaatcaaagacttgaaattaggaaaggatcgcgagagtagttcaaggctctcatccgaaaccaccatcctcttcaaacgaagctcctccaagttcacaccactcgcagccatcgcctcaatccaagggtcaacataacctccccaatcatgagggaccaaattgaaatccgcaaaatggggtttccctttcaaggtaagcgacctaagcctcggaaacctagcaatcactctctcggggcttaccgcgtaacaattccctacaaacaccttctctctactgaacctctcaactctataccaagaattacagaccaaagagaccgcatttcggtccctgtgggaagtcaagaaattgaagacatgctccaacacttcctctgggaagtaattcatcatacactgacacaaacacat

SEQ ID NO: 50 - DNA, 4689 bp, synthetic construct
  source 1..4689: mol_type = other DNA
tcataattcactctttatccatacctccctaaggagttgatacctcacagcatttccatcaacattacctttcaatatcctactggattttgcaagtggtgaagtttcaatgccctcagaaatgatgaagttcatatccatttgatcgagctcatcatcgcttgtgtatgacttcttgagcactgaagatccgatccgtctcagcttagaatggctactaatgtcacctaatagactatccactgaagctgttgaaggtggcttatatgctataggagctgctgtgcaggggtagctcatgactgaatcttcttccgcgtcaaaaggatcctaatgagaaaaatatcataagcagaatctgatttacaaagacttggttctagcatgacaaaatgaacaaagtcaaagagatgagcttacctctgtgttaagagcttcaagtacacattcaggtattggatcagggcaaaactcatccgcgacaaaaacattaagaacccttcttattataattggaccaaatgtgggacagacctgtagagagcacaaataaggggaaagaaaaatacaagcactcgggctgcaaaaatcatgatgctttcaaggttttgaggacaaacctcttttcttattgtcctacttaacagcatatcctttggaagcatcataagatcactcagtgcgtcgaggagatagaaagatttggaagatgtatcatattcttcactcccatcatcatcattatctgctctattttcatcgtgtagtgagttcccaacaaggtcagtgagccatctagaccaattccctatctgcaatcaatgtgatttaaaaatcatcatcatatatatatatatatatatatatatatatatatatatatatatatatatatatatatatataatcaacggataaatactatcttagagtttgcagaatgtgacttaaaagaaaattaatatggtgtaaaaattggtaaaagaatgagggaagaaaaataaaggagagcgaagggtcggatttggaaacctggaaataaggaagtctaaatattagtatggcacatctgctatgaacaaaaaaatgtttgttttcttggggtaataggggagagaagtgacaaatggcaaaataacccgtcaaggtaagcagcagactaacaatcatatctacattttagaacactaattgaaaacgttcaaccatgatccttaccgcatttttcagttgtgcacctgccccaaagctagcttttccagctggaatgggaagcacctcggcatcactgatgggatctgatataggatctgaaggcatctcatcagcagactctcgaaggatagcattgaacatagctacatccaatctagccacacattgttccattatctggtttcaagaacatttatcagcagcaaaatggaccaaaatctatatcatagtttgaactgcattaaacccatccaaagaaagacaggaaatgctggttctaacataagcaaatctacttttcttcagtacaaggaagttcgtgtttggtagtcttttttaacatatgacactaattagtatatgaactaacaactacttaattagtagtcaaattttcttgagaagtgaaacaaagacagataataacatgttatgattacgtaaataggagcatgtacaatatggaaggaagggtgggccagactgtaccaatttagagagcaaacgcaaacagccacattcgtgtccgccagctcgaacagggcaaatcctatcacaggcatctttcaaggcttttttccaaagatccaaggaaaaatttacatgctcttcagcacctgaactagacgtcttactgcaaacattgtttatctcggaacccatgctattacagattgcggttgcagcaccagactgcatatgtggaatgagagtctgaagaaataaataaatgaataaaacagagttgagttagctgataggttactgattcttcagattcaaaagcaataaatgacatctttaagattcattttgtgcacctgccaccaaatagattcaatgatgcgggagaatatccaagcctctgctttttcaagtgctcttataaatgtaacagggtccttccaattgcctaaactatcacaaatatcatcttctacaccgttgctagagtgagattcccacttaagtggggaaaacttcttcttgtccgtgacaacagaatttccaagcatagtttcagtagaaagaggtaactgctgctgcccatggaatttggttatagttgctctcagcaccactgaatttgatagccagaaagttaacctgtggtcagaaaaaataagtgaatatctagaagaaaaacaaataactgtcttgtgcatgtagcttaacttccttccgcaaaaccttaaactgaaaagtacctagcaacatcatgtccacatgcttttgcaatcaaatatattccagaaatagcacttttagcagcacttccccttttcaatacggaggtttctttacatgcatgtagatagaacctggacaggcgtcttgccggagcatggaccttattcatggaacatccatgctcagcaacaactgagtaaaggccaacttcaatagcagcagcttctctcaattctccttcaagcatctttactctttgctccaacttatggattttgctttccaaaagaatgtttgttgtgtcccttggactcgctttttgatcttttctctcattgcttgaaaaacctcgagcacttttcagagtgtcttgtactaaaagctgattacttccgacagatccgtcacccctatttgattctggtgatgacctgactgacattacatgctttaatatctcattatcgggggtactactctttgctccggaactgtccctgtaagcagaaattgcatgagtttccttcaaatcatgttttacagtatcctcttctgaatagcaactgattgactcatcgtttttgcattgcccttcatcttccataggttcttttccttgttcattttgctgataaccctcactgttttcaccatcaacagaaccatttttggtaaccgcagctcttgcatcatcacacagcgaatttacaggtggagtggaagtgttctcttcaatttcactatcggagaaattactaactgtgctcacaaagttattgttatcatctgtgatatgcggaataggacaggctttttcagctggatctgagttctgccgactggattgttcattaacactttcgccattgaccggtatttgttccatacattgtacttgcttgtcctgtttgattatcatgcaggcattctgtgtctcctcagttatatcactcacatttgatgtcgaatactgaatgctactacaaccaccaattttctttgaaatccaagccagatcagaggacaaatcagttgaggagtgcgacaaacttccattcaaactaggtagagattttgtctctggcgtctcgtccatatttgcaagccttttcttataatcaaggacattttcgtcttcatctcgtcgagggctactttttactccctcggatctatcctgcaaccatatgctagctggctatcaaactagctgagatattcataacattggaaaaacaatataatacaatcaccaaatgttacaaagatgcaaacttgtgcagagtgcatttgatttaaaatgcatgccgtggcagtggacattgcatcttatataactataaagaagtaaacatacaaagaagttaaggggattcgacatctactacatatatgaacccccttccgccccctggctccgcccatgcatagtatgtagtgaatgcaaaattgctgcaaaaggacttatgtcatgcattttggaagtaacataaaggaagaataccattcagatataaatgaatgcacaaaagagggatattgatagaaaagtaagcactatacctcttcttgagggggtgaaccatagtttgattcgttagttgaagaagaaacaggcactgataaatgcgaggaaacatcatcatcatcatcatcagtgtatgaagcaaactcagcttcgtcagcatattcttcactcagtaatctagaaagagaaccatttcggtccattgaagcttctctttttaacttgtccctcaaggatgacctcacacgactcctctcacctatctgaattttcaggaaaagaagaggctgtgctgtgttcctataagttcgcttgcagttaatagggggacatatgctcaaactctctctaacaacaccataatctgccaagtcaatgatcgcagttcctaaaagttgacctttcacggtcttatcacgccggggttcatacaagtggaattctacgcaattcttttggaacgcgtttccgtctccacctttaagcgaagtttctcttagtagtgtcacgggaagtctaaatgactcattgaattcaattctcccgtcaccaataccagaaccagttcccgacgatggaacaacttgattagtagatccagagtgcttatcttcgtgttcccattcgattagaacagcacggagcgttctcaatgaatgcgacggtggccaaggtttaatctccttaatatggattagatattcgacttgaactgaggggctattcctagttctgcccttcaatcccaaaaccat

SEQ ID NO: 51 - DNA, 2819 bp, synthetic construct
  source 1..2819: mol_type = other DNA
ctatagaacaagtgtgtcaccatgtctggggagccttttgcagaaatgcaactccccgtgaaggactgatttgcggcttgagctgtgtcgtgggctacatgtcaaagttccgctacatgtcaaagttgcaccttgtgcgataaagttaagcgattttagcaatctagaaagctgcaggaaatgagtgtttggattgagatgcaattagcaatcagcatttaagaagtgtcaaaaatctggacacaggcaacttacactcaaattcgacagagaaatagagaatgtggtggggaaaagggaaaattttgtttcagctacagcagatgtagtagaactagtgacagaaagaacgagagggagatgaagtattacctgcatgagaaaatagatttcagcaatgtcttttggagtcagttgagacggattcaagatatgatataacaagatcagctgcattgcatccagaagataatgctttgccaactgacaatccccccctgtggttacctagcattacaggagacatgtttgttagagatatagtagatatgccctagatccaatatcatatttgatgatttgaacatatttttgtgaacattatttgatataaaaatatatggcatttgatattcttttatcatagttatttttaattgtttgattaacttgataaggtccttgattaaattttgagacttgtcatcgtgatggagattatgataatgagagtaaagtttcttataatttaatctaaatttgttcttgatcgtaggattattaatttggacattagtaatccggttagatcaatatttatgtgatcgtctttatggataaagtagttgatctcattaactaaatcacatagatagatgatgcatatagagatatgatcattgaaccgactcattggataattcctaatggttagaattaccataaactgtcaataggatattttcttgaagaatgtgatgtaaaaatttcctttgacctgagatcgtcacagtaattgacaagttatttattgtgctttgatactagacacctatggccctagggcgatagttgaaaaggatattgggtatgattaaatacttgtagaattagtgattgatcaagatggaatctgtcaacccttggtaatgagtttaagctccatgttgtgatgaattataaatgaccaaactaagaccttggtcagggcaattgaatgaaagaaaaaatgagtttcttaggtcattcaatagtcgattatatttgacatgaacacatagttggtcgcctattaggatttgacagttgaaccatatcctagggtgatccagagctataaggacagaaggaattactacattattcttctactggttcttgagagtaaattgtatacttcatgctatccggttgttagggagtgttgctagacgccacccttgattagtatattgatgtgatcaatttactaccgatttagtattgaacctgtggggtcgcatactaacgagtattctgatctttgctaaaggattaattaacttattatttgataattaaattaaggaatttaattagtcaaatagatttagttattagtccaaatgaaatattattatactctttgctagcacagagattataattaatattgtgaacgaattgaagttttctatttggaatagaataaattatatctcttggttaaaatatatatgtgatatatataattaatatttatttgcacaaagtgtgcatatagtatattaataaaaatttatttggaaatccaatttagaattggaatggataacaaagtccaaaagtggacgtgagattagctcaaacccattaagaatgggattgatattttttaatataaaatattagttaattttcacaatccaatccgatttggaattgaattggaacgtccttaaaggacaagccgacgcacgtttccgattaggaatggtatatacgtttctccaatggaaaattccataagtttatttttggaataaatttttaccctaagtaaatcattacctcaaccaaataggaaaagggtttataggtgatgctacataaagggtgatggaaaaaatttgccatataattttcttgaaaagaaaaaccactttgtgaaagtgaaattgcaatacaaagccaagagagaaaatacagttacaagaaaagtgtttcttgttcttctaatttgagttgagatttttgagaaaaatttcagcacaagtttttcgttcaatttgttcgcgggtttcattgatcaaagagttgatagcaaggatcagtttcggtgtggatagacatagagtcttcgcactatcgaagaaattttgaaacgagactctcttcaccaggtacgtcttagatccgatctttgacatgtaaataaattttaaacacgaaaagatctacctaggattgttattatcttccgttgcgtgttaggaacacctatacgcaatccttcaatgttaaagtagtgcggcaataacttttttttaaactggggactaagatcagtggaaagtctgcttcagctttcttgccaagtaaatcaatatgaaggcggcatatgctaggctaaagaattctatgaacatacttagcttttactaagttataggttacatgatacatcacctctagcagagagtacagtgtgaaccttcctcgtcaaaaagaagctgcatcaacggggaaaaaatggaagatgctatttcaaagaaagaaaatagaagttttacctgcatagaataatccaggaagatttttctccattttgtcaattgcat

SEQ ID NO: 52 - DNA, 6230 bp, synthetic construct
  source 1..6230: mol_type = other DNA
ctaggaattcacgtacttcacatatgttggctctccctcagcacccaacagctgcttaaggtcagaagttactatctctttcagctcagtcctgtgaatacaggaatatagcaattttacatgccgaaacatcatcattaacgtgtagtaggaataaagtagagcagattacaacaaaattgaaaaagtttagaaaccttgaggcttttgcaagttctctatttcggcttccaccaacaaaagtagtatagagataaacattgtttggtgcccgatctggaaacatcatagaagagaagagggtgcctgcatgcagttcagaagtaatgagggacaacagtttccagttgcctatataatttttaatctaatacgtacgcattacctagtgtcttgagaccatgttgttgctccttggaaggtacaagaaccccaaaaccctcaaggggatactttacgttttccctcttaaatgtggttataacaacagatagcggtacataatcaacctgcacatcatatggtcagctagaccgcaacagtgccaatgaacataacagttcatacatatatacacagtacataacatatagcttcagatttagtccttcatataatctcttctcaagacacaacgaaggaaaataggagtccaagtagatagtgcttgtgcacctttttcctcttggaaatgagaccgacttaccttgcatgttcacttttcaaagcattaactggggaaataagtgcgttaccatgtggaaacacattttgatttgtcgaaaagaggttaatttcacaaaacaagaatgtctatactatacgttaacaccaaaaagccacacaagcacaaacctcaggaataaagttgagtagaaatggatttcctctcttagcaatcttcatactcttaacatcacagagtggggcctaatttacagaagcttagtcaataacggcaacatttgacatctgcactggactcaggtatttgtgagtccgtcatcacaagaattgagcaatcacatgtcatgagactgagcttgagtgaagctagttgtacaaaagcaaatacaaggactcaatcattccagtttctcaccgtcataattacagcatcaaatgattcttcttctgattgccttttgtgtggagaggcagaaataattgaccagctatctatcgcagagtcctcagtacagctacaagataattccagaactctagagtttagtctaagttcatcttctctgagatctttgcatattgcatcagtaagtgtctgcaaagcatttggatgagataacaataacatgaaagatgactaacagaaatcataggatcaaggttctaaaagacatcagtgatgacagtagttgactgctcattttatctagctctcacccaaagaattatagattatcattaagaataatttttttatgaataacaacgcccattgaaaaagactaagagaattgggtcgaagtagtcaagtttggtatatcatcccttatacctacctcttatgcttaaatgtaagaaatcacgcaaacaggggcaagaaatgcaactcagaactagtgtctgaagttcatgttgtaattttagacgagtcatctctggcgccaaatggatgggcggcgctcagatgatacatgatgcataaaaaatggggttatgatcgtttaaattttctgccctttaccttcaatgcatgaggaaaaagagaactaacgtatacttacttgcattccgcccaaaaaggaaaaagatccccgctggcgcttcttatttgctgaagttttgggtggcccttgcttcttttcatttttaggggataacttagatcgaatagctccaagtatgactgagccaaacctagcaagtattgacaaagtgccataagagggctatttgcatgataaaatgaatttctcaacaaactgcattctcaatgaacacaacgattttatattagtcctctgctggtgggaggtaggaggtatcttagtcgaggtgctaaaagttggctcggacaccacaattataagaaaaaaaattaagaattttacagtagcacccatgcaactgtaggacaagaaagcaaggagaatgattgccagcccatgtgatggcggaaatttggaagtacataccttttctctaaattccacaactctggaaatgaatggtgcatctgtatcagaaggaagagatgaaattgagtcatagcaataatgatgtcagataaaaagtcatgatcttaatatatggctcatgaaacaaactagctagataccaagtttaaattgtcctcccccattttactttttatacttgagtaactatacaaaggtatattgacctcattatcttatcctctgatgtttgttcaaagaaatataggatggacaaacaagtgatataaatgaatactcactgaaagcgagtcaggatcaccaccacacattccagcaacaaaagggtcaattagatagtcaacaacctagcaggtaacaaataaacaaatggatgttcatgtatagctgatcttaaattaaagttagcaagattttcttttgtgatggactgtcagaagattcgaggaacatagagataaagtcaatcaaaggtgaggaaagacctcctttccaaaatgacgctggaagaatccactgacactgcatataatacgaaaaagcacaaccaggtcaatttaacttgcctatatgatttatttggagattgagaaggcatactggacaaagaaaatgtacttatcactacgtgcaagagcatgcgaatctgatgcagctaataagaaaatcaccatcagcatgtagattatgaaacagataacatgtccatgcagaattgctgctataacaacggggagaggctagatttcaagaggactgaataatagaaagcgcatgcacaatactagctcaggtagaatatcttgtgcatttagagagaaaaaaattcctttgaattccaacacatcatctgatcataaaagctgtaaactcagcagagtgaaatatataagcagtaggctgaagaccattgaaatgtatatgaggccagaatggggtggtaacatgtcccaaatttgacagcaattgacttctgggtgctgcaatatggacaatcttatacattgaaactaacacaaaaattggacagaggaaaccagtttcagtctgcaaactaaaaccgaaagtgtggaattgacagatgctctacaatttgagaactcaaattgaaattcacatgcaaatttaattttttgattatcgagaatagcaagtatctactacacaattattaaaagctttggacctccaagatttcccaacaaaagaactattaaacatgaaataaagtagcatcagaaaagcatggaatttcatggagaggttgaaccgcgtcatatacctttcatgtgagtcagacacctgggagagctttttattcttccataatattggttccagaagcatctgaagctgcagttatgaagatattttataagacaatatgcttgaaaaataaccatcattgcttacaaaagaatagaaaaaagaaatgagatataaagatgaaaaacgaatgtacagatggttatacatttttatcaaggtcttcagtgcattattcactgttaataattactttacgtccgacgtgatgacacacattgaattcaagctgttatgggctgcgggtgttcctctactcagcaaaaagctatatactattttgaactccaaaagaaaacttccaaggttgaatccgaacttcagaaaccaatatattcggaatatgatactctatccagaagtagtgtgtctaaatcacttagaccaaccacattagttcactgatatttaggtctacatgtcaagaaatcacctaactggataaaccatttttcgagacgaaacatttcatgatatttcctagattaatcaaaagccccaactgtatttacctcaacaaactgttgattgatagaaatgatcacctaaaggtacataggctaaattgccaaagttacaattttaatgaagccaaagttacaattattaaacttcttaataattttgcaggcagtttacgatcttttattttgaaataactagaaactctaaaagaaaaaatagtacgacaacaaagaatttaaactgcattttacaggcagttgaagggatctatacagaagttttattgcttaatacaagaaataaattttggcgcacgactctagctgagctatggtaaagatcaactcatggctcaaacagggttaacaagacttgaggatatgcaattgcaactcacatcgagccacatatgatgtttcacagatacttctcaaaggagcaatttgaaagacaaacatgaacatggcaaaatctgtatctaaatcagaaaagttggatgaccaacatacctttgatccagtggaaagaaaattgcttttgatcagatcaattggatttgaaggtaactgaaattaaaaatcaagcttaggacatgattaatcataaaactccaaaagaaccatatcagaagaatggaagtgacactgagcagtgcaggaacatgcagttaagcttgccatggaccaatcttaccagtacaggagtaccatttctggcaatgtagcgcttgttttgtgaaagtggctgcacagagaaagattggagaagcagttcaggcattcaaatttagcccatatagaacctgagcttgaagtctaaattcgtcaaagctttcgatgttctatagcttatccaataggacaaaaaatgaaccaaagacttcttttatgaggtaatgacacaaaaaagcagaaaaccttacaaattgttgcttttctcggagtccaagagaatcaatcaaaaatgtaacatcaccttcactttcagtctgcagaatgtcaaaagccacttctttactatcatcagaacagaatatctaagttggaaacaaagaatccgaaagtccactgacattcacagaatttaaatggaaaagtaggaattaacagaccatagtatttgccccttcatcccatatcaggccatcttggctcacgctacgtaacttccctccagctttcccttctgcttcaaatactgtcacattcaagccatggattttcaacttgtatgctgcagcaagcccacttgaatacgaaataagttagcggcatcttgaaagaataacttgaaaagtaaaggatcaaaaaggaaaacaaaaattagaaaatgataacacattatattgcaacaccattttaacctcaagaaatagaactcaacaataacatttaaaaaaactaatgacctcattctgtcataatgaaaggcctcgatttactctaccatggtttttagaaaagaaatgtttactttgaagtgaaacatgttacctctaaaattaccgacttaagattggtgtttcagctaagttatatccattgaccatatacaaaatttgttcagtgtctttaaccgattgtaacagggttgggttactaaactcatgcgtcgtctcttaatttatctgaatgttaatttacagcatcaatctataaaaactaaaccttttataatttactaccaaaagaggttcaacagcaatatgatctgtcaacatcgcttcaattaattatcaaatatgaggcatctaattatatcgtaaaactaaataactttgaagtttgaatgaacataaacttgcattcttagctatattatccatgagcaatgacaaaagattagtaaaactcggtaaaataaactttttctttaattatcagtaaaactaggtaaatttaactcatgagatggaacaattgcaggaactagaacagcagaacatgcaagacaagagtaatttacggaatataacaattgcaaaagctacagatgcaaaatttgaaagagaatagaccaattgcaaaagcaacagaaatgaaatctgaaagagaataaaacaattgcaaaagctacagaagcaaatttgaaacagaattgtaaaatcaacaaacctgacgcctgcaccaatgactgcgactctcttcgcagaacctacagaaaaagaatattttttccttcaattttgcttggagtataaattacaaatgaaattatatggtatgagaagggaaagagagatttactgtgtttatcttctccggcagaaggagccat

SEQ ID NO: 53 - DNA, 3139 bp, synthetic construct
  source 1..3139: mol_type = other DNA
ttacaacttagattctggtatatggatccttcttaattcttttctcaaaatcttcccagatggattcttcggtatggagctaataaatgctacacgccttattttcttatatggagcaacctgcaacaaaaatttaacgtcacattagcaagttgtggacaatcacactaaacacttcggaattggttcaaggatcaaaggtcatgggcctttatcccatgtgtttcactttcatatgtgagatatatgatatttcaagtgaattgaacttggcgattcacagcataatatgggatttttgagcgaaaatggtagtctacttaaaatcaaatagttaaagaaagcacctgttttgaaataaaatcaatcacttgttctttgtctggagagctttgggtacgtctcacaacaaatgccatgggcacttgaccagcttcttcatcagggtatctgtcaattttgtgtataaaattcatcagcattccacttctaacattttctcaacattcaagttataatcttttcaaatggcgggccttttttttatttcatagctttagtttacttgcatcagatgattgcgagtaaatacaaggcaagttacaagaaaacaaaaaagcaaaatcttacggtatcacagcagcatctactatatctgggtgagacagaagaagttgttcaagttcagcaggagcaacctgaacaatcaagcatgaataagcaggcatttccattgtaaaatgcctgagaagctaaatgttactacaagggaagctagcaacggaacctggtatcctttatatttgattagctccttcagcctatctacaacaaaaaggtaaccatgatggtcaatataacaaagatcaccagtcctcaaccatccacccggcaccagagtttcagaagttgtcttcggatcacccacataacctacaaaaaagtatcacaatatcatttgatcgcttagcagatagctaacaaggaatgctgtctcaatttcaaactagtctgagtagtcctgtaattgaaggtaccttgcataatggttggacctttaatccagagctctccttgttcaccgggaggcagagcaatgccagtttctggatctacaatttttgcttcagtatttgctattagccttcccactgaatccccacgaagcatttcttctgtagtcgcagctcgaaatgctgccccgcttgtctcagttagtccataaccctcaaatcaagactcaaagttcagttacatgttaaaaagcaattccttagcttatcgagaatttggtcttacaccaatcagttaaatatcaaataagccccagtgctacactaatcaaggtgtgacaatatgaattatagtataacattgcgctgtcgatgtcatttcatttcaataaacaatactaacaactgcgccttcttaatcccaagcaggcttggttcggctatacaaatctttatggattatgtcacttaatttagactcgtctcatatcaatattatacaaataaaaataaagtactaggagttctttatattttcagtactatatttctgataggaccaaaggctcctagaagacgtatgacctatcgtacacgtacaaacatatctaacaaacaatcctaagtaattggtatcacctatatagattttttctttccgtcttcctattcctcactaggtttgcaggaatttcaagagttttcgggataatttactttcatatgatttttggtccacatagtctccctttttagctccctacattgactatgatttcacagccattgacggatgtatttggaggccaacactagatatgattaagtcatctcgagcaacctctcattttatccttaatgtggccaaatgtggtcattttttacatgatgccacatctcattccaatactgaaaacattgtctttgaagcaaagtaaggttttctaagaatttcacccatattcttacacggacatgaaccagggcagagtcaagggtgcccctttcatagtaaaattacattatacacgtaatgtcaaaattttatgtatacatagcgatattgaatcccttagtaaaagtcttggctccgccactagatatgaaggtactttacttacttgaaataatattgtatttggaaacttgtatgaaaatgcttggataacatctttgccaagcggagcacctccactaccaaccgctaccaacgaactcaaatcatagccactggtaacacttcctttagccattgccacaacaaccggcggagccaccaccaattgcgtcaccttaaactcctccaccgcgttcaacattttcctcaaatcaaatctctccatcacaactacagtctcactcaacgctaccgatttcaacgcatagtgtaaaccgaacacatgaaacaacggtaccgtatacaacatcaccggcaccggcgacgtccgctcttgtctctgagcataaaaattagcaataattgcaataaaattccgatgactcaacttcactccttttacctcccctgtagtccccgacgaatacataatcgccgccaaatccgattgattcacttccacaggctcgaatttcaactccgtattcgtcatcatcgattcaaactccggtaagtcaataagaactgttccgtgtttcaacttaggaagcttctggaagttcctcgacgtagcaaaagcgattacaggattcgttaacttaatttgtttaaacagctcggattccgtactcataggattagcgggagagactataacgccgagggagagtagtgcaaagtagagaaccggaacgtgagttgaattcggtgaaaggataaatgcgacatcgtttttggagaagcggaatgtgttttgaagggaggaagctagggaaatgacacgtgtacgaaattcggagaaggttaagcggcggccggtggcggagtcgatgaaggcggcggcggtggaatcggggcattgttggagagagagtgcgtaagaagcagcggaaagaggagaagcttctggaggaagtaagagaggtggccggagactgtggaaagttttggttattgaacagaatccgctgtttgggtcgtcggaggaggatgaatttgtagtatccat

SEQ ID NO: 54 - DNA, 1388 bp, synthetic construct
  source 1..1388: mol_type = other DNA
atggatgttgagaatagtgggccctcctatggatgccacgttggaccacaagataggatgataatgaaccggataatgttaagattccggccgattgcaccgaaaccggtcgccgacggttcaggtccgggttctaaaccggagaatgaaaaggcagaaccggttagcagacgaagagggaagagaaagtacgttagagttaagaaaaataggaagtgtaagaatagtgcagagaaagaagaaagaaaagatggatctgataataatggatctattgtaacccttcagttacttccggagagtagcagcagtgttacaaacccgacagataacgtttcttttcaaaaagaaatctctttctctgagctagatcaaccccaatggatgagttttcatgaatatgataataatggtatatccgccttcagttcaacggatcggttagatctgaaggtagtgttaaattgtgtgatcgtctcgtctaaaagtttaattatatgttacagatatcacactttattgcttaattatataatttatgtctcgacacacctccttacatgttctgattttctttcgtgggtcaaacatatatattttcttgttgtttgggttttgttgagattcgaacctatatatgacctatgattgcactaatatatattagcctaaggatgattttcactaagaggattcaaaatatataaaaaagtaaatacaaaaaagcaagagaattcattatctaatatatatatatacacacattaaacataattctgaccttgtataacattgtaatttccggcgaaggggtacagatgaacctctttgcgccccaagctccccctctgctgatatatatcatgttgaattttttggtttatttaatcaaaaagtttaagttgttagcgaaacacatttttattcacttaattaattataaaattatgtctcaacaggtgacaacgcagacaccaagaatggtagagtcttgggtgatggtggatagaataacaaacatatttgtggatagtgaaggcttaggaagtacggacatggagaaaatgaagaatctggagacggacacgtatccagggctcgtgtcggatagctcagatagggtgcggtgggtgaatccggcgtaccggagaatggtgaatcctctagaagacggaggagtgacggcggagatggtggtgaagatggttgtgaaggagaaaataagagaggaagaattacctgcagcttttgcatgcattgtgaggttagtgtatacatggaagaataagaaaaactcacgaacaatgccttgtgatgtgtggaaaatggagtttggagggtttgcatggaggtttgattctaatgctgcacttagcttgggtcgttaa

SEQ ID NO: 55 - DNA, 3339 bp, synthetic construct
  source 1..3339: mol_type = other DNA
tcaagccgtggatagaacagaccgctcataaagttcatatggctgcatcccacttgaagaggccttgacaggttgtcctgaggcaccaaaacacaaatattggttcaccgaccttttatgtcctgcactttgtttgtgagtcgagtctgatgatacaccgtctacctgattagaacaagaaggtccagcgagctcagtctcaatcacgattgctttttgtttgtccaaagaactgcgtgaagacaatgatgaaatcgagaatgaactagcacgaggtgatctacgagggaggctgagatgtggtatggcttgcatgacaccgtttactatcccagaagcaaacttcttctttgctgttggacttggaatgttactactagcagagagcgtccgtttgtggcttgagaatgatgaaggcttggagtatctttctcgaagagggactggtgccacgtccgaattacttgcagaacattgatcaaccaatatcttgaccttcttagtatctctttttttaatttttggccaatggaagaagcgtttcagtttttcagctgcatgatctgcggaaccagatttcttgtcatttctagaacaatagctatcaggactcatgtatcgatgactcggctcacttgtgttgctaatctcagcttttttactgtcctctggtgcatgttcaatgcccgtgtataagaatatttcggtgtcagagattctaaaagaagttccaggactgctacttatgttttgcatcttcaactgggatgcaacaactctccttgcagaataatcgcgatgactaaaaattcccccagcagagatcaactgtcttgtaagtagctcggaggaagcagaacgtggtcgttgccttaggatatcgagtggtgtcattccatccttatcacgagtattgacattaatagagcggacggtcataagtaattccacaagttcagaatgaatatttccaatgacagctaaatgaagagcagttctaccgtcattgttttcagcgttaacaatttcctcaatgttgaaaaattttccacaaactaattgtttcatgagatcaatttgacgatccaatctgcggaaataagggttttggaaaccagtaatggcaacatgaagaaatgtatctccagcattgtttcttgaattgaccaatgaaggtgaagcaacgatgagagcttcaacaacagctaattggcccctcgaagcagccacgtgtaatgcagtattgccttgattgtctacagagttgataatatcagaacttttcagaagatatttaacaacctgcacccataaaatagagcaaatgaaggacatattgatagccaaactgttagcattagcatacttcacatctttagcaacatctaagcttgttatgaggtattgttcaaacatgacaaacaacaaggatatttaaccaccacatcgaagatctataatttactactattatataaaaatacctgaaaatccaactaaaacttctctgttataataatgggatgcaaaccatctgcatagtctttcctatgtactctgactatgtgcaagaatcaagccaagtgaaaatattcaactggtcaagtaaaggtgaaaatttatttgaaagtgctggcgtaatatgcatcactatttagcattggcttaaaccgccttataactataagttaataaaagcaattgttcatctaagatgccaactgagatggacaacactaaggacatctgagactcagacatcctattaggagaacgttctacagtaaaaatattcccgaacagtaaagggacttccaagaaatcaagcaaagcaagatgattatggttttcacttcatactaatacgaatgattatgtcaaaacctcatagttgacacttctgaagtttaataacttaatatggtgtctggaataagaccctcatcctctctgaggtttaaagtggctagctaaaatagaaaagttgctgagatttgttgcaattagaccaccaaatattacttaagtagttgatcttgctatacaaatagtaaacacgtacaatttgttagtcccaaggcccgtgtattcagtggcaaatccaggattacaatacaaagggattccaaaaaaaatcctagaatgtcatgcctagggtcatcttatatcaaggggattcaacagtttatatacaaccaaaaaaattcgtttttgccctatctacccaatacaagttttcagagaagggcattcaattatttagttccaaggtcccttgctaacacatagctccgccactgcctgtattgatttgtaacaaccgagaacgaaaacccaacaacatgaaatctgcaaaagctcaaactggtaagcatacaaatcctacctcgacctggcctttgccagctgccgtatgtaagagagttgcaccttgaatgtctctgtaagccaatatatcatcagaacagtcagcaataagctccttcagcatcttcacattgcctcctctagcagccgcatgaatagctctattcatcatttcccacttataagcagacggaatgtccccattctgttcctccgaccctctaccaccacgcgctataaacctcggtgatacagcaaaatcaaacaaaaccttaaacacatcacaacttttactcctagcagcagcatataatatgtcagtaacaccatactccccttctccaaataccaaaagggagtctttttcgagcaattgtcgaacaaaatccaagtctccagcagaggcagcagtatacaaaagccatccaccatatccagctttaaggagggaatttttccctttcttgttctcacattcaagaagcaacttctttgcaacttgtgaccggcatctagctacatcatcaaactgttcttcatcatcccaaacggactcaagccttcttatcctacgtaatgaagtgagcttgataacgtgattgccgtctaggcgaagaagctctcgtaccaaatcatagtgaccatttgcagctgcccaatcaattggtgcagcataccaccattggtccccagtactctcccaccgaagaggtaaatacgttggaggcatggcaatggcaagaatatcaagacccgaaaaagacttcagctacaaacacaaagaaaaaatctttctttggttaaaactcaagaagatgacacacctaggtcaagcaaaggcaggatggagcttatataataagggttcaactggattcactattttcaacacgaatgtagaggtgtagtaaataatcattaaaatttcgataaatattaaaccacaattacaagtaattaagccctaaccat

SEQ ID NO: 56 - DNA, 3407 bp, synthetic construct
  source 1..3407: mol_type = other DNA
ttatacatcttctttgttttcatgatcggaagatttaagcaatggtttcttgcctatcacatcaacatctcgagaagttgcagtcttccaagcaggatagagcacataatgttgcaaaatgaagagaatgtcaaagaatattgatatctgcaacattcatccaccaaaccaaatagtcagagcctcatgatactatcaatagaactgctaacattaggaaaaaagaggaacttaattaggaagaaattattttttattggttttatgtgaaggtatcccagtatccctagctcaaaccaagaaggccttgcaccatttccagctctaatttgctacttcctccgccccattgactcggcacgaagtttaagaaaggaaggaagacatttaaaatttgtggtctaaaacaaaccatagtatttgtgtggttgtaaatcaatttattaagggtaaagaagaaattatccccacttgtggtattacactgggtatgttgttgttgtttgtaaagaaggaattttaaaaataaattgtttttaaataaggaaaggtagcattctttttgggactgcctaaaaaggaaagagtgccacataaattgggacggagggagtatattttataccattggaagtgtaacccctcattatctcattaattgatctataaagagaacaaagtataataaagtttttaaacagcgaaatcaggtataatagctatagacatgatgatcacagcaaatatatagatggataaaaagtaacattccgtaccagtgataataaagtcttgcctatgtttccataaaagttcacccaggaatctgtggaaaaggagtttcaaacagtatattaagcacataatataacttctcttatactttactagaaatttaactagttctgaagaagtccatatatttgaaagacagaaactcactttgatctatggattgcactgccatctgtccataattcgtcaaacctccaagcaagtccagcaaaatgttgccaatactaaatccaatagtactcttccgctgaaagttcataatagcctatagatttgaaagaggcaagaagttaatccagactgtagaatgaatcttacactaacaagcagaatcaatgaaatacaggactatacacgtatcaaacaattctagtatttaaagtaaagagaaccaaaaagagagcccaattgatcggaaggtgcaagaattttgaaaacaaacaagctgaacgtatgcagttattacctgaggaatgtatttaatcactgtcataacaacctgcaacgtgctgcaatcaagagtcaaacataaaacattagcacttggaacatgcacattaccagtgaatctaaataactcatacaccatgtatataaggtataaaaacttgccttttagaaagaaaatgacaaagaagatatcaagctcataaggtacttcagaagtacactcatcacccccaccccaccccacccgtttttggtctagtggtaagagctcatcacgtgatgtgtcggttaggcggacgtcacagattcgaaccctgcgcagacagaagccaggtatttaagtggaagggtagagggacggccattatccattgagtttcaaaccgtgtaccactagcctcggggatttctcggttatccaaaggaaaaaggtactatggaagtggccagcgaaggctgacgtatgatcatctattactatacaaatctcttaccctttaagtaaacactcaaaaatcggtgccttatttgttagtccttattaatcttggaagaagagatatagatagcacgggcagcgctagccttctaacttcaatcaaaataactgatgagattttcaaggggaggaactatgtttcaatcaggaagggttcttacttgaagcaagagaccagccaaagccaagaatggctggggagagctacgaacacacaaactgcaacagttaaccaagcaacagacacgatcgctatagaagtctttgcaaccttctggtttccacgctgtaaataaagcaatcatcggcaaagtgagtaaaaggcatttatcagggtctcagtccttcacctcatttccagttccctatccatagaaaatttgaacttaaaaaagcaaataggaatggtccacttacatcataaatagcaatctgaaacagggtaaatgctgtcaatagaacagcatggactgagaaagcaacatcattggcagccacaggtatcatctgcagttaaacaatcaaggcaataagcaaggaagtcatcgaactacatgaaagccttcttcgcataatatcaatcaagttaaaaagagatcaaaacactaaaagttcttaagaaaatgagacaagacaaaagtactactactactttgtgaccagttagcaaggaaaccccagtggattgctttccaaaccaaaaattttgctttcaccagccttcttgtactttttctttatggtgtctcaccagcttgtgtgcctcactagcccaccgagcagcttgctataactcataaggacagatgccagataatccttcccaccaaagatgggcttaagctaatgtcattgtagctaagatgtaggcttaagctaatgtcatagtagctaagatgtagattttcctttcttttatgatgggtggtgtccgggccagctcgcacacacttcgagtgttcaactgtatacctgctatctcccaccagcacatgtaccgtgaactctgccaaccaacgcttaggcaaatggaaagaatcacctagtgttttctatgcctcgacctaggatttgaacataaaacctcatggttctaagtagtagctacgatttgaacctgagatcaataggttattactcctatgcttaaatcactcaaatttccgttcagtagtttgttttaaacctcgatcctacttaagtgccaatttaaaattagtaatctatcttaattttttgtccccatatatataattcgaacagaaagcagcggaattaagctagaaaaccacaaaaagcaaaaaagtcaaacctcattatagccatatttctgacgatactgcatctgaacagcagaactaaagaacatggaagcattataaatgagatacgatgagtgcttggtcaaattcaataccacgaaatcgaaattcaatcccaccacactgcgtttcaatacacaaaacaaaaagggtttaaattaaaatccattatggtatgaactttgatgtaaaaaaaatagtaggaaaataaaaagggaggaccttttgcgtcggaaattcaagatgacttgagggtaaaaactaatggaccaagagaaaaaagcaatccatcctaaagtattgtacagaacttccagaggaattgaattccacgccgccat

SEQ ID NO: 57 - DNA, 8579 bp, synthetic construct
  source 1..8579: mol_type = other DNA
atggaggatagtgtgagatcaggagggggtgtattgaagaaaaaaagctcttctggttgtttaattattaaaaagaaagatgacaaaatggtgaagaagaggccaaggtttgttatgaatgatcacgtgtcgaatgatgagttatcggagtcaattcggaggaaaaatggtcaattttttagtaatggttatggaaaaagtggtgttagagaaggagaatttggtaggaatgtgaatttgcttgatcttaatgagtacgaagaatgtgatgtgaaaaggcaattgaatgggtatagttatgagatatcagagtcaattcggagaaaaactggtcaaaattttcgtaatggttctgttgtttatggaaaaagttgtgttagagatggagaatttggtaggaatgcggctttgatagatttaaacgagtacgaagaacgtgatgtgaaaaggcaattggatgggtgtggtgatgagatatcagagtcaattcggagaaaaaatggtcaaaaattccgtaatggttctgttgtttatggaaaaagttgtgttagagatggagaatttgttaggaatgcggatttgcttgatttaaatgagtacgaagaatgtgatgtgaaaaggcaattgaatgggtacagtgatgtcagatcgaacatgattgaaagaagagggagttgtagagagttcgggagtgggtctgggagtgtgatggctgagaagaggaagctttcatgtatggatagttcaagtagtttttcaggtagtaggatgaagggcgatgacaatggctttacgagaaggtatgatttgttagagggtgaagtgcgtatgcccatttcattgaccagagaagccactcatgaagcaatcagattgcaagggaaaaatggggtattaaaggttatgttgaataagaagaagaagattgacttaatgcacaaggattttgatcctgcggaaatagaaaatagaaaaggttctcagtcggcagatgttgtcaagaggaatttactagtccatccgtccttttactcaggttctaaacacccggaaaagcaacctttgtctgttaaaacagagaaaaatgagctgaaactggaaaaacctttgttgggtaaaatcatccgttcagtagcctctgaaaaagatgagactgatacgtcacttaagttggcaccgcctagttcagtgcctgctagctcagcaatgggggtgctaaaggaagaaagcaggtccttagcatctgaagatgttattcctgctaaaagcaaagacggaaaagtgaaacgaggtgggagcactgagaagcagcagttgcgagagcgaataaggggaatgctccttgaagctggatggaccattgattatagaccaagaaagaatagagactatctggatgcagtgtacactaatcctagtggaacatcctattggtcaataattaaggcctatgaggcatttcaaaaacgatctgaggtagattcaggtaaaagtaaacctgatggcagctcttgttcatatgctccaatatcagatgatttaattaataaacttaccaggcaaacacggaaaaagattgagaaagaaatgaaaaagaagagaaaagatgatggcaagagagaagatcgtaaaaagacttctgtgagggagtcttcgttgggcacagacactcttcagcatgaggagaggctcggtagctatataaagaagaaagataagttgctgcaaggcaaattacatgcaatagaccgggagaatggggataattcaagtgacaatttgaatgtaaggagacttaaacaggatacagttgcaaaatcatctggtggagttgcttctaattcaatacatggaagaaaaagtaaattaattgggcgatgtacattgctggtccgtcattctgacaagaggggaagttcagatagtgatggctatgttccatgtactggaaaaagaaccttattgtcctggttgattgactccgggacattgaagttgggacagaaggttcaatatgtgaaccgtagaaggactagagtaaaactagaaggttgggtcacacaagatggtgtccactgtggttgctgtagtaaaatccttccagtttcaaaatttgagctccatgctggaagtaaacggcatcaaccatttcaaaacatagttctagagttcggaggtaccttactagagtgcttaattgacacgtggaatcggcaaaaagaatctgatcaccgggatttctacagtataggcattgatggtgatgatcccgaagatgatgcttgtggtatctgtggtgatggtggggaccttatctgttgtgatggttgtccatcaactttccatcagaactgtctgggcataaaagtatgtatcaatcagaatattctctctttatcttactgctattttatatctatcactgttactccatactgtcatagttttcctttgttgcatgatggatagtgcaaccttaaaagtgccaatctgctccaaagaaatgtagagaagcaaataacggatttattgaagccttgcatcatgcccttctatatttctttggagcagatttgcacttttctggttgcactatccatcttagcaatgtattgtcttcattgtccatgtatagttaaccattaaatttgatcctgcctcttccaccccgcatggactccttaaggaacgcaatagaaactggaaatgagccaggttatctttaccttatccaaaaaaaaaaaagtaaaagaaaggagccaagatacaaagggcaatcaaggataaaactaactctaggcaattcttctgaccaatgaatcatgatcttcagtgtaagctctgtcagctgatcatcctctgaccaaagcttttcagttaaactgatcaatgaactgtttcacagattaatcctatcccattccctccatctaaaaaactgtttaaaattattcatttgtgtcaaccacagtatggaaagacaaaaatgcattgcaattaccatgagaatgaacagtacttttaggatgttaattacttgtgaaagaaattattaggatgttagtttcttagggaagttccatctccatccagacttattgacgaacccaaaacgtggagttttttatttgaacatacatgcttttggtaatttacttatatcatgtacgtgaatctgatgatggatgagctatttttgctaaatctttattatcatgcttgccaaattgatcctataaaggaagtttctgatttttaagcagctgggcatttacatgcttcctcttggtgcagatgcttcctccaggacattggcattgtccaaattgcacttgcaaattttgtggggctgcttgtgaatttcctgaggaggctagtgagagaactgtcaatgaaattctttcttgcagcctctgtgagaaaaaatgtaaagtgtacttttctggaagtttttgtcacattaactcttctacattggagctggattcttagatttccctttgcagatcacaaatcatgcagtttggagacggatgctttatctgctatatccaatgatcaatctgcatcattttgtgggcagaaatgccgggaggtatttctcttatcctggttgtgaattcttttccttgtgtggtgtataacatattatatcatgaagtataggtatcaaatagtggaaactatttcagaatcacaattagtaactccagttttaaattatgttctactttttattccaaggaggactcggcataagctcctacgcactgtttcatgatcattcataggtccaaaaccgttgaatggagatggtagaaagatattttcttgtacttgttagaggatttgctttgaaaggccaaatcttctctgctcctccttgaatgcctgctggagtgtgtagcgtgatgatctttggcatctttcagttcgccaatgattagtgcacgttaatgaattctagaaattgagagcttaggaagaaattcaagagaaaaagaaggaagtgaaggtcgtaaaatgttagaagattctttcttgtggagtctatagagttttaggtgattgacataatttgacttgttgctgcaagattaaaaatgtttaaaatggagctctttgcaattatcaattgaatataagcagtaggagtttaagggctcgtttggccataaaaaaagattccttttttccgaaaaaggtttcattttttttttcgaagttaagtgtttggccatgaatttcgattttcacttgaagatgaatttcataatttttcgaaatttgaaaaactccaaaaatctattttttttttaaattctcacttcagatcactcacaaaaattcaaaaacaaccaaaattatattcatgtccaaacacaactctaattttcaaatactattttcactcgaatttttttcccacttttttccgagattttacaattcttatgtccaaacgcccactatctccaccctcctgacatcttgtattttgatttcttgctggtttctaagacatcctgcaagtgcttgatctttgaacctttcgatttggtggacaatgtaatatcattttctcatagtgatctgagaagtatgatattcgttgactgttttgtgttccgcagatttctaatgctatctaacatctgatactccccctgtcttaaaatggttgtcttatttcgtaactcgagaaagaaaatgatgtctgtcttatggttataagtatattcatcaattcttatggttataagtatattcatcaatctaaaagtttcaacaagagttttttcatgtaatttctgaacatctgaatttattctcagaaaaacaaatatatctctttgatttttttccaaaaatgcgtcaattggctgttgaatcgaagtagtataatcaaattttgatagagggatttacagcttaagttaaattaaagtttttcactggatctgagagacattttctttagttatggtgaaagactgtattccatatatatgtttttttgtctgcctcccatgggatttgtgtttatgacttgatttatttgtcttctctgattatgcagctgtatgaccatttacagaatattcttggtgtcaaacatgaacttgaagcaggattctcatggtccctgattcaacgaacagatttggactcagatatttctcgttgcccctttcctcaacgggtggagtgcaattctaagctagctgttgcggtggctgttattgatgaatgtttcttgccctttgttgacaggagaagtgggataaatattattcataacgtactctacaacactgggtatgcagcttttagaattttcaagggttttgaatttggctgatctaaaatctatgggtactcattatttgtttcacctgggaagagctggctgctgatttgatttggccaatttggtttgggcatatgatcttcacgttttattttcagttattcttgcagtttctccattgggtccatgaaagcaacacatgtaactctgtatattcattgtgtatgtctttcaaaagttaaagcataactttttgttatataaatcagcttttgtagttttacgtcgatgtctccagataatgagtttcatctttacgagacagattactcctttttttgattgcgctcctaagtttgtctggctgactgttaggatgaagctgtacagagcattttgattgtgaagggaggcagtgcacttttaataatttatcatcttttcatctaggttgatgctcagaggaataaataagagagttatgtcattttagaattattttttctattcttttatttcttttttccttgttccttttatttctcctcaacttcatggacaacacaatgcattctatccatatgtcatcttgcccctgtatacaccctcgaaacacattaatcctcctctaataatctgtctttacaataaatcatctatcttttccctcctactatccatttcttctttccttcgttttgcatttggttttcattactgagtagttatttgaaaattttctctttcctctgaagctcaaatttaagtcggctgaatttccgtggcttctacactgctcttctggaaaggggtgacgagataatatcggttgcatctataaggtaggctggattactcaaatttgttcatatgttacagtgcaagtttgtttttcaattatgaagaagtcgagcactagacattattgtgtgttgttttgtaactgaatcatcttaggtttcttgaaaatgcaatggtgtgccttgcataaaggatttatgatctctaacaaaacattgttcttttgagtatgtgcacttaataaactgtgaacttatattcggcagaacatcctgttccagagttggactgaatctatatcaacttatttaggcgtatccagttctccaaatttactggctgtagggtttttcaattttgttattcctttttgctgttatatatttctgctagtggaaacagcctcttgcagaaatgcagggtaagactgcgtacaatagatccttgtggtccagcccttccccggacccgctcatagtgggagcttagtgtactgggctgcccattttatctagaagatgattgtatgttttcttacaagtaaaactcctagccatctaaggggcacaatttaaagaatttatatcggttattaactttttattctcgacttagcatttcatgtagaaatgtagaggaatttactaagccataaaaacatttcttaattttccttttccttatgcgtcatatgtgcttgtgctgtggttattcatgccttaccaatcagtaaatactgttttcaatggttgtctgttaggatccgtggaactcaacttgcggaaatgcctttcattgggacccggaatatatataggcggcaagggatgtgccgtcgactgttgtcagctattgagacagtatgagaattatactttctttgtgcttctaattttttcttctaagttgatgcatattatcagacccactgccgaagtatcccatgttcctttatatgcatgcagctgctattgtagcttttttggttgttcatacaaaagaacttataggagggtgaagtgtccaaattactttttagggctgagggtttcactcttcagaaattgcaaatgacgattgatgtgtctttactaaattggcacaaaatatattaaagcctgtgtttattgcgggacattgtgcatgcaagaaatatgtccaggaatgtgaaattagaggagtaaaatgctgtaggaagaaagctgtctcacttgtattatctttttaatatttgaaatgtatgagacaatcatttaagtatttatccaaagcacatcgtttagtttactgaaacagttgtgttgttaatttgagtaacagtaatattgttgtgacacaagaaacatgatatagctttggtcattgtcatcgtgttttcatttaccactatgagatttattattgtccgacctcttaaggcttaaatttttacgaatgctgcgaagcttatgtgaatgccatgcctgttctaattgtcctgtgcatctgtgcagaagtcatttagaatattccaacagcatgttcatcatctttatcatctatgtcttttatatgtcgtttattattagaccaatagtttttacatcctttatccaggttctctccactttaaaagttgagaagttaatcattcctgcgatatctgagcatccgcataactggaccaaggtttttggatttgaggaacttgaggaatcaaacaagcaagaaatgaaatccatcaatatgttggtatttcccgggacagacatgttacaaaagaagatgttgatggaagacatgcaagagggtaataggataattatatgtccctgcttcgtctttgtttttctttaacatttcgcttcttcattaaagaattttatcttttctatcaatatttgtaactttgacttgtacggcgttttctgcagcttgtgatttacaacatagtcatcctccgccacctgctttggttgaaaaagctgatcaggaatcatctatcaggcatgatcgacatctgcatgatggcgtatgtgtgagcatagtggaaaaaactgatgatgggtttggtcccatggattctggctctccagcttctgctgttcatttaagtgctagcactgtggttagagcacaaggtgattgttgcgagtctgatatccaaatctccagtacggaggcagagaagaattttactgaatcagccacaaatttaatgctctcttctccatctggcgcaagtaacggcagccctgatactgaagatgctgcactaggtcctgcaaatattgctgtcaactctggtatagagcctatcaatcaaaaagtttagaaagaaaatgcctaactaccttggtaacaatctggtcttgtttcattagctgaagtttcttccaggagcacatcggtagaaaacagattgaagaagcttctgatagttctgccactaataacgatcagaaagatatttgtgtggtttgcgtag

SEQ ID NO: 58 - DNA, 4835 bp, synthetic construct
  source 1..4835: mol_type = other DNA
ctaggttggtgatttaccaggaactcgctgtgcaatccaccctaaaccatcgtataaaccgtcaccagtcaaggcactgcaggcttgaatatgccaatcatggttcttgatgctatgaagggaaagcgcatcggttatctctgcaggggtcatggcatctttaaggtcctgtttgttcgcaaagactagtagaaccgcactttgcagatcctcgtgtgggagcaacctgaacaactcgtccttcataatggaaatccgggctctgtcagtactatcgataactacgataacagcatgagttccgcgatagtaagttgcccatgatgtcctcagtctttcttgcccacctagatcccagacctgaaaaaatagcagaaaatagaaagaaaggagtaagataaacagtacaccaaaataaacagaattgttctcatccatttccccagatataaaagaaggcaaaaaacagcatggaaattaacttgctgtctttcgtcaatacactcagccaatggagaaaacacaaggaaaaagtaggaggataatagtaacttttattatgagtacatatcagagcacaaataagcagataatgccaacgatggtttaaaacattcttgtgctagttgaaagggaaaagaaacacgatcaagggacgaactcggatggtatcacaaattttctcattccaccaccaagattcttgaggacaaggtcctacacctttagacatacctaacactttgctactcttttaatgcaacctgacatttcattccataggatgtttccgtccccttctatgtcccacactccctcctcccctagtttttcttgaaaatttttctcttcaaatgagtgtgttttcacaacgtcagctagatacttcaatttgttcataaaagaattccctgccttcaagaggatgaaagtgttttttttttttaaaactcaccctcagaacaattatttctcatcattgtccactgatagccaagatacaatcgatcataagaattcctagccaagtgcacaacaaaggaaaatactccttattgtctggtcggagtatagcgcacaattggagctgactagagcaagacttcaaattactcaatgcaacaaattactcaatgcaacaaacccatgtgcactagatataaacctatctcaagcgtcaggtagaagcaaaagcgtgtcatgcatcctaaaatcaatcagagatttctcgcttcaagcaaacaaatatcctctgttattctggttattcagaggttcaacagaagtattttcttggctctagcattactagttctgagcaaaaaaaaacagttttattcaatgatggggctgcaacacggtatatcatgttgaagtacaatggtactggtccaatatgatacctattttgtgaccttcaaaggttgccagagatgctcaagttaatagctgataaggttgagagatacacatgtacctagaacatcttcgcaactcctcctttgattatagttgtcaagtcaacataagatattcaattaggcaaaagaagtaatttggtcagaaaataagttagtaggttttgggctgttggggttttattttcacaattattaaaagtcaacataattatttcatagagagttacaaattccagtactccatgcccaaaagctacccttacctactcttagaaagagaactttttttttattacgtgcccttctttgacaataagttcctttaattgtttaagcaaaagagtgattccacaattagaggttatggatcaacaaatctaacccttttgactaaaagatacaagagtgacaacaagtacgatgggatgaacttccacaaagagttggggaaaggaaataaagcactgtgcacaaagctggaaaaaatgggagctataattggtaccacattaataatccaaactgcaattgagaatcttatcacgtaaaaccacactgtcgaggtgtcaattggtttaaggagaacatcattgtgaggaaaaagaataactaagatgattccacgcgttttcatcatgaatatgaaattgcacattagaagcctactccatctgtttatttcatatactgtgtcggacagcaaaatgattacatagcaagacaggggagcaggaggaccacaagtcacagaagattgaattacccaatctaagatttttaatacagacagttccttagactcatccaatcaccgaaataactgatgtcaaaggatggtgacaatattatactctatgcagattgacgcgggtcccttttcgattttaactgttgctccattacacgattacgtaaaggtataatatatagtgtggaacatgtttggaactgaatgggtgatgccacaagtcatcacacagatggccgctagaatggaagttgagaaggaggaggtagaacttagaaggagagcatgagaatttacgggatgaagagaaatagaggagtatttatggtgtgtttggtatgaccaaaatcattttttagaataatgttttccacatgaggaagtcatttttggtatttggaccatcacgagagggaaataactaccctcaattatgcagaatttcattctgcttacgaatatcttaactttaaactacttattacttgctccaagtagcacgtatctaccattattaatctttaatactcaaaaattcatcaaaacactctctgatttacaaatattttttatttttatttttattttcaccgggtgtccgagtctcttcgagccccgactaatctcgggggtgcacaagccctcggcaaggagtttcccgcaagtctgatggccctcggaaattgtttgcacccagtgggtttcgaacttgagaccttgaaagggagcctctctgatttactaatattattattaacgtgccttggtgctgatgatatatgtaccaattatatacatatatttgccaaaaaaatatttcctactcaccatccaagactagaaaaaacatttttctacggacaacattttccatggaaagctacttccggcatatcaaacactttaaaggaattgaaaacactcatgtacataaagatggaacttttcaattttttggagcactcacaattttccagtcagcatagattatcagactcttcgtaatcatgtatttcttgtggtgttccctattttcttatacatatcatatgtggctttatcccccatcgcatcaatgaatcctcttcagttaagattaacccttttgagtttgaccatataatctacttccgtcctcacccttcttctattcctgcttcttctccctcctccacctagattctactaaattttaacccacaactttctccatcatttcatcaatcagttagactttgacatgtaatacttttcctcacgatttcagccggaacctgcctcaaatttctaatctacacgtactaaattttgagttggttaacctctatattacctacatctttcctctcctccaccatttaatgaggttaagtaatatctcaatttataggggggataaaaggaagaataggtgcctcttgacttccattttctcataaccccccccccccccccccaaacccaaaaaaaatgcacaaggccacataaaaccaatgtgtaccaaaaccacttgttcatttaaacaatcaacaataaaggatagcctcctcaaaaatacaaacttcaattgttattctctgtataccgacttgtacagaatcatatcctcactcaaagtataatttcaaatataagaacaacatcaaccaatgttagacagatggaaaacatcaaaccaatcattaggtaccagatcaaattgcgcccaggctggacaccactgtcataaaaatgattccttatctttttaacatatgttaggattagaaccctagttttccatgggcttcaatccaactatattgaaccacgagatcacaccagcacatcaaaacaattatccaatgaaataatcaaggctcgtgcaagctaaccgggacaccacccttaaacaaaaataatgattctccacaaatactctcccttcccattttatgtgatagttttgattgcgcacgttgtttaagaaaaaagatagaattttgaaactttccatctaaaacacgtcataaacattagtatggccataaattatctagactaaaatgagaagtttaaagtaaattatttcaaattttttaagacatacacacacagagagagattagaacaatttcaaactataacttcacgcctaatcttaattcaaaaatattaaataatccaattcacggtataaatctatctcagttaagggtgaaataagaagaacatatagaaaggtatctcttttatggactgactaaagggcagcctggtgcactaagctccggcagtcggggaaggcccggaccacaagggtctattctggacagactaaaaaggaaaatatgtcatataaattaggatagagggattagaacaatttcatactaaactttcacgcctaagcttgatacaaaatggttcaaacaatccaactacaaccaagaaaaaatcaagtaaattaagtgtttcagatgtagaatttgtacctcaaatcgtatgttcttataaacaagctcctcaacattactaccaacagtaggatttgtagtaaccacctctcctaaatgcaacttgtaaagcgtcgtcgttttaccagcgttatccaatccaactacaacgatcttatactcttttgctggaaacatcatgaaccagaatctcgatatgaaacctcccat

SEQ ID NO: 59 - DNA, 8384 bp, synthetic construct
  source 1..8384: mol_type = other DNA
atgcagaagagacagagcggaggcggagcagccggtggctccgccactccggtagcaaagagaggccgtccggtcggcagtggaggcagcaacgctgccgctgcatctgctgcctctagtgctgccgccgactcggtagctcctccaacactcctcggcccttcccttcatgttccctatgccttcgctggttagttgcctttatcatatgtgtattttactgaaatattaatatgttccttaaattgatagctttagaatgattttactagtgtaattttgaagtaaattattactccctccgtcccaaattaacaagcacttaaactcaaaaaaattatccgaaataattgtcactctaggaattcaagactaaatttgttaatttttgtatagtagttctgtttaatactgctcaatacgcaatgtacatttaataggggtaacttagtaaatatatcttgtattttattgttttcttaatgagcgtgaaaataactaaaacgacaattaaaataggatggagggagtaaaatttttgagcttttgtactgcgagcagtggcggaggcaggaaatataaaggggttcaaaaaagaaaaaagttaaaaaattgaaagaaattgtgacgaatgagaattgaaccagtgacctcacaaaagttttgaacccccttgaccactaagctatgcttttgggttgtgtcaagcggattcaaaacataatatatagaggtaaaaaaacagattttgccttatatatacagtgttatttttcggcaaagggggttctagtgcaatgacggaggcaagatttccgctaaaggggttcaaaaaagaaaaaaagtcaaaaaaaattgtagctagtgggaattgaaccaataataaatggtaagtaacctcatagacgaagccaagatttcaactttatgggttctagagtttagaatgacgagttaataactaggttctaaacttaatatatggagaatttcttacaaatatacattgttgagcaaaagctactggataccgccgaacctgtagccgggcttctagctccgtccctgagtaacctgctatgataaatcttaaagcttttatacgcctctcgcctttagtaacacatttaataccaagtgttgtagatggaattcagagctggttcttcaggttgttactgctctaggagatcccttcgggaaaagaacttaactattctactccctccggtctattttagttgtcatggttatcttacacaccttttaaaaaactattaataagaagggcttttcgactactacacccttatttatatctttaatcaaatgttttttgtactttcaagaacaattatttaagggcaaaaatggagaaaatggagtaattaattatatcttgattttcttaaataacaaatattttggaccaattgtttttagcaaccacgacaaataaaatggaccaaaggaaatagtatcatcataatgccccttgtatctctagtgatgaaaaggagaggctgcatagagcttttatagaagaggagatcttatgcatcattaacagttgtgaaagcgacaaagctccaggaccggatggtttttctatggggttctacaaaaggtgttggccgattcttaaagatgatttgtcgaatgctctcaactatttccataaccatgaggtatttgagagaagtttgaactgttcttacattgctctcatccctaagaaaattggtgcaaaggaacttaaagattatagacctatcactttgataggcagtttctacaagattctctccaaagctttggcgaatagacttaaagtagtgattctgaagttaatcggctcttttcagatggcttttcttagtgacaggaacattactgatgctgctttgatcgctaatgaatgcttagattcgagggttaagttaggaaagtcggggatcatttgtaagcttgacatagagaaagctttcgaccatgttaattggaagtttttaccgggtatgctgagtcaaatgagttttagctagaaatggattaactggatgagatttgcatttcttcggtaaaattttctgttctaataaatggtactccacgggggttttttctcttcacaaagaggacttaggcaaggagatccgctttctccgtttctctttattctagtcatggaggggcttagcaacatgttacacaaagcttcacaagtaaactagatttcgggcttccacgcatacagtagaggcttggggaatattaaaatttctcacgttttatacattgatgacaccttaattttatgtgatgcggaggaacatcgggtcttattcttgaggatgattttgtctttgtttgaatcggtgtcgggcttgcacataaacttttcaaaaagttccatgtttccggtgaatactgtgccaaatatcagtcaacttgcaacattattgcaatgcaaccacggttcgtttcctacaaaatatctggggttacctttaggagtcaaatacaaattagtgtcaatttggaatgaggtccttgagaagatggaaaaaaggttggcgaattggaagagacaatatctctccctgggtggtagaataactttaatcaacagtgtgcttgacgcactacccacttacaccatgtctctatttccggctccttcttcagtgttgaaacgcatggacaaattaaggagagattttctttggaatgacaataaactatcaaagcctatttgccttgtaaattggaaaacaatcatcaaaaacaaaaggaagggggtctgggaattaaaagccttaagaatcataatgaaagtcttctgatgaagtggcattggagattcaacatggagaaaaatgcactttggacgagaactttgatggaaaagttttcattggacaattcatggcacaccaaagagtctagagtgcctcatagcaccggtttatggaaacatattagagggcattggaacaagtttgagtcaaacatgggttttattcttggcaatggaaggaaaatcaggttttgggaagatgcatggttgggtcatacgccactaaaagaccaattttcagatttttataatctatccttgcgtcacaatggggcaattgcagatttctgtatatcgcaaggctggaatttgagtcttacaaggaatattaatgattgggaaatggagagggtaggcctcttgctacaaatgctaagcaacactgttttgggtcacaacaaaatggactttatcagttggaagggtcaccccaaggacattttttcagttaaaagctgttacaatatgctggggttttgcaacagtgagttatggccttggaaaaaaatatggtacaacatggcacctttaaaggtttcttgcttctcttggattgtggctaaggagacatgcctcacagaaaacaatctccagaagaggggtttccagctgtgcggcagatgtcctttatgtgaagaacaatttgaagatgttacccatctttttttgcattgcaaggttacaagccagttatgaaacatgttcttttcaatatttggtatgtcttggaccatgcctaactccatttcagaaatgctgaactgctggacgagggagggtctatgcaagaaagctcaaaagacttggaacactgttcctcaagttatctggtggtccgtctggcttgaaagaaacaatagaatttttgaaggcaaaagaagaaatttacagtgccttaaacttaattgcattttgttgatcgctttttggtgcaaactggcttttgcaccagatgtagatgatattttagacgcaattgctgatttgctcaacctgtaattacgcctgttgcactggcttagtgcaaatatttatataatatattatttacccataaaaaaaagaaatagtattctaatagtaaaaatccaacaatttggtcaacccaaggtaagatgaaacatgattcatgtaaattgaaggcgtttcggaaaaattaattgacctgttgaatgtggggttggcgaattggaagagacaatatctctccctgggtggtaaaataactttaatcaacagtgtgcttgacgcactacccacttacaccatgtctctatttccggctccttcttcagtgttgaaacgcatggacaaattaaggagagattttctttggaatgacaataaactatcaaagcctatttgccttgtaaattggaaaacagtcatcaaagacaaaaggaaggggggtctgggaattaaaagccttaagaatcataatgaaagtcttctgatgaagtggcattggagattcaacatggagaaaaatgcactttggacgagaactttgatggaaaagttttcattggacaattcatggcacaccaaagagtctagagtgcctcatagcaccggtttatggaaacatattagagggcattggaacaagtttgagtcaaacatgggttttattcttggcaatggaaggaaaatcaggttttgggaagatgcatggttgggtcatacgccactaaaagaccaattttcagatttttataatctatccttgcgtcacaatggggcaattgcagatttctgtatatcgcaaggctggaatttgagtcttacaaggaatattaatgattgggaaatggagagggtaggcctcttgctacaaatgctaagcaacactgttttggatcacaacaaaatggactttatcagttggaagggtcaccccaaggacattttttcagttaaaagctgttacaatatgctggggttttgcaacagtgagttatggccttggaaaaaaatatggtacaacatggcacctttaaaggtttcttgcttctcttggattgtggctaaggaggcatgcctcacagaaaacaatctccagaagaggggtttcaagctgtgcggcagatgtcctttatgtgaagaacaatttgaagatgttacccatctttttttgcattgcaaggttacaagccagttatgaaacatgttcttttcaatatttggtatgtcttggaccatgcctaactccatttcagaaatgctgaactgctggacgagggagggtctatgcaagaaagctcaaaagacttggaacactgttcctcaagttatctggtggtccgtctggcttgaaagaaacaatagaatttttgaaggcaaaagaaaaaatttacagtgccttaaacttaattgcattttgttgatcgctttttggtgcaaactggcttttgcaccagatgtagatgatattttagacgcaattgttgatttactcaacctgtaattacgcctgttgcactggcttagtgcaaatatttatataatatattatttacccataaaaaaaaagaaatagtattctaacagtaacaatccaacaatttggtcaacccaaggtaagatgaaacatgattcatgtaaattgaaggcgtttcggaaaaattaattgacctgttgaatttgaggaaataatacaggcatttgtagaagatgatgtgggtaaactgttgcacttttcagctacttatgattctcatttggttaccaatcttttgtttttctttacagaacagaataataaaaggattgtcctggcacttcaaagtggattgaagagtgagctgacgtgggcattaaacactctcactttgctatcatttaaagagaaagatgaagtccggaaagatgcaacccctcttgcaaaaattcctgggctgcttgatgctctacttcaagttgtatgtttctgaatgtctgccgtgcaacctgcggttcataaataacttttcattagtttattcctatttaatgtcttagtgataggaatgatctgttcaacagatagatgactggcgtgatatagccttaccaagggtgcttgtgaaggcaccacgtgttcggcttttgggtgcaaattcagctgttactggatttggaaatgaatatgaggccttgaactcaaataatcccttaccccatcctaggtaattcagcatgcgccttcgccccctccatcttcttctggtttcctttggttgtaaatcttagttttccgatgcacgagatctgcattgcagaattcctagcctatatctaattgcctgcattactttgtccatttatccttgaccacagtgctggatctggatcttccaaagaggcatctgttcataagagtacttccaagactcgccctggagcctggagttttgaagaagatggcctatttaatttggatgaggaggggcgggctgagaagcagctgtgtgctgttgctgcttcaaacattatccgcaacttctctaacatgccagataatgaagtcatcatggcgcagcataggcattgcttggaaaccatatttcaatgtcttgaagattatgttacaggtggggaaatagatttagacttaatgttttaagttttttctgtgtttggttgtcatctatcatgtggcgttttgtattatgttctccttatctaaggtatttactatatcttctcaaactatcttcctggttataattgagaagaatgttgatcgttaatgatgtctgtaattgactttttagaccaataaatactgttgagaaagttcctctcattgaagagggaaagtctcctgtttattcctcggaaacctccctaataattcagcacattccaaagttctcatagtcaaattccctagctgtttagtcacccttttactgttgttgcttgttaacctattgaaagtttatgacttatgtggcttcactcttatcctttgtgatgaaaagcatcaatttgagattcatgtgattgcttggcataatcttaaagaaattgcttgaactctgttggagtgtcgaatacattgacttagtctggccatttagttgtttcttagtcttaaaataccgtcttgccctattctatattttttatagtgttggttatgaatttgcatggcatgcccttccaccagaactctttagtctttggttatcttaccatcttattcctagtgtgtctttcgtctttttgttatcttaccttgctgttgttactagttgtgttactgtttccttttgcatcgttccttgagccgagggtctattgaaaacagccttcctatcgtcacaaggtaggggtaaggtctgcgtcacactactctccctagaccccacttgtgggatgagatttcactgggttgttgttgtttcccttctgccaaaaaatggaaaaaataaataaacctttaaaatagaaaacaaacaaattatttggtcctatttcatgaagaagataggagaatgaatatgtgatttgttatgcctgaaatgtcagatgctcttgcttaaaatattttcttgactcatttccatttaaaacattttctgttctacctattctgattttggacttcttatttgacagaggatgaggaacttgtcacaaatgccctcgagactgtagtgaatttggcgctgttgctgaatcttcggatctttagctcctcaaaaccttcttttatcaaaatgacgtaagcatttggcttgtcagttgtttgctttggtattattaggtctaatacaagtagactcttttctgtaatggtattttctaaattgagttgtttgttcctgatccagggagaaacgtgcagtccaggctctcatgggaatgctgggatctgccgtgaaggcctggcacgctggcgctgctgaattaattggtcgtcttataataaatcccgacaatgaacctttcctacttccttttgcttcacaggttgatattccttttgacaactgaaatactatatgtccttctgtaccttatgaatgctcagtaaactcactaaacacaattcatgagacatgcagatatataaacgtttagtcgatataatgagcctaccacctactgatgctcaagcagctgctgtaggtgcattatataaccttacagaaataaatatggacagtcgtttgaagctcgccagtgaacgatggtgagagaaactttttgacctattaataggccatatattacggttactgtctatctgaatgacttactcaccgcatataacttcagggcaattgatcgactgttgaaagtgattaagacaccacatccaacaccagaagtttgcaggaaagcagcaactattttggaaagcctcgtgttggagccacagaacaagcccctcttactggtgtatgaaaatgcttttgcagagatgctctttggggatgcaagatactcggacatatttgcaaggatattatatgaactaacatcacggccaagcaacaaagtcgcatcagcttgtggaatctggggcatgtaa

SEQ ID NO: 60 - DNA, 3362 bp, synthetic construct
  source 1..3362: mol_type = other DNA
ttaggaagggtcgcacccaatgggtgtgatgtaaacaacctaccataatgcaaatattagtaaatgtttccatcgcccaaacacgtgacttataggttacatggacaatatacaaaatgcttccatatttgctccaaatactcagctttgaaggtgcgtctgcattgttttatataatcatatgtcgataatcgacaacaacaaagcaatcaagtctttaactttggatttgggatttgtgtatccatcatcatctttcaaaaatgtgtcgaccaatctgataaaatttaaagttcgttcgaggataaacattggtactgcagtagggcgcaagagttccttatttatatctttccacgcatttgtgatttttttccgcatctcaacatatgcttcttgctttgaaactccatattcttcatgtagcactcaacaaatgaagcaacatgttctctttgttgttcaacctacaacataatatatttcaatttcttcattaatattatacaatattttaccattttaatcagtcgtttctaaagttccataatttactttaagtaaatgtgatcattgaagttgtaatttatcaagcatgtctatttattatatcatgcaaaagtgccacctagactcgaaattagactgagaaatgctttttttaagtcaagggtctatcaggaacaatatttctacctccacaaggtaggataagatctgcgtacacatctctcttcctagacccgtactataagaatacactggatatgctatttttgttattgtagttgtttttgtattgtatttagggacaaaatttgcgaattacgatgactattgttgtataaaatgaaaattatattgttatacttacttcatgatcagccatatcatccattaatctacagattgttgatgcagctcgaagaatcaaaggttcatttgccaaccattcaaaagcctcttttgttataatttcctccataacaaccaaagaaattattccatacatcgtaccggtagaggttacaactccatttttcatatactcctcacattttggaatgtagttagcattcaaccattcagcttccttataataggctgccaccaactttttcatctaaaaaagtaacagaaaatgtactaatagttcaaaattgcgattcttccctacaacttggtttacttaattaactattaagtagtaattaatgtaacttaatcaaggattatctctttttttgcatagtagattcgatctactttaccttctttggccaatacttcttccatttcattgaaaacgtcgagaaggccttgataaataggtctcagatatgtcggtaacgaatccatggcaccttcgtcccatctgcaaagtctaagttgtcataatatatttctctataacaacacttcactatagcatcttaaaaaaatgtcagaacaaatgacgttgttatagaaacattttattatatataattttacctttggatcgcatccgtgaaaagcacaagctcatcaaaagttgcataagcatcataagtgtcatcaataatggagttcattttgactacttttgttatcatacttcttgaacgaccatattgaggttcaaatatattcccaccgtccaaaagtaagcttctactaatctgtcctttgcatatggaaatttgtttgcgcaatccaaatctttccaccagctatagatatcataacagtattaacatacttaaactttaggtgttattaattagcgcgagttacagtcaaacatatctataatagtctcgtttctttcaaattttttgataacataaaagattgattataaagaaaacttgactgttatagtgaactgttgttataaaggatgtattttattgagaggtctgactgtaccataattatgaagatgataagaatagtagatgtaccttgttagctcgttaagctcttttcgatgaagcttttgtaacatgttgaagtcaatttcaatcaaagcaaatcattatgtgttccaatgttttcatatatgtatatgtatttccttgctcccacccttggtatagttttgcgaataggatggcttaaggcttcggtaacttgtaccttaagcgcgttactcaaattcgggatcacggactcgagatgagttggggtaaatataagagcttcttctagaatttcctcgtcgtgcactctcgtatgtgctgcttcatacaaactcagtaatccttgaacatcattagtatgattttccttgaattttctgtcatggttagtgaattgcttgaacacatctggttgaacaccatgtttatattttgaaagaaatgaatttcattagcatcaagaaattaaaccctaagttattacaacaacaaaaaaaaaattcagtgaaatcctacaagtggagtctgcagagtatagtgtgtacgcagatcttacctttaccttgtaaaggttgagagcctgttatcaatagaccctagactcaaggaaaacataatcgcaacagtgttaaaagaaatatagtcgtggagaagccatggtaaaagaagtaaattgtaccccaagttattattgacaataacaacagcaacccagtagttttcgacaagtggagtcttaaataatattcctataataactatttgatttcacagctagttaagaaatttacaatttcaacattatagttttttacgatatttaagtataaggacaaaataaaaaagtatagtgcataattcttctatatagaagcagatctcgaaacttgctattctctgcacaaataaggtgcgataactaaaaaaaagaggaaggcgagtctttgagcaacggtaaagttgttactgtgtgacctacaatttatgtgttcgagccataaaatcagttactaatgctagcattagtgtaagttacctacattacaccctttgggtgcggctcgtcctgaccctgtgtatacacgagatatttagtattagggtaagttgcatactttacaccccttgggggtggcccgtcctgaccctgcgtatacacaacatattttatgcgccaaactgccttttaacaacaaaaaaaatgaagttgtgcgggaaaggacagaaatgtagggtacctgaagacatgtaatttccttgttgcctcgcaagtcgaaaacgaagagcagccacataaaggttgtgttcatcgttatcttcactattttgagacaaataaaagatgttttgaatggagttttcaatctcataattgaaatgatatgctaatcccaatctttgaattgtgtcaatcaagactagtttttgtgtactattatctagagtttctaccaacat

SEQ ID NO: 61 - DNA, 3155 bp, synthetic construct
  source 1..3155: mol_type = other DNA
aagcttccagaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtggatcctgcaggctagcgtgcactctagactcgacgaactgacgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactatgataaattatcgcgcgcggtgtcatctatgttactagatcgggaattcctcgagcaactatttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcagctcataaaaggtaccaaacaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttttgagcgggactctggcgatcgccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactagt

SEQ ID NO: 62 - DNA, 1243 bp, synthetic construct
  source 1..1243: mol_type = other DNA
attagcatcaaccgggttagcaattggggcattattgcgatcagcgggaggcaccttgttgctgagcaccaaattattctttttgccgtgatggccagactcaacgtcaacgttcaagtgagcagaccaaaagtttaacttttttgggatggcttgaaattaagactttaaataataagcataaaatgaggtgttatggagattcggatcaaaccaccactattatccctagccccacggttgatgccaaactgtttatttcaaaatcggataacaattacatttgtatgcggttttaagaatatgtgatttaattcaacacgaacaattaagaatatcaaataatcaaattagagataaaataatcatccaaacgaattgcgatattatggctaaccatgagcttagactgaccgatgagctcgctcttggttgaccttcgagcccagccgtgaatgaagtacataacaaatgagcaaaaactttaacaatagctaaaaggcagaaagtaaacttgtattgctttgatttacatattacaacatgtgttacaaaagaaaaacttccaccctttatatagtggagagtttcatccccagtataaatctaaaaaaggtaaaaatcttcctttgctggtaattactaattcatgatcgacatcgagtgagatttgcaccgtaatatccggttgattgcgatatcacgatcctctatctgtcatgtgtaaccgtttatcatgcctcccgagatcttagaattcattcttggaccggggtgcatgcccaataacaggcacattgttcgcccttcatgaatctcaatatggagggtctttaacctcgattataattttgtgtatatgtactcttcctctattttctcgtcgaaaaatcggagtaaacattatccccgattttaccatacacatacgttttgcctaaataaaatgacaaaagagaaagggtgacgaaaactgtcactatgcctaactaaccctcgtagataccgtcttccgcgaaccctacacacccctaaggccctaaccccttcttcaccttactttattaattacctcactcacttttgtcctcttatgtaaccgtttccagtttaattacaaaaaaattcagattactttgaagcatcacttctctccctttcatataaactcagtttttttgttcgagtctttatacatatgtctttatcataactaaagtagtagcttaaagtgaaggtgaac

SEQ ID NO: 63 - DNA, 436 bp, synthetic construct
  source 1..436: mol_type = other DNA
ttcatgaatctcaatatggagggtctttaacctcgattataattttgtgtatatgtactcttcctctattttctcgtcgaaaaatcggagtaaacattatccccgattttaccatacacatacgttttgcctaaataaaatgacaaaagagaaagggtgacgaaaactgtcactatgcctaactaaccctcgtagataccgtcttccgcgaaccctacacacccctaaggccctaaccccttcttcaccttactttattaattacctcactcacttttgtcctcttatgtaaccgtttccagtttaattacaaaaaaattcagattactttgaagcatcacttctctccctttcatataaactcagtttttttgttcgagtctttatacatatgtctttatcataactaaagtagtagcttaaagtgaaggtgaac

SEQ ID NO: 64 - DNA, 1500 bp, synthetic construct
  source 1..1500: mol_type = other DNA
aaaatatttgaaagaaaatattgtagctaaatgaatatttttccttcaagttattaaaagttgtggcaatataggttaaattagccacatgtttcttgctttaatagaattttgtagctaatcattaacttttaccacgagttgaaacttaatataacaacaataaccttttaaccataataaaccatttaaatcaaatattactaaataaataactttgcttcaagttctataaaatcatggcaatagtcattacgataaaatgatataaccacgaatatattgcaacgataaattctgtaactaatcattagtttttgcgacgaggtaaattttccgtcacagtagcaatcttctaggcacattaaaaatttgaaacaaaattttgtagtcaaataaatatttatcttcttattttaagaaaataaaaatagttagataatagttactactatttgtcatgaaaatatcaatagatacaaatttaaagtgactataaatttacgagtttactatactttagtcgtacagtttgcaataatagtattttaaccacaattagttatatgtacaaaatttcataagtgaataactttttttcaatgacaaaaataagagttgctcaaacaatatcaagttacaaaaatttaattttaactgtaaaagttatatttttccaaaataacataaactatagtaattatatatagtttgaagtattaataaaatttaaatatgcaaaagttaattttaataaaccatttgtatgcctacttgtagcctctaaactattttatttgctttatttatcaaactcatattttattttattgcaccttgttagttttggacgttaattatatatatttggtgtaaaatttaaaatatattaacatttgtggagaatttatgtatgcctggttcttaactattttttttttatataactggttagagtaatttcttatatttcagtatttatttttaaataagtcctcataaattgaagactttaaaagtttttgtgtcattcctctttttatttaagaaattgaagaattccgctaaatttcatattccgctgttatttaactgtttattcccttgttaatataattggtaagaagttttaaaataaaggagttaatgatttctaggttcatggcttgcctagcttctacgagtaagcgccatcacgactcccgaggataaggaaatccgggtcgtagcattcactcacaaaaattactaaaaacaaagtttacccttctcccaaaagtaaatttcatatttggctccacataatgtgttcaatgagtcaagtgaagtacttttcattgacaaaaaaaagttgctgaaaaatgcatatctcatattttttttttttagagaaatcccatttcttgcctaaacgaaatgcctataaaagagcatatatttgcaacaacagtttgcagaaactatcaagtcaaataatcccccctttaattccctcccaaa

SEQ ID NO: 65 - DNA, 1000 bp, synthetic construct
  source 1..1000: mol_type = other DNA
tttacgagtttactatactttagtcgtacagtttgcaataatagtattttaaccacaattagttatatgtacaaaatttcataagtgaataactttttttcaatgacaaaaataagagttgctcaaacaatatcaagttacaaaaatttaattttaactgtaaaagttatatttttccaaaataacataaactatagtaattatatatagtttgaagtattaataaaatttaaatatgcaaaagttaattttaataaaccatttgtatgcctacttgtagcctctaaactattttatttgctttatttatcaaactcatattttattttattgcaccttgttagttttggacgttaattatatatatttggtgtaaaatttaaaatatattaacatttgtggagaatttatgtatgcctggttcttaactattttttttttatataactggttagagtaatttcttatatttcagtatttatttttaaataagtcctcataaattgaagactttaaaagtttttgtgtcattcctctttttatttaagaaattgaagaattccgctaaatttcatattccgctgttatttaactgtttattcccttgttaatataattggtaagaagttttaaaataaaggagttaatgatttctaggttcatggcttgcctagcttctacgagtaagcgccatcacgactcccgaggataaggaaatccgggtcgtagcattcactcacaaaaattactaaaaacaaagtttacccttctcccaaaagtaaatttcatatttggctccacataatgtgttcaatgagtcaagtgaagtacttttcattgacaaaaaaaagttgctgaaaaatgcatatctcatattttttttttttagagaaatcccatttcttgcctaaacgaaatgcctataaaagagcatatatttgcaacaacagtttgcagaaactatcaagtcaaataatcccccctttaattccctcccaaa

SEQ ID NO: 66 - DNA, 1500 bp, synthetic construct
  source 1..1500: mol_type = other DNA
accagaatcaccatacaaagctatttccagactcggaatcccaaatagacatcaatagcactgaaatgcacttcaacccaatcgtatgaaattcttccaaaaaggctaacttccacaataagtgccaaaacgatctcgggtaattccaaacccgatctggacacacacccaagtccaaaataatcatacaaacctgttaaaacattcaaatcccgattccgaggtcaattactgaaaaatcctatcctagttaattcttccaactaaaagcttccaaaatgagaattctatttccaaataaactctgaacttcccggaattcaattttgactatgcgtacaagtcataatacctgaattgaagctgcagatagcctcaaatcgctaaacaacgtgctaggtctcaaaacgccctgtcgggtcgttacattataggtgattaactacgtatatacttgtgcaagtgttctatcataattcaggccaactagtaagagtagaaacaatgaatggcacataacaaacgatcaccacgaaatgtacatgatataactcacacaaggtaggcacgctactagacaattaccaataacaacaatgcctaggacatcacaagatatgacaaatcaatccttactatcacggttgagttgtaacgtgcaagaatatttcactctttttagggcactaagatcactccaccaacatttcaagagaatccctggcactgctaaaaagccctctacactgtagtgaatttttcttagttatctaaagttaattattcatttagtattctttacattaggttcccccttctaggtcctgcacgtaactagattgaatggattggtccactctattattatagagtaattattaaatttttatttgactaggcatcactagttgcactatcaacaaagtattagttctagccttctgggtacttcatacctatgcaaatgataattttattaaaacaatagatgtacatggatataaatacctatgaaaattaaataaattataactaagaaaaaaactttaaagttcactcctaagatatcgggttattacatgaccaaacacaatttgtttatcaaatactttcaaaagaatttgccaaacgtaaattatttttctccaaagtgacttatgaattactatgttgataaaatacttttcaaagtaactaatgtttagaagtcaaggatgggcttcttttgattattgaagtttgtagcaattgtatgtagttatagtcagggtgaccaccagcatctcatatagcaatacacaagtggattagcgcattttaaatttcaattagttcatgcaaatatacacgtaatagcattataagccactttcacaacaggcagattagtggttttgaaatttcaaccaatgatatatactataaattgatcaagcacaaaccttaattgagcaacacaatttcttacagcaataactatcacatataacaataactgcc

SEQ ID NO: 67 - DNA, 500 bp, synthetic construct
  source 1..500: mol_type = other DNA
ataaatacctatgaaaattaaataaattataactaagaaaaaaactttaaagttcactcctaagatatcgggttattacatgaccaaacacaatttgtttatcaaatactttcaaaagaatttgccaaacgtaaattatttttctccaaagtgacttatgaattactatgttgataaaatacttttcaaagtaactaatgtttagaagtcaaggatgggcttcttttgattattgaagtttgtagcaattgtatgtagttatagtcagggtgaccaccagcatctcatatagcaatacacaagtggattagcgcattttaaatttcaattagttcatgcaaatatacacgtaatagcattataagccactttcacaacaggcagattagtggttttgaaatttcaaccaatgatatatactataaattgatcaagcacaaaccttaattgagcaacacaatttcttacagcaataactatcacatataacaataactgcc

SEQ ID NO: 68 - DNA, 1500 bp, synthetic construct
  source 1..1500: mol_type = other DNA
taatacttattttgtaagaattgcaatattgttatttttgttatggacttaaatattaaccatgttataatcttaagtttatattattagaaaaacttagtttttgaaagactaatatgaacattagtacttatttcaaaaataagcacctagatatatgaaattactttaagtacttatttaaaataattaagtaccacacatacatacatatctctacaaactgttaaagttttctatatgagtacttattttaaaataagagcttaaatataataaattatgttaaatttcttatttaaaataataaaggaccaaacatgcataaaataaagtatgagcttaataagtcaagaagctaattgataagcattgatgccaaatgcacttactaacttttctatattgtaggaaaaatctaacttttatatttaaaatttatttttgtaaaacttctctaatttttggacaaactcttatattgattttttaatcaaagccaaaatatttatttaactatgaaaaaattttaacaactaatttattatggtaaataatattgatatggtaacttcaagcacatgacaaaaattataactaactgcagaagtttattgtctctctgaatcttgtggctatatcataacaaatacttgtagctaataagccaacgatgttctcggtttcatataatttgaattttaaaatagtttttaaatttaatatttatttcaaatcattattgtggctaacatgttataatcgcagtaatatttggagatgcaatacttatatttagctacaaaattttattgtatcagaataagtttgtagctattaagttagtttttgccacaaatttttataattgaagcaaaaatacctattcagctacagtattttgtatcgagtaatattttgtgactagaagattaatattgctacagtaatttcagacgtgtggcaaaaactcataattagctacaaaatattgtcgtagcaataattttttatatctattaatgcaattattactacatgcttttataacttgaggcaaaaatatctaatagctataacattttgttagaagtaatttttgtggctataaaattggtattgctacagtaatttcaaatgcgtggcaaaaaaatacgattaactacgaaattttattgtagcaataacntattgtagcaataaatttgtagctatttgggtaatattgctacgacagttagcaattatagcaaaaatgctaaattagctttgttaatttaattttgtagctaaacttttttatgaaattttaatttttgtggctattgttaggtattagctacaattttcatatatgtagctaagaatttgtagctatatatacataatgttgtagtggcaaattctaacattgtacgcttggctgcccttttttttttggctacaaaactctaaagtaaaggaactagaaaactcgtttggcgagagaaagagagagag

SEQ ID NO: 69 - DNA, 500 bp, synthetic construct
  source 1..500: mol_type = other DNA
caataattttttatatctattaatgcaattattactacatgcttttataacttgaggcaaaaatatctaatagctataacattttgttagaagtaatttttgtggctataaaattggtattgctacagtaatttcaaatgcgtggcaaaaaaatacgattaactacgaaattttattgtagcaataacntattgtagcaataaatttgtagctatttgggtaatattgctacgacagttagcaattatagcaaaaatgctaaattagctttgttaatttaattttgtagctaaacttttttatgaaattttaatttttgtggctattgttaggtattagctacaattttcatatatgtagctaagaatttgtagctatatatacataatgttgtagtggcaaattctaacattgtacgcttggctgcccttttttttttggctacaaaactctaaagtaaaggaactagaaaactcgtttggcgagagaaagagagagag

SEQ ID NO: 70 - DNA, 2001 bp, synthetic construct
  source 1..2001: mol_type = other DNA
cttttacctaaacttaaatattggccttatgcacgggcatcaactcgcgtattactaagaagaaagaagaacggctcctcgtctcccatgtcggcctctggaactcttaatataaccgccgacagacaaaaccggctggtctctcactgtttcttatcctcagaattattatgaacataaactaaagtacctatctcttgttttctcatttcacaccatttttttcttcacttcacacttggtctcaatcaaataaaaaacagcaaccgccttagcttgttgtcttcttcactactccacttgtccctagagttccccaaaaatatcaagctaagaaaaagttaactaaagaagagaatatatgggagctgaggtttctttcataagctgaccgactctttatttggcggcgcttattttatttattacttcaaaagaaaagttctttttgttttccttttttataattttcaactcccaacttgtgtgtgttgtctaaatgtctatatataaatatgtatataacggtttgtttaagaaacttgcattggtactggtttgttcttgtcaaccttcttgactagtatatatattgtaaacttttgtgtctctgtattggagatttttgtttgagttctctttttgaatattgcatgtgtatttacaaggtggcgaattcaagagacaaaatatctaacaggagaagacatgaagatagggatagagatgataattataatgtgttggaatcgatgttttcgggtaatattaatagggaacaggagatgtctgtaatggtttctgcactaaaacatgttgttgctggtgaagatagattgaatcaccaaatcttgactgatgaaaatggtgcagctgagaataatcttggttcaggtttttcttcagtttcttggggggttggtgagaaaagaggacgtgaacaacggcaaagtagtcagcaattcttacatattggaagttctgatatgagatcatcaggtatttttattttactttacttttttttttgaaaaaaatgacaccgccgtaaaaataataaccgaaaaaagtgtataaaatttgtataatttttgcgttatatatatacattctgtatgttatatacaaaaattatacaaatagttaacaaatttgtatataaaaattatacaaattttatacatttttcggctaccacatataaatagtttctggcgccggctaaaagtgataataccccttttttccttaaagttttggattttcttttagtttatgttgtgttttcagtgggtattttgaaaacataattaactagtggccaatgtagcattatacggattcaattgaatccatagctttcgaggcataaaataaatttatgtgtaaaatttcattaaagttataagatatagtagaatttgaacccatataacagatatgaacccataactttaaaaatacaatatgttcaatgctagaatcttaaatgttcaacccacaaaatttaaattttggatccgcctctgattattaaaagtatattttctctaacggtttaattaacatttagataagatgatcaaacattaatactatgatattagagtgggtataagttccgggctgaagcttaaatatttagatgaaacagacacacaattcaatagtttggatgtgttgcaggaatcattattgctaaataacagctaaatgtagatgagaataaatggattaaaagatttggttgatcttctatgtgagttgtattcattctgcattttatttagaagtaaattgctgcagatgcctttgtttttagttttgattcttgccattttctccattaaaaaaatgcacttgaaacatacaatataaatcaagtgtgtgacttttcactatattgatttttagtacattattaatgaaagatacaattaggtggatgtgtacgtgcttcattatcacattttcattattttgaatagcagattttacaca

SEQ ID NO: 71 - DNA, 2001 bp, synthetic construct
  source 1..2001: mol_type = other DNA
ttgtttatgttgtatgtattggtttagtgagttatgatataatgttttcattgaaagtgtgacatgatacgagtattttcaaatggacgtgagttagcatttcagattttcacttcatttgtcctatttatattaaacccacaaaatgaaaatacccgtatttatgtttatacactttcataaatttcaagaatttagacaaaaagatttagctttaaaattttgatatggtaattttgtacgctattttaaaactattatgttaatatttatcctcttttttaaatttcacaaaaataaatacataaaatatttatttgtaatttttgtttttcttttaataattacattttaaaatacttagttatggatatacctcatagggggtataattaagtgtatgatttttataatatagggggtgtcagtgtttgatcttaaaatacaaaagggtgtatccgaaactgaatcgtattatatgggtgtttagtataattaacactattatatataagtaatattgatatttaaatctccgcacttaattaaatactgtctcgtaaaattaacgtctatgtgtactgacaataatgtaatatttttttaaccttgttataataggtaatttgtattatttttgaattgatatctcttttttaatgaaaggttacatataattatcatttagatgataagagcgtgcataaattcattaacaatatctaacatatagagggatgaagtagtactattaattttctaaaaaatgagaagcaaaaaaggaatattcttctatgtcggcctctagcttagctaagaactcaacgccgactgacaaacaactcctaaagcggctctaattttttctctctaaaatattcttatccacatataatagatctcccttcacacttactaatactgctgtcctttattctcttcacattttgtccaaaagaaataatgacaagtcaacaataaaaaacaatgttttcatcaacaagattaatctctccttcttacacgcaagcaaagatggatctagaatttgcggagatacatgaacaaactgttcaactagtgccacccaggggcgaatttataggtaaatatatggggcacttgaacctattatttttccatcaaactaagtattttatgtatatattttttagaattggtctagtattatctgctgacactcatgctccaaaaaggctaaatggtgcacttggttgaatattgaattatttacctaaaggactacgaatcgaatctcacttaatactttttttttcctccttttttaatagtgcactcatattataaaaatcctatatccgcctatgatgtcaccatcaaacttttgattttaacggttataagtaaattatatcatcgtttttaatatatatagacaaatattttaaaaaaaattgccgatgtgtgacctctatctcctagcataggtccgccaacttggagagaagaatacaaattaaggaagctcatgagctgactagccgactctatattcttgtctttattattttacaaaaaagtttgactttacttttttgtttttttgttttaagtaaattttcaactcccaacttaggttagtctaagtctctatatatataacggttggttcaagaaagcctaaaaaagagggggttagctggttggtataattagtttgttcatcttgtcaactttcttgaaaagcaaaagtttttaatattttgttttcctctttttgaatcttgaagtttgattcaaacttctcattttatagaatactctgttaagactttttagttagtatagtatactagctatatactagattaacttttttttttttatttataaagtcccttaatttagtgaagttttgttacatgtgtatactcaaggtggcaaattttagaaacaaagccatttataagccaataagagatgatgtgatgttttctgggattaatagagaagaggaga

SEQ ID NO: 72 - DNA, 2001 bp, synthetic construct
  source 1..2001: mol_type = other DNA
aggagcgttcaaagtttagatgatttattatggtatatgtgcatttttcaccacttgttgccaatataatactacattttcctctcgagacgacaaagaatgataatgaagataccttttcttgctgaagattttgtcagtatactgcaactgattttagcaactttgctcttcatattataagtcaattatatactgtcgaatctttctattgtttaatcagtcgcccctcatgcatgtaaattaaagtataatatatataaagtataacttgaatgtggatttttcccccacatatataataaaacaatgaaaggaaaagatctatatatgtgtctttgatttttagtgggccaaacaaaaaaaaaagaagcagcaatatgtcagtattcaggagaaattcaaaaatagtcagatttacaactgatcattcaaaaatagttcagtttcaaaagtaatcaaaatttagccacttttcatgtaaagataaaaaagagcgaaaatattgttcaaaacccgaaaaatacgccagtatattatactggagttcaagtataagtatgcttgaactccaacatattatattggagttccaggataagtatgttggaactccagcataatatgatggagttccaacataagtacactagaactctagcataatatactggagtttcagcaagtataattgtccagtataatatactggagtttggagcacaagtgctccagtctccagtatattaataatggagtcagcaaagtataccggtccagcataatatgctggagttcatacacaggtgcaccgaaatccagtatattatgctggaccggtctctgttgcagcaaaatagtggctatttttcattgactttgtaaacactgactatttttgaatgaccagtccgaaaactagctataccgtgctatttttacatgaaaaacggaatacatactataatatatcatgttgtttatataaattgaaaagacgaataactcgaataaaaattcaaaatagtcaaatcgatcaagtgtgtaccatatttggaacgtgtaaatagcaaatctctatgtcggtggggcaccctctctgttataatacagctccagataagtacagtctaaattgaagtttcaacttgtgttaaaatatttaatctgatattatctgcatgctacataattattgtacgagcttggactttggggctcatgcttttgtccttcgtttcgagaacgcaaataagttcggtgcactaagctttcgctgtgcgcgggtccggataagggtggaccacaagagtctattgtatatagttttaccctacatttctgcaagaggctgtttctatggcttgaaccggtgacctcctggtcacatgacagcaactttatcagttactccaagactcccttttacaaacttcacttattattacgcaaaaagaaaagaaaattgttgtggcttagttggataggacactttatttgacgcaattcttgaaatggtaaaatacacttaaaaatcaagcattttttatattctctgaatcaggtcaactattcagaagtgtttccaagttgtcatacaaatactttttccccccaagttgcagaggtcataacttaacttacttgacgtcaaaaatttcatttaaggtgttcaaacttaaaataagtaaaaaaaatctccgataaagggtgtttaatatatcttatatatcattaaaatttaatattttacctatatatataatataattttcagacaaagggtggtcagttgaccaaacttagggctatgtaccttcgcccatcatgactatatatatatatatatatatatatatatatattgttagtagcccttcaaagttgcataattattagagagaatacacatttaaatattgaccaaacttgtctataaatttggatatttttgctactttaattttctgaagcaaataagaaagaagatcatttgagaaa

SEQ ID NO: 73 - DNA, 2001 bp, synthetic construct
  source 1..2001: mol_type = other DNA
tgtcgcaggagcttatccaccaccttttgtatgatattttcttttagttggttatttgatttgtccttttctctatccaatcgaacaattataagaaatgcaccaatattacttttatctatcccaatttatgtgatatttttattttctgagattcaaattttttaattttgattatatacttagacataaaatcttttaacttttccgaaataaaaattacatatttgaaaattacgtaaaaagtactataactcacaaaaaatattaattttaaactatttaaaaatatatataaaaagattacagtcaaataataactcgtttgactctcaagatacggataccgtcacataaatgggacgaaggtggtatattgagtgtcctttcataaacatagtactagtattttttgggaagctccatgaaaatgatattttttaatttttattagtttattttaattttttattttatccataatgatatttttataatgacataaatattttcgaacttagtaactgttgctcaaagtttgcatatttattaaaattcaataagcaagtgcaaaacgtttattgcaaactcaaaaaattaaatgagttatttcataaaatttaaatcttgaatttgcatacagtaacttatttaaaattatatattttaaattaatttttaatatgtacccgtgggagttgggtcggaggaagtataatggagacagaaccagctaagataatcaataattatactaaaaggaatttagtactttctttttggtgttctctctgtttcagtttatgtgaacctattcttttttagtccgttaaaaaaaatgatctttttctaaatttagtaataatttagcctaaacttataattctacccttaatgagaaacttttataaccatacaaatattctgagtcttctttttaacttgtttagtataccaaatttcaaaaatctttattttttttttaaattttatgctcaatcaaatatgttcacgtaaattagaacggatgaattatcacgtaactaatctaagtaatcacagttgtcttacatcaagctggtcataggtaaggggaaacactaattccaagaagtcgtaacgtgacctaaattaaaactgccaccctttggacaaaaacaaagggaacccattttgccccattgtgacaaccaaatgacacaacatggcatatattgtgacatacaccacccaatcaccaaatccaactaataaatgtcttactttttgcatctaaggttgtaacctaactaaatataatgaataaaatttgtgaaagattaggattcaaatatataaatagctaaaattatagatttcttttttttctatttaaatagcttatttggtcacatttctaaaattagcttattttttttaaaaaaaattatttttctcaaaagttatttaaaaaaaatacttttgagaagcagtttgtatttgagtaatcaatttgaaaagcatttttgagtagcaattaatctttggtcaaacttttaaaaattatttgtaaatatatatatatatatatatatatatatatatatatatattgaacaaaaagtataaatattttgtgtcgacacttgcgacaaatagatgggtcacattcaatccaacatctcattagcttctactacgtgtttggattactcccccaacttgatggctgattgatcatcgaatttttgccacactatcacttcttatggtccaaatacttctcatacttttctatacataaaattcttcaaacccctcgaaaggtcaattatcaactttcacgtgatcttggaaacttgcaactttcttgccatgcattgtcacttgcttgaccaacttgcatcacctttaatattagtaataaaatttttttactttccaatctcaagatctataaatagaaagttttctttccttagttcttgtttcactatcaaatgaagaggcaaagaggaaaga

SEQ ID NO: 74 - DNA, 2001 bp, synthetic construct
  source 1..2001: mol_type = other DNA
ttttatacccatactcgtgatcatattagcgatccccctaatcgtagtgacggccactcctgcaatttcggacctttgtacggtacaatagcgaatagggctcctaaaaaacgtaaacctgtaaatgataaagataacagctaaaaaaatgcaaccatcctttgaaacaatattttttttaaaaaaaaaaaaaagttcttgcatgcaaatttaacttcgtatactaatctccaatgcgatttagaacttaaaaattactattcctaaacaattagaaaaactgcatttcttaatctaaggatgttttgtgtctgtcctattgaaacaactttagactacagatcgttaaattgtaatcaaccttcttttagtgtaaactgacgcggctttaccctttggtcagttaaataatccttacatttggtaaatgcttatatttactcacgcgattaagagtaatgacctcgcaaaggtagagttcaactgcacctcactttgtattctttgaaatagccctcaaacattacgtcaatgaaataacgatatgatctcgattttgaatttctgagtcccaggttagtatatatataattacattcctattatcatggcagatatttcaaaaaaaagaaaaaagattaaaaaaagaaagaaaaactgacctagaaatatctccagtccgcttcctagaaatataatgcatgaacactagaagataggacatttaagagagtcacatggttgtttatggcgagaaatattgctaaaaataaaaaaaatatgatattatatgtcacataaagttggataatgtaaatgttggaggataaaacacttcaagcccaatcataggccgactggtgaatggctgcttaaatttcaaattgatttacaccctaccaaatacaaaaatataaactctttggtattgttactatttggcaatcaatcatattcataattggcatgtgagttgaagctaattgatgaagcgaaatttgatgcgtgagtagtctctttattgcaccacactaactgttgagatattttgctcaacttatttgaagaaaaacaaaaataaaaatgaactctttggacactttcaatctttcattgccatttatcaagataatctgatgtaactaaaaaaataataggaccagtacccaaaatccagatcttttcccagcaacaggaaacctttatcaaattttagtccccatttagactacctaatcaaagaagatactctgcattcgacttaattcacgcacattgtcaataagacaaagaccttagcgaagccaaccaaactctttcttttactttttcatattgctccatcagttttaatttatatcacacagtttaattcggtatgggatttaaaaaaaaacttttaaatcttgtggtctttaaagtataaaagataaaagttttgtggagccataatatttgtgtgactataaaaatttctaaaataaaatatttataattatattattttcaaatatagaatatataatttttttggaacggactaataaaagaattgtattatatactatatcaattgaaacggagggagtagatggttatccgcacgcatctttagtagtgacagttattcgatcttataataggttggcctatgcatgctgatgtattgtcttttagtagctacatattgagttgaattaccttttaccatgcctgcgaccccatgcaaacaagagcttaggtcctacgaggaatgtccttctcagcacgccagatgcacatacatgaaccaaggcaaaaaagcaaaggaaatttaacctttttgccattaattacatttaaactggactagctcattcccccagctctcttacccacaagctccttctataaattcagaaaccaactgcaccccccacattcatcataactcgatctaactttagttattgagagacaaaccaattacagaagcttaatccttagagaaattaagagaga

SEQ ID NO: 75 - DNA, 2001 bp, synthetic construct
  source 1..2001: mol_type = other DNA
gaattttggtatttttcgaaaatttgaaaaactccaaaaggttatttttcaaaattttcccttcaaatcactcacaaaatttcaaaaacagctccaatttatattcatatccaaacacaactataattttcaaatactattttgaaaaataattttacttttttccgaaattttacaatttttatgtccaaacacccacgtagttctttcaataaaatttctaacatgttatggcaagacatgtaggacgagagttgatgtttttttttcttttgggcttttcaaatcgagttctgcaatgaattggatttaacaataatagccatctatgactagttattaaaagaatttgataccagaccattttaaccagatttgtcatttgtaggaatttgggcttttgtatttatacccgattttggggtcacaattgaagctataactacttcgcaaaaaaacttgcaaacctacccactttacaattaacttcaggcttatcgggtctgaagttaaaaaaaaatagtctgaagtgcaacgcacttaaggccaattaagtctaaagtgtaaaaactaaggccaaatatgtctgaagtacaaccaatggttttatacatttaagaccaaatagatgtgaagtgtaaccaatagtttcacacacttaaggtcaaaaagtctaaagtgaaaaatttgcacttcagacttaaggacaaataggtcagaagtgcaaccaatagtttcatatacttaaggccaataagtctgaagtgaaaaaattgcactttagatgcacttaaggccaaataggtctgaagtgcaaccaacattttcatgtacttaagaccaaagaggcctgaagtctttggacttaagtgcaaattgcccaaaaataaaaatttgttcttcgaatttttacaattcaatacacgatttaacacctaaatctactccaaatgagaaaaataaagcttcgaaacagcttaccactgtaaaacatcataaactaccttaaaatatgttcacaaaacaccatataaaatcactatataagaagaagaagaagaatgaggaggaggaggcctgaagttgtttaaactttggatactacttaaaaaaaattaaaaagtgggtataggttaaatgaggaggaccaaatagggcactccgtaaaatttttacatagtaattaccggtcaagaaagttaagtaagagggatgccacactctattgagattaccccaataaatcaccgaactccactgcttcatcaaactacagaacatggggtctttctctctctctccctccctccctccctccctctccaactctatctccctcaatacgcacaagcagtactttttcattttactttatatttgtgaaggggaaccttgaaacaattataaacttatttctatgtgacatgtcacggattcgagctgtggaatcagccactaagactagcattagggtagactttctacatcacactccttagggtgcggccattatcgcacctgcatgagcccggaatatcttgggcgccgagcttcccttttttatatttgtctgtatttttgaataaaaagaaacaaaggtaggaagtgtttaggaattttatttcttatggataatgtataaaatcctccccggcctattaccatattaccaactacacactttttttttccgggaattctattaccccatgaactattttaaagtataataaacaccaccctaagtgatgatgtggcatagagagtatgcacactctcttaagggcaagtggaagttacaaaaataattttaaaattgattaacatgtacaagtgtcatcttttgattggacattacacaattaattatatctaatttattagtctccttcgtcttctaaatctcttcttgcacgaacaagggagacatcgtttcagcggcgaaatgcctgagcgaaaatccaatacaacgcaaatcaaccgagaaatcaaaagcagata

SEQ ID NO: 76 - DNA, 2001 bp, synthetic construct
  source 1..2001: mol_type = other DNA
gttttagagagtactttggcaagaaaaacaagtgtgagagatcacccaaaacatcatattttcttcttctctttatttttctggcaattttgatcatgaatattttcatagtttatttacccattgtcatgagtagctaaatcctttgtctagggttttgatggaacctattgaagaatgaacttcttgattatgttaatatagtttgccagtttaatctctatttgttcaactacgtgtttgttgtagttatttgacaggatcctcaattagctgtgcctatttggtatgcataactcgggagagagtgcatatttaggtaattgttgaacaacatcactcccaaagtataagagggatctataactgcgggtttaaaggcggaattagggataacgaagtcttgggtgcaatctaaagtgaactgtaataaacaaagccaggtagcgtatctcgggagagtgcgtctagtaaattatcgtgattactcgggagagattcacggtaaaaatagtgttcatggttgatagagatgtgttggtaaatctatatgaaacataaacagaagggattccatcaatatgggaaatcactaccttagaaccttctcattattgttcacaacttaagcatatttagtttacaactatttattgactttcaatcttagttattaaatataccatcgactgttattcacaacatttggggaagttgattctaaagaatttagtaagtccaacgaaagtaattgataggttaattctctgtggattcgactctcgacataaatattcagattatatttgcaacgtgtgcattgtccttttataaggtatagttgggcatgatcactaatacttacattattttttctaatacttgccaaatgaccccaacaaattttcagtctaaatggccggctagtcgtaatagtttaacgtattttaaacgtgcgaaaggggcttcttgactttatcattttatggtttcagccggtagatggggacaacctacaaattatgtggccaaggggacaacccaaatgtgaaaaactaattattatttcgttcatttcaatttatgtgtcttattttattcaattttttttatctttttaaatagtagtaaatttttaataccaatgtttgtattttactcttaatgacacccgttataaggaatcatatggcaatatatatatatatatatatattgacttgaagagtactttttatagcttatacacacctttaatttaaggtaaacaatattaaaattcattttacattttagtccatttaagctatacccacctttcatttaaggtaatattaaaattctttttacattttagtccagttaatctagaacatatcagatgaaactgatcagagagtaacgatttaaattttctctgaagcagcttcagtcatatgaggcgcaatatcaaatttctcctttttggctaacgaaataaaaataaatgcgttagaagtctctggtccgaatagtagttgttcgtttaaaacgtttataaatttaaagttctactgtagccgaacacgtttgtgtataactatatatatagacaaaattaatagtcagccaccaaatcagtaggttaatcttctaaaatgaggctatttaactaaagctatagtctatgtacaatttcaggcaaccctatgctacttcaacaaccttggtccccttgaagatctcaatatcccaccttatcaccttatcttgttgatgctacaacgtattgtatttgattcttttacttttatcctgagtagtggatttatcggaaacagtctctctaccatcacaaggtaggagtaaggtgtgtgtacactctaccactcccaaaccccactttgtgggattacattgtctttgttattgttgttgttgtcgtcgtcgtcggcggcttgaagttctcattatattagcagccttaagttcttgttcattcttgtttcttatactgccaga

SEQ ID NO: 77 - DNA, 2001 bp, synthetic construct
  source 1..2001: mol_type = other DNA
tttaataagcagcggtttatttccgggcgaaaagggtaagtcggcccctagcagtgagaaaccaaaaagaaattgttccttatattgtgcgcatgtcaacttttttggcttggcttgtagacgtatacacgtggaattaatgagagaagataataggagtattatagtagctaggtacatagtatttagcaaatctttcatggctttgacttcccacgaaattaaagcagtaatcttcataagctgcatattatctccattcttatcaccttttgtacctacccacataaagcccgactgtttggttggtcaaaggtcgaaatatctatgcaacttggccaatatgctttctatataataaaatatgcatggcctaccatttaatttgtatagcaacaaaatgtggcaaataaaaaccctaccagtataatttctgagctttcaacctaaattaaagaagtctatatcatgctcagcctatatattttgaccatcgagagcaaaagcatgacgatgattccttgtatttaagagtaccgtggcttaattgttaatgaagtgagtgataattatcggagattagagtttaaattattagcagtagttatattttatctgtctatgttatggtggcaaagttaccaaattcatttgctggttgaaggtaattaccgatgtttaatttgatgaaataatcgaggtgcacgctaacagtaggtcattagcctcctataatgctgaaaaggatgccctaaacgtattctaaatttctatgtcataaccatacgtcaagggttatactattatactactacagtccaacactcgagaaaaattaggacattgagtttttattttttatttttaattggcgaatactgttaagttggttgcaaattatatactatctttttcttgttaaaaaaatacacaaatatagagacaaattgactaaatgcaatcaaaccaaacgctatatcaaacagaatctgctgtatttaagacttattcaaagttgagatctcaacatctcagccttaaaaagttgagatgggtaatattggtttttaaataattatatgacaaaaaagaggtaggttgtttattatataaagatggatggtgattctccagatatcttttggcaaacataaattaaaccaaaattcaacgccttaaaggattaactaaggctccaaatgtgaattgcgtaacgctgcatgtccccaaaaagtgaggtcggcattagctgaaatatcttttcaggattatcccaaaagaaaatacctagtcacccccggtacttataccaaatcgagcaatttaaatctaaaaattataattaaaattgaaaatacatatcacttaatcttaatcaagtgataaaaatttacacgtcatatatttcttgaatttgtggtaacactattaaaaaaaaactccgcctaaatgatctaaagtcaaatttgaccccaaaaaaaatctaaactaaagtcaatgctccaactcaaaccggcgtcggatacggggcgtcactggtcagtcctatcaggactcgtgagacttgtaattcttatccactaattctcataaattcaatcatattacgttatatttttagaaaacccatataaagatgaagtagaattagaatttaatatgttttctatatatagatccgaaattagaaaagaagaaagggcagagatctgggttcttttatcattgatcttctgttttccaattgtcttaattcagtagctgtcatgcatgcactagataaaagttatcactaattttcagtgggttccacaagtgcatgtaagtcttaggattttactactgctacttgtttggtgacttttatatatattgtttcatagatatcaagcattgggtgattataacaacaaagatttcagattcgactaattcaaattcgtgtcggataagattatataaggaattgagaacataaaataaggatctttcattgaaaaa

SEQ ID NO: 78 - DNA, 2001 bp, synthetic construct
  source 1..2001: mol_type = other DNA
tggtatgaaatttgaattgcattgagtattcttcgtgttgctgttgtgtgtttactttgagactatgagacggtatcccgagagatccccctgcatgtttactttgggactacggatttgtattccgggagatccccgtgcacttttatattggaactacgggattgcacccggtagattcccccaatattgggtatttatatttgggactacggatcggtattctgggagatccccgcgcactatgagttggactacgagacagcacccgggagatccactggatatttatatttgggactacatgatggtatcctgggagatccctggttgttatctctgtgttaagttgtattccttctgtgattatttggctctgttatagttgttgttatttttactatcctgtgttacttcttattgtttgcacttaattatattgtcttattctatattgttataccttgtttttcatctaaactcagtagggccatgactttcctcgtcactacccgaccgaggttaggcttggcacttactgagtactgctgtggtgtactcatgccctttctgcgcatgttttttcatgtacagatccaggtacttcgactcagccttactatccttaaggcgaggcgattctccagagacttcaaagtatatcttccgagtccgcaaaccgagaagtccctttctattctcccatatagtattagcccttctgtatttacttttgtttaaacattctggagttagacacttgtagttatccaacagcttgtgatttcattagattccgggttttgggaaatattgtttcagttcgagagtttgtattgtatatgtcgagcggcatctaaacgcttcattatgttatttctgtagtttttgactagttttattctgttattttctgtttccgtaatttgttaagcttacctagtcgtagagattaggtgctgtcacgatagttcacgaaggacgaactggggtcgtgacaagtttacttagattataggagtttatgggatatctacagtattttttttttatatacattttattgattaatatgatagacagttcaagaataaaagttacaccgctagtacgtaagaaatttattcttaacaaatttaatgaacccaagttgtggttgaaaaaaaaagttgagtttgaaaaaaaaacatttgtatcaagttggggttgaaaaaaacaaattgaaccaagttgaagatgaaaagagaggagagttcaaaaaaaaaaaaaaagaaaaaaaaaaaaaagaggagaattcaagttgaagatgaactctttaaccactgtcttcctctttctctctcctggtataaaccgacattctttaaggagttcgaaaggtcgaaagcagatgcatgtaacttcatcattttcagtcaaagtgcaattaacggtgggaacatatctctctgagtcggccgctgtattgttgtttttttggcaggcatcaaatctttcttttttatttactatttaaagaaatttaataaaaatacaaagctgagtatatttaactattctttgtacaatatagagattgacaccaaacttcgtgcatgatctattagcagaaagtagcacgaagattcaactattctcttcacggctgttcaagtttgaattttcctccttatattatatagccgtttgtaagggaatttttatcagttatctgtaaaaagaatcagttgtctactttgtgcatgtgctccaaatcaagtaaaataccataagggagcccatcaattcagaaatatacgatttgggttggtcttatttttgtgctacgaaaagtgttcgacgaaattgattttgtcagagttcaaagtcggtggcttcagattctgcagaaacggtgttgtgttgaagaaaagctgaaaagggtatttataattcaccaaaaaagcatctgggttggtgttattttgtcgtaattttgtctagtttgaagtgggtcgtaca

SEQ ID NO: 79 - DNA, 2000 bp, synthetic construct
  source 1..2000: mol_type = other DNA
acaacaacaacattattattaaaaacaactgcggaggcatgattgttgttgttgttattattgttttacgtaatattgttaaaaaacaacttcagatgcatgattaattaagctaggatcgcagtaaaatttcataagtgggatctggagaggatagatcagcgtacgcaaatcttatccctacattatagaggtagatgggttatttccgatagacactcggctcaaagaaagcgattccaaaacatattcgaagaaaggaaaaaaatatgtcgaaagtaccgacaaaaataatacgataatactaataaaagaatagaaaaaaaaaacaatacggaacaatattacattttaaaactcagtccatatcaaacaacatcacaaaaatagataaggacaatgtgacaaactaatcacccctgccctacattttaatatttgcagtgtacaattaacggcatgcgtcacgcccttagacttttgtgtgaatttacaagtattttattcctcaattatatccatcaatttatatggttaacatttatttatttattttgggtgcaaattcacatggtttacttaatggcacagtttcttgaattataaacaaattcatgttacccctgttccaatatcgtctgcactaaaacagacaaaaaaaaatgtttactcgcatatattaggcgcggcccttccttgaatctttacacagatactttgtgtatcagactgtcttttacttgcacacgttattttcgtcaaatcaatgagtgtaatgcatgcgtattcaatataaactttgtactcccttccgtcctatttagttatactttggctaaaatagttgttttaaaataatcatggtttaaaaaaaaaacaagaaagagttacttatattttttgcaattttaccctttctactctaataaatgttcctcaaagaagtaaaagagattaaggggagaaattaaaaaatagccagatttataagtggtcattctaaaatagtcacagtttcaaaagtaattgaaatttagtcacttttcatgtaaagataaatttgaacgaaaacattgttcaaaatccgaaaaaatacttcaatataatatactggaattccagtataatataccggtccagtatattatactggaactttccgtgtgttggagttctaacataatatgttggaagttcatacacaggtgcaccgatctccagtacattatgctggaacttttcgtgttgcagcaaaataatgattatttttcaatgactttgcaaacgctggctatttttgaatgactagtccgaaaactggctagccggtgctatttttatgagattgagagatctaagaataaataagggcaattcaatcaaataactcatattagtaatgttttcttaaagtatgcgtgcaaaaggctaagtaggagagagtattgattaatcgtgattaagtgtatatgctatcacttaaatttgagactcttaaacttgaacttattaatttaatgtaaacatcaggtgcgagtaagtattttcatgttaacagacacagtctcccaaacacaaaaaaatggaatgacctgtgacaagctgctagcaaaacataggccctgctaacaccaaaagactgttaaagttaaacttattaatttaatataaatatcaggtacgaataagtattttcctaactacagccgtcgactagtcttcaaattgctagcaaaacataggccctaataacaccaaagttcctatattaagtgctatatattgtcactttattttgagacggttaaaattaaacttattaatttaatataaacatcaggtacgagtaagtattttcataactacagtcgtcgactagtcttcaaattgctagcaaaacataggccctaaaaacaccaaaattcctatctctctgtgtatatatatgtaaagcacatatctaattttgaaataaaacattcatagattagctttttcaa

SEQ ID NO: 80 - DNA, 2001 bp, synthetic construct
  source 1..2001: mol_type = other DNA
taaaaactgagtatgtcaacatgaggatcaactggcagatatacttacaaaaggattgtgaagagctcaacatgtacatttgctgaacaagctagggttgaagaatctgtatcaaccatcagcttgagagggagtgttaatcaacatggttaccactagtttatttataaagtgtaaatgctaaaccatagctagtgagttagttaatagttagttgagtttgttataaatattagtcagctgtacagtttaacatagcttctctttcagaaatgaaaattgctcttctctcatttcctctcttctagattcttcttctccctccttctcttagctcagatctctcttatgacagctaacaataaatacgaatatttcttgtaacggttgctcattgaatgttgtctttctcaaccgatatctttctttcaagttttccccccgattcgagtatttttgaaactcactcagcaccggtcacatattcgtaatcggtgccagctatttgcttactcatatcttatttgacttcattgtcacgtgtcagacagaagtatgtgcgcatataccatcaagtctcaatttgaaataaaatcaacttaagcagttaaaagtcaaatctcttttagttcggtctttaaaataataatttaaataatgaacctataaaacacgcaactcacactgaatataggggcagacataaaagccgaaagactgaattccgaaccggaccgaattatttcggtatttcgatatcggtttattcagtatttcggtactatttcggtataggatttttagttattcggtatttcggtacgatcctcggtattgaaatttcgatatttcggtataccgaaataccgaataatttaagtacaccttccttcactgcccagcccgttatcaattttcagcccaagtttctaacttgttatttctttcccttagccagtagcctactaagattaagcccaacgccccaacctaacattagaaattattataattagaaaagtataaagaaagtactcacattctactgctatgctcatgtagtgatttctattagaaattattagaagtgaaggtactgcccacattttcttgttgctatactcattatcacgcaattagaaattttctaatgaattagaattcagtagttcagcacagaggcggatgtagcgtattacctacgggttcaactgaacctataactttcgacacagagtaaaaatttatatgtaaaaattctttaaaattgtaaaaatcgtagatatgaacccataactttaaaaatataatgggtaacattaaaattgaacccatagaatttaaatcctggattcgcctctggttcagcattgtttagttcacaaaaatatggtacgatgccgaaccgtatcgaaaccataccgaaccaaacaagaagatatcgaacaataccgaactactttggtacagtatttggtatgcacacttgatatatcgaataccgaaataccgaaccgtaattttcgaataccgtaccgaaataccgaacactcacccataactaaacattaaaaagctagaactcaggtgtttaatgactaaacggaagtaagatctagataatccgtcactctgttgatttgtaaggctatcgacatgcaaaagtggaagcaaaatggagccgaaattttaacaaaaatgctgaaccaataccatgaaattgatgaatggtgggaccctatttcactcttttagaatttgcgtaagaccagaaaataacttcaatcgaaatcaaaataaataccaacccttttaggccccaaatcactacgtgtgatttgcaaacgtcattagccttatgtaaacagtgacctcatgccaacatattatcgcagcctataaatcttagtttacatttcattttctttcaaacacacacacctcacaatagaactaagttgtaagagtttcattttctttgttctttctcacaaaccaaaa

SEQ ID NO: 81 - DNA, 2001 bp, synthetic construct
  source 1..2001: mol_type = other DNA
ttaactaccagaaatcacataaatacataaaatcaaaagtctatcacggaatatcaataaaaagagaaacataaaatgacaataagtaacaatttcaatttgaaacttatcaaataaatcttacttgttacttttcaaatgagtgaaaaaatcgaatcatgccagattaatatatgttaagacaaaacttaaagtaggaagattttgaatttttatagtttaactttaaatattttgcaccaatgcaagcttaacgcaagatactgctcatactattttcttttctgtttttgtaatgaagaatgatttttcttgcataaagattcttatctttttcgtgcatttgacgggattttttatttcttttttctttttctgtcaacaagaaatttatttttgttgtttcgtaacattttttcgatggcttgaaaccgaaaaactaaatcgattaaaccgaaccgaacatgaataaatcgaattgaaccgaaattattatggttcgattttgattattaaaatcacaaaccaaaaatcgaataaaccgaacctaacttctacaaaatcgaacagaaccgaccgacgcttacctgctcatttgtcaggtgagacaaatattaagtagccatcagaagatcattatatatatatatatggaatctctattgtgactccacctgtctcattttttaagtaggatttttattcaacttggtagaaaaagttaaatcgttactaattgacctatgtgttcatataaaaaattctaatatgtggtcgaaatttcaaagaccggcgcaagcgtcaagataaattaccaatggcaataagttgagtcttgaaatttgcaaacaatcacaacttgtattagcttcgactgagaaaaaaaatggaagaccaagtgaaactgctaggagcttttccaagtccctttagttatagggtaatttgggctctgaaacacaagggtatcaactatgaatacatagaggaagatctttcaaataagagccatgatcttttgacatacaaccctatctataagatgattcctgttcttgtacatgctggaaaaccaatagcagagtccacagtcatccttgaatacatcgaagagacatggcctcagaatcctttgctaccaaaggatcctcatgaaagggctcaggctagattctggatcaagttcggagaagataaggtaagttaatcatgttagtatgtttactttaggtccagtgtttgccaagagtttttaaatctaaatatcagaaggaaattaatgtagagtacttctagctagcattaaaatatttttactgaggattcatataattggccccaacttggtagtacatgctagttattgtcaagtgttaaatcgagtaaattacctcatctaaaattttaagcgctaaatgaaggatactatactttatttataataggtctgcgacacaccatcacttgcaggcttaattcatttttcttgagccaaaaacatgaaaattttgttcatatgtcacaacaatataatgagacttgaagaactcagctcttatattattagacatggatcaaatttacttagttattataggcctgtaacaacaagtaacatgttatatctttttaatatagagcccagaatttttcgcaatatttcacaagataggggaagagcaagtcaaggcaactgaaaaagcaaaggaagtgttgaaaattatagaagagcaaggtcttggagagaagaagttttttagcggggacacaattggattaagtgacatagtctttggatggatagcgttatggctggaagtcatacaagaagctgctgaagtaaaggtcttcgactcagttagtacttttcctcgtttacatgcttggatacataactttaagcaactccctgtaatcaaacaaaataccccacatcgggatgcaatgctagcttatttcaaacgtcgtcgagaaatggttgtagcagcggcacaaggttga

SEQ ID NO: 82 - DNA, 2001 bp, synthetic construct
  source 1..2001: mol_type = other DNA
atcagtcctagcctcgaacgacttcgaagaacgttgtcagacaatcgagcatagccaacaaaaagccgaaatatccgtgaccaaccgaatattacggctggaatctcggcatgtatcgataaggaaccaacaatcagcgaatcagaagattttttacctttttatagagttgtaccaaaaataggactcttctactatataaagggggtctaatcattcatttgacatattgtaacacgcactcataagcaatacattattatttttatccgtcttttaagttcttgttctttttcatccataccggtctttgtgagttcggttcgagagtgactatttcacttaggctgaaactatccaactcttgtggtttaaatttatttggtctttgtttattcaatagaaacttaatttattgctttgtatcaagttaatccgcgtatccttaaaaccacttataaatttaattgttatccgattttgagggtaaacacctatatctatcattctattgtaatagttgatagggtttattatcaataaaacctcctagtttaatttagctgttccttcttctcttccaaatatttcactgttccattgcagctgcatcagataactaaggagaacaatttggatggtaggcccagagaaagatatgatctaagactagagagaaattcaccaagaaaatcgtactccttattaaaattttgtttaaacggtcaaagtagtacttctcgtcatcctaaatcctagtactatttaatggcggcttttatgtttctggaaaaaagtagttgcaaggagaaattaatttatggatgctgcgtttaactatagattggtgttaaacaatatcaaaactcaaaaaggttagcttcaaactaaataatatttagacattaattattatcctgtaatatttaaaattttcatcagaatactttccaatttgctaatattattattaattctaatatatatgctttttttttttgagaaatagagaaatatttattttctacgaaagatgactttcgtcttcttccgccaaaatataggtgaaaaatcgaagacgtccatagcagataaaatagagggcactcctacttgtagtaattgatcacacttaacatgaaagagctataatgttagttattcgttcggacgaactcgataatttttgtttaacttgtattagaaaatttattaaatatatataaatattttaattacagactcattaacttaaaagaagatatagactcattaacttaaaagaagatatagattccaacacaagttcaaaattcataaacgtcaaatcttggctaaatttctgaacatgaatgcattcctttaaaatatagataataagttaggatgttgtcactttcttaaagcatattccgactgagtctggtagaatctcataaactttaggccttatctcttcaattaggcaattacttacctccgctctactttaagaaaattcaatggagtacaccattattaagttcatataaaaataaaattatattaattctgtctcttgttggttcgctctatctttttctgttttcctgcttcaaccataacatatacaagaactacattttccaagctagatatatctaacatgactgactttgtaaatttcttttgccaagttaaagaaaaaaaatgatgttatccaaataataaagagaaagagccctaatgaaaaaaatgatttactattagagttgttcagctaatcacatcaattatggttttcatcaagtatgactaatggcggctcttatctcacgtgatgtgacattgaaattctttgactttaacactaatgtcatatgctttcaaattaataatccgataaagtctgctaacatgtgactttccaatttttttcttttacaaattgcagacttttcaactcttattccctattaaaacccatccatactattccttgtttctcaccaaaacccaaaa

SEQ ID NO: 83 - DNA, 2001 bp, synthetic construct
  source 1..2001: mol_type = other DNA
tttcaaattactaagttgctaaaataaatacggctaataagtaccattatttacatgagtaaatactaaaagaaaaataagattcattttatctaaactatggaaaaactaaaaaatagatatccaacactgttttcattcatagtacaattgaattgaatgtcttttaatttattagtattagtatggatttgattttggtttagaatttatttgagttactaacatttatggactataaaacttattggagcatccaaaaattataagttccaacttgaaataatacattagaagataaaactatgaaaaaacttaagaaatatttataaactacactacaataaatatttttatgtagtaaatatatttaaaacttctatacttataatgtcgggtttgtttgatttcggtttgactttttttagttagaaccaaatcaaaccaaaccatagtcgggtttttttctcggtttggcgcggatttccgggttgatgcgatttgtcgatttcgtttgtacacccctaaatattacaatagattttttaaattggcagcatatataagttaatttcttaagttttgtatccctcagtataaagaatttattacactatcgggacatctttcctattatagcagaatttttttcttgtttcaaaattaaaaatctcacattttaagtaggcttgcccataattatttgggtgacctaatagtataagaaattcttcacattgacgagtattgaacttaaaactcttttattataaatttaaccttgatgccgttccatttaccgactcttcaaaccacatgaccagaatcaattctggaatccttcatcactaatcaatatctgttcgagggcatctcatagtcaaaatgtcgtagcaatatcataaaggaatgggaaaaaaaaatgaaaataaatcgcctctggtcgaagctcataatgaaaatttcggccccgcaaactttatcaagttctgctctcaggggttattttggtttgaggtataaaaagatttgatctcggtataaaatgtcgtatattacttatataatgtttggtttctattataaaattcttcccataactaatatagatgtctaataacaaatgctaatttccgaaaggaatatcggtcggaattcctcggaaattggctcgtcggtaacgtcgctaccagattttccatcgaaaatccctcctcgatgagccaatttccaatgggacattcgtcggaaatttacgtttttggtagaacttataactaatactacacaaattttggtttccacctatctaaagacaaaaatgccttaaataaggcagtcttacacaatgcttcaagcattattctacaacaattcatgcattattaatattcactcttgtattattattcccgatccatgcatgtattgtaattacggcatagctagacgcgcgggaaaccaaattaacgacctctaaacaacaataacaacccagtagaatctcaccggtggagtttggggagggtagagcgtacgcagaccttacccctatctcggaggtatagagagactgtttccgtgattgacgacgacctttaaattatttgtttatgaggtttgattaacttggacaagattgtttgtattcatattttaggcttcagttcacttaattaaactacctatatgagaacatcacatgaagtactaatactaagagaaaagactcaatgtaaggcggcttttaaacaattctcacgtttaactgtctcattcacaaaattagactgacttgtattatttcttttataagttcaactttttgcgtattcagaaagattctttaatttggccaactggctactatagggactcttttcttccagttatataatccttgtctctcaaattcaagatttgtaaaccaattcgttatccctttcttcaactttaacttctatatacttgagattttatctctatatatagctaaataaa

SEQ ID NO: 84 - DNA, 2001 bp, synthetic construct
  source 1..2001: mol_type = other DNA
actatactatagtgtgatgatccaattggtcatttttgagatctaaaatttcttttctctatttgagacctctcatatgttcattagtaatatatgaattgtgggtatggtggcacgggttccgagggatttgaagccttatggaacacttgaactagaacttaaagccttaaattacgagaattgaccaaagttaatattttgagtaaacaaacttagattcatgatttgaaagtttcattaggttcgtatgacaattttggacttaattatatgttcaaagtaggatccgaggggttagggtataattcgacactatccaaaaaagttgtaaacttatgagttcataaattggatttgaactttaattttgagttgtgatgtttctatatatgttttgaacccttgaacaagttcgtatagaatatttaggcaagattcaaagtccaaattggactcgactaaattcagacttcttagctataactaagtaaattgtgttcttttgcgtggaaaaagtataacaccccatgcaagacaaaagtttgtaattcctcggtcttaatttatgtagcagtaatagattcagcatcgagtataaaaagaaaataaatttttgaaatttatgatctaaagtaagccatatacgtttgcgtaattacaaaccatctcattaagataaaataaaaagtttaaagttaaaatatttttaaataaaaaaatatgtcattctttttagataagctaattaaagaaaagtatatcagataaaatggaacaaaggaataccaagattaattgatctactttcaaattcaagtattacctcagctcaacaagaataattttttttggggggtaagtaattaatttggtatttgtttagactttacccttaccttttgtgagctaaagaggctggttccaatagaccgtcgatttaaaaaagccttttcaaaagaggtttgtaaaatacaggaattaaaaaaagatgaaaatactaaaaaagaaaaaggaaaaatattataatagtagtaaagaaaccaaagcaaaagaagcaacaatagtaataaaattaaagagtaagatgatgatggtgatgacaatgataatactaataataataataataatgatgatgatgatgatgatgatgatgatgatgatgatgatgatgatgatgatgatgatgatgatgatggggagaaaagggagaagataaggtgatctaaactagactatgttcttccacagaaaagagagaaatcactcgacgacctactaacattctactctaatcctcgactttcatgctcttctatctcgggtcgtgtcctaggtaagttgaaaatgtggcatgtctaattacctctctccaattcttcttcggtctacctctactctccttagacctatcattgccaacctctcgcactagggcatccatgctcctcctctttacatactaaaagtatcccagtcgcacttctcgcatcttgtcctccacagaggtcactcccactctgtcgtgaatatctttgttcctattcttatctctcctaatatgtccgccatatcacctgaaattacttaattttgtcatctgtcactcaccatttaattttttaaaaggtccaaatttaccccgatcaacttttgagtatagttcagaattacaagctagaaaaataatagtattaaatcaaagtctacggaggacgaagttgctcaatctcgcatgccgtatagtataggggcaacttcgccccttctccgaatattcgaccattttttctcataaaattctatgtctctccatcacaacatcagatttgaactttctacattttttttttcttcacttgtgacaaaaaatacactgttttaattccatgacgtgcttcactgggtgtgactttggctttttaagtctttgtcggtcatcttctgcttgtctatataagaagcagcctaatggttccttaaacacacaatttcagc

SEQ ID NO: 85 - DNA, 2001 bp, synthetic construct
  source 1..2001: mol_type = other DNA
ttatgtgacattttttttctttttatgatgtttaaaaaaactagtatctcattaattaattaattatatgtctgaaaatattaatttttttactttaaactttctattctatctttagtgttatgatttttatatataaccatggaaatgccatgacttatttagtatcacgcgttttaaaaatttttcttttattttttaaaatttcatatattccagtcaaatagattcacataaaatgagacggatggggtaaatagtaaatcttaaagctagcaaactatgttcaacatcatttgatgggtttcttttttcaggataatattttatgtgtatttggtctttttggattgttttattaaaagaaaaactccttgagatgcatggtaagccaattgatcgtcattgttattttgaaataacaagtaccatatcatatgaggccgtgattatctaaagacagataaggaaattgggctatcaaaataaatatgtaagagttatgttcctcatcattaagtatgtgaaacaaaattgtcaacttgtacaacgattctggaatctacttggcgttttcaaagctctctgagccacaacaagttcttgcaattaagttttataaataatttccatgtcctcatatttcactatcttccagatatcccataagactacgtacgacaaacggcaccataaatattaatcataacaggaagactaaactcaatttctaatttccctattaccagaaaaattagaacaaataacaagaagactgtgatactccatgaagaaaaccattaaaacattccgggttcttactagaatgtggagtaatatatgctttcctgttttggaaagtgctaaacccacaggctaaggtaagaaaagtagtcccacatgctagaagcttcataggccccgactattataagctactatacgttgcttgacaaaagtggaaatgcaaatcgaattctcaaacttgcatctcactttccagcctaggccacaaaataaaagacgccataaattaattagttcgtggatcaagaggtcacaatttggacaacagtaacaaggttccatgtcaatctcctagttaatggtgttcctttggatttccctatcccttttaattagctactccctccgtctcaaattatctgtcgtgatttctaaagttattgtctaaatttatttttcatattagtagttcaagactaaattaattatttcttttccattttacccttaatatgatattgttcttgaagatgaagataatacataaataggaagattatatatatcttaaaacataaataagggtaaaatagtccaaacctcttctcattaatattttcttaattaagtggcgtgtaaaaaagaaacacgacagataatttgaggggagaatatttgtaaagaaaaacaacaatttgcttctaaacagacccaaaaggaatgttagcccaatcctggtaaaacataaatgtcagcaagacagtgctatgtttaattatcagaaattaacagtactacttgcaatcggaacacgactattaaacaaacaaaggtattatctaccagtacatttctctttcattaaattcctgtggtcagacttagttactgcccctttgagaattcttgtcctactacttcatcctcaactttccgtccaatatattattctggtcatttgtttatttctgaaaagcttggaattacatatatacatcctagtccatttaattaagataaaatttcctcattctccctgacatcaatatgtgctaaaagtcgcccttgtcactcaccctagttcacactcgagctttgggattggcattctctccatttcattataaattcaagccaccacacaacacagagtcacagagctaaatatagcatatatatatatatatatatatatatatatacctccctgcatatcttcctcagagtcaaattaagcttcaagcaaaacaacca

SEQ ID NO: 86 - DNA, 2001 bp, synthetic construct
  source 1..2001: mol_type = other DNA
cataatgtattttctcaaagaattatataatatgtgtatactattaaaatacattgtatatgatagtaatccacaagtataatataatctatatataccattaagatatactagtaatatattcatatataccatcatcattcatcatgtttttaataattatacaacataatgaataattaaatttaatatagtactacgtataagtgtaaaaatagaggtacaatcagttacttgaaggaggagtggaataagaaataaattgaagacaaaagttggattgaatagacgcaaaaggaaaggttttagggtgaggaaaatttataattgtacagtattcaaaaagaggagaaagtgagggttttagggtacacggaattaaataggggagagaaaattattgggaaacttcaaaagttgtggattaccattaattactaatcctattttaatgggattggtccaacattataggtttatgctagaaatataacgtagttaataagctgccatatttgttactccctccggtccacaatatgtgatcaatttaccttttgattttggtccaaaataagtgtttaaaatagcacggtctagccagttttcggactggtcattcaaaaatagccagcgtttaccaagtcaatgaaaaataaccactattttgctgcaacagagaccggtccaacataatatattggagtttggtgcacctgtgtatgaacttccagataatatactggagcaccggtgctccaaactccagtatattatgctagaccgatatattatactagaactccagtatattatgctggagtatttttccggattttgaacaatattttcgttaagatatatctttacatgaaaagtggctaaatttcgattacctttgaaactgtggctatttttgaatgaccacttgtaaatctggcaatttttgaatttctcccccatttatataatcaagaaaaaatttgatttttttttaaaattatccttatatacgtatccctaaaaagttttttactcctcacattaaattatgctgcaacatttaattaagggtaatttagtcacactaactatttttgtctagaatttatatttccttaatgggtgtgcccaaggtaaattgaccacttatactggaccggagagagtaattgcttgctgctactagaatttagtaaaattttcttaaatattgccaaatatgttttttccacgtgttaatttggacgaatccagtaaattttgcttaactttatatttgtattaaaaaaattagtaactatatataaatattttaattatggacccggtaactaaaacgaagatacaaattccaagacaaattcagaaatcataaacatcaaatcttgtttgtacctatgaatgcattccttaaaatatactacaagttagggtgttgtcactttcttgaagcatattccgactgagtcttgtagaatctcatgagcgttaggccttatcgcttcaattaggcaattacttacctcctctgtactttaagaaaattcaatgtagtacaccattattaagttcatttcataaataaaattaattctgtcacctctataacatataagaacgtatattttccaagctagatatatctaacatgaatgactttgtgaaattcttttgccaagttaaagaaaaaaaatggtgtccaaagaataaagagaaaaggcccctaataaaaaaataatttattaattagagttgttcaaacaatcacgtcaataatggttttcatcaagaatgactagtggcggctcttatctcgagcgatgtggcatgattttgactttaacactaatatcaaatgctttctatatcaattatcctatagagtctgctaacaagtaattcaattctttaatttcttttacaaattccagacttttcaagtcttcttccctattaaaacccacccactctattccttgtttctcaccaaaacccaaa

SEQ ID NO: 87 - DNA, 2001 bp, synthetic construct
  source 1..2001: mol_type = other DNA
atgaacgggtcgattttagggtatttgacatttaatcctctcggtctcacggtaggcattcggatcgatatttaaggaaaataaaatataaacaaactaatatatatatatatatatatatacacgacaccttaagatattagaagttggccaagaaatttaaatgaacaataacaaaagatttgctttttgcatctcgttgtccatgagattttctttgtctgctcacgacatttccaccttaaccatctaaatcaaaagttaatttgttcctacaatacgatatcgcaatcttattacgccctccattgcattttatggaaggtattattttttgtggagttatactattttttctttagttataattttcttatttttttttaaaaaaacattttgaatcattagctatgtagtactgttcgtgtaatttataaatatatataaaaaaatttaaaaaaatgaggaattaatagtaaattcacatcgaaaatcaagtgttttgactatcctactccacaccctgtcacataaattaggaaaaagaaagaaaaaaagaaaagaaaagaaataccttccaatttactgcttccctttaagaacatgaaaaatcttgctaatagattgctagtagttgttttctaaataactcacgtctctatcttgtctgccaccttgaaaatatatgactgtttctatcttttctttagataataatagttgagcaatcaatatctaaaactgtttttttctttcatcttaatctaatcattaagtaatcaatgtatttttaaccagtgctttatttttgctttgggtaagattgtttcatctatataacatttgttcctcaccaattactaccattaaacatgaacaaaatgtttcatggacagtgcattcttttcccttataaatcacgtatctatctatttctccaccttaaatccatatatatatatatatatatatatatatatatatatgtttcatggacagtgcattcttttcccttataaatcacgtatctatctatttctccaccttaaatccatatatatatatatatatatatatatatatatatatatatatatatatatatatatatatatatatatatatatatatacttttaatattgtatcctattaaggatcaatattttttcttctcgttttctgcgttgagtaaggtattaggtagattggttttaaagttgtacctccctcttaactcctcgtctttaataacgttaaatttagactgcttgacttatctcatgaataatgcaacgtgttccatataattcagatctccatctcttttactaccttgaatatatataatattgttatgaagttaaactaaaagaacaaaaaaatatagagagaaataacaacttttaaataacaataatagtaaaactaattcatacattgtgagacttgatctcttatgaaagtctcaaactttacagtaaaatgaaggaaaactaccggttatatccatttataagtatataattataaaaattggtgaattcataaaatattattaatattagtcaattagctatttgtaacccaaaaaaggttaaagttttgcttttatttgagtgtgtattattagaatagattaagtacattttaagaagcttgaatttcagttttgggatgatttggtggagttttaaggtggtttgaattaaaaattaaaagtagaagatgattaaaatttcatggtaaaggacaacagtaacttagcaagaccttatacacgtaatgagctaacactaatcttccatctccaataatgaaaatcatgagtttcgtggatcacgttttatactagcttttcatgcagctaactagaacataatatattatttaataataataaaaaactagaatataatctatataaagactacagcctctcagcactttctcaccacttccttttcaatacttactttctactatattgaagaagaaaata

SEQ ID NO: 88 - DNA, 2001 bp, synthetic construct
  source 1..2001: mol_type = other DNA
ataaagtaaaaatgataaagttgaagaacaattgttgagcacggcaaacgagaaaagcagagtagaatattctattgaagtaaataataatgtgtttacaaatgattggggtcccctttatataggagggaaaaaccctaatatggtacattcccaactatggtgaagaattctattggtattgttgtataacaacctagtacggactcgtactattttgtacaaatcttatcccataatttatgccttgatccacgtgttcttgagaaattcccgctctttcttgagtgtcatcgaaattgtactgcccttgaggcagatcatgacgggtctccgattttctcctcgaggtgcgcacatcctggccagggtcgattcttacttcgagcttcccgaactcgagctcggggtatgatcaagccttcgaaatcgagctctccaatttcgaccgtatacaattttaagaattgaatatactttgatcttttcttgaaaaagtatttatttattttatcttctcgcctagcgcaatgaaatcaatatttattattttcaaaaacttacatttttctttagttttattttaataatatttttatattttttctaaaattatattcttattaatataggtacacgcgcagagcgcataccttaatactagcttgagggtacgtgttttgcatgtgtatcccatattctatatctatatctatactatattaaaaacactaacgctcttggcgaaatgccgttcgcttattttatccttaaaaaatattatatattggatatattatcctttaacatatataatcatcttcaggaacttatatttttattttattatttaaatataaatttttaataaattagtactcattgagatagaatacacgcgcatcgcgcgtaccttgaggctagtagaaaagaaaagtatgatagtatatgagtaacgtgttggaaattaagggaaggaaagaaaattttctttacatccaaatttgaaagttatttgacgtcatcgagatgacggccatgttcaagttttccacaaataatgtgaaaagaaaaagaagaagacacacactgtgtttggttttattatagttttttcttttagagaattgattgtacaaattattataagaaatagtataatataagatttagaaataagattattagaaaaaacaaacatcaaaaagtatttattttaatttctttttccaatggacattcccattcttctgaaacaacatagataaaaatatgaaagcaaaaattatcagatcgtttaatgcagataatattaattaacacattaaccataaccaatattttatttaaaaaaagcacaactgctagatcaaaaaagtgtttaacttcatgcattgacaattttttaaaataattttgcagcatcaggtaaaatattttttcttcgtttcccaccctgtgtacggtatacacgggtacttatattggggcccaactaaattcggattcgtccggagaagtcccacattggggcataaagcgctccctgacgaaggcgactccatacccatggacttgaacccgagacctttggttaaggatgaacgagacctccggtgccaggtaaaatattggtaactgcttatataagtttaatatggtaacctgagccgaaggtctatcggaaacagactttctgccctatcagggtaggggtaaggtctgcatacacagtaccctctccaaaccccacttagtgagactttagtgggtagttgttgttgttattgttatggtaacctgggaaacaggataaataactatctataacaggatatattacattgatattaccatgtcaaaaaattaagcaagtacatgaataatcgccgtgaaatcttcaagatttctcctataaatacccttggtagtaaatctagtttttccattcaaagtacaacatttctcctatagtcatggaatttgttctcttttcacaaa

SEQ ID NO: 89 - DNA, 2001 bp, synthetic construct
  source 1..2001: mol_type = other DNA
aaatttttgctaaagtactctctttttattagttgaaaaagaaattactctttctataacatgaaaataaagtactcatttgttgaaatgaaagaaaatatttttttctacatcatgaaaagatactcgttatggggacatggcgaataggggtgggttggtggggatatggggaatagggggaagattgaaaaagagttttgggaaagtttttttttgttttgatagggaaaatattttcctccaatttgagaaaaatgagttcatgggcgtggggtggaggtgggtagctggaggtgtggggtgggttggtggggatatgtggaataggggaagattggaaaagagttttgggaaagtatttttttttgttttgatggggaaaacattttcctctaattgatgaaaaatgagttcactagaaaaatattttccaaaatatttaatccaaccaaacatgataaaattaaaaaatatttaaaaatattttcgttcgtaccaaacacacccacagtggaactgattaatctgtgtgttacgtaatttgtgccgcttcatgcatttatttcgtgcgacgatttggaattcttcaataaataactaatagtgaaagatcatataatactagtaatactagctttaaaaaaatgtaagtgtcgggagggtgacggaaatgacaattatgtcataaacacacgttgttcatacttcacataggggcgactctaaggctttgggaccataatagtagcagcagtagtaatagtagtcgtaatcatagtaacagtagtggtaataatcgtagtggtgatagtggtggtaatggtagtgatggtagtggtagtaataatagtagcagtagtgatagtaataataatagtaatagtagatagtggtggtagtggtagtagtaatagtggtagtagatattcaccaataagcgtgctacttaatagaataaactacaacaatcgtttaaattgatcaggagtaaatacttttgacaccgtcaatactcaaaacttaagctattacgttatatatgtacgcttattctttttctcgtttgtgatgatagccaaatcaaaattttcaatttgagttctagacctcgacattcgatagtccaccactttattttttgtgctctttttcctaatttgtttttgaaattcgcttttttgtcttaaatgatttaaaagtaaattttattgttagtgtaaatattttataaaataaatttaagagcctctcaatatgatttcatcttaggccacgaaatccgttgagcacaggggcggagccaccttatgggatgcgtgacaccgcttcatcggaaagatttattaaatacatatatagacatatgcaaaaccaaataatacataaaaacgataaaggtgacaacacttgaaataaaataactcattgtccaactggttaagcgtttcaacttcttagtatgtcgggagtacgaatccttttggtggaacccttatgtttttattgttttgaagacattatttattaaaaaagaattaaaatatattctatattagtaaggacctctaacaaaattaagcaacaacaaaagtcacttgaacaagaattcatttgttagtcaatttgacaataaatatactcaaatcattcttggaaaaagacatatttggtactagtatttctaacaagattattattaatacatttagaaaataaaaactcgttgtaaagatttatagtaatgttttgaattttattttttttaataaatacaatgtcattatagttattcatttgttcacttctttgtttacctattacaaaatatgatacggacttacgaaaaattttgcgtacgacactggttgagctgcccctgtgttcacattggaagaataatatcatggcacatttcttctctataaattcaaggttagcatggtcgaataaagcatccaaaacaaatatcgcatatccaatctaaagaaacaaaaaaaa

SEQ ID NO: 90 - DNA, 2001 bp, synthetic construct
  source 1..2001: mol_type = other DNA
aagggaaaaaaataaaaatagaagagaaatgcaagggtttgctttcttgtaccaaatattgaaagaaaaaaaatgttacaaccatttcatttgcaaaaaaaaaaaataaaaaaataaattgaaattgaaattcaaaagtaaagctgtttggttcggaacagaagtcgattttgaaaatcgaaccgacgcgttttacgaaaatgaaaacgaccatcgctagattttcacaaactaaaacaattttttccagaaaccttatacccataagcaaattcaacacataaattctccccaattatttttaagaaaatttagaactattttttaagaaattaaattaaaccaactcaactattcaagcaacgcattaagtataacggattcttaaagtctcgttcttaattctcgaaatgtgtcgagaaatcacaaccgttaaatctaatgcctcaaagtaaagaaaatgaataaggagtgtagcaaaaatatcaatgtggacaagatggtcgtatataatttgtacaaagaaaattcaaatgggaatctcttacccagtcaaaacatgcatattctcttggattctaggcaagctttcccattccaaagtcaatggaacgaaccaattattgctctctaagtctttttctttcactaaagagtaaattcaatatcttgacaattaggcactaagtaaactgaagaaatagaatcccaatatttttccttcctcgtaacaataacattaaagactcatcaatgtgactcgaccaaaggttcagtagcttcattttcacaaataggggtgacaatttgaatccaaatccatctagcccgtccaaagattaatggttgagctacaaacattttaatttatgagttaatttaggctgagcccaagttaactcattatttttttataactcattctgacccattaaacagcccaaatataactcatgaaactcacgcaaaacaaagaatctcagtccaatttatctagaaatttacttaatcaccccaatttcacttttttttttgtatttttaattttctgttcttttatttttctgcaccacccaccctacccctatcccccccaaaccccctcaaaaattaatttttacctttttttttttttaatattttcaatttttttttttgtatttctgcaccacccacccgccctaaccccctacccccggcaaaaaccctcaattttttttactttttttgtttattttttttaattttttattttttttttatttttctacaccaccctaaaagtatagtacaaaattcagatttgaactcctttttattggtagattaatcaaagtagaaaaaaaaagacagtttgaaaactacctcattctataaggttttcagaactcgtttgcttactgagaaaatgaattattcacagttaaaatatattttaatgttcaatataagtaatatcagcataatttttataccacatctaacgagggataacaattcagggattagctataccggaataaaacaagtaaaatgataaaattgtccttctaccaaagccttttaaaggaaaatcaaggttaatattagaaacaaaacattatcaaagttaatttagtttaaaaccaaataaatgctttatacgtatatcttattactaatgcagtaaaccaaataaatcagaggtactaaatgatctcaatattagtacaatttagttacgtactaaaatttctaacataattttattctcgccaaccctacaagtatagtactccaacccctttctcttggcaaaataatccaataggaaataaagacggtaagaaaattggcacacgttatatatgacttttgacactatttaagccagccaatggcattagtggttgcatactttgaatgtagagaaataactaataaagactgttgagagttgtttcttcttaccccttttgaagtttattttcctcagattttcacagttccaagcaagcacta